SEQUENCE LISTING (2560 sequences)

SEQ ID NO: 1 - protein, 119 aa, synthetic construct
  REGION 1..119: note = A001, VH
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSS

SEQ ID NO: 2 - protein, 119 aa, synthetic construct
  REGION 1..119: note = A002, VH
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSS

SEQ ID NO: 3 - protein, 119 aa, synthetic construct
  REGION 1..119: note = V002, VH
QVQLVQSGAEVKKPGASVKVSCKASGFNIKDYYMHWVRQAPGQRLEWMGWIDLENAETIYDAKFQDRVTITRDTSASTAYMELSSLRSEDTAVYYCARDAYGRYFYDEWGQGTLVTVSS

SEQ ID NO: 4 - protein, 119 aa, synthetic construct
  REGION 1..119: note = V003, VH
QVQLVQSGAEVKKPGASVKVSCKASGFDIKDYYMHWVRQAPGQRLEWMGWIELENADTIYDAKFQGRVTITRDTSASTAYMELSSLRSEDTAVYYCARDAYGRYFYDVWGQGTLVTVSS

SEQ ID NO: 5 - protein, 119 aa, synthetic construct
  REGION 1..119: note = V004, VH
QVQLVQSGAEVKKPGASVKVSCKASGFDIKDYYMHWVRQAPGQRLEWMGWIELENDDTIYDAKFQGRVTITRDTSASTAYMELSSLRSEDTAVYYCARDAYGRYFYDVWGQGTLVTVSS

SEQ ID NO: 6 - protein, 119 aa, synthetic construct
  REGION 1..119: note = V005, VH
QVQLVQSGAEVKKPGASVKVSCKASGFNIDDYYMHWVRQAPGQRLEWMGWIDLENDNTIYDAKFQGRVTITRDTSASTAYMELSSLRSEDTAVYYCARDAYGRYFYDVWGQGTLVTVSS

SEQ ID NO: 7 - protein, 119 aa, synthetic construct
  REGION 1..119: note = V006, VH
QVQLVQSGAEVKKPGASVKVSCKASGFNDKDYYMHWVRQAPGQRLEWMGWIDLENAETIYDAKFQDRVTITRDTSASTAYMELSSLRSEDTAVYYCARDAYGRYFYDVWGQGTLVTVSS

SEQ ID NO: 8 - protein, 119 aa, synthetic construct
  REGION 1..119: note = V007, VH
QVQLVQSGAEVKKPGASVKVSCKASGFNIDDYYMHWVRQAPGQRLEWMGWIDLENANTIDDAKFQGRVTITRDTSASTAYMELSSLRSEDTAVYYCARDAYGRYFYDVWGQGTLVTVSS

SEQ ID NO: 9 - protein, 119 aa, synthetic construct
  REGION 1..119: note = V008, VH
QVQLVQSGAEVKKPGASVKVSCKASGFNIKDYYMHWVRQAPGQRLEWIGWIDLENAETVYDAKFQDRVTITRDTSASTAYMELSSLRSEDTAVYYCARDAYGRYFYDVWGQGTLVTVSS

SEQ ID NO: 10 - protein, 119 aa, synthetic construct
  REGION 1..119: note = V009, VH
QVQLVQSGAEVKKPGASVKVSCKASGFDIKDYYMHWVRQAPGQRLEWIGWIELENADTVYDAKFQGRVTITRDTSASTAYMELSSLRSEDTAVYYCARDAYGRYFYDVWGQGTLVTVSS

SEQ ID NO: 11 - protein, 119 aa, synthetic construct
  REGION 1..119: note = V010, VH
QVQLVQSGAEVKKPGASVKVSCKASGFDIKDYYMHWVRQAPGQRLEWIGWIELENDDTVYDAKFQGRVTITRDTSASTAYMELSSLRSEDTAVYYCARDAYGRYFYDVWGQGTLVTVSS

SEQ ID NO: 12 - protein, 119 aa, synthetic construct
  REGION 1..119: note = V011, VH
QVQLVQSGAEVKKPGASVKVSCKASGFNIDDYYMHWVRQAPGQRLEWIGWIDLENDNTVYDAKFQGRVTITRDTSASTAYMELSSLRSEDTAVYYCARDAYGRYFYDVWGQGTLVTVSS

SEQ ID NO: 13 - protein, 119 aa, synthetic construct
  REGION 1..119: note = V012, VH
QVQLVQSGAEVKKPGASVKVSCKASGFNDKDYYMHWVRQAPGQRLEWIGWIDLENAETVYDAKFQDRVTITRDTSASTAYMELSSLRSEDTAVYYCARDAYGRYFYDVWGQGTLVTVSS

SEQ ID NO: 14 - protein, 119 aa, synthetic construct
  REGION 1..119: note = V013, VH
QVQLVQSGAEVKKPGASVKVSCKASGFNIDDYYMHWVRQAPGQRLEWIGWIDLENANTVDDAKFQGRVTITRDTSASTAYMELSSLRSEDTAVYYCARDAYGRYFYDVWGQGTLVTVSS

SEQ ID NO: 15 - protein, 112 aa, synthetic construct
  REGION 1..112: note = A001 and V002-V007, VL
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIK

SEQ ID NO: 16 - protein, 112 aa, synthetic construct
  REGION 1..112: note = A002 and V008-V013, VL
DIVMSQSPDSLAVSLGERATINCKSSQSLLNARTGKNYLAWYQQKPGQPPKLLIYWASTRSSGVPDRFSGSGSGTDFTLTISSLQAEDVAVYYCVQSYFRRTFGGGTKVEIK

SEQ ID NO: 17 - protein, 9 aa, synthetic construct
  REGION 1..9: note = A001, A002, V002, and V008, CDRH 1, VH
FNIKDYYMH

SEQ ID NO: 18 - protein, 9 aa, synthetic construct
  REGION 1..9: note = V003, V004, V009, and V010, CDRH1, VH
FDIKDYYMH

SEQ ID NO: 19 - protein, 9 aa, synthetic construct
  REGION 1..9: note = V005, V007, V011, and V013, CDRH1, VH
FNIDDYYMH

SEQ ID NO: 20 - protein, 9 aa, synthetic construct
  REGION 1..9: note = V006 and V012, CDRH1, VH
FNDKDYYMH

SEQ ID NO: 21 - protein, 17 aa, synthetic construct
  REGION 1..17: note = A001, CDRH2, VH
WIDLENANTIYDAKFQG

SEQ ID NO: 22 - protein, 17 aa, synthetic construct
  REGION 1..17: note = A002, CDRH2, VH
WIDLENANTVYDAKFQG

SEQ ID NO: 23 - protein, 17 aa, synthetic construct
  REGION 1..17: note = V002 and V006, CDRH2, VH
WIDLENAETIYDAKFQD

SEQ ID NO: 24 - protein, 17 aa, synthetic construct
  REGION 1..17: note = V003, CDRH2, VH
WIELENADTIYDAKFQG

SEQ ID NO: 25 - protein, 17 aa, synthetic construct
  REGION 1..17: note = V004, CDRH2, VH
WIELENDDTIYDAKFQG

SEQ ID NO: 26 - protein, 17 aa, synthetic construct
  REGION 1..17: note = V005, CDRH2, VH
WIDLENDNTIYDAKFQG

SEQ ID NO: 27 - protein, 17 aa, synthetic construct
  REGION 1..17: note = V007, CDRH2, VH
WIDLENANTIDDAKFQG

SEQ ID NO: 28 - protein, 17 aa, synthetic construct
  REGION 1..17: note = V008 and V012, CDRH2, VH
WIDLENAETVYDAKFQD

SEQ ID NO: 29 - protein, 17 aa, synthetic construct
  REGION 1..17: note = V009, CDRH2, VH
WIELENADTVYDAKFQG

SEQ ID NO: 30 - protein, 17 aa, synthetic construct
  REGION 1..17: note = V010, CDRH2, VH
WIELENDDTVYDAKFQG

SEQ ID NO: 31 - protein, 17 aa, synthetic construct
  REGION 1..17: note = V011, CDRH2, VH
WIDLENDNTVYDAKFQG

SEQ ID NO: 32 - protein, 17 aa, synthetic construct
  REGION 1..17: note = V013, CDRH2, VH
WIDLENANTVDDAKFQG

SEQ ID NO: 33 - protein, 12 aa, synthetic construct
  REGION 1..12: note = A001, A002, and V003-V013, CDRH3, VH
ARDAYGRYFYDV

SEQ ID NO: 34 - protein, 12 aa, synthetic construct
  REGION 1..12: note = V002, CDRH3, VH
ARDAYGRYFYDE

SEQ ID NO: 35 - protein, 17 aa, synthetic construct
  REGION 1..17: note = A001, A002, and V002-V013, CDRL1, VL
KSSQSLLNARTGKNYLA

SEQ ID NO: 36 - protein, 7 aa, synthetic construct
  REGION 1..7: note = A001 and V002-V007, CDRL2, VL
WASTRES

SEQ ID NO: 37 - protein, 7 aa, synthetic construct
  REGION 1..7: note = A002 and V008-V013, CDRL2, VL
WASTRSS

SEQ ID NO: 38 - protein, 8 aa, synthetic construct
  REGION 1..8: note = A001 and V002-V007, CDRL3, VL
KQSYSRRT

SEQ ID NO: 39 - protein, 8 aa, synthetic construct
  REGION 1..8: note = A002 and V008-V013, CDRL3, VL
VQSYFRRT

SEQ ID NO: 40 - protein, 28 aa, synthetic construct
  source 1..28: note = C-terminal is amidated; note = CD3eN27 peptide H2N-QDGNEEMGSITQTPYQVSISGTTVILT[K/SCBiot(dPEG4)]-amide; note = Description of Artificial Sequence: Syntheticpeptide
  MOD_RES 28: note = Lys/SCBiot(dPEG4)
QDGNEEMGSITQTPYQVSISGTTVILTK

SEQ ID NO: 41 - protein, 14 aa, synthetic construct
  source 1..14: note = Description of Artificial Sequence: Syntheticpeptide; note = C-term is amidated; note = CD3eN13 peptide H2N-QDGNEEMGGITQT[K/SCBiot(dPEG4)]-amide
  MOD_RES 14: note = Lys/SCBiot(dPEG4)
QDGNEEMGGITQTK

SEQ ID NO: 42-50 - skipped (9 entries)

SEQ ID NO: 51 - protein, 15 aa, synthetic construct
  source 1..15: note = Human IgG1 hinge, reference
EPKSCDKTHTCPPCP

SEQ ID NO: 52-60 - skipped (9 entries)

SEQ ID NO: 61 - protein, 110 aa, synthetic construct
  source 1..110: note = Human IgG1 CH2, reference
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 62-70 - skipped (9 entries)

SEQ ID NO: 71 - protein, 106 aa, synthetic construct [sequence omitted]

SEQ ID NO: 72 - protein, 106 aa, synthetic construct
  source 1..106: note = Human IgG1 CH3 (356E and 358M)
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 106 aa, synthetic construct
  source 1..106: note = Human IgG1 CH3 (356D, 358L, and V422I)
GQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPG

SEQ ID NO: 74 - protein, 106 aa, synthetic construct
  source 1..106: note = Human IgG1 CH3 (356D, 358L, and A431G)
GQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPG

SEQ ID NO: 75-80 - skipped (6 entries)

SEQ ID NO: 81 - protein, 107 aa, synthetic construct [sequence omitted]

SEQ ID NO: 82 - protein, 106 aa, synthetic construct
  source 1..106: note = Human IgG CL
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 83-90 - skipped (8 entries)

SEQ ID NO: 91 - protein, 98 aa, synthetic construct
  source 1..98: note = Human IgG1 CH1, reference 1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 92 - protein, 98 aa, synthetic construct
  source 1..98: note = Human IgG1 CH1, reference2
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 93-110 - skipped (18 entries)

SEQ ID NO: 111 - protein, 26 aa, synthetic construct
  source 1..26: note = Consensus sequence, FRH1, VH
QVQLVQSGAEVKKPGASVKVSCKASG

SEQ ID NO: 112 - protein, 9 aa, synthetic construct
  REGION 1..9: note = Consensus sequence, VH CDRH1
  SITE 2: note = misc_feature - Xaa can be N or D
  SITE 3: note = misc_feature - Xaa can be I or D
  SITE 4: note = misc_feature - Xaa can be K or D
FXXXDYYMH

SEQ ID NO: 113 - protein, 14 aa, synthetic construct
  source 1..14: note = Consensus sequence, FRH2, VH
  SITE 13: note = misc_feature - Xaa can be M or I
WVRQAPGQRLEWXG

SEQ ID NO: 114 - protein, 17 aa, synthetic construct
  REGION 1..17: note = Consensus sequence, VH CDRH2
  SITE 3: note = MISC_FEATURE - Xaa can be D or E
  SITE 6: note = MISC_FEATURE - Xaa can be N or E
  SITE 7: note = MISC_FEATURE - Xaa can be A, D, or G
  SITE 8: note = misc_feature - Xaa can be N, E, or D
  SITE 10: note = MISC_FEATURE - Xaa can be I or V
  SITE 11: note = MISC_FEATURE - Xaa can be Y or D
  SITE 17: note = MISC_FEATURE - Xaa can be G or D
WIXLEXXXTXXDAKFQX

SEQ ID NO: 115 - protein, 30 aa, synthetic construct
  source 1..30: note = Consensus sequence, FRH3, VH
RVTITRDTSASTAYMELSSLRSEDTAVYYC

SEQ ID NO: 116 - protein, 12 aa, synthetic construct
  REGION 1..12: note = Consensus Sequence, CDRH3
  SITE 1: note = MISC_FEATURE - Xaa can be A or G
  SITE 4: note = MISC_FEATURE - Xaa can be A or Q
  SITE 12: note = MISC_FEATURE - Xaa can be V or E
XRDXYGRYFYDX

SEQ ID NO: 117 - protein, 11 aa, synthetic construct
  source 1..11: note = Consensus sequence, FRH4, VH
WGQGTLVTVSS

SEQ ID NO: 118-120 - skipped (3 entries)

SEQ ID NO: 121 - protein, 23 aa, synthetic construct
  source 1..23: note = Consensus sequence, FRL1, VL
  SITE 5: note = misc_feature - Xaa can be T or S
DIVMXQSPDSLAVSLGERATINC

SEQ ID NO: 122 - protein, 17 aa, synthetic construct
  source 1..17: note = Consensus sequence, CDRL1, VL
KSSQSLLNARTGKNYLA

SEQ ID NO: 123 - protein, 15 aa, synthetic construct
  source 1..15: note = Consensus sequence, FRL2, VL
WYQQKPGQPPKLLIY

SEQ ID NO: 124 - protein, 7 aa, synthetic construct
  source 1..7: note = Consensus sequence, CDRL2, VL
  SITE 6: note = misc_feature - Xaa can be E or S
WASTRXS

SEQ ID NO: 125 - protein, 32 aa, synthetic construct
  source 1..32: note = Consensus sequence, FRL3, VL
GVPDRFSGSGSGTDFTLTISSLQAEDVAVYYC

SEQ ID NO: 126 - protein, 8 aa, synthetic construct
  source 1..8: note = Consensus sequence, CDRL3, VL
  SITE 1: note = misc_feature - Xaa can be K or V
  SITE 5: note = misc_feature - Xaa can be S or F
  SITE 6: note = misc_feature - Xaa can be R or L
XQSYXXRT

SEQ ID NO: 127 - protein, 10 aa, synthetic construct
  source 1..10: note = Consensus sequence, FRL4, VL
FGGGTKVEIK

SEQ ID NO: 128-209 - skipped (82 entries)

SEQ ID NO: 210 - protein, 119 aa, synthetic construct; identical to SEQ ID NO: 3
  REGION 1..119: note = V002, ADI-67445, VH

SEQ ID NO: 211 - protein, 26 aa, synthetic construct
  source 1..26: note = V002, ADI-67445, FRH1, VH
QVQLVQSGAEVKKPGASVKVSCKASG

SEQ ID NO: 212 - protein, 9 aa, synthetic construct
  REGION 1..9: note = V002, ADI-67445, CDRH1, VH
FNIKDYYMH

SEQ ID NO: 213 - protein, 14 aa, synthetic construct
  source 1..14: note = V002, ADI-67445, FRH2, VH
WVRQAPGQRLEWMG

SEQ ID NO: 214 - protein, 17 aa, synthetic construct
  source 1..17: note = V002, ADI-67445, CDRH2, VH
WIDLENAETIYDAKFQD

SEQ ID NO: 215 - protein, 30 aa, synthetic construct; identical to SEQ ID NO: 115
  source 1..30: note = V002, ADI-67445, FRH3, VH

SEQ ID NO: 216 - protein, 12 aa, synthetic construct
  source 1..12: note = V002, ADI-67445, CDRH3, VH
ARDAYGRYFYDE

SEQ ID NO: 217 - protein, 11 aa, synthetic construct
  source 1..11: note = V002, ADI-67445, FRH4, VH
WGQGTLVTVSS

SEQ ID NO: 218-219 - skipped (2 entries)

SEQ ID NO: 220 - protein, 112 aa, synthetic construct; identical to SEQ ID NO: 15
  source 1..112: note = V002, ADI-67445, VL

SEQ ID NO: 221 - protein, 23 aa, synthetic construct
  source 1..23: note = V002, ADI-67445, FRL1, VL
DIVMTQSPDSLAVSLGERATINC

SEQ ID NO: 222 - protein, 17 aa, synthetic construct
  source 1..17: note = V002, ADI-67445, CDRL1, VL
KSSQSLLNARTGKNYLA

SEQ ID NO: 223 - protein, 15 aa, synthetic construct
  source 1..15: note = V002, ADI-67445, FRL2, VL
WYQQKPGQPPKLLIY

SEQ ID NO: 224 - protein, 7 aa, synthetic construct
  source 1..7: note = V002, ADI-67445, CDRL2, VL
WASTRES

SEQ ID NO: 225 - protein, 32 aa, synthetic construct; identical to SEQ ID NO: 125
  source 1..32: note = V002, ADI-67445, FRL3, VL

SEQ ID NO: 226 - protein, 8 aa, synthetic construct
  source 1..8: note = V002, ADI-67445, CDRL3, VL
KQSYSRRT

SEQ ID NO: 227 - protein, 10 aa, synthetic construct
  source 1..10: note = V002, ADI-67445, FRL4, VL
FGGGTKVEIK

SEQ ID NO: 228-249 - skipped (22 entries)

SEQ ID NO: 250 - DNA, 357 bp, synthetic construct
  source 1..357: mol_type = other DNA; note = V002, ADI-67445, VH
caggtgcagctggtgcagtctggggctgaggtgaagaagcctggggcctcagtgaaggtctcctgcaaggcttctggattcaacatcaaggactactatatgcactgggtgcgacaggctcctggacaaaggcttgagtggatgggatggatcgaccttgaaaatgctgaaacaatctatgacgctaagtttcaggacagggtcaccataaccagggacacgtccgccagcacagcctacatggagctgagcagcctgagatctgaagacacggcggtgtactactgcgcgagagatgcttacgggagatatttttacgacgagtggggccaaggaaccctggtcaccgtctcctca

SEQ ID NO: 251-259 - skipped (9 entries)

SEQ ID NO: 260 - DNA, 336 bp, synthetic construct
  source 1..336: mol_type = other DNA; note = V002, ADI-67445, VL
gacatcgtgatgacccagtctccagactccctggctgtgtctctgggcgagagggccaccatcaactgcaagtccagccagagtcttttaaacgctagaaccggaaagaactacttagcttggtaccagcagaaaccaggacagcctcctaagctgctcatttactgggcatctacccgggaatccggggtccctgaccgattcagtggcagcgggtctgggacagacttcactctcactatcagtagcctgcaggctgaagatgtggcagtttattactgtaagcaatcttattctcggagaactttcggcggagggaccaaggtcgagatcaaa

SEQ ID NO: 261-309 - skipped (49 entries)

SEQ ID NO: 310 - protein, 119 aa, synthetic construct; identical to SEQ ID NO: 4
  REGION 1..119: note = V003, ADI-67446, VH

SEQ ID NO: 311 - protein, 26 aa, synthetic construct
  source 1..26: note = V003, ADI-67446, FRH1, VH
QVQLVQSGAEVKKPGASVKVSCKASG

SEQ ID NO: 312 - protein, 9 aa, synthetic construct
  source 1..9: note = V003, ADI-67446, CDRH1, VH
FDIKDYYMH

SEQ ID NO: 313 - protein, 14 aa, synthetic construct
  source 1..14: note = V003, ADI-67446, FRH2, VH
WVRQAPGQRLEWMG

SEQ ID NO: 314 - protein, 17 aa, synthetic construct
  source 1..17: note = V003, ADI-67446, CDRH2, VH
WIELENADTIYDAKFQG

SEQ ID NO: 315 - protein, 30 aa, synthetic construct; identical to SEQ ID NO: 115
  source 1..30: note = V003, ADI-67446, FRH3, VH

SEQ ID NO: 316 - protein, 12 aa, synthetic construct
  source 1..12: note = V003, ADI-67446, CDRH3, VH
ARDAYGRYFYDV

SEQ ID NO: 317 - protein, 11 aa, synthetic construct
  source 1..11: note = V003, ADI-67446, FRH4, VH
WGQGTLVTVSS

SEQ ID NO: 318-319 - skipped (2 entries)

SEQ ID NO: 320 - protein, 112 aa, synthetic construct; identical to SEQ ID NO: 15
  source 1..112: note = V003, ADI-67446, VL

SEQ ID NO: 321 - protein, 23 aa, synthetic construct
  source 1..23: note = V003, ADI-67446, FRL1, VL
DIVMTQSPDSLAVSLGERATINC

SEQ ID NO: 322 - protein, 17 aa, synthetic construct
  source 1..17: note = V003, ADI-67446, CDRL1, VL
KSSQSLLNARTGKNYLA

SEQ ID NO: 323 - protein, 15 aa, synthetic construct
  source 1..15: note = V003, ADI-67446, FRL2, VL
WYQQKPGQPPKLLIY

SEQ ID NO: 324 - protein, 7 aa, synthetic construct
  source 1..7: note = V003, ADI-67446, CDRL2, VL
WASTRES

SEQ ID NO: 325 - protein, 32 aa, synthetic construct; identical to SEQ ID NO: 125
  source 1..32: note = V003, ADI-67446, FRL3, VL

SEQ ID NO: 326 - protein, 8 aa, synthetic construct
  source 1..8: note = V003, ADI-67446, CDRL3, VL
KQSYSRRT

SEQ ID NO: 327 - protein, 10 aa, synthetic construct
  source 1..10: note = V003, ADI-67446, FRL4, VL
FGGGTKVEIK

SEQ ID NO: 328-349 - skipped (22 entries)

SEQ ID NO: 350 - DNA, 357 bp, synthetic construct
  source 1..357: mol_type = other DNA; note = V003, ADI-67446, VH
caggtgcagctggtgcagtctggggctgaggtgaagaagcctggggcctcagtgaaggtctcctgcaaggcttctggattcgacatcaaggactactatatgcactgggtgcgacaggctcctggacaaaggcttgagtggatgggatggatcgagcttgaaaatgctgatacaatctatgacgctaagtttcagggcagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 351-359 - skipped (9 entries)

SEQ ID NO: 360 - DNA, 336 bp, synthetic construct; identical to SEQ ID NO: 260
  source 1..336: mol_type = other DNA; note = V003, ADI-67446, VL

SEQ ID NO: 361-409 - skipped (49 entries)

SEQ ID NO: 410 - protein, 119 aa, synthetic construct; identical to SEQ ID NO: 6
  source 1..119: note = V004, ADI-67447, VH

SEQ ID NO: 411 - protein, 26 aa, synthetic construct
  source 1..26: note = V004, ADI-67447, FRH1, VH
QVQLVQSGAEVKKPGASVKVSCKASG

SEQ ID NO: 412 - protein, 9 aa, synthetic construct
  source 1..9: note = V004, ADI-67447, CDRH1, VH
FNIDDYYMH

SEQ ID NO: 413 - protein, 14 aa, synthetic construct
  source 1..14: note = V004, ADI-67447, FRH2, VH
WVRQAPGQRLEWMG

SEQ ID NO: 414 - protein, 17 aa, synthetic construct
  source 1..17: note = V004, ADI-67447, CDRH2, VH
WIDLENDNTIYDAKFQG

SEQ ID NO: 415 - protein, 30 aa, synthetic construct; identical to SEQ ID NO: 115
  source 1..30: note = V004, ADI-67447, FRH3, VH

SEQ ID NO: 416 - protein, 12 aa, synthetic construct
  source 1..12: note = V004, ADI-67447, CDRH3, VH
ARDAYGRYFYDV

SEQ ID NO: 417 - protein, 11 aa, synthetic construct
  source 1..11: note = V004, ADI-67447, FRH4, VH
WGQGTLVTVSS

SEQ ID NO: 418-419 - skipped (2 entries)

SEQ ID NO: 420 - protein, 112 aa, synthetic construct; identical to SEQ ID NO: 15
  source 1..112: note = V004, ADI-67447, VL

SEQ ID NO: 421 - protein, 23 aa, synthetic construct
  source 1..23: note = V004, ADI-67447, FRL1, VL
DIVMTQSPDSLAVSLGERATINC

SEQ ID NO: 422 - protein, 17 aa, synthetic construct
  source 1..17: note = V004, ADI-67447, CDRL1, VL
KSSQSLLNARTGKNYLA

SEQ ID NO: 423 - protein, 15 aa, synthetic construct
  source 1..15: note = V004, ADI-67447, FRL2, VL
WYQQKPGQPPKLLIY

SEQ ID NO: 424 - protein, 7 aa, synthetic construct
  source 1..7: note = V004, ADI-67447, CDRL2, VL
WASTRES

SEQ ID NO: 425 - protein, 32 aa, synthetic construct; identical to SEQ ID NO: 125
  source 1..32: note = V004, ADI-67447, FRL3, VL

SEQ ID NO: 426 - protein, 8 aa, synthetic construct
  source 1..8: note = V004, ADI-67447, CDRL3, VL
KQSYSRRT

SEQ ID NO: 427 - protein, 10 aa, synthetic construct
  source 1..10: note = V004, ADI-67447, FRL4, VL
FGGGTKVEIK

SEQ ID NO: 428-449 - skipped (22 entries)

SEQ ID NO: 450 - DNA, 357 bp, synthetic construct
  source 1..357: mol_type = other DNA; note = V004, ADI-67447, VH
caggtgcagctggtgcagtctggggctgaggtgaagaagcctggggcctcagtgaaggtctcctgcaaggcttctggattcaacatcgatgactactatatgcactgggtgcgacaggctcctggacaaaggcttgagtggatgggatggatcgaccttgaaaatgacaacacaatctatgacgctaagtttcagggcagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 451-459 - skipped (9 entries)

SEQ ID NO: 460 - DNA, 336 bp, synthetic construct; identical to SEQ ID NO: 260
  source 1..336: mol_type = other DNA; note = V004, ADI-67447, VL

SEQ ID NO: 461-509 - skipped (49 entries)

SEQ ID NO: 510 - protein, 119 aa, synthetic construct; identical to SEQ ID NO: 6
  source 1..119: note = V005, ADI-67448, VH

SEQ ID NO: 511 - protein, 26 aa, synthetic construct
  source 1..26: note = V005, ADI-67448, FRH1, VH
QVQLVQSGAEVKKPGASVKVSCKASG

SEQ ID NO: 512 - protein, 9 aa, synthetic construct
  source 1..9: note = V005, ADI-67448, CDRH1, VH
FNIDDYYMH

SEQ ID NO: 513 - protein, 14 aa, synthetic construct
  source 1..14: note = V005, ADI-67448, FRH2, VH
WVRQAPGQRLEWMG

SEQ ID NO: 514 - protein, 17 aa, synthetic construct
  source 1..17: note = V005, ADI-67448, CDRH2, VH
WIDLENDNTIYDAKFQG

SEQ ID NO: 515 - protein, 30 aa, synthetic construct; identical to SEQ ID NO: 115
  source 1..30: note = V005, ADI-67448, FRH3, VH

SEQ ID NO: 516 - protein, 12 aa, synthetic construct
  source 1..12: note = V005, ADI-67448, CDRH3, VH
ARDAYGRYFYDV

SEQ ID NO: 517 - protein, 11 aa, synthetic construct
  source 1..11: note = V005, ADI-67448, FRH4, VH
WGQGTLVTVSS

SEQ ID NO: 518-519 - skipped (2 entries)

SEQ ID NO: 520 - protein, 112 aa, synthetic construct; identical to SEQ ID NO: 15
  source 1..112: note = V005, ADI-67448, VL

SEQ ID NO: 521 - protein, 23 aa, synthetic construct
  source 1..23: note = V005, ADI-67448, FRL1, VL
DIVMTQSPDSLAVSLGERATINC

SEQ ID NO: 522 - protein, 17 aa, synthetic construct
  source 1..17: note = V005, ADI-67448, CDRL1, VL
KSSQSLLNARTGKNYLA

SEQ ID NO: 523 - protein, 15 aa, synthetic construct
  source 1..15: note = V005, ADI-67448, FRL2, VL
WYQQKPGQPPKLLIY

SEQ ID NO: 524 - protein, 7 aa, synthetic construct
  source 1..7: note = V005, ADI-67448, CDRL2, VL
WASTRES

SEQ ID NO: 525 - protein, 32 aa, synthetic construct; identical to SEQ ID NO: 125
  source 1..32: note = V005, ADI-67448, FRL3, VL

SEQ ID NO: 526 - protein, 8 aa, synthetic construct
  source 1..8: note = V005, ADI-67448, CDRL3, VL
KQSYSRRT

SEQ ID NO: 527 - protein, 10 aa, synthetic construct
  source 1..10: note = V005, ADI-67448, FRL4, VL
FGGGTKVEIK

SEQ ID NO: 528-549 - skipped (22 entries)

SEQ ID NO: 550 - DNA, 357 bp, synthetic construct; identical to SEQ ID NO: 450
  source 1..357: mol_type = other DNA; note = V005, ADI-67448, VH

SEQ ID NO: 551-559 - skipped (9 entries)

SEQ ID NO: 560 - DNA, 336 bp, synthetic construct; identical to SEQ ID NO: 260
  source 1..336: mol_type = other DNA; note = V005, ADI-67448, VL

SEQ ID NO: 561-609 - skipped (49 entries)

SEQ ID NO: 610 - protein, 119 aa, synthetic construct; identical to SEQ ID NO: 7
  source 1..119: note = V006, ADI-67449, VH

SEQ ID NO: 611 - protein, 26 aa, synthetic construct
  source 1..26: note = V006, ADI-67449, FRH1, VH
QVQLVQSGAEVKKPGASVKVSCKASG

SEQ ID NO: 612 - protein, 9 aa, synthetic construct
  source 1..9: note = V006,ADI-67449, CDRH1, VH
FNDKDYYMH

SEQ ID NO: 613 - protein, 14 aa, synthetic construct
  source 1..14: note = V006, ADI-67449, FRH2, VH
WVRQAPGQRLEWMG

SEQ ID NO: 614 - protein, 17 aa, synthetic construct
  source 1..17: note = V006, ADI-67449, CDRH2, VH
WIDLENAETIYDAKFQD

SEQ ID NO: 615 - protein, 30 aa, synthetic construct; identical to SEQ ID NO: 115
  source 1..30: note = V006, ADI-67449, FRH3, VH

SEQ ID NO: 616 - protein, 12 aa, synthetic construct
  source 1..12: note = V006, ADI-67449, CDRH3, VH
ARDAYGRYFYDV

SEQ ID NO: 617 - protein, 11 aa, synthetic construct
  source 1..11: note = V006, ADI-67449, FRH4, VH
WGQGTLVTVSS

SEQ ID NO: 618-619 - skipped (2 entries)

SEQ ID NO: 620 - protein, 112 aa, synthetic construct; identical to SEQ ID NO: 15
  source 1..112: note = V006, ADI-67449, VL

SEQ ID NO: 621 - protein, 23 aa, synthetic construct
  source 1..23: note = V006, ADI-67449, FRL1, VL
DIVMTQSPDSLAVSLGERATINC

SEQ ID NO: 622 - protein, 17 aa, synthetic construct
  source 1..17: note = V006, ADI-67449, CDRL1, VL
KSSQSLLNARTGKNYLA

SEQ ID NO: 623 - protein, 15 aa, synthetic construct
  source 1..15: note = V006, ADI-67449, FRL2, VL
WYQQKPGQPPKLLIY

SEQ ID NO: 624 - protein, 7 aa, synthetic construct
  source 1..7: note = V006, ADI-67449, CDRL2, VL
WASTRES

SEQ ID NO: 625 - protein, 32 aa, synthetic construct; identical to SEQ ID NO: 125
  source 1..32: note = V006, ADI-67449, FRL3, VL

SEQ ID NO: 626 - protein, 8 aa, synthetic construct
  source 1..8: note = V006, ADI-67449, CDRL3, VL
KQSYSRRT

SEQ ID NO: 627 - protein, 10 aa, synthetic construct
  source 1..10: note = V006, ADI-67449, FRL4, VL
FGGGTKVEIK

SEQ ID NO: 628-649 - skipped (22 entries)

SEQ ID NO: 650 - DNA, 357 bp, synthetic construct
  source 1..357: mol_type = other DNA; note = V006, ADI-67449, VH
caggtgcagctggtgcagtctggggctgaggtgaagaagcctggggcctcagtgaaggtctcctgcaaggcttctggattcaacgataaggactactatatgcactgggtgcgacaggctcctggacaaaggcttgagtggatgggatggatcgaccttgaaaatgctgagacaatctatgacgctaagtttcaggatagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 651-659 - skipped (9 entries)

SEQ ID NO: 660 - DNA, 336 bp, synthetic construct; identical to SEQ ID NO: 260
  source 1..336: mol_type = other DNA; note = V006, ADI-67449, VL

SEQ ID NO: 661-709 - skipped (49 entries)

SEQ ID NO: 710 - protein, 119 aa, synthetic construct; identical to SEQ ID NO: 8
  source 1..119: note = V007, ADI-67450, VH

SEQ ID NO: 711 - protein, 26 aa, synthetic construct
  source 1..26: note = V007, ADI-67450, FRH1, VH
QVQLVQSGAEVKKPGASVKVSCKASG

SEQ ID NO: 712 - protein, 9 aa, synthetic construct
  source 1..9: note = V007, ADI-67450, CDRH1, VH
FNIDDYYMH

SEQ ID NO: 713 - protein, 14 aa, synthetic construct
  source 1..14: note = V007, ADI-67450, FRH2, VH
WVRQAPGQRLEWMG

SEQ ID NO: 714 - protein, 17 aa, synthetic construct
  source 1..17: note = V007, ADI-67450, CDRH2, VH
WIDLENANTIDDAKFQG

SEQ ID NO: 715 - protein, 30 aa, synthetic construct; identical to SEQ ID NO: 115
  source 1..30: note = V007, ADI-67450, FRH3, VH

SEQ ID NO: 716 - protein, 12 aa, synthetic construct
  source 1..12: note = V007, ADI-67450, CDRH3, VH
ARDAYGRYFYDV

SEQ ID NO: 717 - protein, 11 aa, synthetic construct
  source 1..11: note = V007, ADI-67450, FRH4, VH
WGQGTLVTVSS

SEQ ID NO: 718-719 - skipped (2 entries)

SEQ ID NO: 720 - protein, 112 aa, synthetic construct; identical to SEQ ID NO: 15
  source 1..112: note = V007, ADI-67450, VL

SEQ ID NO: 721 - protein, 23 aa, synthetic construct
  source 1..23: note = V007, ADI-67450, FRL1, VL
DIVMTQSPDSLAVSLGERATINC

SEQ ID NO: 722 - protein, 17 aa, synthetic construct
  source 1..17: note = V007, ADI-67450, CDRL1, VL
KSSQSLLNARTGKNYLA

SEQ ID NO: 723 - protein, 15 aa, synthetic construct
  source 1..15: note = V007, ADI-67450, FRL2, VL
WYQQKPGQPPKLLIY

SEQ ID NO: 724 - protein, 7 aa, synthetic construct
  source 1..7: note = V007, ADI-67450, CDRL2, VL
WASTRES

SEQ ID NO: 725 - protein, 32 aa, synthetic construct; identical to SEQ ID NO: 125
  source 1..32: note = V007, ADI-67450, FRL3, VL

SEQ ID NO: 726 - protein, 8 aa, synthetic construct
  source 1..8: note = V007, ADI-67450, CDRL3, VL
KQSYSRRT

SEQ ID NO: 727 - protein, 10 aa, synthetic construct
  source 1..10: note = V007, ADI-67450, FRL4, VL
FGGGTKVEIK

SEQ ID NO: 728-749 - skipped (22 entries)

SEQ ID NO: 750 - DNA, 357 bp, synthetic construct
  source 1..357: mol_type = other DNA; note = V007, ADI-67450, VH
caggtgcagctggtgcagtctggggctgaggtgaagaagcctggggcctcagtgaaggtctcctgcaaggcttctggattcaacatcgatgactactatatgcactgggtgcgacaggctcctggacaaaggcttgagtggatgggatggatcgaccttgaaaatgctaacacaatcgatgacgctaagtttcagggcagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 751-759 - skipped (9 entries)

SEQ ID NO: 760 - DNA, 336 bp, synthetic construct; identical to SEQ ID NO: 260
  source 1..336: mol_type = other DNA; note = V007, ADI-67450, VL

SEQ ID NO: 761-809 - skipped (49 entries)

SEQ ID NO: 810 - protein, 119 aa, synthetic construct; identical to SEQ ID NO: 9
  source 1..119: note = V008, ADI-70325, VH

SEQ ID NO: 811 - protein, 26 aa, synthetic construct
  source 1..26: note = V008, ADI-70325, FRH1, VH
QVQLVQSGAEVKKPGASVKVSCKASG

SEQ ID NO: 812 - protein, 9 aa, synthetic construct
  source 1..9: note = V008, ADI-70325, CDRH1, VH
FNIKDYYMH

SEQ ID NO: 813 - protein, 14 aa, synthetic construct
  source 1..14: note = V008, ADI-70325, FRH2, VH
WVRQAPGQRLEWIG

SEQ ID NO: 814 - protein, 17 aa, synthetic construct
  source 1..17: note = V008, ADI-70325, CDRH2, VH
WIDLENAETVYDAKFQD

SEQ ID NO: 815 - protein, 30 aa, synthetic construct; identical to SEQ ID NO: 115
  source 1..30: note = V008, ADI-70325, FRH3, VH

SEQ ID NO: 816 - protein, 12 aa, synthetic construct
  source 1..12: note = V008, ADI-70325, CDRH3, VH
ARDAYGRYFYDV

SEQ ID NO: 817 - protein, 11 aa, synthetic construct
  source 1..11: note = V008, ADI-70325, FRH4, VH
WGQGTLVTVSS

SEQ ID NO: 818-819 - skipped (2 entries)

SEQ ID NO: 820 - protein, 112 aa, synthetic construct; identical to SEQ ID NO: 16
  source 1..112: note = V008, ADI-70325, VL

SEQ ID NO: 821 - protein, 23 aa, synthetic construct
  source 1..23: note = V008, ADI-70325, FRL1, VL
DIVMSQSPDSLAVSLGERATINC

SEQ ID NO: 822 - protein, 17 aa, synthetic construct
  source 1..17: note = V008, ADI-70325, CDRL1, VL
KSSQSLLNARTGKNYLA

SEQ ID NO: 823 - protein, 15 aa, synthetic construct
  source 1..15: note = V008, ADI-70325, FRL2, VL
WYQQKPGQPPKLLIY

SEQ ID NO: 824 - protein, 7 aa, synthetic construct
  source 1..7: note = V008, ADI-70325, CDRL2, VL
WASTRSS

SEQ ID NO: 825 - protein, 32 aa, synthetic construct; identical to SEQ ID NO: 125
  source 1..32: note = V008, ADI-70325, FRL3, VL

SEQ ID NO: 826 - protein, 8 aa, synthetic construct
  source 1..8: note = V008, ADI-70325, CDRL3, VL
VQSYFRRT

SEQ ID NO: 827 - protein, 10 aa, synthetic construct
  source 1..10: note = V008, ADI-70325, FRL4, VL
FGGGTKVEIK

SEQ ID NO: 828-849 - skipped (22 entries)

SEQ ID NO: 850 - DNA, 357 bp, synthetic construct
  source 1..357: mol_type = other DNA; note = V008, ADI-70325, VH
caggtgcagctggtgcagtctggggctgaggtgaagaagcctggggcctcagtgaaggtctcctgcaaggcttctggattcaacatcaaggactactatatgcactgggtgaggcaggctcctggacaaaggcttgagtggataggatggatcgaccttgaaaatgctgagacagtctatgacgctaagtttcaggatagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 851-859 - skipped (9 entries)

SEQ ID NO: 860 - DNA, 336 bp, synthetic construct
  source 1..336: mol_type = other DNA; note = V008, ADI-70325, VL
gacatcgtgatgtcccagtctccagactccctggctgtgtctctgggcgagagggccaccatcaactgcaagtccagccagagtcttttaaacgctagaaccggaaagaactacttagcttggtaccagcagaaaccaggacagcctcctaagctgctcatttactgggcatctacccggtcgtccggggtccctgaccgattcagcggcagcgggtctgggacagacttcactctcactatcagtagcctgcaggctgaagatgtggcagtttattactgtgtgcaatcttattttcggagaactttcggcggagggaccaaggtcgagatcaaa

SEQ ID NO: 861-909 - skipped (49 entries)

SEQ ID NO: 910 - protein, 119 aa, synthetic construct; identical to SEQ ID NO: 10
  source 1..119: note = V009, ADI-70326, VH

SEQ ID NO: 911 - protein, 26 aa, synthetic construct
  source 1..26: note = V009, ADI-70326, FRH1, VH
QVQLVQSGAEVKKPGASVKVSCKASG

SEQ ID NO: 912 - protein, 9 aa, synthetic construct
  source 1..9: note = V009, ADI-70326, CDRH1, VH
FDIKDYYMH

SEQ ID NO: 913 - protein, 14 aa, synthetic construct
  source 1..14: note = V009, ADI-70326, FRH2, VH
WVRQAPGQRLEWIG

SEQ ID NO: 914 - protein, 17 aa, synthetic construct
  source 1..17: note = V009, ADI-70326, CDRH2, VH
WIELENADTVYDAKFQG

SEQ ID NO: 915 - protein, 30 aa, synthetic construct; identical to SEQ ID NO: 115
  source 1..30: note = V009, ADI-70326, FRH3, VH

SEQ ID NO: 916 - protein, 12 aa, synthetic construct
  source 1..12: note = V009, ADI-70326, CDRH3, VH
ARDAYGRYFYDV

SEQ ID NO: 917 - protein, 11 aa, synthetic construct
  source 1..11: note = V009, ADI-70326, FRH4, VH
WGQGTLVTVSS

SEQ ID NO: 918-919 - skipped (2 entries)

SEQ ID NO: 920 - protein, 112 aa, synthetic construct; identical to SEQ ID NO: 16
  source 1..112: note = V009, ADI-70326, VL

SEQ ID NO: 921 - protein, 23 aa, synthetic construct
  source 1..23: note = V009, ADI-70326, FRL1, VL
DIVMSQSPDSLAVSLGERATINC

SEQ ID NO: 922 - protein, 17 aa, synthetic construct
  source 1..17: note = V009, ADI-70326, CDRL1, VL
KSSQSLLNARTGKNYLA

SEQ ID NO: 923 - protein, 15 aa, synthetic construct
  source 1..15: note = V009, ADI-70326, FRL2, VL
WYQQKPGQPPKLLIY

SEQ ID NO: 924 - protein, 7 aa, synthetic construct
  source 1..7: note = V009, ADI-70326, CDRL2, VL
WASTRSS

SEQ ID NO: 925 - protein, 32 aa, synthetic construct; identical to SEQ ID NO: 125
  source 1..32: note = V009, ADI-70326, FRL3, VL

SEQ ID NO: 926 - protein, 8 aa, synthetic construct
  source 1..8: note = V009, ADI-70326, CDRL3, VL
VQSYFRRT

SEQ ID NO: 927 - protein, 10 aa, synthetic construct
  source 1..10: note = V009, ADI-70326, FRL4, VL
FGGGTKVEIK

SEQ ID NO: 928-949 - skipped (22 entries)

SEQ ID NO: 950 - DNA, 357 bp, synthetic construct
  source 1..357: mol_type = other DNA; note = V009, ADI-70326, VH
caggtgcagctggtgcagtctggggctgaggtgaagaagcctggggcctcagtgaaggtctcctgcaaggcttctggattcgacatcaaggactactatatgcactgggtgaggcaggctcctggacaaaggcttgagtggataggatggatcgagcttgaaaatgctgacacagtctatgacgctaagtttcagggcagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 951-959 - skipped (9 entries)

SEQ ID NO: 960 - DNA, 336 bp, synthetic construct; identical to SEQ ID NO: 860
  source 1..336: mol_type = other DNA; note = V009, ADI-70326, VL

SEQ ID NO: 961-1009 - skipped (49 entries)

SEQ ID NO: 1010 - protein, 119 aa, synthetic construct; identical to SEQ ID NO: 11
  source 1..119: note = V010, ADI-70327, VH

SEQ ID NO: 1011 - protein, 26 aa, synthetic construct
  source 1..26: note = V010, ADI-70327, FRH1, VH
QVQLVQSGAEVKKPGASVKVSCKASG

SEQ ID NO: 1012 - protein, 9 aa, synthetic construct
  source 1..9: note = V010, ADI-70327, CDRH1, VH
FDIKDYYMH

SEQ ID NO: 1013 - protein, 14 aa, synthetic construct
  source 1..14: note = V010, ADI-70327, FRH2, VH
WVRQAPGQRLEWIG

SEQ ID NO: 1014 - protein, 17 aa, synthetic construct
  source 1..17: note = V010, ADI-70327, CDRH2, VH
WIELENDDTVYDAKFQG

SEQ ID NO: 1015 - protein, 30 aa, synthetic construct; identical to SEQ ID NO: 115
  source 1..30: note = V010, ADI-70327, FRH3, VH

SEQ ID NO: 1016 - protein, 12 aa, synthetic construct
  source 1..12: note = V010, ADI-70327, CDRH3, VH
ARDAYGRYFYDV

SEQ ID NO: 1017 - protein, 11 aa, synthetic construct
  source 1..11: note = V010, ADI-70327, FRH4, VH
WGQGTLVTVSS

SEQ ID NO: 1018-1019 - skipped (2 entries)

SEQ ID NO: 1020 - protein, 112 aa, synthetic construct; identical to SEQ ID NO: 16
  source 1..112: note = V010, ADI-70327, VL

SEQ ID NO: 1021 - protein, 23 aa, synthetic construct
  source 1..23: note = V010, ADI-70327, FRL1, VL
DIVMSQSPDSLAVSLGERATINC

SEQ ID NO: 1022 - protein, 17 aa, synthetic construct
  source 1..17: note = V010, ADI-70327, CDRL1, VL
KSSQSLLNARTGKNYLA

SEQ ID NO: 1023 - protein, 15 aa, synthetic construct
  source 1..15: note = V010, ADI-70327, FRL2, VL
WYQQKPGQPPKLLIY

SEQ ID NO: 1024 - protein, 7 aa, synthetic construct
  source 1..7: note = V010, ADI-70327, CDRL2, VL
WASTRSS

SEQ ID NO: 1025 - protein, 32 aa, synthetic construct; identical to SEQ ID NO: 125
  source 1..32: note = V010, ADI-70327, FRL3, VL

SEQ ID NO: 1026 - protein, 8 aa, synthetic construct
  source 1..8: note = V010, ADI-70327, CDRL3, VL
VQSYFRRT

SEQ ID NO: 1027 - protein, 10 aa, synthetic construct
  source 1..10: note = V010, ADI-70327, FRL4, VL
FGGGTKVEIK

SEQ ID NO: 1028-1049 - skipped (22 entries)

SEQ ID NO: 1050 - DNA, 357 bp, synthetic construct
  source 1..357: mol_type = other DNA; note = V010, ADI-70327, VH
caggtgcagctggtgcagtctggggctgaggtgaagaagcctggggcctcagtgaaggtctcctgcaaggcttctggattcgacatcaaggactactatatgcactgggtgagacaggctcctggacaaaggcttgagtggataggatggatcgagcttgaaaatgacgacacagtctatgacgctaagtttcagggcagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 1051-1059 - skipped (9 entries)

SEQ ID NO: 1060 - DNA, 336 bp, synthetic construct; identical to SEQ ID NO: 860
  source 1..336: mol_type = other DNA; note = V010, ADI-70327, VL

SEQ ID NO: 1061-1109 - skipped (49 entries)

SEQ ID NO: 1110 - protein, 119 aa, synthetic construct; identical to SEQ ID NO: 12
  source 1..119: note = V011, ADI-70328, VH

SEQ ID NO: 1111 - protein, 26 aa, synthetic construct
  source 1..26: note = V011, ADI-70328, FRH1, VH
QVQLVQSGAEVKKPGASVKVSCKASG

SEQ ID NO: 1112 - protein, 9 aa, synthetic construct
  source 1..9: note = V011, ADI-70328, CDRH1, VH
FNIDDYYMH

SEQ ID NO: 1113 - protein, 14 aa, synthetic construct
  source 1..14: note = V011, ADI-70328, FRH2, VH
WVRQAPGQRLEWIG

SEQ ID NO: 1114 - protein, 17 aa, synthetic construct
  source 1..17: note = V011, ADI-70328, CDRH2, VH
WIDLENDNTVYDAKFQG

SEQ ID NO: 1115 - protein, 30 aa, synthetic construct; identical to SEQ ID NO: 115
  source 1..30: note = V011, ADI-70328, FRH3, VH

SEQ ID NO: 1116 - protein, 12 aa, synthetic construct
  source 1..12: note = V011, ADI-70328, CDRH3, VH
ARDAYGRYFYDV

SEQ ID NO: 1117 - protein, 11 aa, synthetic construct
  source 1..11: note = V011, ADI-70328, FRH4, VH
WGQGTLVTVSS

SEQ ID NO: 1118-1119 - skipped (2 entries)

SEQ ID NO: 1120 - protein, 112 aa, synthetic construct; identical to SEQ ID NO: 16
  source 1..112: note = V011, ADI-70328, VL

SEQ ID NO: 1121 - protein, 23 aa, synthetic construct
  source 1..23: note = V011, ADI-70328, FRL1, VL
DIVMSQSPDSLAVSLGERATINC

SEQ ID NO: 1122 - protein, 17 aa, synthetic construct
  source 1..17: note = V011, ADI-70328, CDRL1, VL
KSSQSLLNARTGKNYLA

SEQ ID NO: 1123 - protein, 15 aa, synthetic construct
  source 1..15: note = V011, ADI-70328, FRL2, VL
WYQQKPGQPPKLLIY

SEQ ID NO: 1124 - protein, 7 aa, synthetic construct
  source 1..7: note = V011, ADI-70328, CDRL2, VL
WASTRSS

SEQ ID NO: 1125 - protein, 32 aa, synthetic construct; identical to SEQ ID NO: 125
  source 1..32: note = V011, ADI-70328, FRL3, VL

SEQ ID NO: 1126 - protein, 8 aa, synthetic construct
  source 1..8: note = V011, ADI-70328, CDRL3, VL
VQSYFRRT

SEQ ID NO: 1127 - protein, 10 aa, synthetic construct
  source 1..10: note = V011, ADI-70328, FRL4, VL
FGGGTKVEIK

SEQ ID NO: 1128-1149 - skipped (22 entries)

SEQ ID NO: 1150 - DNA, 357 bp, synthetic construct
  source 1..357: mol_type = other DNA; note = V011, ADI-70328, VH
caggtgcagctggtgcagtctggggctgaggtgaagaagcctggggcctcagtgaaggtctcctgcaaggcttctggattcaacatcgatgactactatatgcactgggtgagacaggctcctggacaaaggcttgagtggataggatggatcgaccttgaaaatgataacacagtctatgacgctaagtttcagggcagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 1151-1159 - skipped (9 entries)

SEQ ID NO: 1160 - DNA, 336 bp, synthetic construct; identical to SEQ ID NO: 860
  source 1..336: mol_type = other DNA; note = V011, ADI-70328, VL

SEQ ID NO: 1161-1209 - skipped (49 entries)

SEQ ID NO: 1210 - protein, 119 aa, synthetic construct; identical to SEQ ID NO: 13
  source 1..119: note = V012, ADI-70329, VH

SEQ ID NO: 1211 - protein, 26 aa, synthetic construct
  source 1..26: note = V012, ADI-70329, FRH1, VH
QVQLVQSGAEVKKPGASVKVSCKASG

SEQ ID NO: 1212 - protein, 9 aa, synthetic construct
  source 1..9: note = V012, ADI-70329, CDRH1, VH
FNDKDYYMH

SEQ ID NO: 1213 - protein, 14 aa, synthetic construct
  source 1..14: note = V012, ADI-70329, FRH2, VH
WVRQAPGQRLEWIG

SEQ ID NO: 1214 - protein, 17 aa, synthetic construct
  source 1..17: note = V012, ADI-70329, CDRH2, VH
WIDLENAETVYDAKFQD

SEQ ID NO: 1215 - protein, 30 aa, synthetic construct; identical to SEQ ID NO: 115
  source 1..30: note = V012, ADI-70329, FRH3, VH

SEQ ID NO: 1216 - protein, 12 aa, synthetic construct
  source 1..12: note = V012, ADI-70329, CDRH3, VH
ARDAYGRYFYDV

SEQ ID NO: 1217 - protein, 11 aa, synthetic construct
  source 1..11: note = V012, ADI-70329, FRH4, VH
WGQGTLVTVSS

SEQ ID NO: 1218-1219 - skipped (2 entries)

SEQ ID NO: 1220 - protein, 112 aa, synthetic construct; identical to SEQ ID NO: 16
  source 1..112: note = V012, ADI-70329, VL

SEQ ID NO: 1221 - protein, 23 aa, synthetic construct
  source 1..23: note = V012, ADI-70329, FRL1, VL
DIVMSQSPDSLAVSLGERATINC

SEQ ID NO: 1222 - protein, 17 aa, synthetic construct
  source 1..17: note = V012, ADI-70329, CDRL1, VL
KSSQSLLNARTGKNYLA

SEQ ID NO: 1223 - protein, 15 aa, synthetic construct
  source 1..15: note = V012, ADI-70329, FRL2, VL
WYQQKPGQPPKLLIY

SEQ ID NO: 1224 - protein, 7 aa, synthetic construct
  source 1..7: note = V012, ADI-70329, CDRL2, VL
WASTRSS

SEQ ID NO: 1225 - protein, 32 aa, synthetic construct; identical to SEQ ID NO: 125
  source 1..32: note = V012, ADI-70329, FRL3, VL

SEQ ID NO: 1226 - protein, 8 aa, synthetic construct
  source 1..8: note = V012, ADI-70329, CDRL3, VL
VQSYFRRT

SEQ ID NO: 1227 - protein, 10 aa, synthetic construct
  source 1..10: note = V012, ADI-70329, FRL4, VL
FGGGTKVEIK

SEQ ID NO: 1228-1249 - skipped (22 entries)

SEQ ID NO: 1250 - DNA, 357 bp, synthetic construct
  source 1..357: mol_type = other DNA; note = V012, ADI-70329, VH
caggtgcagctggtgcagtctggggctgaggtgaagaagcctggggcctcagtgaaggtctcctgcaaggcttctggattcaacgacaaggactactatatgcactgggtgaggcaggctcctggacaaaggcttgagtggataggatggatcgaccttgaaaatgctgagacagtctatgacgctaagtttcaggacagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 1251-1259 - skipped (9 entries)

SEQ ID NO: 1260 - DNA, 336 bp, synthetic construct; identical to SEQ ID NO: 860
  source 1..336: mol_type = other DNA; note = V012, ADI-70329, VL

SEQ ID NO: 1261-1309 - skipped (49 entries)

SEQ ID NO: 1310 - protein, 119 aa, synthetic construct; identical to SEQ ID NO: 14
  source 1..119: note = V013, ADI-70330, VH

SEQ ID NO: 1311 - protein, 26 aa, synthetic construct
  source 1..26: note = V013, ADI-70330, FRH1, VH
QVQLVQSGAEVKKPGASVKVSCKASG

SEQ ID NO: 1312 - protein, 9 aa, synthetic construct
  source 1..9: note = V013, ADI-70330, CDRH1, VH
FNIDDYYMH

SEQ ID NO: 1313 - protein, 14 aa, synthetic construct
  source 1..14: note = V013, ADI-26913, FRH2, VH
WVRQAPGQRLEWIG

SEQ ID NO: 1314 - protein, 17 aa, synthetic construct
  source 1..17: note = V013, ADI-70330, CDRH2, VH
WIDLENANTVDDAKFQG

SEQ ID NO: 1315 - protein, 30 aa, synthetic construct; identical to SEQ ID NO: 115
  source 1..30: note = V013, ADI-70330, FRH3, VH

SEQ ID NO: 1316 - protein, 12 aa, synthetic construct
  source 1..12: note = V013, ADI-70330, CDRH3, VH
ARDAYGRYFYDV

SEQ ID NO: 1317 - protein, 11 aa, synthetic construct
  source 1..11: note = V013, ADI-70330, FRH4, VH
WGQGTLVTVSS

SEQ ID NO: 1318-1319 - skipped (2 entries)

SEQ ID NO: 1320 - protein, 112 aa, synthetic construct; identical to SEQ ID NO: 16
  source 1..112: note = V013, ADI-70330, VL

SEQ ID NO: 1321 - protein, 23 aa, synthetic construct
  source 1..23: note = A003, ADI-26913, FRL1, VL
DIVMSQSPDSLAVSLGERATINC

SEQ ID NO: 1322 - protein, 17 aa, synthetic construct
  source 1..17: note = V013, ADI-70330, CDRL1, VL
KSSQSLLNARTGKNYLA

SEQ ID NO: 1323 - protein, 15 aa, synthetic construct
  source 1..15: note = V013, ADI-70330, FRL2, VL
WYQQKPGQPPKLLIY

SEQ ID NO: 1324 - protein, 7 aa, synthetic construct
  source 1..7: note = V013, ADI-70330, CDRL2, VL
WASTRSS

SEQ ID NO: 1325 - protein, 32 aa, synthetic construct; identical to SEQ ID NO: 125
  source 1..32: note = V013, ADI-70330, FRL3, VL

SEQ ID NO: 1326 - protein, 8 aa, synthetic construct
  source 1..8: note = V013, ADI-70330, CDRL3, VL
VQSYFRRT

SEQ ID NO: 1327 - protein, 10 aa, synthetic construct
  source 1..10: note = V013, ADI-70330, FRL4, VL
FGGGTKVEIK

SEQ ID NO: 1328-1349 - skipped (22 entries)

SEQ ID NO: 1350 - DNA, 357 bp, synthetic construct
  source 1..357: mol_type = other DNA; note = V013, ADI-70330, VH
caggtgcagctggtgcagtctggggctgaggtgaagaagcctggggcctcagtgaaggtctcctgcaaggcttctggattcaacatcgatgactactatatgcactgggtgaggcaggctcctggacaaaggcttgagtggataggatggatcgaccttgaaaatgctaacacagtcgatgacgctaagtttcagggcagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 1351-1359 - skipped (9 entries)

SEQ ID NO: 1360 - DNA, 336 bp, synthetic construct; identical to SEQ ID NO: 860
  source 1..336: mol_type = other DNA; note = V013, ADI-70330, VL

SEQ ID NO: 1361-1409 - skipped (49 entries)

SEQ ID NO: 1410 - protein, 119 aa, synthetic construct
  source 1..119: note = V014, ADI-76357, VH
QVQLVQSGAEVKKPGASVKVSCKASGFDIKDYYMHWVRQAPGQRLEWMGWIDLENANTIYDAKFQGRVTITRDTSASTAYMELSSLRSEDTAVYYCARDQYGRYFYDVWGQGTLVTVSS

SEQ ID NO: 1411 - protein, 26 aa, synthetic construct
  source 1..26: note = V014, ADI-76357, FRH1, VH
QVQLVQSGAEVKKPGASVKVSCKASG

SEQ ID NO: 1412 - protein, 9 aa, synthetic construct
  source 1..9: note = V014, ADI-76357, CDRH1, VH
FDIKDYYMH

SEQ ID NO: 1413 - protein, 14 aa, synthetic construct
  source 1..14: note = V014, ADI-76357, FRH2, VH
WVRQAPGQRLEWMG

SEQ ID NO: 1414 - protein, 17 aa, synthetic construct
  source 1..17: note = V014, ADI-76357, CDRH2, VH
WIDLENANTIYDAKFQG

SEQ ID NO: 1415 - protein, 30 aa, synthetic construct; identical to SEQ ID NO: 115
  source 1..30: note = V014, ADI-76357, FRH3, VH

SEQ ID NO: 1416 - protein, 12 aa, synthetic construct
  source 1..12: note = V014, ADI-76357, CDRH3, VH
ARDQYGRYFYDV

SEQ ID NO: 1417 - protein, 11 aa, synthetic construct
  source 1..11: note = V014, ADI-76357, FRH4, VH
WGQGTLVTVSS

SEQ ID NO: 1418-1419 - skipped (2 entries)

SEQ ID NO: 1420 - protein, 112 aa, synthetic construct; identical to SEQ ID NO: 15
  source 1..112: note = V014, ADI-76357, VL

SEQ ID NO: 1421 - protein, 23 aa, synthetic construct
  source 1..23: note = V014, ADI-76357, FRL1, VL
DIVMTQSPDSLAVSLGERATINC

SEQ ID NO: 1422 - protein, 17 aa, synthetic construct
  source 1..17: note = V014, ADI-76357, CDRL1, VL
KSSQSLLNARTGKNYLA

SEQ ID NO: 1423 - protein, 15 aa, synthetic construct
  source 1..15: note = V014, ADI-76357, FRL2, VL
WYQQKPGQPPKLLIY

SEQ ID NO: 1424 - protein, 7 aa, synthetic construct
  source 1..7: note = V014, ADI-76357, CDRL2, VL
WASTRES

SEQ ID NO: 1425 - protein, 32 aa, synthetic construct; identical to SEQ ID NO: 125
  source 1..32: note = V014, ADI-76357, FRL3, VL

SEQ ID NO: 1426 - protein, 8 aa, synthetic construct
  source 1..8: note = V014, ADI-76357, CDRL3, VL
KQSYSRRT

SEQ ID NO: 1427 - protein, 10 aa, synthetic construct
  source 1..10: note = V014, ADI-76357, FRL4, VL
FGGGTKVEIK

SEQ ID NO: 1428-1449 - skipped (22 entries)

SEQ ID NO: 1450 - DNA, 357 bp, synthetic construct
  source 1..357: mol_type = other DNA; note = V014, ADI-76357, VH
caggtgcagctggtgcagtctggggctgaggtgaagaagcctggggcctcagtgaaggtttcctgcaaggcttctggatttgacattaaagattattacatgcattgggtgcgccaggcccccggacaaaggcttgagtggatgggatggatcgacttggagaacgcgaacacaatctatgatgctaagttccagggcagagtcaccattaccagggacacatccgcgagcacagcctacatggagctgagcagcctgagatctgaagacacggcggtgtactactgcgcacgtgaccagtatggccggtacttctatgacgtttggggccagggcaccctggtcaccgtctcctca

SEQ ID NO: 1451-1459 - skipped (9 entries)

SEQ ID NO: 1460 - DNA, 336 bp, synthetic construct; identical to SEQ ID NO: 260
  source 1..336: mol_type = other DNA; note = V014, ADI-76357, VL

SEQ ID NO: 1461-1509 - skipped (49 entries)

SEQ ID NO: 1510 - protein, 119 aa, synthetic construct
  source 1..119: note = V015, ADI-76358, VH
QVQLVQSGAEVKKPGASVKVSCKASGFNIKDYYMHWVRQAPGQRLEWMGWIDLENADTIYDAKFQGRVTITRDTSASTAYMELSSLRSEDTAVYYCARDQYGRYFYDVWGQGTLVTVSS

SEQ ID NO: 1511 - protein, 26 aa, synthetic construct
  source 1..26: note = V015, ADI-76358, FRH1, VH
QVQLVQSGAEVKKPGASVKVSCKASG

SEQ ID NO: 1512 - protein, 9 aa, synthetic construct
  source 1..9: note = V015, ADI-76358, CDRH1, VH
FNIKDYYMH

SEQ ID NO: 1513 - protein, 14 aa, synthetic construct
  source 1..14: note = V015, ADI-76358, FRH2, VH
WVRQAPGQRLEWMG

SEQ ID NO: 1514 - protein, 17 aa, synthetic construct
  source 1..17: note = V015, ADI-76358, CDRH2, VH
WIDLENADTIYDAKFQG

SEQ ID NO: 1515 - protein, 30 aa, synthetic construct; identical to SEQ ID NO: 115
  source 1..30: note = V015, ADI-76358, FRH3, VH

SEQ ID NO: 1516 - protein, 12 aa, synthetic construct
  source 1..12: note = V015, ADI-76358, CDRH3, VH
ARDQYGRYFYDV

SEQ ID NO: 1517 - protein, 11 aa, synthetic construct
  source 1..11: note = V015, ADI-76358, FRH4, VH
WGQGTLVTVSS

SEQ ID NO: 1518-1519 - skipped (2 entries)

SEQ ID NO: 1520 - protein, 112 aa, synthetic construct; identical to SEQ ID NO: 15
  source 1..112: note = V015, ADI-76358, VL

SEQ ID NO: 1521 - protein, 23 aa, synthetic construct
  source 1..23: note = V015, ADI-76358, FRL1, VL
DIVMTQSPDSLAVSLGERATINC

SEQ ID NO: 1522 - protein, 17 aa, synthetic construct
  source 1..17: note = V015, ADI-76358, CDRL1, VL
KSSQSLLNARTGKNYLA

SEQ ID NO: 1523 - protein, 15 aa, synthetic construct
  source 1..15: note = V015, ADI-76358, FRL2, VL
WYQQKPGQPPKLLIY

SEQ ID NO: 1524 - protein, 7 aa, synthetic construct
  source 1..7: note = V015, ADI-76358, CDRL2, VL
WASTRES

SEQ ID NO: 1525 - protein, 32 aa, synthetic construct; identical to SEQ ID NO: 125
  source 1..32: note = V015, ADI-76358, FRL3, VL

SEQ ID NO: 1526 - protein, 8 aa, synthetic construct
  source 1..8: note = V015, ADI-76358, CDRL3, VL
KQSYSRRT

SEQ ID NO: 1527 - protein, 10 aa, synthetic construct
  source 1..10: note = V015, ADI-76358, FRL4, VL
FGGGTKVEIK

SEQ ID NO: 1528-1549 - skipped (22 entries)

SEQ ID NO: 1550 - DNA, 357 bp, synthetic construct
  source 1..357: mol_type = other DNA; note = V015, ADI-76358, VH
caggtgcagctggtgcagtctggggctgaggtgaagaagcctggggcctcagtgaaggtttcctgcaaggcttctggatttaatatcaaagattattatatgcattgggtgcgccaggcccccggacaaaggcttgagtggatgggatggatcgatctcgaaaatgcggatacaatctatgatgcaaagttccagggcagagtcaccattaccagggacacatccgcgagcacagcctacatggagctgagcagcctgagatctgaagacacggcggtgtactactgcgcccgcgatcaatacggaaggtatttctatgatgtctggggccagggcaccctggtcaccgtctcctca

SEQ ID NO: 1551-1559 - skipped (9 entries)

SEQ ID NO: 1560 - DNA, 336 bp, synthetic construct; identical to SEQ ID NO: 260
  source 1..336: mol_type = other DNA; note = V015, ADI-76358, VL

SEQ ID NO: 1561-1609 - skipped (49 entries)

SEQ ID NO: 1610 - protein, 119 aa, synthetic construct
  source 1..119: note = V016, ADI-76359, VH
QVQLVQSGAEVKKPGASVKVSCKASGFDIKDYYMHWVRQAPGQRLEWMGWIDLENANTIYDAKFQGRVTITRDTSASTAYMELSSLRSEDTAVYYCGRDAYGRYFYDVWGQGTLVTVSS

SEQ ID NO: 1611 - protein, 26 aa, synthetic construct
  source 1..26: note = V016, ADI-76359, FRH1, VH
QVQLVQSGAEVKKPGASVKVSCKASG

SEQ ID NO: 1612 - protein, 9 aa, synthetic construct
  source 1..9: note = V016, ADI-76359, CDRH1, VH
FDIKDYYMH

SEQ ID NO: 1613 - protein, 14 aa, synthetic construct
  source 1..14: note = V016, ADI-76359, FRH2, VH
WVRQAPGQRLEWMG

SEQ ID NO: 1614 - protein, 17 aa, synthetic construct
  source 1..17: note = V016, ADI-76359, CDRH2, VH
WIDLENANTIYDAKFQG

SEQ ID NO: 1615 - protein, 30 aa, synthetic construct; identical to SEQ ID NO: 115
  source 1..30: note = V016, ADI-76359, FRH3, VH

SEQ ID NO: 1616 - protein, 12 aa, synthetic construct
  source 1..12: note = V016, ADI-76359, CDRH3, VH
GRDAYGRYFYDV

SEQ ID NO: 1617 - protein, 11 aa, synthetic construct
  source 1..11: note = V016, ADI-76359, FRH4, VH
WGQGTLVTVSS

SEQ ID NO: 1618-1619 - skipped (2 entries)

SEQ ID NO: 1620 - protein, 112 aa, synthetic construct
  source 1..112: note = V016, ADI-76359, VL
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSLRTFGGGTKVEIK

SEQ ID NO: 1621 - protein, 23 aa, synthetic construct
  source 1..23: note = V016, ADI-76359, FRL1, VL
DIVMTQSPDSLAVSLGERATINC

SEQ ID NO: 1622 - protein, 17 aa, synthetic construct
  source 1..17: note = V016, ADI-76359, CDRL1, VL
KSSQSLLNARTGKNYLA

SEQ ID NO: 1623 - protein, 15 aa, synthetic construct
  source 1..15: note = V016, ADI-76359, FRL2, VL
WYQQKPGQPPKLLIY

SEQ ID NO: 1624 - protein, 7 aa, synthetic construct
  source 1..7: note = V016, ADI-76359, CDRL2, VL
WASTRES

SEQ ID NO: 1625 - protein, 32 aa, synthetic construct; identical to SEQ ID NO: 125
  source 1..32: note = V016, ADI-76359, FRL3, VL

SEQ ID NO: 1626 - protein, 8 aa, synthetic construct
  source 1..8: note = V016, ADI-76359, CDRL3, VL
KQSYSLRT

SEQ ID NO: 1627 - protein, 10 aa, synthetic construct
  source 1..10: note = V016, ADI-76359, FRL4, VL
FGGGTKVEIK

SEQ ID NO: 1628-1649 - skipped (22 entries)

SEQ ID NO: 1650 - DNA, 357 bp, synthetic construct
  source 1..357: mol_type = other DNA; note = V016, ADI-76359, VH
caggtgcagctggtgcagtctggggctgaggtgaagaagcctggggcctcagtgaaggtttcctgcaaggcttctggattcgacatcaaagattattacatgcattgggtgcgccaggcccccggacaaaggcttgagtggatgggatggatcgatctagaaaatgctaacacaatatatgatgcgaagttccagggcagagtcaccattaccagggacacatccgcgagcacagcctacatggagctgagcagcctgagatctgaagacacggcggtgtactactgcggccgtgacgcctatggccgatatttctatgacgtctggggccagggcaccctggtcaccgtctcctca

SEQ ID NO: 1651-1659 - skipped (9 entries)

SEQ ID NO: 1660 - DNA, 336 bp, synthetic construct
  source 1..336: mol_type = other DNA; note = V016, ADI-76359, VL
gacatcgtgatgacccagtctccagactccctggctgtgtctctgggcgagagggccaccatcaactgcaagtccagccagagtcttttaaacgctagaaccggaaagaactacttagcttggtaccagcagaaaccaggacagcctcctaagctgctcatttactgggcatctacccgggaatccggggtccctgaccgattcagtggcagcgggtctgggacagacttcactctcactatcagtagcctgcaggctgaagatgtggcagtttattactgtaagcaatcttattctctgagaactttcggcggagggaccaaggtcgagatcaaa

SEQ ID NO: 1661-1709 - skipped (49 entries)

SEQ ID NO: 1710 - protein, 119 aa, synthetic construct
  source 1..119: note = V017, ADI-76360, VH
QVQLVQSGAEVKKPGASVKVSCKASGFNIKDYYMHWVRQAPGQRLEWMGWIDLENADTIYDAKFQGRVTITRDTSASTAYMELSSLRSEDTAVYYCGRDAYGRYFYDVWGQGTLVTVSS

SEQ ID NO: 1711 - protein, 26 aa, synthetic construct
  source 1..26: note = V017, ADI-76360, FRH1, VH
QVQLVQSGAEVKKPGASVKVSCKASG

SEQ ID NO: 1712 - protein, 9 aa, synthetic construct
  source 1..9: note = V017, ADI-76360, CDRH1, VH
FNIKDYYMH

SEQ ID NO: 1713 - protein, 14 aa, synthetic construct
  source 1..14: note = V017, ADI-76360, FRH2, VH
WVRQAPGQRLEWMG

SEQ ID NO: 1714 - protein, 17 aa, synthetic construct
  source 1..17: note = V017, ADI-76360, CDRH2, VH
WIDLENADTIYDAKFQG

SEQ ID NO: 1715 - protein, 30 aa, synthetic construct; identical to SEQ ID NO: 115
  source 1..30: note = V017, ADI-76360, FRH3, VH

SEQ ID NO: 1716 - protein, 12 aa, synthetic construct
  source 1..12: note = V017, ADI-76360, CDRH3, VH
GRDAYGRYFYDV

SEQ ID NO: 1717 - protein, 11 aa, synthetic construct
  source 1..11: note = V017, ADI-76360, FRH4, VH
WGQGTLVTVSS

SEQ ID NO: 1718-1719 - skipped (2 entries)

SEQ ID NO: 1720 - protein, 112 aa, synthetic construct; identical to SEQ ID NO: 1620
  source 1..112: note = V017, ADI-76360, VL

SEQ ID NO: 1721 - protein, 23 aa, synthetic construct
  source 1..23: note = V017, ADI-76360, FRL1, VL
DIVMTQSPDSLAVSLGERATINC

SEQ ID NO: 1722 - protein, 17 aa, synthetic construct
  source 1..17: note = V017, ADI-76360, CDRL1, VL
KSSQSLLNARTGKNYLA

SEQ ID NO: 1723 - protein, 15 aa, synthetic construct
  source 1..15: note = V017, ADI-76360, FRL2, VL
WYQQKPGQPPKLLIY

SEQ ID NO: 1724 - protein, 7 aa, synthetic construct
  source 1..7: note = V017, ADI-76360, CDRL2, VL
WASTRES

SEQ ID NO: 1725 - protein, 32 aa, synthetic construct; identical to SEQ ID NO: 125
  source 1..32: note = V017, ADI-76360, FRL3, VL

SEQ ID NO: 1726 - protein, 8 aa, synthetic construct
  source 1..8: note = V017, ADI-76360, CDRL3, VL
KQSYSLRT

SEQ ID NO: 1727 - protein, 10 aa, synthetic construct
  source 1..10: note = V017, ADI-76360, FRL4, VL
FGGGTKVEIK

SEQ ID NO: 1728-1749 - skipped (22 entries)

SEQ ID NO: 1750 - DNA, 357 bp, synthetic construct
  source 1..357: mol_type = other DNA; note = V017, ADI-76360, VH
caggtgcagctggtgcagtctggggctgaggtgaagaagcctggggcctcagtgaaggtttcctgcaaggcttctggatttaacataaaggactattatatgcattgggtgcgccaggcccccggacaaaggcttgagtggatgggatggatcgacctggaaaacgcagacacaatatatgacgctaagttccagggcagagtcaccattaccagggacacatccgcgagcacagcctacatggagctgagcagcctgagatctgaagacacggcggtgtactactgcggccgggacgcctatggtagatatttttatgatgtctggggccagggcaccctggtcaccgtctcctca

SEQ ID NO: 1751-1759 - skipped (9 entries)

SEQ ID NO: 1760 - DNA, 336 bp, synthetic construct; identical to SEQ ID NO: 1660
  source 1..336: mol_type = other DNA; note = V017, ADI-76360, VL

SEQ ID NO: 1761-1809 - skipped (49 entries)

SEQ ID NO: 1810 - protein, 119 aa, synthetic construct
  source 1..119: note = V018, ADI-76361, VH
QVQLVQSGAEVKKPGASVKVSCKASGFDIKDYYMHWVRQAPGQRLEWMGWIDLEEGNTIYDAKFQGRVTITRDTSASTAYMELSSLRSEDTAVYYCARDAYGRYFYDVWGQGTLVTVSS

SEQ ID NO: 1811 - protein, 26 aa, synthetic construct
  source 1..26: note = V018, ADI-76361, FRH1, VH
QVQLVQSGAEVKKPGASVKVSCKASG

SEQ ID NO: 1812 - protein, 9 aa, synthetic construct
  source 1..9: note = V018, ADI-76361, CDRH1, VH
FDIKDYYMH

SEQ ID NO: 1813 - protein, 14 aa, synthetic construct
  source 1..14: note = V018, ADI-76361, FHR2, VH
WVRQAPGQRLEWMG

SEQ ID NO: 1814 - protein, 17 aa, synthetic construct
  source 1..17: note = V018, ADI-76361, CDRH2, VH
WIDLEEGNTIYDAKFQG

SEQ ID NO: 1815 - protein, 30 aa, synthetic construct; identical to SEQ ID NO: 115
  source 1..30: note = V018, ADI-76361, FRH3, VH

SEQ ID NO: 1816 - protein, 12 aa, synthetic construct
  source 1..12: note = V018, ADI-76361, CDRH3, VH
ARDAYGRYFYDV

SEQ ID NO: 1817 - protein, 11 aa, synthetic construct
  source 1..11: note = V018, ADI-76361, FRH4, VH
WGQGTLVTVSS

SEQ ID NO: 1818-1819 - skipped (2 entries)

SEQ ID NO: 1820 - protein, 112 aa, synthetic construct; identical to SEQ ID NO: 1620
  source 1..112: note = V018, ADI-76361, VL

SEQ ID NO: 1821 - protein, 23 aa, synthetic construct
  source 1..23: note = V018, ADI-76361, FRL1, VL
DIVMTQSPDSLAVSLGERATINC

SEQ ID NO: 1822 - protein, 17 aa, synthetic construct
  source 1..17: note = V018, ADI-76361, CDRL1, VL
KSSQSLLNARTGKNYLA

SEQ ID NO: 1823 - protein, 15 aa, synthetic construct
  source 1..15: note = V018, ADI-76361, FRL2, VL
WYQQKPGQPPKLLIY

SEQ ID NO: 1824 - protein, 7 aa, synthetic construct
  source 1..7: note = V018, ADI-76361, CDRL2, VL
WASTRES

SEQ ID NO: 1825 - protein, 32 aa, synthetic construct; identical to SEQ ID NO: 125
  source 1..32: note = V018, ADI-76361, FRL3, VL

SEQ ID NO: 1826 - protein, 8 aa, synthetic construct
  source 1..8: note = V018, ADI-76361, CDRL3, VL
KQSYSLRT

SEQ ID NO: 1827 - protein, 10 aa, synthetic construct
  source 1..10: note = V018, ADI-76361, FRL4, VL
FGGGTKVEIK

SEQ ID NO: 1828-1849 - skipped (22 entries)

SEQ ID NO: 1850 - DNA, 357 bp, synthetic construct
  source 1..357: mol_type = other DNA; note = V018, ADI-76361, VH
caggtgcagctggtgcagtctggggctgaggtgaagaagcctggggcctcagtgaaggtttcctgcaaggcttctggattcgatatcaaggattattatatgcattgggtgcgccaggcccccggacaaaggcttgagtggatgggatggatcgatctagaagaaggtaacacaatctatgacgcgaagttccagggcagagtcaccattaccagggacacatccgcgagcacagcctacatggagctgagcagcctgagatctgaagacacggcggtgtactactgcgctcgagatgcttacggtcggtacttttatgacgtttggggccagggcaccctggtcaccgtctcctca

SEQ ID NO: 1851-1859 - skipped (9 entries)

SEQ ID NO: 1860 - DNA, 336 bp, synthetic construct; identical to SEQ ID NO: 1660
  source 1..336: mol_type = other DNA; note = V018, ADI-76361, VL

SEQ ID NO: 1861-1909 - skipped (49 entries)

SEQ ID NO: 1910 - protein, 119 aa, synthetic construct
  source 1..119: note = V019, ADI-76362, VH
QVQLVQSGAEVKKPGASVKVSCKASGFNIKDYYMHWVRQAPGQRLEWMGWIDLEEGDTIYDAKFQGRVTITRDTSASTAYMELSSLRSEDTAVYYCARDAYGRYFYDVWGQGTLVTVSS

SEQ ID NO: 1911 - protein, 26 aa, synthetic construct
  source 1..26: note = V019, ADI-76362, FRH1, VH
QVQLVQSGAEVKKPGASVKVSCKASG

SEQ ID NO: 1912 - protein, 9 aa, synthetic construct
  source 1..9: note = V019, ADI-76362, CDRH1, VH
FNIKDYYMH

SEQ ID NO: 1913 - protein, 14 aa, synthetic construct
  source 1..14: note = V019, ADI-76362, FRH2, VH
WVRQAPGQRLEWMG

SEQ ID NO: 1914 - protein, 17 aa, synthetic construct
  source 1..17: note = V019, ADI-76362, CDRH2, VH
WIDLEEGDTIYDAKFQG

SEQ ID NO: 1915 - protein, 30 aa, synthetic construct; identical to SEQ ID NO: 115
  source 1..30: note = V019, ADI-76362, FRH3, VH

SEQ ID NO: 1916 - protein, 12 aa, synthetic construct
  source 1..12: note = V019, ADI-76362, CDRH3, VH
ARDAYGRYFYDV

SEQ ID NO: 1917 - protein, 11 aa, synthetic construct
  source 1..11: note = V019, ADI-76362, FRH4, VH
WGQGTLVTVSS

SEQ ID NO: 1918-1919 - skipped (2 entries)

SEQ ID NO: 1920 - protein, 112 aa, synthetic construct; identical to SEQ ID NO: 1620
  source 1..112: note = V019, ADI-76362, VL

SEQ ID NO: 1921 - protein, 23 aa, synthetic construct
  source 1..23: note = V019, ADI-76362, FRL1, VL
DIVMTQSPDSLAVSLGERATINC

SEQ ID NO: 1922 - protein, 17 aa, synthetic construct
  source 1..17: note = V019, ADI-76362, CDRL1, VL
KSSQSLLNARTGKNYLA

SEQ ID NO: 1923 - protein, 15 aa, synthetic construct
  source 1..15: note = V019, ADI-76362, FRL2, VL
WYQQKPGQPPKLLIY

SEQ ID NO: 1924 - protein, 7 aa, synthetic construct
  source 1..7: note = V019, ADI-76362, CDRL2, VL
WASTRES

SEQ ID NO: 1925 - protein, 32 aa, synthetic construct; identical to SEQ ID NO: 125
  source 1..32: note = V019, ADI-76362, FRL3, VL

SEQ ID NO: 1926 - protein, 8 aa, synthetic construct
  source 1..8: note = V019, ADI-76362, CDRL3, VL
KQSYSLRT

SEQ ID NO: 1927 - protein, 10 aa, synthetic construct
  source 1..10: note = V019, ADI-76362, FRL4, VL
FGGGTKVEIK

SEQ ID NO: 1928-1949 - skipped (22 entries)

SEQ ID NO: 1950 - DNA, 357 bp, synthetic construct
  source 1..357: mol_type = other DNA; note = V019, ADI-76362, VH
caggtgcagctggtgcagtctggggctgaggtgaagaagcctggggcctcagtgaaggtttcctgcaaggcttctggatttaacatcaaggattattatatgcattgggtgcgccaggcccccggacaaaggcttgagtggatgggatggatcgatcttgaggaaggtgatacaatttatgatgccaagttccagggcagagtcaccattaccagggacacatccgcgagcacagcctacatggagctgagcagcctgagatctgaagacacggcggtgtactactgcgcacgtgatgcatatggacgttatttttatgacgtttggggccagggcaccctggtcaccgtctcctca

SEQ ID NO: 1951-1959 - skipped (9 entries)

SEQ ID NO: 1960 - DNA, 336 bp, synthetic construct; identical to SEQ ID NO: 1660
  source 1..336: mol_type = other DNA; note = V019, ADI-76362, VL

SEQ ID NO: 1961-2109 - skipped (149 entries)

SEQ ID NO: 2110 - protein, 119 aa, synthetic construct; identical to SEQ ID NO: 1
  REGION 1..119: note = A001, ADI-26906, VH

SEQ ID NO: 2111 - protein, 26 aa, synthetic construct
  source 1..26: note = A001, ADI-26906, FRH1, VH
QVQLVQSGAEVKKPGASVKVSCKASG

SEQ ID NO: 2112 - protein, 9 aa, synthetic construct
  REGION 1..9: note = A001, ADI-26906, CDRH1, VH
FNIKDYYMH

SEQ ID NO: 2113 - protein, 14 aa, synthetic construct
  source 1..14: note = A001, ADI-26906, FRH2, VH
WVRQAPGQRLEWMG

SEQ ID NO: 2114 - protein, 17 aa, synthetic construct
  source 1..17: note = A001, ADI-26906, CDRH2, VH
WIDLENANTIYDAKFQG

SEQ ID NO: 2115 - protein, 30 aa, synthetic construct; identical to SEQ ID NO: 115
  source 1..30: note = A001, ADI-26906, FRH3, VH

SEQ ID NO: 2116 - protein, 12 aa, synthetic construct
  source 1..12: note = A001, ADI-26906, CDRH3, VH
ARDAYGRYFYDV

SEQ ID NO: 2117 - protein, 11 aa, synthetic construct
  source 1..11: note = A001, ADI-26906, FRH4, VH
WGQGTLVTVSS

SEQ ID NO: 2118-2119 - skipped (2 entries)

SEQ ID NO: 2120 - protein, 112 aa, synthetic construct; identical to SEQ ID NO: 15
  source 1..112: note = A001, ADI-26906, VL

SEQ ID NO: 2121 - protein, 23 aa, synthetic construct
  source 1..23: note = A001, ADI-26906, FRL1, VL
DIVMTQSPDSLAVSLGERATINC

SEQ ID NO: 2122 - protein, 17 aa, synthetic construct
  source 1..17: note = A001, ADI-26906, CDRL1, VL
KSSQSLLNARTGKNYLA

SEQ ID NO: 2123 - protein, 15 aa, synthetic construct
  source 1..15: note = A001, ADI-26906, FRL2, VL
WYQQKPGQPPKLLIY

SEQ ID NO: 2124 - protein, 7 aa, synthetic construct
  source 1..7: note = A001, ADI-26906, CDRL2, VL
WASTRES

SEQ ID NO: 2125 - protein, 32 aa, synthetic construct; identical to SEQ ID NO: 125
  source 1..32: note = A001, ADI-26906, FRL3, VL

SEQ ID NO: 2126 - protein, 8 aa, synthetic construct
  source 1..8: note = A001, ADI-26906, CDRL3, VL
KQSYSRRT

SEQ ID NO: 2127 - protein, 10 aa, synthetic construct
  source 1..10: note = A001, ADI-26906, FRL4, VL
FGGGTKVEIK

SEQ ID NO: 2128-2149 - skipped (22 entries)

SEQ ID NO: 2150 - DNA, 357 bp, synthetic construct
  source 1..357: mol_type = other DNA; note = A001, ADI-26906, VH
caggtgcagctggtgcagtctggggctgaggtgaagaagcctggggcctcagtgaaggtctcctgcaaggcttctggattcaacatcaaggactactatatgcactgggtgcgacaggctcctggacaaaggcttgagtggatgggatggatcgaccttgaaaatgctaacacaatctatgacgctaagtttcagggcagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 2151-2159 - skipped (9 entries)

SEQ ID NO: 2160 - DNA, 336 bp, synthetic construct; identical to SEQ ID NO: 260
  source 1..336: mol_type = other DNA; note = A001, ADI-26906, VL

SEQ ID NO: 2161-2209 - skipped (49 entries)

SEQ ID NO: 2210 - protein, 119 aa, synthetic construct; identical to SEQ ID NO: 2
  source 1..119: note = A002, ADI-50024, VH

SEQ ID NO: 2211 - protein, 26 aa, synthetic construct
  source 1..26: note = A002, ADI-50024, FRH1, VH
QVQLVQSGAEVKKPGASVKVSCKASG

SEQ ID NO: 2212 - protein, 9 aa, synthetic construct
  source 1..9: note = A002, ADI-50024, CDRH1, VH
FNIKDYYMH

SEQ ID NO: 2213 - protein, 14 aa, synthetic construct
  source 1..14: note = A002, ADI-50024, FRH2, VH
WVRQAPGQRLEWIG

SEQ ID NO: 2214 - protein, 17 aa, synthetic construct
  source 1..17: note = A002, ADI-50024, CDRH2, VH
WIDLENANTVYDAKFQG

SEQ ID NO: 2215 - protein, 30 aa, synthetic construct; identical to SEQ ID NO: 115
  source 1..30: note = A002, ADI-50024, FRH3, VH

SEQ ID NO: 2216 - protein, 12 aa, synthetic construct
  source 1..12: note = A002, ADI-50024, CDRH3, VH
ARDAYGRYFYDV

SEQ ID NO: 2217 - protein, 11 aa, synthetic construct
  source 1..11: note = A002, ADI-50024, FRH4, VH
WGQGTLVTVSS

SEQ ID NO: 2218-2219 - skipped (2 entries)

SEQ ID NO: 2220 - protein, 112 aa, synthetic construct; identical to SEQ ID NO: 16
  source 1..112: note = A002, ADI-50024, VL

SEQ ID NO: 2221 - protein, 23 aa, synthetic construct
  source 1..23: note = A002, ADI-50024, FRL1, VL
DIVMSQSPDSLAVSLGERATINC

SEQ ID NO: 2222 - protein, 17 aa, synthetic construct
  source 1..17: note = A002, ADI-50024, CDRL1, VL
KSSQSLLNARTGKNYLA

SEQ ID NO: 2223 - protein, 15 aa, synthetic construct
  source 1..15: note = A002, ADI-50024, FRL2, VL
WYQQKPGQPPKLLIY

SEQ ID NO: 2224 - protein, 7 aa, synthetic construct
  source 1..7: note = A002, ADI-50024, CDRL2, VL
WASTRSS

SEQ ID NO: 2225 - protein, 32 aa, synthetic construct; identical to SEQ ID NO: 125
  source 1..32: note = A002, ADI-50024, FRL3, VL

SEQ ID NO: 2226 - protein, 8 aa, synthetic construct
  source 1..8: note = A002, ADI-50024, CDRL3, VL
VQSYFRRT

SEQ ID NO: 2227 - protein, 10 aa, synthetic construct
  source 1..10: note = A002, ADI-50024, FRL4, VL
FGGGTKVEIK

SEQ ID NO: 2228-2249 - skipped (22 entries)

SEQ ID NO: 2250 - DNA, 357 bp, synthetic construct
  source 1..357: mol_type = other DNA; note = A002, ADI-50024, VH
caggtgcagctggtgcagtctggggctgaggtgaagaagcctggggcctcagtgaaggtctcctgcaaggcttctggattcaacatcaaggactactatatgcactgggtgcgacaggctcctggacaaaggcttgagtggataggatggatcgaccttgaaaatgctaacacagtctatgacgctaagtttcagggcagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 2251-2259 - skipped (9 entries)

SEQ ID NO: 2260 - DNA, 336 bp, synthetic construct; identical to SEQ ID NO: 860
  source 1..336: mol_type = other DNA; note = A002, ADI-50024, VL

SEQ ID NO: 2261-2309 - skipped (49 entries)

SEQ ID NO: 2310 - protein, 119 aa, synthetic construct
  source 1..119: note = A003, ADI-26913, VH
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDQYGRYFYDVWGQGTLVTVSS

SEQ ID NO: 2311 - protein, 26 aa, synthetic construct
  source 1..26: note = A003, ADI-26913, FRH1, VH
QVQLVQSGAEVKKPGASVKVSCKASG

SEQ ID NO: 2312 - protein, 9 aa, synthetic construct
  source 1..9: note = A003, ADI-26913, CDRH1, VH
FNIKDYYMH

SEQ ID NO: 2313 - protein, 14 aa, synthetic construct
  source 1..14: note = A003, ADI-26913, FRH2, VH
WVRQAPGQRLEWMG

SEQ ID NO: 2314 - protein, 17 aa, synthetic construct
  source 1..17: note = A003, ADI-26913, CDRH2, VH
WIDLENANTIYDAKFQG

SEQ ID NO: 2315 - protein, 30 aa, synthetic construct; identical to SEQ ID NO: 115
  source 1..30: note = A003, ADI-26913, FRH3, VH

SEQ ID NO: 2316 - protein, 12 aa, synthetic construct
  source 1..12: note = A003, ADI-26913, CDRH3, VH
ARDQYGRYFYDV

SEQ ID NO: 2317 - protein, 11 aa, synthetic construct
  source 1..11: note = A003, ADI-26913, FRH4, VH
WGQGTLVTVSS

SEQ ID NO: 2318-2319 - skipped (2 entries)

SEQ ID NO: 2320 - protein, 112 aa, synthetic construct; identical to SEQ ID NO: 15
  source 1..112: note = A003, ADI-26913, VL

SEQ ID NO: 2321 - protein, 23 aa, synthetic construct
  source 1..23: note = A003, ADI-26913, FRL1, VL
DIVMTQSPDSLAVSLGERATINC

SEQ ID NO: 2322 - protein, 17 aa, synthetic construct
  source 1..17: note = A003, ADI-26913, CDRL1, VL
KSSQSLLNARTGKNYLA

SEQ ID NO: 2323 - protein, 15 aa, synthetic construct
  source 1..15: note = A003, ADI-26913, FRL2, VL
WYQQKPGQPPKLLIY

SEQ ID NO: 2324 - protein, 7 aa, synthetic construct
  source 1..7: note = A003, ADI-26913, CDRL2, VL
WASTRES

SEQ ID NO: 2325 - protein, 32 aa, synthetic construct; identical to SEQ ID NO: 125
  source 1..32: note = A003, ADI-26913, FRL3, VL

SEQ ID NO: 2326 - protein, 8 aa, synthetic construct
  source 1..8: note = A003, ADI-26913, CDRL3, VL
KQSYSRRT

SEQ ID NO: 2327 - protein, 10 aa, synthetic construct
  source 1..10: note = A003, ADI-26913, FRL4, VL
FGGGTKVEIK

SEQ ID NO: 2328-2349 - skipped (22 entries)

SEQ ID NO: 2350 - DNA, 357 bp, synthetic construct
  source 1..357: mol_type = other DNA; note = A003, ADI-26913, VH
caggtgcagctggtgcagtctggggctgaggtgaagaagcctggggcctcagtgaaggtctcctgcaaggcttctggattcaacatcaaggactactatatgcactgggtgcgacaggctcctggacaaaggcttgagtggatgggatggatcgaccttgaaaatgctaacacaatctatgacgctaagtttcagggcagggtcaccataaccagggacacgtccgccagcacagcctacatggagctgagcagcctgagatctgaagacacggcggtgtactactgcgcgagagatcagtacgggagatatttttacgacgtgtggggccaaggaaccctggtcaccgtctcctca

SEQ ID NO: 2351-2359 - skipped (9 entries)

SEQ ID NO: 2360 - DNA, 336 bp, synthetic construct; identical to SEQ ID NO: 260
  source 1..336: mol_type = other DNA; note = A003, ADI-26913, VL

SEQ ID NO: 2361-2409 - skipped (49 entries)

SEQ ID NO: 2410 - protein, 119 aa, synthetic construct
  source 1..119: note = A004, ADI-26920, VH
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCGRDAYGRYFYDVWGQGTLVTVSS

SEQ ID NO: 2411 - protein, 26 aa, synthetic construct
  source 1..26: note = A004, ADI-26920, FRH1, VH
QVQLVQSGAEVKKPGASVKVSCKASG

SEQ ID NO: 2412 - protein, 9 aa, synthetic construct
  source 1..9: note = A004, ADI-26920, CDRH1, VH
FNIKDYYMH

SEQ ID NO: 2413 - protein, 14 aa, synthetic construct
  source 1..14: note = A004, ADI-26920, FRH2, VH
WVRQAPGQRLEWMG

SEQ ID NO: 2414 - protein, 17 aa, synthetic construct
  source 1..17: note = A004, ADI-26920, CDRH2, VH
WIDLENANTIYDAKFQG

SEQ ID NO: 2415 - protein, 30 aa, synthetic construct; identical to SEQ ID NO: 115
  source 1..30: note = A004, ADI-26920, FRH3, VH

SEQ ID NO: 2416 - protein, 12 aa, synthetic construct
  source 1..12: note = A004, ADI-26920, CDRH3, VH
GRDAYGRYFYDV

SEQ ID NO: 2417 - protein, 11 aa, synthetic construct
  source 1..11: note = A004, ADI-26920, FRH4, VH
WGQGTLVTVSS

SEQ ID NO: 2418-2419 - skipped (2 entries)

SEQ ID NO: 2420 - protein, 112 aa, synthetic construct; identical to SEQ ID NO: 1620
  source 1..112: note = A004, ADI-26920, VL

SEQ ID NO: 2421 - protein, 23 aa, synthetic construct
  source 1..23: note = A004, ADI-26920, FRL1, VL
DIVMTQSPDSLAVSLGERATINC

SEQ ID NO: 2422 - protein, 17 aa, synthetic construct
  source 1..17: note = A004, ADI-26920, CDRL1, VL
KSSQSLLNARTGKNYLA

SEQ ID NO: 2423 - protein, 15 aa, synthetic construct
  source 1..15: note = A004, ADI-26920, FRL2, VL
WYQQKPGQPPKLLIY

SEQ ID NO: 2424 - protein, 7 aa, synthetic construct
  source 1..7: note = A004, ADI-26920, CDRL2, VL
WASTRES

SEQ ID NO: 2425 - protein, 32 aa, synthetic construct; identical to SEQ ID NO: 125
  source 1..32: note = A004, ADI-26920, FRL3, VL

SEQ ID NO: 2426 - protein, 8 aa, synthetic construct
  source 1..8: note = A004, ADI-26920, CDRL3, VL
KQSYSLRT

SEQ ID NO: 2427 - protein, 10 aa, synthetic construct
  source 1..10: note = A004, ADI-26920, FRL4, VL
FGGGTKVEIK

SEQ ID NO: 2428-2449 - skipped (22 entries)

SEQ ID NO: 2450 - DNA, 357 bp, synthetic construct
  source 1..357: mol_type = other DNA; note = A004, ADI-26920, VH
caggtgcagctggtgcagtctggggctgaggtgaagaagcctggggcctcagtgaaggtctcctgcaaggcttctggattcaacatcaaggactactatatgcactgggtgcgacaggctcctggacaaaggcttgagtggatgggatggatcgaccttgaaaatgctaacacaatctatgacgctaagtttcagggcagggtcaccataaccagggacacgtccgccagcacagcctacatggagctgagcagcctgagatctgaagacacggcggtgtactactgcggtagagatgcgtacgggagatatttttacgacgtgtggggccaaggaaccctggtcaccgtctcctca

SEQ ID NO: 2451-2459 - skipped (9 entries)

SEQ ID NO: 2460 - DNA, 336 bp, synthetic construct; identical to SEQ ID NO: 1660
  source 1..336: mol_type = other DNA; note = A004, ADI-26920, VL

SEQ ID NO: 2461-2509 - skipped (49 entries)

SEQ ID NO: 2510 - protein, 119 aa, synthetic construct
  source 1..119: note = A005, ADI-26921, VH
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSS

SEQ ID NO: 2511 - protein, 26 aa, synthetic construct
  source 1..26: note = A005, ADI-26921, FRH1, VH
QVQLVQSGAEVKKPGASVKVSCKASG

SEQ ID NO: 2512 - protein, 9 aa, synthetic construct
  source 1..9: note = A005, ADI-26921, CDRH1, VH
FNIKDYYMH

SEQ ID NO: 2513 - protein, 14 aa, synthetic construct
  source 1..14: note = A005, ADI-26921, FRH2, VH
WVRQAPGQRLEWMG

SEQ ID NO: 2514 - protein, 17 aa, synthetic construct
  source 1..17: note = A005, ADI-26921, CDRH2, VH
WIDLEEGNTIYDAKFQG

SEQ ID NO: 2515 - protein, 30 aa, synthetic construct; identical to SEQ ID NO: 115
  source 1..30: note = A005, ADI-26921, FRH3, VH

SEQ ID NO: 2516 - protein, 12 aa, synthetic construct
  source 1..12: note = A005, ADI-26921, CDRH3, VH
ARDAYGRYFYDV

SEQ ID NO: 2517 - protein, 11 aa, synthetic construct
  source 1..11: note = A005, ADI-26921, FRH4, VH
WGQGTLVTVSS

SEQ ID NO: 2518-2519 - skipped (2 entries)

SEQ ID NO: 2520 - protein, 112 aa, synthetic construct; identical to SEQ ID NO: 1620
  source 1..112: note = A005, ADI-26921, VL

SEQ ID NO: 2521 - protein, 23 aa, synthetic construct
  source 1..23: note = A005, ADI-26921, FRL1, VL
DIVMTQSPDSLAVSLGERATINC

SEQ ID NO: 2522 - protein, 17 aa, synthetic construct
  source 1..17: note = A005, ADI-26921, CDRL1, VL
KSSQSLLNARTGKNYLA

SEQ ID NO: 2523 - protein, 15 aa, synthetic construct
  source 1..15: note = A005, ADI-26921, FRL2, VL
WYQQKPGQPPKLLIY

SEQ ID NO: 2524 - protein, 7 aa, synthetic construct
  source 1..7: note = A005, ADI-26921, CDRL2, VL
WASTRES

SEQ ID NO: 2525 - protein, 32 aa, synthetic construct; identical to SEQ ID NO: 125
  source 1..32: note = A005, ADI-26921, FRL3, VL

SEQ ID NO: 2526 - protein, 8 aa, synthetic construct
  source 1..8: note = A005, ADI-26921, CDRL3, VL
KQSYSLRT

SEQ ID NO: 2527 - protein, 10 aa, synthetic construct
  source 1..10: note = A005, ADI-26921, FRL4, VL
FGGGTKVEIK

SEQ ID NO: 2528-2549 - skipped (22 entries)

SEQ ID NO: 2550 - DNA, 357 bp, synthetic construct
  source 1..357: mol_type = other DNA; note = A005, ADI-26921, VH
caggtgcagctggtgcagtctggggctgaggtgaagaagcctggggcctcagtgaaggtctcctgcaaggcttctggattcaacatcaaggactactatatgcactgggtgcgacaggctcctggacaaaggcttgagtggatgggatggatcgaccttgaagagggcaacacaatctatgacgctaagtttcagggcagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 2551-2559 - skipped (9 entries)

SEQ ID NO: 2560 - DNA, 336 bp, synthetic construct; identical to SEQ ID NO: 1660
  source 1..336: mol_type = other DNA; note = A005, ADI-26921, VL

(19) United States
(12) Patent Application Publication
(10) Pub. No.: US 2024/0317858 A1
(43) Pub. Date: Sep. 26, 2024

(54) ANTI-CD3 ANTIBODIES

(71) Applicant: ADIMAB, LLC, Lebanon, NH (US)

(72) Inventors: Michael Benjamin BATTLES, Lebanon, NH (US); Catherine Yue LIU, Lebanon, NH (US)

(21) Appl. No.: 18/692,097

(22) PCT Filed: Sep. 16, 2022
(86) PCT No.: PCT/US2022/076522

§ 371 Date: Mar. 14, 2024

Related U.S. Application Data
(60) Provisional application No. 63/245,499, filed on Sep. 17, 2021.

(51) Int. Cl. C07K 16/28

(52) U.S. Cl. CPC C07K 16/2809; C07K 2317/31; C07K 2317/33; C07K 2317/522; C07K 2317/524; C07K 2317/526; C07K 2317/565; C07K 2317/567; C07K 2317/734; C07K 2317/92; C07K 2317/94

(57) ABSTRACT

The present disclosure includes anti-CD3 binding domains and antibodies and/or antigen-binding fragments including such domains. Also included are multispecific antibodies and antibody fragments. The present disclosure further includes nucleic acids and vectors encoding such antibodies or antibody fragments and cells comprising such nucleic acids. Further included are pharmaceutical compositions, in vivo methods, and manufacturing methods relating to anti-CD3 antibodies or antigen-binding fragments.

### CROSS-REFERENCE TO RELATED APPLICATIONS

This application claims priority to U.S. Provisional Application No. 63/245,499, filed on Sep. 17, 2021, entitled “Anti-CD3 Antibodies”, the contents of which are incorporated by reference in their entirety herein.

### SEQUENCE DISCLOSURE

The contents of the electronic sequence listing (1160430.003213.xml; Size: 964,969 bytes; and Date of Creation: Sep. 15, 2022) is herein incorporated by reference in its entirety.

### BACKGROUND

Cell proliferative disorders, such as cancer, are characterized by the uncontrolled growth of cell subpopulations. They are the leading cause of death in the developed world and the second leading cause of death in developing countries, with a total number of new cancer cases per year expected to rise to 23.6 million by 2030. The National Cancer Institute estimates that almost 2 million new cases of cancer will be diagnosed in the U.S. and greater than 600,000 Americans will die of cancer in 2018. Cancer care thus represents a significant and ever-increasing societal burden.

The idea of using the cytotoxic capacity of T cells to kill tumor cells through use of CD3 targeting bispecific antibodies dates back to the mid-1980s. (Staerz et al. Nature 1985 314: 628-32). Many bispecific antibodies developed to date contain a first binding site specific to CD3 for T-cell recruitment and activation, and a second binding site for a targeted disease-associated antigen, such as an antigen produced by a tumor cell. CD3 bispecific antibodies trigger the CD3 surface receptor on T cells by binding to their second target protein expressed on tumors such that available T cells can bind to target-expressing cells via bridging by the CD3 bispecific antibody, irrespective of the peptide/MHC specificity of their T-cell receptor. (See, e.g., Bassan, 2012, Blood 120:5094-95). Bridging of T cells and tumor cells using CD3 bispecific antibodies can induce dramatic regression of advanced-stage malignancies and, in some cases, lead to complete remission. Currently, more than 25 different CD3 bispecific antibodies are in clinical development for treatment of hematologic malignancies or solid cancers by targeting CD19, CD20, CD33, and CD123, or EpCAM, HER2, PSMA, and CEA, respectively. (See, e.g., Liu et al. Front Imnmunol 2017 8:38).

While bispecific antibodies have shown considerable benefits over monospecific antibodies for the treatment and the detection of cancer, broad commercial application of bispecific antibodies has been hampered by the lack of efficient/low-cost production methods, the lack of stability of bispecific polypeptides and the lack of long half-lives in humans. A large variety of methods have been developed over the last few decades to produce bispecific monoclonal antibodies. However, many candidate bispecific antibodies with exquisite selectivity and high potency toward the target of interest often have problems in downstream development and clinical efficacy activities, including polyspecific binding; off-target binding; nonspecific binding; poor expression levels or profiles in eukaryotic host cells, such as mammalian host cells and yeast cells; poor chemical and physical properties, such as poor stability during storage (e.g., poor/low “shelf-life” stability), poor (low) solubility, poor (high) viscosity, propensity to aggregate, and the like; and poor clinical and biophysical profiles, such as poor pharmacokinetic profiles, poor pharmacodynamic profiles, fast or poor in vivo clearance rates, short circulation half-life, some of which result in termination of their development.

Certain techniques and assays exist to assess many of the aforementioned developability characteristics for discovered antibodies in the context of downstream development activities (“post-discovery antibodies”), such as CIC, SIC, BVP-ELISA, TMA, and other assays; however, such assays are typically not amenable to high-throughput formats in early antibody discovery platforms. Furthermore, assessment of these attributes typically requires milligram to gram quantities of protein, thus often imposing a defacto limitation on the number of leads that can be pragmatically considered for development, and consequently reducing the likelihood of program success. Consequently, significant resources are often expended attempting to fix poorly behaving lead candidates with few backups available in later stages of development.

A variety of anti-CD3 antibodies are known in the art, including monoclonal and bispecific antibody formats. See, e.g., U.S. Pat. Nos. 7,262,276; 7,635,472; 7,862,813; 9,587,021; and 10,174,124. However, many of these anti-CD3 antibodies possess developability issues, such as those outlined above. Therefore, such an anti-CD3 antibody is not an ideal candidate, e.g., for designing a multispecific antibody for clinical purposes. Accordingly, there is an unmet need for the provision for anti-CD3 antibodies that display desirable developability properties and which are safe and efficacious in, for example, binding specifically to CD3 expressed on T-cells, activating T-cells, and (re)-directing the activated T-cells to kill target cells.

### SUMMARY

One aspect of the present disclosure provides anti-CD3 antibodies and antigen-binding fragments, e.g., those that display desirable developability properties.

In some embodiments, the present disclosure provides an antibody or antigen-binding fragment that includes: a complementarity determining region (CDR) comprising: (i) the amino acid sequence of a CDR contained in any of the variable domain sequences listed in Table 1A or 1B. In some embodiments, the present disclosure provides an antibody or antigen-binding fragment that comprises: a CDR comprising an amino acid sequence selected from any of those listed in Table 2A or 2B.

In some embodiments, the present disclosure provides an antibody or antigen-binding fragment which comprises (A) a heavy chain variable domain (VH) polypeptide comprising: (a) a VH CDR1 (CDRH1) comprising the amino acid sequence of: (i) the CDRH1 contained in any one of Antibody Nos. V002-V019 and A001-A005; (ii) the CDRH1 contained in any one of SEQ ID NOS: 210, 310, 410, 510, 610, 710, 810, 910, 1010, 1110, 1210, 1310, 1410, 1510, 1610, 1710, 1810, 1910, 2110, 2210, 2310, 2410, and 2510; (iii) FX₁X₂X₃DYYMH (SEQ ID NO: 112), wherein X₁ is N or D, X₂ is I or D, and X₃ is K or D; and/or (iv) any one of SEQ ID NOS: 212, 312, 412, 512, 612, 712, 812, 912, 1012, 1112, 1212, 1312, 1412, 1512, 1612, 1712, 1812, 1912, 2112, 2212, 2312, 2412, and 2512; (b) a VH CDR2 (CDRH2) comprising the amino acid sequence of: (i) the CDRH2 contained in any one of Antibody Nos. V002-V019 and A001-A005; (ii) the CDRH2 contained in any one of SEQ ID NOS: 210, 310, 410, 510, 610, 710, 810, 910, 1010, 1110, 1210, 1310, 1410, 1510, 1610, 1710, 1810, 1910, 2110, 2210, 2310, 2410, and 2510; (iii) WIX₄LEX₅X₆X₇TX₈X₉DAKFQX₁₀ (SEQ ID NO: 114), wherein X₄ is D or E, X₅ is N or E, X₆ is A, D, or G, X₇ is N, E, or D, X₈ is I or V, X₉ is Y or D, and Xia is G or D; and/or (iv) any one of SEQ ID NOS: 214, 314, 414, 514, 614, 714, 814, 914, 1014, 1114, 1214, 1314, 1414, 1514, 1614, 1714, 1814, 1914, 2114, 2214, 2314, 2414, and 2514; and/or (c) a VH CDR3 (CDRH3) comprising the amino acid sequence of: (i) the CDRH3 contained in any one of Antibody Nos. V002-V019 and A001-A005; (ii) the CDRH3 contained in any one of SEQ ID NOS: 210, 310, 410, 510, 610, 710, 810, 910, 1010, 1110, 1210, 1310, 1410, 1510, 1610, 1710, 1810, 1910, 2110, 2210, 2310, 2410, and 2510; (iii) X₁₁RDX₁₂YGRYFYDX₁₃ (SEQ ID NO: 116), wherein X₁₁ is A or G, X₁₂ is A or Q, and X₁₃ is V or E; and/or (iv) any one of SEQ ID NOS: 216, 316, 416, 516, 616, 716, 816, 916, 1016, 1116, 1216, 1316, 1416, 1516, 1616, 1716, 1816, 1916, 2116, 2216, 2316, 2416, and 2516; and/or (B) a light chain variable domain (VL) polypeptide comprising: (a) a VL CDR1 (CDRL1) comprising the amino acid sequence of: (i) the CDRL1 contained in any one of Antibody Nos. V002-V019 and A001-A005; (ii) the CDRL1 contained in any one of SEQ ID NOS: 220, 320, 420, 520, 620, 720, 820, 920, 1020, 1120, 1220, 1320, 1420, 1520, 1620, 1720, 1820, 1920, 2120, 2220, 2320, 2420, and 2520; and/or (iii) any one of SEQ ID NOS: 122, 222, 322, 422, 522, 622, 722, 822, 922, 1022, 1122, 1222, 1322, 1422, 1522, 1622, 1722, 1822, 1922, 2122, 2222, 2322, 2422, and 2522; (b) a VL CDR2 (CDRL2) comprising the amino acid sequence of: (i) the CDRL2 contained in any one of Antibody Nos. V002-V019 and A001-A005; (ii) the CDRL2 contained in any one of SEQ ID NOS: 220, 320, 420, 520, 620, 720, 820, 920, 1020, 1120, 1220, 1320, 1420, 1520, 1620, 1720, 1820, 1920, 2120, 2220, 2320, 2420, and 2520; (iii) WASTRX₁₄S (SEQ ID NO: 124), wherein X₁₄ is E or S; and/or (iv) any one of SEQ ID NOS: 224, 324, 424, 524, 624, 724, 824, 924, 1024, 1124, 1224, 1324, 1424, 1524, 1624, 1724, 1824, 1924, 2124, 2224, 2324, 2424, and 2524; and/or (c) a VL CDR3 (CDRL3) comprising the amino acid sequence of: (i) the CDRL3 contained in any one of Antibody Nos. V002-V019 and A001-A005; (ii) the CDRL3 contained in any one of SEQ ID NOS: 220, 320, 420, 520, 620, 720, 820, 920, 1020, 1120, 1220, 1320, 1420, 1520, 1620, 1720, 1820, 1920, 2120, 2220, 2320, 2420, and 2520; (iii) X₁₅QSYX₁₆X₁₇RT (SEQ ID NO: 126), wherein X₁₅ is K or V, X₁₆ is S or F, and X₁₇ is R or L; and/or (iv) any one of SEQ ID NOS: 226, 326, 426, 526, 626, 726, 826, 926, 1026, 1126, 1226, 1326, 1426, 1526, 1626, 1726, 1826, 1926, 2126, 2226, 2326, 2426, and 2526 or an antibody or antigen-binding fragment which comprises a combination of one or more of the foregoing CDRs.

In certain embodiments, the anti-CD3 antibody or antigen-binding fragment may not comprise: (i) at least one of the CDR-H1, CDR-H2, CDR-H3, CDR-L1, CDR-L2, and CDR-L3 contained in A001; or (ii) at least one of SEQ ID NOS: 2112, 2114, 2116, 2122, 2124, and 2126. In particular embodiments, the anti-CD3 antibody or antigen-binding fragment may not comprise: (i) at least one of the CDRH1 and CDRH2 contained in A001; or (ii) at least one of SEQ ID NOS: 2112 and 2114.

In certain embodiments, the anti-CD3 antibody or antigen-binding fragment may not comprise: (i) at least one of the CDR-H1, CDR-H2, CDR-H3, CDR-L1, CDR-L2, and CDR-L3 contained in A002; or (ii) at least one of SEQ ID NOS: 2212, 2214, 2216, 2222, 2224, and 2226. In particular embodiments, the anti-CD3 antibody or antigen-binding fragment may not comprise: (i) at least one of the CDRH1 and CDRH2 contained in A002; or (ii) at least one of SEQ ID NOS: 2212 and 2214.

In certain embodiments, the anti-CD3 antibody or antigen-binding fragment may not comprise: (i) at least one of the CDR-H1, CDR-H2, CDR-H3, CDR-L1, CDR-L2, and CDR-L3 contained in A003; or (ii) at least one of SEQ ID NOS: 2312, 2314, 2316, 2322, 2324, and 2326. In particular embodiments, the anti-CD3 antibody or antigen-binding fragment may not comprise: (i) at least one of the CDRH1 and CDRH2 contained in A003; or (ii) at least one of SEQ ID NOS: 2312 and 2314.

In certain embodiments, the anti-CD3 antibody or antigen-binding fragment may not comprise: (i) at least one of the CDR-H1, CDR-H2, CDR-H3, CDR-L1, CDR-L2, and CDR-L3 contained in A004; or (ii) at least one of SEQ ID NOS: 2412, 2414, 2416, 2422, 2424, and 2426. In particular embodiments, the anti-CD3 antibody or antigen-binding fragment may not comprise: (i) at least one of the CDRH1 and CDRH2 contained in A004; or (ii) at least one of SEQ ID NOS: 2412 and 2414.

In certain embodiments, the anti-CD3 antibody or antigen-binding fragment may not comprise: (i) at least one of the CDR-H1, CDR-H2, CDR-H3, CDR-L1, CDR-L2, and CDR-L3 contained in A005; or (ii) at least one of SEQ ID NOS: 2512, 2514, 2516, 2522, 2524, and 2526. In particular embodiments, the anti-CD3 antibody or antigen-binding fragment may not comprise: (i) at least one of the CDRH1 and CDRH2 contained in A005; or (ii) at least one of SEQ ID NOS: 2512 and 2514.

In certain embodiments, the anti-CD3 antibody or antigen-binding fragment may comprise: (A) a VH polypeptide comprising said CDRH1, said CDRH2, and said CDRH3, as described above; and/or (B) a VL polypeptide comprising said CDRL1, said CDRL2, and said CDRL3, as described above. In particular embodiments, the anti-CD3 antibody or antigen-binding fragment may comprise: (A) a VH polypeptide comprising said CDRH1, said CDRH2, and said CDRH3, as described above; and (B) a VL polypeptide comprising said CDRL1, said CDRL2, and said CDRL3, as described above.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V002; or (ii) a CDRH1 comprising SEQ ID NO: 212, a CDRH2 comprising SEQ ID NO: 214, a CDRH3 comprising SEQ ID NO: 216, a CDRL1 comprising SEQ ID NO: 222, a CDRL2 comprising SEQ ID NO: 224, and a CDRL3 comprising SEQ ID NO: 226.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V003; or (ii) a CDRH1 comprising SEQ ID NO: 312, a CDRH2 comprising SEQ ID NO: 314, a CDRH3 comprising SEQ ID NO: 316, a CDRL1 comprising SEQ ID NO: 322, a CDRL2 comprising SEQ ID NO: 324, and a CDRL3 comprising SEQ ID NO: 326.

In particular embodiments, the anti-CD3 antibody or antigen-binding fragment may comprise: (i) the CDRH1, the CDRH2, the CDR1H3, the CDRL1, the CDRL2, and the CDR-L3 contained in Antibody No. V004; or (ii) a CDRH1 comprising SEQ ID NO: 412, a CDRH2 comprising SEQ ID NO: 414, a CDRH3 comprising SEQ ID NO: 416, a CDRL1 comprising SEQ ID NO: 422, a CDRL2 comprising SEQ ID NO: 424, and a CDRL3 comprising SEQ ID NO: 426.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V005; or (ii) a CDRH1 comprising SEQ ID NO: 512, a CDRH2 comprising SEQ ID NO: 514, a CDRH3 comprising SEQ ID NO: 516, a CDRL1 comprising SEQ ID NO: 522, a CDRL2 comprising SEQ ID NO: 524, and a CDRL3 comprising SEQ ID NO: 526.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V006; or (ii) a CDRH1 comprising SEQ ID NO: 612, a CDRH2 comprising SEQ ID NO: 614, a CDRH3 comprising SEQ ID NO: 616, a CDRL1 comprising SEQ ID NO: 622, a CDRL2 comprising SEQ ID NO: 624, and a CDRL3 comprising SEQ ID NO: 626.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V007; or (ii) a CDRH1 comprising SEQ ID NO: 712, a CDRH2 comprising SEQ ID NO: 714, a CDRH3 comprising SEQ ID NO: 716, a CDRL1 comprising SEQ ID NO: 722, a CDRL2 comprising SEQ ID NO: 724, and a CDRL3 comprising SEQ ID NO: 726.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V008; or (ii) a CDRH1 comprising SEQ ID NO: 812, a CDRH2 comprising SEQ ID NO: 814, a CDRH3 comprising SEQ ID NO: 816, a CDRL1 comprising SEQ ID NO: 822, a CDRL2 comprising SEQ ID NO: 824, and a CDRL3 comprising SEQ ID NO: 826.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V009; or (ii) a CDRH1 comprising SEQ ID NO: 912, a CDRH2 comprising SEQ ID NO: 914, a CDRH3 comprising SEQ ID NO: 916, a CDRL1 comprising SEQ ID NO: 922, a CDRL2 comprising SEQ ID NO: 924, and a CDRL3 comprising SEQ ID NO: 926.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V010; or (ii) a CDRH1 comprising SEQ ID NO: 1012, a CDRH2 comprising SEQ ID NO: 1014, a CDRH3 comprising SEQ ID NO: 1016, a CDRL1 comprising SEQ ID NO: 1022, a CDRL2 comprising SEQ ID NO: 1024, and a CDRL3 comprising SEQ ID NO: 1026.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V011; or (ii) a CDRH1 comprising SEQ ID NO: 1112, a CDRH2 comprising SEQ ID NO: 1114, a CDRH3 comprising SEQ ID NO: 1116, a CDRL1 comprising SEQ ID NO: 1122, a CDRL2 comprising SEQ ID NO: 1124, and a CDRL3 comprising SEQ ID NO: 1126.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V012; or (ii) a CDRH1 comprising SEQ ID NO: 1212, a CDRH2 comprising SEQ ID NO: 1214, a CDRH3 comprising SEQ ID NO: 1216, a CDRL1 comprising SEQ ID NO: 1222, a CDRL2 comprising SEQ ID NO: 1224, and a CDRL3 comprising SEQ ID NO: 1226.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V013; or (ii) a CDRH1 comprising SEQ ID NO: 1312, a CDRH2 comprising SEQ ID NO: 1314, a CDRH3 comprising SEQ ID NO: 1316, a CDRL1 comprising SEQ ID NO: 1322, a CDRL2 comprising SEQ ID NO: 1324, and a CDRL3 comprising SEQ ID NO: 1326.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V014; or (ii) a CDRH1 comprising SEQ ID NO: 1412, a CDRH2 comprising SEQ ID NO: 1414, a CDRH3 comprising SEQ ID NO: 1416, a CDRL1 comprising SEQ ID NO: 1422, a CDRL2 comprising SEQ ID NO: 1424, and a CDRL3 comprising SEQ ID NO: 1426.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V015; or (ii) a CDRH1 comprising SEQ ID NO: 1512, a CDRH2 comprising SEQ ID NO: 1514, a CDRH3 comprising SEQ ID NO: 1516, a CDRL1 comprising SEQ ID NO: 1522, a CDRL2 comprising SEQ ID NO: 1524, and a CDRL3 comprising SEQ ID NO: 1526.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V016; or (ii) a CDRH1 comprising SEQ ID NO: 1612, a CDRH2 comprising SEQ ID NO: 1614, a CDRH3 comprising SEQ ID NO: 1616, a CDRL1 comprising SEQ ID NO: 1622, a CDRL2 comprising SEQ ID NO: 1624, and a CDRL3 comprising SEQ ID NO: 1626.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V017; or (ii) a CDRH1 comprising SEQ ID NO: 1712, a CDRH2 comprising SEQ ID NO: 1714, a CDRH3 comprising SEQ ID NO: 1716, a CDRL1 comprising SEQ ID NO: 1722, a CDRL2 comprising SEQ ID NO: 1724, and a CDRL3 comprising SEQ ID NO: 1726.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V018; or (ii) a CDRH1 comprising SEQ ID NO: 1812, a CDRH2 comprising SEQ ID NO: 1814, a CDRH3 comprising SEQ ID NO: 1816, a CDRL1 comprising SEQ ID NO: 1822, a CDRL2 comprising SEQ ID NO: 1824, and a CDRL3 comprising SEQ ID NO: 1826.

In particular embodiments, the anti-CD3 antibody or antigen-binding fragment may comprise: (i) the CDRH1, the CDRH2, the CDRH3, the CDRL1, the CDRL2, and the CDR-L3 contained in Antibody No. V019; or (ii) a CDRH1 comprising SEQ ID NO: 1912, a CDRH2 comprising SEQ ID NO: 1914, a CDRH3 comprising SEQ ID NO: 1916, a CDRL1 comprising SEQ ID NO: 1922, a CDRL2 comprising SEQ ID NO: 1924, and a CDRL3 comprising SEQ ID NO: 1926.

In some embodiments, in the anti-CD3 antibody or antigen-binding fragment may comprise: (A) the VH polypeptide may comprise: (a) a VH framework region 1 (FRH1) comprising the amino acid sequence of: (i) the FRH1 contained in any one of Antibody Nos. V002-V019 and A001-A005; (ii) the FRH1 contained in any one of SEQ ID NOS: 210, 310, 410, 510, 610, 710, 810, 910, 1010, 1110, 1210, 1310, 1410, 1510, 1610, 1710, 1810, 1910, 2110, 2210, 2310, 2410, and 2510; and/or (iii) any one of SEQ ID NOS: 111, 211, 311, 411, 511, 611, 711, 811, 911, 1011, 1111, 1211, 1311, 1411, 1511, 1611, 1711, 1811, 1911, 2111, 2211, 2311, 2411, and 2511; (b) a VH framework region 2 (FRH2) comprising the amino acid sequence of: (i) the FRH2 contained in any one of Antibody Nos. V002-V019 and A001-A005; (ii) the FRH2 contained in any one of SEQ ID NOS: 210, 310, 410, 510, 610, 710, 810, 910, 1010, 1110, 1210, 1310, 1410, 1510, 1610, 1710, 1810, 1910, 2110, 2210, 2310, 2410, and 2510; (iii) WVRQAPGQRLEWX₁₈G (SEQ ID NO: 113), wherein X₁₈ is M or I; and/or (iv) any one of SEQ ID NOS: 213, 313, 413, 513, 613, 713, 813, 913, 1013, 1113, 1213, 1313, 1413, 1513, 1613, 1713, 1813, 1913, 2113, 2213, 2313, 2413, and 2513; (c) a VH framework region 3 (FRH3) comprising the amino acid sequence of: (i) the FRH3 contained in any one of Antibody Nos. V002-V019 and A001-A005; (ii) the FRH3 contained in any one of SEQ ID NOS: 210, 310, 410, 510, 610, 710, 810, 910, 1010, 1110, 1210, 1310, 1410, 1510, 1610, 1710, 1810, 1910, 2110, 2210, 2310, 2410, and 2510; and/or (iii) any one of SEQ ID NOS: 115, 215, 315, 415, 515, 615, 715, 815, 915, 1015, 1115, 1215, 1315, 1415, 1515, 1615, 1715, 1815, 1915, 2115, 2215, 2315, 2415, and 2515; and/or (d) a VH framework region 4 (FRH4) comprising the amino acid sequence of: (i) the FRH4 contained in any one of Antibody Nos. V002-V019 and A001-A005; (ii) the FRH4 contained in any one of SEQ ID NOS: 210, 310, 410, 510, 610, 710, 810, 910, 1010, 1110, 1210, 1310, 1410, 1510, 1610, 1710, 1810, 1910, 2110, 2210, 2310, 2410, and 2510; and/or (iii) any one of SEQ ID NOS: 117, 217, 317, 417, 517, 617, 717, 817, 917, 1017, 1117, 1217, 1317, 1417, 1517, 1617, 1717, 1817, 1917, 2117, 2217, 2317, 2417, and 2517; and/or (B) the VL polypeptide comprises: (a) a VL framework region 1 (FRL1) comprising the amino acid sequence of: (i) the FRL1 contained in any one of Antibody Nos. V002-V019 and A001-A005; (ii) the FRL1 contained in any one of SEQ ID NOS: 220, 320, 420, 520, 620, 720, 820,920, 1020, 1120, 1220, 1320, 1420, 1520, 1620, 1720, 1820, 1920, 2120, 2220, 2320, 2420, and 2520; (iii) DIVMX₁₉QSPDSLAVSLGERATINC (SEQ ID NO: 121), wherein X₁₉ is T or S; and/or (iv) any one of SEQ ID NOS: 221, 321, 421, 521, 621, 721, 821, 921, 1021, 1121, 1221, 1321, 1421, 1521, 1621, 1721, 1821, 1921, 2121, 2221, 2321, 2421, and 2521; (b) a VL framework region 2 (FRL2) comprising the amino acid sequence of: (i) the FRL2 contained in any one of Antibody Nos. V002-V019 and A001-A005; (ii) the FRL2 contained in any one of SEQ ID NOS: 220, 320, 420, 520, 620, 720, 820, 920, 1020, 1120, 1220, 1320, 1420, 1520, 1620, 1720, 1820, 1920, 2120, 2220, 2320, 2420, and 2520; and/or (iii) any one of SEQ ID NOS: 123, 223, 323, 423, 523, 623, 723, 823, 923, 1023, 1123, 1223, 1323, 1423, 1523, 1623, 1723, 1823, 1923, 2123, 2223, 2323, 2423, and 2523; (c) a VL framework region 3 (FRL3) comprising the amino acid sequence of: (i) the FRL3 contained in any one of Antibody Nos. V002-V019 and A001-A005; (ii) the FRL3 contained in any one of SEQ ID NOS: 220, 320, 420, 520, 620, 720, 820, 920, 1020, 1120, 1220, 1320, 1420, 1520, 1620, 1720, 1820, 1920, 2120, 2220, 2320, 2420, and 2520; and/or (iii) any one of SEQ ID NOS: 125, 225, 325, 425, 525, 625, 725, 825, 925, 1025, 1125, 1225, 1325, 1425, 1525, 1625, 1725, 1825, 1925, 2125, 2225, 2325, 2425, and 2525; and/or (d) a VL framework region 4 (FRL4) comprising the amino acid sequence of: (i) the FRL4 contained in any one of Antibody Nos. V002-V019 and A001-A005; (ii) the FRL4 contained in any one of SEQ ID NOS: 220, 320, 420, 520, 620, 720, 820, 920, 1020, 1120, 1220, 1320, 1420, 1520, 1620, 1720, 1820, 1920, 2120, 2220, 2320, 2420, and 2520; and/or (iii) any one of SEQ ID NOS: 127, 227, 327, 427, 527, 627, 727, 827, 927, 1027, 1127, 1227, 1327, 1427, 1527, 1627, 1727, 1827, 1927, 2127, 2227, 2327, 2427, and 2527 or the antibody or antigen-binding fragment may comprise a VH and/or a VL that comprises any combination of the foregoing VH and VL framework regions.

In some embodiments, in the anti-CD3 antibody or antigen-binding fragment may comprise: (i) the FRH1, the FRH2, the FRH3, the FLRH4, the FRL1, the FRL2, the FRL3, and the FRL4 contained in any one of Antibody Nos. A001, V002-V007, A003-A005, and V014-V019; and/or (ii) a FR-H1 comprising any one of SEQ ID NOS: 111, 211, 311, 411, 511, 611, 711, 1411, 1511, 1611, 1711, 1811, 1911, 2111, 2311, 2411, and 2511, a FR-H2 comprising any one of SEQ ID NOS: 213, 313, 413, 513, 613, 713, 1413, 1513, 1613, 1713, 1813, 1913, 2113, 2313, 2413, and 2513, a FR-H3 comprising any one of SEQ ID NOS: 115, 215, 315, 415, 515, 615, 715, 1415, 1515, 1615, 1715, 1815, 1915, 2115, 2315, 2415, and 2515, a FR-H4 comprising any one of SEQ ID NOS: 117, 217, 317, 417, 517, 617, 717, 1417, 1517, 1617, 1717, 1817, 1917, 2117, 2317, 2417, and 2517, a FR-L1 comprising any one of SEQ ID NOS: 221, 321, 421, 521, 621, 721, 1421, 1521, 1621, 1721, 1821, 1921, 2121, 2321, 2421, and 2521, a FR-L2 comprising any one of SEQ ID NOS: 123, 223, 323, 423, 523, 623, 723, 1423, 1523, 1623, 1723, 1823, 1923, 2123, 2323, 2423, and 2523, a FR-L3 comprising any one of SEQ ID NOS: 125, 225, 325, 425, 525, 625, 725, 1425, 1525, 1625, 1725, 1825, 1925, 2125, 2325, 2425, and 2525, and a FR-L4 comprising any one of SEQ ID NOS: 127, 227, 327, 427, 527, 627, 727, 1427, 1527, 1627, 1727, 1827, 1927, 2127, 2327, 2427, and 2527.

In certain embodiments, in the anti-CD3 antibody or antigen-binding fragment may comprise: (i) the FRH1, the FRH2, the FRH3, the FLRH4, the FRL1, the FRL2, the FRL3, and the FRL4 contained in any one of Antibody Nos. A002 and V008-V013; and/or (ii) a FR-H1 comprising any one of SEQ ID NOS: 111, 811, 911, 1011, 1111, 1211, 1311, and 2211, a FR-H2 comprising any one of SEQ ID NOS: 813, 913, 1013, 1113, 1213, 1313, and 2213, a FR-H3 comprising any one of SEQ ID NOS: 115, 815, 915, 1015, 1115, 1215, 1315, and 2215, a FR-H4 comprising any one of SEQ ID NOS: 117, 817, 917, 1017, 1117, 1217, 1317, and 2217, a FR-L1 comprising any one of SEQ ID NOS: 821, 921, 1021, 1121, 1221, 1321, and 2221, a FR-L2 comprising any one of SEQ ID NOS: 123, 823, 923, 1023, 1123, 1223, 1323, and 2223, a FR-L3 comprising any one of SEQ ID NOS: 125, 825, 925, 1025, 1125, 1225, 1325, and 2225, and a FR-L4 comprising any one of SEQ ID NOS: 127, 827, 927, 1027, 1127, 1227, 1327, and 2227.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V002; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 211, 212, 213, 214, 215, 216, 217, 221, 222, 223, 224, 225, 226, and 227, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V003; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 311, 312, 313, 314, 315, 316, 317, 321, 322, 323, 324, 325, 326, and 327, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V004; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 411, 412, 413, 414, 415, 416, 417, 421, 422, 423, 424, 425, 426, and 427, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V005; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 511, 512, 513, 514, 515, 516, 517, 521, 522, 523, 524, 525, 526, and 527, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V006; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 611, 612, 613, 614, 615, 616, 617, 621, 622, 623, 624, 625, 626, and 627, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V007; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 711, 712, 713, 714, 715, 716, 717, 721, 722, 723, 724, 725, 726, and 727, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V008; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 811, 812, 813, 814, 815, 816, 817, 821, 822, 823, 824, 825, 826, and 827, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V009; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 911, 912, 913, 914, 915, 916, 917, 921, 922, 923, 924, 925, 926, and 927, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V010; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1011, 1012, 1013, 1014, 1015, 1016, 1017, 1021, 1022, 1023, 1024, 1025, 1026, and 1027, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V011; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1111, 1112, 1113, 1114, 1115, 1116, 1117, 1121, 1122, 1123, 1124, 1125, 1126, and 1127, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V012; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1211, 1212, 1213, 1214, 1215, 1216, 1217, 1221, 1222, 1223, 1224, 1225, 1226, and 1127, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V013; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1311, 1312, 1313, 1314, 1315, 1316, 1317, 1321, 1322, 1323, 1324, 1325, 1326, and 1327, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V014; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1411, 1412, 1413, 1414, 1415, 1416, 1417, 1421, 1422, 1423, 1424, 1425, 1426, and 1427, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V015; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1511, 1512, 1513, 1514, 1515, 1516, 1517, 1521, 1522, 1523, 1524, 1525, 1526, and 1527, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V016; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1611, 1612, 1613, 1614, 1615, 1616, 1617, 1621, 1622, 1623, 1624, 1625, 1626, and 1627, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V017; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1711, 1712, 1713, 1714, 1715, 1716, 1717, 1721, 1722, 1723, 1724, 1725, 1726, and 1727, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V018; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1811, 1812, 1813, 1814, 1815, 1816, 1817, 1821, 1822, 1823, 1824, 1825, 1826, and 1827, respectively.

In particular embodiments, in the anti-CD3 antibody or antigen-binding fragment may comprise: (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V019; or (ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1911, 1912, 1913, 1914, 1915, 1916, 1917, 1921, 1922, 1923, 1924, 1925, 1926, and 1927, respectively.

In some embodiments, the antibody or antigen-binding fragment may include: a VH including an amino acid sequence selected from any of those listed in Table 1A or an amino acid sequence at least 80%, at least 85%, at least 90%, at least 92%, at least 94%, at least 95%, at least 96%, at least 97%, at least 98%, at least 99%, or 100% identical thereto; and/or a VL including an amino acid sequence selected from any of those listed in Table 1B or an amino acid sequence at least 80%, at least 85%, at least 90%, at least 92%, at least 94%, at least 95%, at least 96%, at least 97%, at least 98%, at least 99%, or 100% identical thereto.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 210; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 220.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 310; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 320.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 410; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 420.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 510; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 520.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 610; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 620.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 710; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 720.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 810; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 820.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 910; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 920.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1010; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1020.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1110; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1120.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1210; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1220.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1310; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1320.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1410; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1420.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1510; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1520.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1610; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1620.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1710; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1720.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1810; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1820.

In certain embodiments, (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1910; and/or (B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1920.

In particular embodiments, the anti-CD3 antibody or antigen-binding fragment may comprise a VH polypeptide and a VL polypeptide comprising the amino acid sequences of: SEQ ID NOS: 210 and 220, respectively. In particular embodiments, the anti-CD3 antibody or antigen-binding fragment may comprise a VH polypeptide and a VL polypeptide comprising the amino acid sequences of: SEQ ID NOS: 310 and 320, respectively. In particular embodiments, the anti-CD3 antibody or antigen-binding fragment may comprise a VH polypeptide and a VL polypeptide comprising the amino acid sequences of: SEQ ID NOS: 410 and 420, respectively. In particular embodiments, the anti-CD3 antibody or antigen-binding fragment may comprise a VH polypeptide and a VL polypeptide comprising the amino acid sequences of: SEQ ID NOS: 510 and 520, respectively. In particular embodiments, the anti-CD3 antibody or antigen-binding fragment may comprise a VH polypeptide and a VL polypeptide comprising the amino acid sequences of: SEQ ID NOS: 610 and 620, respectively. In particular embodiments, the anti-CD3 antibody or antigen-binding fragment may comprise a VH polypeptide and a VL polypeptide comprising the amino acid sequences of: SEQ ID NOS: 710 and 720, respectively. In particular embodiments, the anti-CD3 antibody or antigen-binding fragment may comprise a VH polypeptide and a VL polypeptide comprising the amino acid sequences of: SEQ ID NOS: 810 and 820, respectively. In particular embodiments, the anti-CD3 antibody or antigen-binding fragment may comprise a VH polypeptide and a VL polypeptide comprising the amino acid sequences of: SEQ ID NOS: 910 and 920, respectively. In particular embodiments, the anti-CD3 antibody or antigen-binding fragment may comprise a VH polypeptide and a VL polypeptide comprising the amino acid sequences of: SEQ ID NOS: 1010 and 1020, respectively. In particular embodiments, the anti-CD3 antibody or antigen-binding fragment may comprise a VH polypeptide and a VL polypeptide comprising the amino acid sequences of: SEQ ID NOS: 1110 and 1120, respectively. In particular embodiments, the anti-CD3 antibody or antigen-binding fragment may comprise a VH polypeptide and a VL polypeptide comprising the amino acid sequences of: SEQ ID NOS: 1210 and 1220, respectively. In particular embodiments, the anti-CD3 antibody or antigen-binding fragment may comprise a VH polypeptide and a VL polypeptide comprising the amino acid sequences of: SEQ ID NOS: 1310 and 1320, respectively. In particular embodiments, the anti-CD3 antibody or antigen-binding fragment may comprise a VH polypeptide and a VL polypeptide comprising the amino acid sequences of: SEQ ID NOS: 1410 and 1420, respectively. In particular embodiments, the anti-CD3 antibody or antigen-binding fragment may comprise a VH polypeptide and a VL polypeptide comprising the amino acid sequences of: SEQ ID NOS: 1510 and 1520, respectively. In particular embodiments, the anti-CD3 antibody or antigen-binding fragment may comprise a VH polypeptide and a VL polypeptide comprising the amino acid sequences of: SEQ ID NOS: 1610 and 1620, respectively. In particular embodiments, the anti-CD3 antibody or antigen-binding fragment may comprise a VH polypeptide and a VL polypeptide comprising the amino acid sequences of: SEQ ID NOS: 1710 and 1720, respectively. In particular embodiments, the anti-CD3 antibody or antigen-binding fragment may comprise a VH polypeptide and a VL polypeptide comprising the amino acid sequences of: SEQ ID NOS: 1810 and 1820, respectively. In particular embodiments, the anti-CD3 antibody or antigen-binding fragment may comprise a VH polypeptide and a VL polypeptide comprising the amino acid sequences of: SEQ ID NOS: 1910 and 1920, respectively.

In some embodiments, the anti-CD3 antibody or antigen-binding fragment may comprise one or more of an antibody constant region, a CH1 domain, a hinge, a CH2 domain, and/or a CH3 domain, which optionally are individually of, or derived from an IgG or human IgG, further optionally of or derived from a human IgG1, IgG4, IgG2, or IgG3.

In some embodiments, the anti-CD3 antibody or antigen-binding fragment may comprise a fragment crystallizable (Fe) region, optionally of, or derived from, a human IgG1, IgG4, IgG2, or IgG3.

In certain embodiments, when the anti-CD3 antibody or antigen-binding fragment comprise one or more of an antibody constant region, constant domain, and/or an Fe region of, or derived from a human IgG1, the antibody constant region, constant domain, and/or the Fe region may comprise one or more of the following amino acid modifications: N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V305I, P396L, S239D, 1332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y M428L, N434S, L328F, M252Y, S254T, T256E, or any combination thereof, according to EU numbering.

In certain embodiments, when the anti-CD3 antibody or antigen-binding fragment comprises one or more of an antibody constant region, constant domain, and/or an Fe region of, or derived from a human IgG4, the antibody constant region, constant domain, and/or the Fe region may comprise one or more of the following amino acid modifications: E233P, F234V, L235A, G237A, E318A, S228P, L236E, S241P, L248E, T394D, M252Y, S254T, T256E, N297A, N297Q, or any combination thereof, according to EU numbering.

In certain embodiments, when the anti-CD3 antibody or antigen-binding fragment comprises one or more of an antibody constant region, constant domain, and/or Fe region of, or derived from a human IgG2, the antibody constant region, constant domain, and/or the Fe region may comprise one or more of the following amino acid modifications: P238S, V234A, G237A, H268A, H268Q, H268E, V309L, N297A, N297Q, A330S, P331S, C232S, C233S, M252Y, S254T, T256E, or any combination thereof, according to EU numbering.

In certain embodiments, when the anti-CD3 antibody or antigen-binding fragment comprises one or more of an antibody constant region, constant domain, and/or Fe region of, or derived from a human IgG3, the antibody constant region, constant domain, and/or Fe region may comprise E235Y, according to EU numbering.

In some embodiments, the anti-CD3 antibody or antigen-binding fragment may comprise an IgG, IgA, IgE, IgD, or IgM, optionally IgG1, IgG4, IgG2, or IgG3.

In some embodiments, the anti-CD3 antibody or antigen-binding fragment may comprise an antibody fragment selected from the group consisting of: a fragment, antigen-binding (Fab) region; an Fab₂; an Fab₃; an Fab′ fragment; an F(ab′)₂; a variable fragment (Fv); a single-chain Fv (scFv) fragment; a diabody; a triabody; a minibody; a scFv-Fc; a scFv2-Fc2; scFv-IgG; a monovalent IgG (or a half IgG); and/or a chimeric antigen receptor (CAR) comprising an antigen-binding region comprising said VH polypeptide and/or said VL polypeptide, a transmembrane domain, and at least one intracellular signaling domain (optionally derived from a T-cell receptor, further optionally CD3ζ).

In some embodiments, the anti-CD3 antibody or antigen-binding fragment may bind to (i) human CD3, (ii) non-human primate CD3, optionally monkey, further optionally cynomolgus and/or rhesus CD3, and/or (iii) rodent CD3, optionally mouse CD3. In some embodiments, the anti-CD3 antibody or antigen-binding fragment may bind to CD3εδ. In some embodiments, the anti-CD3 antibody or antigen-binding fragment may comprise or may be comprised in a multispecific (e.g., bispecific, trispecific, tetraspecific, etc) antibody or antibody fragment having at least (a) a first antigen-binding region specific to CD3, comprising said VH polypeptide and/or said VL polypeptide, and (b) a second antigen-binding region. In certain embodiments, the multispecific antibody or antibody fragment may include or comprise one or more scFvs.

In certain embodiments, the second antigen-binding region is specific to or specifically binds to an oncology target; a target molecule expressed on cancer cells; an immune-oncology target; a target molecule expressed on immune cells; a neurodegenerative disease targets; an autoimmune disorder target (optionally a self-reactive immune molecule or a target molecule expressed on an immune cell expressing a self-reactive immune molecule); an infectious disease target; an inflammatory disease target (optionally an inflammatory cytokine or chemokine or a receptor thereof); an infectious disease target (optionally a target molecule of a virus, bacterium, or a fungus); a target molecule expressed on infected cells (optionally infected with a virus, a bacterium, or fungus); a metabolic disease target; a cognitive disorder target; a blood-brain barrier target; or a blood disease target.

In certain embodiments, the second antigen-binding region is specific to or specifically binds to one or more of: 17-IA, 4-1BB, 4Dc, 6-keto-PGF1a, 8-iso-PGF2a, 8-oxo-dG, Al Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM 15, ADAM 17/T ACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BFM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL 14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-1 (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxin1, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin, alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, a metalloprotease, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibiting substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1 Apo-2, DR4), TNFRSFIOB (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSFIIB (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF 18 (GITR AITR), TNFRSF19 (TROY TAJ, TRADE), TNFRSF19L (RELT), TNFRSFIA (TNF R1 CD120a, p55-60), TNFRSFIB (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF 5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TLIA/VEGI), TNFSF18 (GITR Ligand AITR Ligand, TL6), TNFSFIA (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (fit-1), VEGF, VEGFR, VEGFR-3 (flt-4), VEGI, VFM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3), a hormone receptor, and a growth factor.

In certain embodiments, the second antigen-binding region is specific to or specifically binds to one or more of: BCMA, CTLA4 (cytotoxic T lymphocyte antigen-4), PD1 (programmed cell death protein 1), PD-L1 (programmed cell death ligand 1), LAG-3 (lymphocyte activation gene-3), TIM-3, CD20, CD2, CD19, Her2, EGFR, EpCAM, FcyRIIIa (CD16), FcyRIIa (CD32a), FcyRIIb (CD32b), FcyRI (CD64), Toll-like receptors (TLRs), TLR4, TLR9, cytokines, IL-2, IL-5, IL-13, IL-6, IL-17, IL-12, IL-23, TNFα, TGFβ, cytokine receptors, IL-2R, chemokines, chemokine receptors, growth factors, VEGF, and HGF.

In certain embodiments, the multispecific antibody or antibody fragment further comprises a third antigen-binding region. In certain embodiments, the multispecific antibody or antibody fragment is trispecific.

In certain embodiments, the multispecific antibody or antibody fragment comprises a multispecific format selected from the group consisting of: Fab-Fc-scFv, scFv2-Fc2, scFv-IgG, “bottle-opener”, Mab-scFv, Mab-Fv, Dual scFv, central Fv, central scFv, one-arm central scFv, Fab-Fab, Fab-Fv, mAb-Fv, mAb-Fab, DART, BiTE, common light chain-IgG, TandAb, Cross-Mab, SEED, BEAT, TrioMab, and DuetMab.

In certain embodiments, the multispecific antibody or antibody fragment comprises one or more of the following: at least one CLκ-preferring variant CH1 domain, optionally a CLκ-preferring variant CH1 domain described in WO2021067404; at least one CLλ-preferring variant CH1 domain, optionally a CLλ-preferring variant CH1 domain described in WO2021067404; at least one pair of a variant CH1 domain and a variant CL domain which preferentially pair with each other, optionally a pair described in WO2022150787; and/or at least one pair of a variant CH3 domain and another variant CH3 domain which preferentially pair with each other, optionally a pair described in WO2022150785.

In some embodiments, the anti-CD3 antibody or antigen-binding fragment may exhibit a reduced PSR score, optionally relative to its parent antibody, optionally any one or more of or all of Antibody Nos. A001, A002, A003, A004, and/or A005.

In some embodiments, the anti-CD3 antibody or antigen-binding fragment may exhibit a PSR score lower than 0.33, lower than about 0.30, lower than about 0.20, lower than about 0.15, lower than about 0.12, lower than about 0.10, lower than about 0.08, lower than about 0.06, lower than about 0.05, lower than about 0.04, lower than about 0.03, lower than about 0.02, or lower than about 0.01. In some embodiments, the anti-CD3 antibody or antigen-binding fragment may exhibit a PSR score of about ≥0.10 and <0.33 or a PSR score of about <0.10.

In some embodiments, the anti-CD3 antibody or antigen-binding fragment may exhibit a hydrophobic interaction chromatography (HIC) retention time of < about 10.5 minutes, < about 10.0 minutes, < about 9.5 minutes, < about 9.0 minutes, or < about 8.5 minutes.

In some embodiments, the anti-CD3 antibody or antigen-binding fragment may exhibit (i) a reduced affinity-capture self-interaction nanoparticle spectroscopy (AC-SINS) Δλmax relative to any one or more of or all of Antibody Nos. A001-A005; (ii) an AC-SINS Δλmax lower than about 20.0 nm, lower than about 15.0 nm, lower than about 12.0 nm, lower than about 10.0 nm, lower than about 8.0 nm, lower than about 6.0 nm, lower than about 5.0 nm, lower than about 4.0 nm, lower than about 3.0 nm, lower than about 2.0 nm, or lower than about 1.0 nm; and/or (iii) an AC-SINS Δλmax of about ≥5.0 nm and <20.0 or an AC-SINS Δλmax of about <5.0 m.

In some embodiments, the anti-CD3 antibody or antigen-binding fragment may exhibit (i) a higher dynamic light scattering (DLS) diffusion interaction parameter (kD) relative to any one or more of or all of Antibody Nos. A001-A005; (ii) a DLS kD of about 5 mL/g or higher, about 10 mL/g or higher, about 15 mL/g or higher, about 20 mL/g or higher, about 25 mL/g or higher, about 30 mL/g or higher, or about 35 mL/g or higher; and/or (iii) a DLS kD of between about 10 mL/g and about 40 mL/g, between about 15 mL/g and about 40 mL/g, or between about 20 mL/g and about 40 mL/g. In certain embodiments, the DLS kD is measured using a 10 mM histidine buffer, optionally of pH 6.0.

In some embodiments, the anti-CD3 antibody or antigen-binding fragment may comprise: (i) a melting temperature (Tm) of about 65° C. or higher; or (ii) a Tm of about 70° C. or higher, about 75° C. or higher, about 80° C. or higher; and/or (iii) a Tm of between about 70° C. and about 90° C., between about 75° C. and about 85° C. In certain embodiments, the anti-CD3 antigen-binding fragment may be a Fab.

In some embodiments, the anti-CD3 antibody or antigen-binding fragment may not significantly aggregate or multimerize. In certain embodiments, the anti-CD3 antibody or antigen-binding fragment may exhibit (i) % monomer of about 90% or higher, about 95% or higher, about 97% or higher, about 98% or higher, about 99% or higher, by size exclusion chromatography (SEC); and/or (ii) % monomer between about 90% and about 100%, between about 95% and about 100%, between about 97% and about 100%, between about 98% and about 100%, between about 99% and about 100%, by SEC. In certain embodiments, the anti-CD3 antigen-binding fragment may be a Fab or an IgG, optionally IgG1.

In certain embodiments, the anti-CD3 antibody or antigen-binding fragment may not significantly aggregate or multimerize in an acidic condition or when exposed to acidic stress, optionally in a pH of about 6 or lower, about 5 or lower, about 4 or lower, or about 3.5 or lower. In particular embodiments, aggregation or multimerization in an acidic condition may be assessed by SEC as described above and herein.

In some embodiments, the anti-CD3 antibody or antigen-binding fragment may not exhibit significant amount of heavy-light chain mispairing, optionally as determined by absence of a peak of unpaired heavy chain and/or a peak of unpaired light chain measured by liquid chromatography-mass spectrometry (LC-MS), optionally when the anti-CD3 antibody or antigen-binding fragment is produced recombinantly, further optionally in mammalian cells, yet further optionally Chinese hamster ovary (CHO) cells.

In certain embodiments, the anti-CD3 antibody or antigen-binding fragment may bind to CD3-expressing cells, optionally wherein the CD3-expressing cells are: (i) T cells; (ii) human cells, non-human primate (optionally monkey, further optionally cynomolgus and/or rhesus) cells, and/or rodent (optionally mouse) cells; and/or (iii) primary cells or cell line cells.

In certain embodiments, the anti-CD3 antibody or antigen-binding fragment may to CD3 with an equilibrium dissociation constant (Kd) of: (i) about 1.0×10⁶ M or lower, about 5.0×10⁷ M or lower, about 1.0×10⁷ M or lower, about 5.0×10⁸ M or lower, about 1.0×10⁸ M or lower, about 5.0×10⁹ M or lower, about 1.0×10⁹ M or lower, about 5.0×10¹⁰ M or lower, or about 1.0×10¹⁰ M or lower; (ii) between about 1.0×10⁶ M and about 1.0×10¹¹ M, between about 1.0×10⁷ M and about 1.0×10¹¹ M, or between about 1.0×10⁸ M and about 1.0×10¹⁰ M. In some cases, such binding may be measured by surface plasmon resonance (SPR), optionally using a BIACORE® system, or measured by bio-layer interferometry (BLI), further optionally using an Octet® system. In some cases, the CD3 is human, non-human primate (optionally monkey, further optionally cynomolgus and/or rhesus), and/or rodent (optionally mouse) CD3.

In certain embodiments, the anti-CD3 antibody or antigen-binding fragment, upon binding to CD3 on a cell, may elicit activation of and/or enhance cytotoxic function(s) of the cell, optionally a T cell.

In certain embodiments, the anti-CD3 antibody or antigen-binding fragment upon binding to CD3 on a cell, may not elicit cytokine production by the cell to levels capable of inducing cytokine release syndrome (CRS).

In certain embodiments, the anti-CD3 antibody or antigen-binding fragment may comprise or may be comprised in a multispecific antibody or antibody fragment having at least (a) a first antigen-binding region specific to CD3, comprising said VH polypeptide and/or said VL polypeptide, and (b) a second antigen-binding region specific to a second antigen; and upon binding to (i) CD3 on a first cell, optionally a T cell, and (ii) the second antigen expressed on a second cell, the first cell exhibits cytotoxicity to the second cell.

One aspect of the present disclosure provides nucleic acids (e.g., one or more nucleic acids).

In some embodiments, a nucleic acid (e.g., one or more isolated or recombinant nucleic acids, such as a nucleic acid or a combination of two or more nucleic acids) is provided and may encode any of the anti-CD3 antibodies or antigen-binding fragments according to any of the embodiments described herein. Such nucleic acids may include mRNA, e.g., for delivery to cells and expression on anti-CD3 antibodies or antigen-binding fragments.

In some embodiments, said nucleic acid may comprise: (A) a VH polypeptide-encoding nucleic acid sequence at least 80%, at least 85%, at least 90%, at least 92%, at least 94%, at least 95%, at least 96%, at least 97%, at least 98%, at least 99%, or 100% identical to SEQ ID NO: 250, 350, 450, 550, 650, 750, 850, 950, 1050, 1150, 1250, 1350, 1450, 1550, 1650, 1750, 1850, or 1950 or mRNA version of any of the foregoing; and/or (B) a VL polypeptide-encoding nucleic acid sequence at least 80%, at least 85%, at least 90%, at least 92%, at least 94%, at least 95%, at least 96%, at least 97%, at least 98%, at least 99%, or 100% identical to SEQ ID NO: 260, 360, 460, 560, 660, 760, 860, 960, 1060, 1160, 12560, 1360, 1460, 1560, 1660, 1760, 1860, or 1960 or mRNA version of any of the foregoing.

In certain embodiments, said nucleic acid may comprise: the VH polypeptide-encoding and VL polypeptide-encoding nucleic acid sequences of: (I) SEQ ID NOS: 250 and 260, respectively; (II) SEQ ID NOS: 350 and 360, respectively; (III) SEQ ID NOS: 450 and 460, respectively; (IV) SEQ ID NOS: 550 and 560, respectively; (V) SEQ ID NOS: 650 and 660, respectively; (VI) SEQ ID NOS: 750 and 760, respectively; (VII) SEQ ID NOS: 850 and 860, respectively; (VIII) SEQ ID NOS: 950 and 960, respectively; (IX) SEQ ID NOS: 1050 and 1060, respectively; (X) SEQ ID NOS: 1150 and 1160, respectively; (XI) SEQ ID NOS: 1250 and 1260, respectively; (XII) SEQ ID NOS: 1350 and 1360, respectively; (XIII) SEQ ID NOS: 1450 and 1460, respectively; (XIV) SEQ ID NOS: 1550 and 1560, respectively; (XV) SEQ ID NOS: 1650 and 1660, respectively; (XVI) SEQ ID NOS: 1750 and 1760, respectively; (XVII) SEQ ID NOS: 1850 and 1860, respectively; or (XVIII) SEQ ID NOS: 1950 and 1960, respectively; or mRNA version of any of the foregoing.

Another aspect of the present disclosure provides vectors (e.g., one or more vectors, such as a vector or a combination of two or more vectors) and/or constructs that includes any of the nucleic acids according to the present disclosure.

In certain embodiments, the vectors may be expression vectors. In certain embodiments, the vectors may comprise a plasmid, a viral vector (optionally adenoviral, lentiviral, or retroviral), a lipid-based vector, a self-replicating RNA vector, a virus-like particle, a polymer-based vector, and/or a nanoparticle, optionally a lipid-based nanoparticle.

Another aspect of the present disclosure provides cells comprising, transfected with, transformed with, or transduced with any of the nucleic acids and/or vectors and/or constructs.

In some embodiments, the cell is (i) a mammalian cell, optionally a human, non-human primate, monkey, rabbit, rodent, hamster, rat, or mouse cell or a yeast cell or (ii) a non-mammalian, optionally plant, bacterial, fungal, yeast, protozoa, or an insect cell.

In certain embodiments, the isolated or recombinant cell is an immune cell or a hybridoma.

Another aspect of the present disclosure provides pharmaceutical compositions that include: (i) an anti-CD3 antibody or antigen-binding fragment according to any of the embodiments described herein, a nucleic acid according to any of the embodiments described herein, a vector according to any of the embodiments described herein, and/or a cell according to any of the embodiments described herein optionally including an antibody or antigen-binding fragment according to any of the embodiments described herein; and (ii) a pharmaceutically acceptable carrier and/or an excipient.

Another aspect of the present disclosure provides in vivo methods using at least one of the anti-CD3 antibodies or antigen-binding fragments or nucleic acids encoding or cells which comprise and/or express any of the anti-CD3 antibodies or antigen-binding fragments or nucleic acids encoding them disclosed herein.

In some embodiments, the method is a method of treating a subject in need of such treatment. In some embodiments, the method is a method of treating or preventing a disease, a disorder, or a condition in a subject (e.g., in a mammal) in need of such treatment. In any of such methods, in certain embodiments, the method may comprise: administering an effective amount of: (i) an anti-CD3 antibody or antigen-binding fragment according to any of the embodiments described herein; (ii) a nucleic acid according to any of the embodiments described herein; (iii) a vector according to any of the embodiments described herein; (iv) an isolated or recombinant cell according to any of the embodiments described herein (optionally, an immune cell, T cell, and/or a natural killer (NK) cell); and/or (v) the pharmaceutical composition according to any of the embodiments described herein.

In some embodiments, the method is a method of eliciting cytotoxicity to a cell expressing a target molecule of interest. In certain embodiments, the method may comprise administering to the subject an effective amount of: (i) any of the multispecific antibodies or antibody fragments described herein; (ii) a nucleic acid encoding such a multispecific antibody or antibody fragment; (iii) a vector comprising such a nucleic acid; (iv) one or more isolated or recombinant cells comprising, transfected with, transformed with, or transduced with such a nucleic acid or vector; and/or (v) the pharmaceutical composition comprising (A) such a multispecific antibody or antibody fragment, such a nucleic acid, such a vector, such one or more cells and (B) a pharmaceutically acceptable carrier and/or excipient.

In certain embodiments of any of the methods described above, the subject may be (i) a mammal, optionally a human, a non-human primate, a monkey, a horse, a cow, sheep, a goat, a pig, a dog, a cat, a rabbit, a rodent, a hamster, a rat, or a mouse; or (ii) a non-mammalian vertebrate, optionally a bird, fish, an amphibian, or a reptile. In certain embodiments, the subject may comprise or have a risk of developing a disease, disorder, or a condition. In certain embodiments of any of the methods described above, the method may further comprise administering to the subject an additional agent (e.g., any of those described herein), optionally an adjuvant or a therapeutic agent.

In certain embodiments of any of the methods described above, the disorder may include one or more of a proliferative disorder, an oncological disorder, cancer or a neoplastic condition, an immuno-oncological disorder, a neurological disorder, a neurodegenerative disorder, an infectious disease, and an autoimmune disorder, an autoimmune disorder, or another disease.

In certain embodiments of any of the methods described above, the disease, disorder, or condition may comprise cancer.

In particular embodiments, the cancer may be a solid cancer, optionally chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof.

In particular embodiments, the cancer may be a liquid cancer, optionally chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmacytoma of bone, extraosseous plasmacytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

In certain embodiments of any of the methods described above, the disease, disorder, or condition may comprise an autoimmune or inflammatory disease.

In particular embodiments, the autoimmune or inflammatory disease may be psoriasis, rheumatoid arthritis, autoimmune arthritis, type I diabetes, systemic lupus erythematosus, myasthenia gravis, multiple sclerosis, scleroderma, inflammatory bowel disease, Crohn's disease, ulcerative colitis, Guillain-Barre syndrome, chronic inflammatory demyelinating polyneuropathy, pemphigus vulgaris, Sjogren syndrome, Addison disease, Behçet's disease, Schmidt syndrome, celiac disease, dermatomyositis, autoimmune vitiligo, Graves' disease, Hashimoto thyroiditis, Kawasaki disease, pernicious anemia, autoimmune vasculitis, or fibrosis.

In certain embodiments of any of the methods described above, the disease, disorder, or condition may comprise a neurodegenerative disease.

In particular embodiments, the neurodegenerative disease may be Alzheimer's disease, Huntington's disease, Parkinson's disease, amyotrophic lateral sclerosis, Friedreich ataxia, Lewy body disease, spinal muscular atrophy, motor neuron disease, multiple sclerosis, Batten disease, Creutzfeldt-Jakob disease.

In certain embodiments of any of the methods described above, the disease, disorder, or condition may comprise an infectious disease.

In particular embodiments, the infectious disease may be a viral, bacterial, fungal, yeast, protozoan, prion or parasitic disease. In some cases, the viral disease may be human immunodeficiency virus (HIV), hepatitis virus (optionally hepatitis A, B, or C virus), human papillomavirus (HPV), herpes simplex virus (HSV) (optionally HSV-1 or HSV-2), enterovirus, human cytomegalovirus, adenovirus, rhinovirus, Pox virus, Influenza virus, coronavirus (optionally MERS-CoV, SARS-CoV, or SARS-CoV-2, or common human coronavirus), norovirus, West Nile Virus, Zika virus, poliovirus, Ebola virus, or dengue virus (DENV) infection. In some cases, the bacterial disease may be Salmonella, Escherichia coli, Mycobacterium tuberculosis, methicillin-resistant Staphylococcus aureus (MRSA), Clostridium difficile, Streptococcus pneumoniae, Klebsiella pneumoniae, Pseudomonas aeruginosa, Helicobacter pylori, Neisseria gonorrhoeae, Vibrio vulnificus. In some cases, the fungal disease may be Aspergillosis, Candida, Candida auris, Cryptococcus neoformans, Pneumocystis jiroveccii, Mucoromycetes, Taloromyces, ringworm, Blastomyces, Coccidioides, Cryptococcus gattii, Histoplasma, Paracoccidioides, or Sporothrix infection.

One aspect of the present disclosure provides methods of manufacturing an anti-CD3 antibody or antigen-binding fragment according to any of the embodiments described herein.

In some embodiments, the method may comprise: (a) culturing cells comprising a nucleic acid encoding the anti-CD3 antibody or antigen-binding fragment in a condition that allows for expression of said anti-CD3 antibody or antigen-binding fragment, and (b) harvesting and purifying the anti-CD3 antibody or antigen-binding fragment from the cell culture from (a).

Another aspect of the present disclosure provides methods of manufacturing an isolated or recombinant cell according to any of the embodiments described herein or the population of such cells.

In some embodiments, the method may comprise: introducing a nucleic acid according to any of the embodiments described herein and/or a vector according to any of the embodiments described herein into one or more cells. In certain embodiments, the introducing may occur in vitro, ex vivo, or in vivo.

Any of the anti-CD3 antibodies and antigen-binding fragments according to the present disclosure, any of the nucleic acids according to the present disclosure, any of the vectors according to the present disclosure, any of the isolated or recombinant cells according to the present disclosure or a population of such cells, and/or any of the pharmaceutical compositions according to the present disclosure may be for use in medicine or in the preparation of a medicament for use in medicine.

Any of the anti-CD3 antibodies and antigen-binding fragments according to the present disclosure, any of the nucleic acids according to the present disclosure, any of the vectors according to the present disclosure, any of the isolated or recombinant cells according to the present disclosure or a population of such cells, and/or any of the pharmaceutical compositions according to the present disclosure may be for use in treating a disease, disorder, or condition, optionally any of the diseases, disorders, or conditions described herein.

The present disclosure further encompasses use of any of the anti-CD3 antibodies and antigen-binding fragments according to the present disclosure, any of the nucleic acids according to the present disclosure, any of the vectors according to the present disclosure, any of the isolated or recombinant cells according to the present disclosure or a population of such cells, and/or any of the pharmaceutical compositions according to the present disclosure for the manufacture of a medicament for treatment of a disease, disorder, or condition, optionally any of the diseases, disorders, or conditions described herein.

### BRIEF DESCRIPTION OF THE FIGURES

The foregoing and other objects, features, and advantages of particular embodiments of the disclosure will be apparent from the following description and illustrations in the accompanying figures.

FIG. 1 is a graph showing normalized PSR scores for anti-CD3 antibodies plotted against antibody K_{D} values.

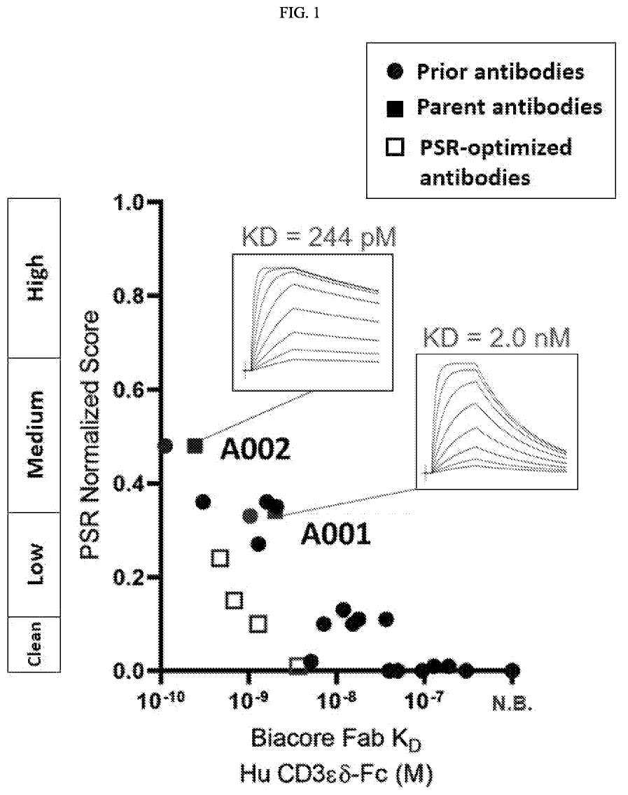

FIG. 2 is a graph showing AC-SINS values for anti-CD3 antibodies plotted against antibody K_{D} values.

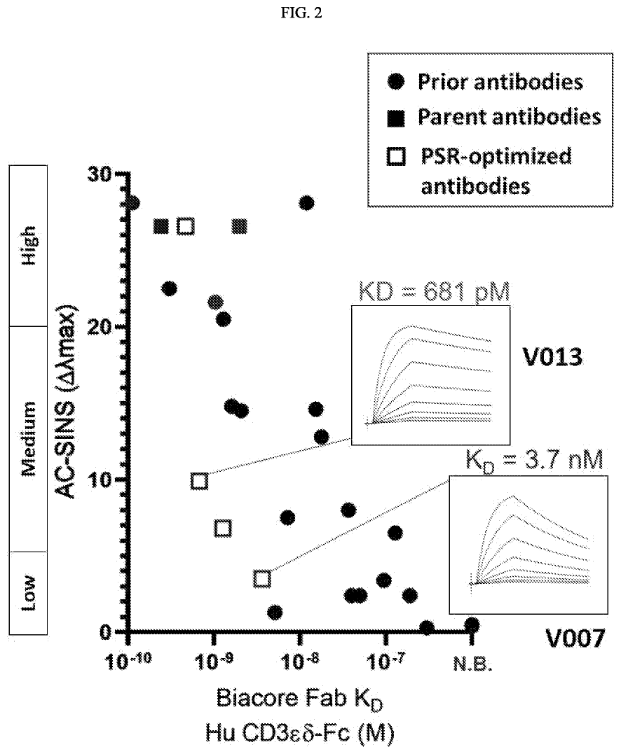

FIG. 3 is a graph showing normalized PSR scores for anti-CD3 antibodies plotted against antibody K_{D} values.

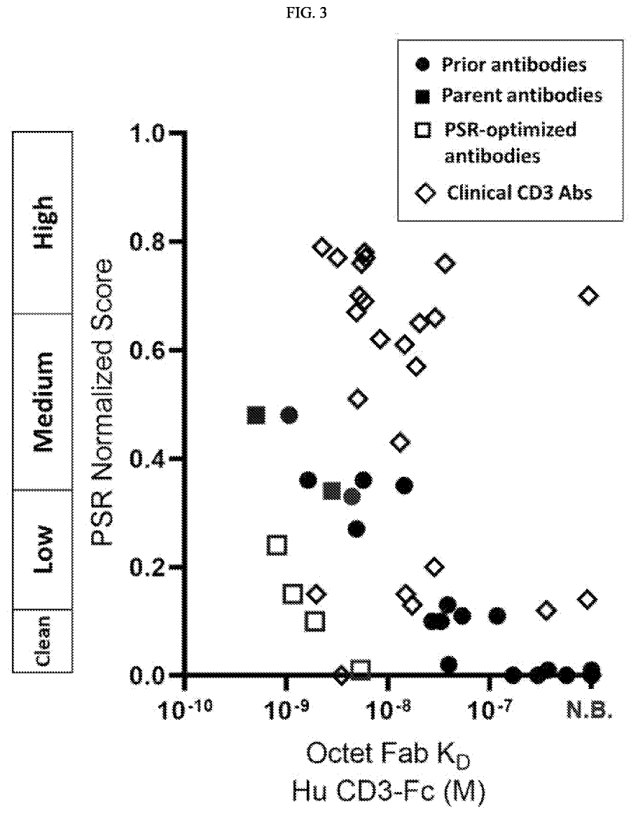

FIG. 4 is a graph showing AC-SINS values for anti-CD3 antibodies plotted against antibody K_{D} values.

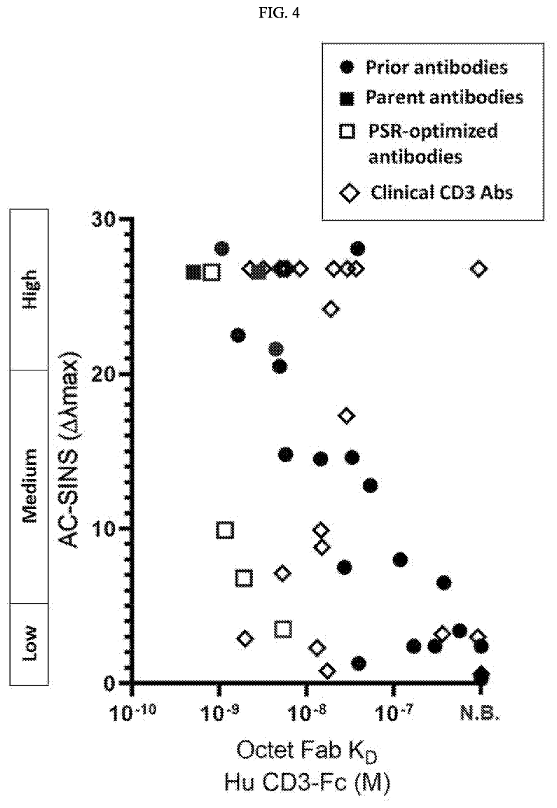

### DETAILED DESCRIPTION

The present disclosure generally relates to anti-CD3 antibodies and antigen-binding fragments which may have one or more improved developability properties.

Some embodiments of the disclosure may relate to anti-CD3 antibodies and antigen-binding fragments having reduced polyspecificity. Some embodiments of the disclosure may relate to anti-CD3 antibodies and antigen-binding fragments having reduced tendency of self-interaction. Some embodiments of the disclosure may relate to anti-CD3 antibodies and antigen-binding fragments having improved tolerance to low pH stress. Some embodiments of the disclosure may relate to anti-CD3 antibodies and antigen-binding fragments having reduced propensity to aggregate or multimerize. Some embodiments of the disclosure may relate to anti-CD3 antibodies and antigen-binding fragments having improved binding to target cells (CD3-expressing cells). Some embodiments of the disclosure may relate to anti-CD3 antibodies and antigen-binding fragments having increased binding preference at an acidic pH. Some embodiments of the disclosure may relate to anti-CD3 antibodies and antigen-binding fragments having improved cytokine release syndrome (CRS) risk profiles, in some instances due to pH-dependent antigen binding profiles.

### Developability of Anti-CD3 Antibodies and Antigen-Binding Fragments

The term “developable” or “developability” refers to the extent to which one or more polypeptides in a plurality of polypeptides possess desirable characteristics for manufacture, storage, off-target binding, etc., such as, e.g., desirable binding specificity, for example binding to a cognate antigen at desirable affinity and not significantly to non-cognate antigens; desirable expression, for example, in mammalian cells; solubility; viscosity; aggregation; chemical and/or physical stability; desirable shelf-life; melting temperature; pharmacokinetic profiles; circulation half-life; and clearance characteristics. Such characteristics may serve as indicia, independently, as combinations of sub-sets of such indicia, or in totality, for the likelihood that such one or more polypeptides may be successfully developed as a therapeutic candidate, and ultimately an approved drug. Generally, polypeptides with desirable developability characteristics possess one or more of relatively high solubility, relatively low viscosity, relatively low propensity for aggregation, relatively high chemical stability, relatively high physical stability, relatively long shelf life, relatively high melting temperature, relatively long circulation half-life, relatively slow rate of clearance, and the like. By contrast polypeptides with undesirable developability characteristics generally possess one or more of: relatively low solubility, relatively high viscosity, relatively high propensity for aggregation, relatively poor chemical stability, relatively poor physical stability, relatively short shelf life, relatively low melting temperature, relatively short circulation half-life, relatively fast rate of clearance, and the like.

The tendency of antibodies to bind multiple targets is referred to as “polyspecificity,” which, with target-specific therapeutic antibodies, may be associated with negative clinical outcomes. CD3 binding domains of the present disclosure may exhibit reduced polyspecificity [e.g., as assessed by interaction with polyspecific reagent (PSR)]. Such domains may be engineered from starting domains by substituting various domain amino acid residues with residues having charged side chains. Residues may be substituted with amino acid residues having negatively charged side chains, e.g., Asp and Glu residues. Residues selected for substitution may be selected from those not predicted to specifically interact with CD3 amino acid residues targeted by the CD3 binding domains.

Methods and assays that may be employed to ascertain the degree to which polypeptides, such as anti-CD3 antibodies and/or antigen-binding fragments as described herein, possess desirable developability characteristics are available in the art, and include, one or more of; polyspecificity reagent (PSR) assays (WO 2014/179363 and Xu et al., Protein Eng Des Sel, Vol. 26, pages 663-670 (2013)); SMP and SCP assays and the like; cross interaction chromatography (CIC); self-interaction chromatography (SIC); hydrophobic interaction chromatography (HIC); size exclusion chromatography (SEC); dynamic light scattering (DLS) spectroscopy; photon correlation spectroscopy; quasi-elastic light scattering, circular dichroism (CD), viscosity measurements; whole cell binding; tissue micro array methodologies; ELISA assays such as BVP ELISA assays; AC-SINS assays (Liu et al; MAbs, Vol. 6, pages 483-492 (2014); melting temperature (Tm) assays; differential scanning calorimetiy or differential scanning fluorometry (DSF); and the like (see, e.g., He et al., J. Pharm. Sci., Vol. 100(4), pp. 1330-1340 (2011); Wagner et al., Pharm. Develop. & Technol (posted online 2012; hyper-text transfer protocol: informahealthcare.com/doi/abs/10.3109/10837450.2011.649851); Hotzel et al., MAbs, Vol. 4(6), pages 753-7601 (2012); Weiqiang et al., J. Pharm. Sci., Vol. 101(5), pp. 1701-1720 (2012); Banks et al., J. Pharm. Sci., Vol. 101(8), pp. 2720-2732 (2012); Lie et al., J. Pharm. Sci., Vol. 94(9), pp. 1928-1948 (2005); and Payne et al., Biopolymers, Vol. 85(5), pp. 527-533 (2006)).

In some embodiments, antibodies that are identified as possessing decreased developability are so detected by virtue of their interaction with a PSR and, as such, are referred to as “polyspecific” polypeptides. Such polyspecific antibodies may be referred to as relatively “undevelopable” or relatively “non-developable”.

A “developability profile” refers to an index that may be assigned to antibodies upon assessing their developability. A developability profile is a measure or metric by which developability of anti-CD3 antibodies may be assessed, compared, and/or ranked. Such developability profiles serve as a measure of the degree of interaction of CD3 binders and antibodies comprising them. The degree of interaction may be assessed by any number of means available in the art that provides an output value that correlates with a strength or affinity of a polypeptide for a moiety to which it is bound. Exemplary means include flow cytometry means, such as fluorescence-activated cell sorting (FACS); enzyme-linked immunosorbent assay (ELISA); quantitative immunoaffinity assays or immunoprecipitation assays; mammalian two-hybrid or yeast two-hybrid assays, and the like. In the context of FACS, as demonstrated in the Examples, a degree of interaction between polypeptides in the plurality and the PSR may be ascertained by generating a mean fluorescence intensity (MFI) for each polypeptide-PSR interaction that is detected, and then ordering the MFI in either ascending or descending order, thereby ranking the polypeptides in the plurality according to the relative degree of interaction between each detected polypeptide and the PSR. Such a ranking provides for a ranking of polypeptides of the plurality such that those polypeptides possessing enhanced developability are readily ascertained, as are those polypeptides possessing decreased developability.

A developability profile may also take the form of a normalized score, for example, by normalizing developability of anti-CD3 antibodies described herein to the developability of a standard (or control) antibody, e.g., anti-HEL antibody.

In some embodiments, anti-CD3 antibodies or antigen-binding fragment as described herein may exhibit reduced polyspecificity or tendency to bind to multiple molecules or epitopes. In some embodiments, polyspecificity may be determined based on the poly-specificity reagent (PSR) score obtained by a PSR assay. In some embodiments, PSR scores may be determined as described in Examples. In some embodiments, anti-CD3 antibodies and/or antigen-binding fragments as described herein may display a PSR score of between about 0.0 and about 0.45. In some embodiments, the PSR is between about 0.0 and about 0.4. In some embodiments, the PSR is between about 0.0 and about 0.35. In some embodiments, the PSR is between about 0.0 and about 0.3. In some embodiments, the PSR is between about 0.0 and about 0.25. In some embodiments, the PSR is between about 0.0 and about 0.2. In some embodiments, the PSR is between about 0.0 and about 0.15. In some embodiments, the PSR is between about 0.0 and about 0.1. In some embodiments, 0.0≤PSR score <0.10 is considered as “clean PSR”. In some embodiments, 0.10≤PSR score <0.33 is considered as “low PSR”. In some embodiments, 0.33≤PSR score <0.66 is considered as “medium PSR”. In some embodiments, 0.66≤PSR score ≤1.00 is considered as “high PSR”. In some embodiments, a high PSR score is indicative of decreased (or poor) developability. Generally, the lower the PSR score the more favorable the developability of the antibody.

In some embodiments, anti-CD3 antibodies or antigen-binding fragment as described herein may exhibit reduced hydrophobicity. In some embodiments, hydrophobicity may be determined based on the retention time observed during HIC. In some embodiments, HIC may be performed and retention times may be obtained as described in Examples. In some embodiments, anti-CD3 antibodies or antigen-binding fragment as described herein may exhibit an HIC retention time of less than about 10.5 minutes (a clean to low HIC score). In some embodiments, anti-CD3 antibodies or antigen-binding fragment as described herein may exhibit 10.5 minutes ≤HIC retention time <11.5 minutes (a medium HIC score). 11.5 minutes ≤HIC retention time may be considered a high HIC score. Generally, the lower the HIC retention time the more favorable the developability of the antibody.

In some embodiments, anti-CD3 antibodies or antigen-binding fragment as described herein may exhibit reduced tendency for self-interaction. In some embodiments, tendency for self-interaction may be determined based on Δλmax values observed during affinity-capture self-interaction nanoparticle spectroscopy (AC-SINS). In some embodiments, AC-SINS may be performed and Δλmax values may be obtained as described in Examples. In some embodiments, anti-CD3 antibodies and/or antigen-binding fragments as described herein may display Δλmax of between about 0.0 nm and about 15.0 nm. In some embodiments, Δλmax of between about 0.0 nm and about 10.0 nm. In some embodiments, Δλmax of between about 0.0 nm and about 7.5 nm. In some embodiments, Δλmax of between about 0.0 nm and about 5.0 nm. In some embodiments, Δλmax of between about 0.0 nm and about 3.0 nm. In some embodiments, Δλmax of between about 0.0 nm and about 2.0 nm. In some embodiments, Δλmax of between about 0.0 nm and about 1.0 nm. In some embodiments, 0.0 nm≤Δλmax<5.0 nm is considered as “low self-interaction”. In some embodiments, 5.0 nm≤Δλmax<20.0 nm is considered as “medium self-interaction”. In some embodiments, 10.0 nm≤Δλmax is considered as “high self-interaction”. Generally, the lower the tendency for self-interaction the more favorable the developability of the antibody.

In some embodiments, anti-CD3 antibodies or antigen-binding fragment as described herein may exhibit reduced viscosity. In some embodiments, viscosity may be determined based on diffusion interaction parameter (kD) values observed during dynamic light scattering (DLS). In some embodiments, DLS may be performed and kD values may be obtained as described in Examples, e.g., using 10 mM histidine buffer (pH about 6). In some embodiments, anti-CD3 antibodies and/or antigen-binding fragments as described herein may display kD of about 5 mL/g or higher. In some embodiments, the kD may be about 10 mL/g or higher. In some embodiments, the kD may be about 15 mL/g or higher. In some embodiments, the kD may be about 20 mL/g or higher. In some embodiments, the kD may be about 25 mL/g or higher. In some embodiments, Δλmax<20 mL/g may be considered to be associated with high viscosity or high opalescence. Generally, the lower the viscosity the more favorable the developability of the antibody.

In some embodiments, anti-CD3 antibodies or antigen-binding fragment as described herein may exhibit reduced chance of heavy-light chain mispairing (including failure to pair). In some embodiments, heavy-light chain mispairing may be determined based on the presence of a heavy chain peak (indicating a heavy chain which did not successfully pair with a light chain) and/or a light chain peak (indicating a light chain which did not successfully pair with a heavy chain) observed during liquid chromatography-mass spectrometry (LC-MS). In some embodiments, LC-MS may be performed as described in Examples. In some embodiments, anti-CD3 antibodies and/or antigen-binding fragments as described herein may display no heavy chain peak or light chain peak. Generally, the smaller the heavy chain peak and light chain peak the more favorable the developability of the antibody.

In some embodiments, anti-CD3 antibodies or antigen-binding fragment as described herein may exhibit reduced propensity to aggregate. In some embodiments, propensity to aggregate may be determined based on % monomer values (i.e., % of antibody species (e.g., IgG or Fab) that are existing in its full size without aggregation or multimerization, among proteins from antibody production and optionally purification) observed during size exclusion chromatography (SEC). In some embodiments, SEC may be performed as described in Examples. In some embodiments, anti-CD3 antibodies and/or antigen-binding fragments as described herein may display % monomer in SEC of about 95% or higher, which indicates that the antibody is substantially existing as a monomer, i.e., not aggregating. In some embodiments, the % monomer may be about 97% or higher. In some embodiments, the % monomer may be about 98% or higher. In some embodiments, the % monomer may be about 99% or higher. In some embodiments, the % monomer may be about 99.5% or higher. Generally, the larger the % monomer value the more favorable the developability of the antibody.

In some embodiments, anti-CD3 antibodies or antigen-binding fragment as described herein may exhibit improved tolerance to an acidic environment or acidic stress, such as a pH of about 6 or below, about 5 or below, about 4 or below, or about 3.5 or below. In some embodiments, tolerance to low pH may be determined based on propensity to aggregate when exposed to low pH. In some embodiments, propensity to aggregate under a low pH may be determined based on % monomer values (e.g., monomer IgG or monomer Fab) observed during SEC after exposure to low pH. In some embodiments, such SEC for low pH tolerance test may be performed as described in Examples. In some embodiments, anti-CD3 antibodies and/or antigen-binding fragments as described herein may display % monomer in SEC of about 95% or higher after exposure to a low pH, which indicates that the antibody is substantially existing as a monomer, i.e., not aggregating. In some embodiments, the % monomer may be about 96% or higher. In some embodiments, the % monomer may be about 97% or higher. In some embodiments, the % monomer may be about 98% or higher. In some embodiments, the % monomer may be about 99% or higher. Generally, the larger the % monomer value after exposure to a low pH, the higher the tolerance to acidic stress, i.e., the more favorable the developability of the antibody. Without wishing to be bound by theory, improved tolerance to acidic stress may help provide longer shelf-life and/or improved in vivo stability (e.g., in an acidic cancer microenvironment).

In some embodiments, anti-CD3 antibodies and/or antigen-binding fragments as described herein may display a Tm of about 65° C. or higher. In some embodiments, Tm may be determined using DSF, which may be performed as described in Examples, or any other appropriate methods. Generally, the higher the Tm is the more stable the antibody, i.e., mode developable.

In some embodiments, anti-CD3 antibodies and/or antigen-binding fragments as described herein may be further modified to minimize effector function, e.g., a silent Fe.

The term “cytokine release syndrome” (or “CRS”) refers to a pro-inflammatory, positive feedback loop between cytokines and immune cells leading to excessive or uncontrolled release of pro-inflammatory cytokines by cells within the immune system (see, e.g., Lee et al., Blood, Vol. 124, pages 188-195 (2014) and Tisoncik et al., Microbiol Mol Biol Rev, Vol. 76, pages 16-32 (2012). Upon stimulation and activation, T cells release a series of cytokines to a level and degree that generates untoward biological/physiological effects or varying degree and severity, including acute inflammation characterized by, e.g., rubor (redness), swelling or edema, calor (heat), dolor (pain), and “functio laesa” (loss of function). When localized in skin or other tissue, biological/physiological effects comprise increased blood flow, enabling vascular leukocytes and plasma proteins to reach extravascular sites of injury, increasing local temperatures and generation of pain, tissue edema and extravascular pressure and a reduction in tissue perfusion. Other biological/physiological effects comprise organ and system dysfunction, such as cardiac dysfunction, adult respiratory distress syndrome, neurologic toxicity, renal and/or hepatic failure, and disseminated intravascular coagulation. Elevated levels of IFNγ, IL-6, TNFα, TGFbeta, IL-2, granulocyte macrophage-colony-stimulating factor (GM-CSF), IL-10, IL-8, IL-5, and/or fractalkine are implicated as predictive and/or causative of CRS or the propensity to elicit CRS upon T-cell stimulation.

In certain embodiments, the anti-CD3 antibodies and/or antigen-binding fragments described herein are detuned and/or modified to reduce the likelihood or severity of CRS induced by the antibody. Non-limiting exemplary modifications may include silent Fe regions (e.g., removing the Fe completely or modifying the Fc region to reduce or eliminate effector function), and/or masking (e.g., a polypeptide mask that is positioned such that it reduces or inhibits the ability of the antibody or antigen-binding fragment to specifically bind CD3).

### Anti-CD3 Antibodies and Antigen-Binding Fragments

“Cluster of Differentiation 3” or “CD3”, generally refers to any native CD3 from any vertebrate source, including mammals such as primates (e.g., humans and non-human primates) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD3γ protein (NCBI RefSeq No. NP_000064), which is 182 amino acids in length. “CD3εN27” and “CD3εN13” refer to the N-terminal 27 amino acids and the N-terminal 13 amino acids, respectively, of CD3, and optionally containing chemical modifications or conjugations made thereto.

An “anti-CD3 antibody” refers to an antibody or an antigen-binding fragment capable of binding to CD3, e.g., CD3ε and/or CD3γ, e.g., human CD3ε and/or CD3γ with sufficient affinity and/or specificity such that the antibody is useful as a diagnostic and/or therapeutic agent in targeting CD3. In some embodiments, an anti-CD3 antibody binds to CD3 with a dissociation constant (K_{D}) of about 100×10⁻⁹ M or less, about 50×10⁻⁹ M or less, about 25×10⁻⁹ M or less, about 20×10⁻⁹ M or less, or about 10×10⁻⁹ M or less. In some embodiments, an anti-CD3 antibody binds to CD3 with a dissociation constant (K_{D}) of about 5×10⁻⁹ M or less. In some embodiments, an anti-CD3 antibody binds to CD3 with a dissociation constant (K_{D}) of about 2.5×10⁻⁹ M or less. In some embodiments, an anti-CD3 antibody binds to CD3 with a dissociation constant (K_{D}) of about 1×10¹⁰ M or less. In some embodiments, K_{D} is measured by surface plasmon resonance, e.g., using a BIACORE® system, biolayer interferometry measurements using, e.g., using a FORTEBIO Octet® HTX instrument (Pall Life Sciences), or solution-affinity ELISA. In some embodiments, the K_{D} is measured using an scFv fragment of the anti-CD3 antibody. In some embodiments, the monovalent K_{D} is measured. In some embodiments, the anti-CD3 antibody binds to an epitope of CD3 that is conserved among CD3 from different species, e.g., human and cynomolgus cross-reactive.

The term “antibody” is used herein in the broadest sense and encompasses various antibody structures, including but not limited to monoclonal antibodies, polyclonal antibodies, multispecific antibodies (e.g., bispecific antibodies), whole antibodies, and antibody fragments (preferably those fragments that exhibit the desired antigen-binding activity (i.e., antigen-binding fragments)).

A “monoclonal antibody” or “mAb” refers to an antibody obtained from a population of substantially homogeneous antibodies, i.e., the individual antibodies comprising the population are identical and/or bind the same epitope, except for possible variant antibodies (e.g., containing naturally occurring mutations or arising during production of a monoclonal antibody preparation), such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

With regard to multispecific antibodies (e.g., bispecific, trispecific, tetraspecific, and so on), such antibodies comprise at least two different antigen-binding regions which recognize and specifically bind to at least two different antigens. With regard to bispecific antibodies, such antibodies comprise two different antigen-binding regions which recognize and specifically bind to at least two different antigens or epitopes. A “bispecific antibody” is a type of multispecific antibody and comprises two different antigen-binding regions which recognize and specifically bind to at least two different antigens or at least two epitopes. The at least two epitopes may or may not be within the same antigen. A bispecific antibody may target, for example, two different surface receptors on the same or different (e.g., an immune cell and a cancer cell) cells.

A “different antigen” may refer to different and/or distinct proteins, polypeptides, or molecules; as well as different and/or distinct epitopes, which epitopes may be contained within one protein, polypeptide, or one molecule.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The terms “intact antibody” and “whole antibody” or the like are used herein interchangeably and refer to an antibody having a structure substantially similar to a native antibody. In some instances, an antibody comprises heavy (H) and light (L) chains interconnected by disulfide bonds. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. For example, an intact IgG (or IgD or IgE) antibody comprises two immunoglobulin heavy chains and two immunoglobulin light chains. Therefore, in some instances, an antibody according to the present disclosure may comprise two pairs of heavy and light chains interconnected by disulfide bonds, or an antigen-binding fragment(s) thereof. Some intact antibody comprises multiple units each comprising two pairs of heavy and light chains interconnected by disulfide bonds. For example, an intact IgA comprises two units and an intact IgM comprises five units. Therefore, in other instances, an antibody according to the present disclosure may instead comprise multiple (e.g., two, three, four, five, and so on) units each comprising two pairs of heavy and light chains interconnected by disulfide bonds, or an antigen-binding fragment(s) thereof.

Each heavy chain is comprised of: a heavy chain variable domain (VH); and a heavy chain constant region (CH), which is typically comprised of domains CH1, CH2 and CH3. Each light chain is comprised of: a light chain variable domain (VL); and a light chain constant domain (CL). The VH and VL can be further subdivided into regions of hypervariability, termed complementarity determining regions (CDRs), interspersed with regions that are more conserved, termed framework regions (FRs). Each VH and VL polypeptide is composed of three CDRs and four FRs, arranged from amino-terminus to carboxy-terminus in the following order: FR1, CDR1, FR2, CDR2, FR3, CDR3, FR4. CDRs in a heavy chain are designated “CDRH1,” “CDRH2,” and “CDRH3,” respectively, and the CDRs in a light chain are designated “CDRL1,” “CDRL2,” and “CDRL3.” In certain embodiments of the disclosure, the FRs of the antibody (or antigen-binding fragment) may be identical to the human germline sequences or may be naturally or artificially modified. An amino acid consensus sequence may be defined based on a side-by-side analysis of two or more CDRs.

The numbering of amino acid residues in antibody variable and/or constant domains may be performed by any appropriate numbering schemes, methods, and definitions, e.g., based on numbering schemes such as EU numbering (as described in Kabat et al., Sequences of Proteins of Immunological Interest, 5th Ed. Public Health Service, National Institutes of Health, Bethesda, Md. (1991)), IMGT numbering, Kabat numbering, Chothia numbering, Martin numbering, Gelfand numbering, or Honneger's numbering); or structurally (see e.g., NCBI online tool, IgBlast; Dondelinger et al., Front Immunol. 2018 Oct. 16; 9:2278).

According to IMGT (the international ImMunoGeneTics information system for immunoglobulins or antibodies, T cell receptors, MH, immunoglobulin superfamily IgSF and MhSF), the CH1 domain, the hinge region, the CH2 domain, and the CH3 domain correspond to the amino acid positions 118-215, 216-230, 231-340, and 341-446, respectively (EU numbering). The terms “CH1 domain”, “hinge”, “CH2 domain”, and “CH3” are used in a broad sense herein to encompass any naturally occurring, corresponding heavy chain constant domain and/or region allotypes and variants thereof, which may comprise fewer or more amino acids (e.g., a CH1 domain may comprise a portion of a hinge region) and/or amino acid modification(s).

An exemplary CH1 domain of a human IgG1 may comprise the amino acid sequence of SEQ ID NO: 91 or 92; an exemplary hinge of a human IgG1 may comprise the amino acid sequence of SEQ ID NO: 51; and a CH2 domain of a human IgG1 may comprise the amino acid sequences of SEQ ID NOS: 61. An exemplary CH3 domain of a human IgG1 may comprise the amino acid sequence of SEQ ID NO: 71, 72, 73, or 74, and a C-terminal K may be added to any of such CH3 sequences. Any variants of such exemplary sequences may be used in conjunction with anti-CD3 variable sequences described herein.

“Fc region” is a C-terminal region of an immunoglobulin heavy chain that contains at least a portion of the constant region, including native sequence Fe regions and variant Fe regions. A human IgG heavy chain Fe region can extend from Cys226, or from Pro230, to the carboxyl-terminus of the heavy chain. However, the C-terminal lysine (Lys447) of the Fe region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

There are two major light chain isotypes, kappa (κ) and lambda (λ), and the corresponding light chain constant domains are called kappa CL domain (CLκ domain) and lambda CL domain (CLλ domain), respectively.

According to IMGT, the CLκ domain is the amino acid positions 108-214 (EU numbering). An exemplary CLκ domain of a human IgG may comprise the amino acid sequence of SEQ ID NO: 81. According to IMGT, the CLλ domain is the amino acid positions 107-215 (EU numbering). An exemplary CLλ domain of a human IgG may comprise the amino acid sequence of SEQ ID NO: 82.

The terms “CLκ domain” “CLλ domain” are used in a broad sense herein to encompass any naturally occurring, corresponding light chain constant domain and/or region allotypes and variants thereof, which may comprise fewer or more amino acids and/or amino acid modification(s).

Various standard sequences (corresponding to different allotypes) of the constant domains of human IgG1, IgG2, IgG3, and IgG4 are known in the field and may be found for example in Vidarsson et al., Front Immunol. 2014 Oct. 20; 5:520 and U.S. Pat. No. 9,150,663, the disclosures of which are hereby incorporated by reference herein in their entirety herein. Again these reference sequences are intended to be exemplary as Applicant intends for human IgG1, IgG2, IgG3, and IgG4 sequences to include any naturally occurring human IgG1, IgG2, IgG3, and IgG4 allotype.

An “antigen-binding fragment” or “antigen-binding antibody fragment” refers to a portion of an intact antibody or to a combination of portions derived from one or more intact antibody that binds the antigen to which the intact antibody binds (in this case, CD3). An antigen-binding fragment of an antibody includes any naturally occurring, enzymatically obtainable, synthetic, or genetically engineered polypeptide or glycoprotein that specifically binds an antigen to form a complex, including an antibody fragment. Exemplary antigen-binding fragments include, but are not limited to, Fv, Fab, Fab′, Fab′-SH, F(ab′)₂; diabodies; linear antibodies; single-chain antibody molecules (e.g., single-chain variable fragment (scFv), half antibody, nanobody or VH only, or VL only); and multispecific antibodies formed from antibody fragments. In some embodiments, the antigen-binding fragments of the anti-CD3 antibodies described herein are scFvs. The term “half molecule” or “half antibody” when referring to IgG, IgE, or IgD, which may also be referred to as “half IgG”, “half IgE”, or “half IgD”, respectively, refers to a set of one heavy chain and one light chain of the referenced antibody.

An “antigen-binding region” refers to a portion of an antibody or antigen-binding fragment with specificity for an antigen.

### Multispecific Antibodies and Antibody Fragments

Multispecific antibodies comprising at least one anti-CD3 antibody and/or antigen-binding fragment disclosed herein may be prepared according to a variety of techniques including, but are not limited to, recombinant co-expression of two immunoglobulin heavy chain-light chain pairs having different specificities (see, Milstein and Cuello, Nature 305: 537 (1983)), WO 93/08829, and Traunecker et al., EMBO J. 10: 3655 (1991)), “knob-in-hole” engineering (see, e.g., U.S. Pat. No. 5,731,168); immunoglobulin crossover (also known as Fab domain exchange or CrossMab format) technology (see, e.g., WO2009/080253; Schaefer et al., Proc. Natl. Acad. Sci. USA, 108:11187-11192 (2011)); engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); leucine zippers (see, e.g., Kostelny et al., J. Immunol, 148(5):1547-1553 (1992)); “diabody” technology (see, e.g., Hollinger et al., Proc. Natl. Acad. Sci. USA, 90:6444-6448 (1993)); single-chain Fv (scFv) dimers (see, e.g. Gruber et al., J. Immunol, 152:5368 (1994)); and trispecific antibodies as described, e.g., in Tutt et al. J. Immunol 147: 60 (1991).

A variety of multispecific antibody formats may be used in the context of multispecific antibodies and antibody fragments described herein. Non-limiting examples of multispecific and bispecific formats include, e.g., Fab-Fc-scFv (“bottle-opener”) (XENCOR), Mab-scFv (XENCOR), Mab-Fv (XENCOR), Dual scFv (XENCOR), central Fv (XENCOR), central scFv(XENCOR), one-arm central scFv (XENCOR), Fab-Fab (XENCOR), Fab-Fv (XENCOR), mAb-Fv (XENCOR), mAb-Fab (XENCOR), DART (MACROGENICS), BiTE (AMGEN/MICROMET), KiTE, common light chain-IgG (Genentech), TandAb (SFFIMED) Cross-Mab (ROCHE), SEED (EMD SERONO), BEAT (GLENMARK), TrioMab (TRION PHARMA/FRESENIUS BIOTECH), DuetMab (MEDIMMUNE), and others, as disclosed, e.g., in (WO2021067404; WO2022150787; WO2022150785; WO 95/09917; WO 2008/119566; WO 2008/119567; WO2011/121110; WO 2010/037835; WO 2007/042261; WO 2007/110205; WO 2011/121110; WO 2012/055961; WO 2012/16067; WO 2016/086189; WO 2016/182751; WO 2015/006749; WO 2014/049003; WO 2013/177101; WO 2015/128509; U.S. Pat. No. 7,951,917; US 2009/0252729; US 2014/0348839; U.S. Pat. No. 7,183,076; Mazor et al., Mabs, Vol. 7, pages 377-389 (2015); Muda et al., Protein Engineering, Design, & Selection, Vol. 24, pages 447-454 (2011); and Del Bano et al., Antibodies, Vol. 5, pages 1-23 (2016). In some embodiments, the anti-CD3 scFv fragments described herein comprise one or more variable domains of a multispecific (e.g., bispecific), antibody.

In some embodiments, a multispecific antibody or antibody fragment may comprise one or more engineered, variant constant domains which facilitate efficient polypeptide heterodimerization (e.g., a first heavy chain and a second heavy chain that is different from the first heavy chain) for bispecific antibody formation.

In certain embodiments, a multispecific antibody or antibody fragment may comprise at least one CLκ-preferring variant CH1 domain and/or a CLλ-preferring variant CH1 domain. A CLκ-preferring variant CH1 domain preferentially pairs with a CLκ domain rather than a non-CLκ domain (such as a CU domain). A CU-preferring variant CH1 domain preferentially pairs with a CLλ domain rather than a non-CLλ domain (such as a CLκ domain). In particular embodiments, such a CLκ-preferring variant CH1 domain and/or a CLκ-preferring variant CH1 domain may be selected from those described in WO2021067404.

In certain embodiments, a multispecific antibody or antibody fragment may comprise at least one pair of a CH1 domain and a CL domain which preferentially pair with each other. In a preferentially-pairing pair of CH1 and CL domains: the CH1 domain prefers to pair with the CL domain rather than another given CL domain such as a wildtype CL domain; and/or the CL domain prefers to pair with the CH1 domain rather than another given CH1 domain such as a wildtype CH1 domain. One or both of the CH1 domain and the CL domain may be variant domain(s). In particular embodiments, such a preferentially-pairing pair of a CH1 domain and a CL domain may be selected from the pairs described in WO2022150787.

In certain embodiments, a multispecific antibody or antibody fragment may comprise at least one pair of a first CH3 domain and a second CH3 domain that differs from the first CH3 which preferentially pair (i.e., form a heterodimer) with each other. In such a preferentially-pairing pair, the first CH3 domain prefers to pair with the second CH3 domain rather than another first CH3 domain; and/or the second CH3 domain prefers to pair with the first CH3 domain rather than with another second CH3 domain. One or both of the CH3 domains may be variant domain(s). In particular embodiments, such a preferentially-pairing pair of a first and second CH3 domains may be selected from the pairs described in WO2022150785.

### Targets

In certain embodiments, the anti-CD3 antibodies and/or antigen-binding fragments as described herein are contained in a multispecific antibody, in particular, a bispecific antibody that has binding specificity for a second antigen. Such a second antigen may be a different target altogether than the first target, or a different epitope present on the same target. In some embodiments, the binding specificities are to two different epitopes of CD3 (e.g., CD3ε or CD3γ). In other embodiments, one of the binding specificities is for CD3 (e.g., CD3ε or CD3γ) and the other is for a different biological molecule (e.g., a cell surface antigen, e.g., a tumor antigen).

Non-limiting examples of a second antigen toward which a bispecific antibody comprising anti-CD3 antibodies and/or antigen-binding fragments as described herein, comprises targets selected from the group consisting of: 17-IA, 4-1BB, 4Dc, 6-keto-PGF1a, 8-iso-PGF2a, 8-oxo-dG, Al Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL 14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxin1, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-IR, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin, alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein LI, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, a metalloprotease, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), P1GF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL RI Apo-2, DR4), TNFRSFIOB (TRAIL R2 DR5, KILLER, TRICK-2A, TRICK-B), TNFRSF10C (TRAIL R3 DcRI, LIT, TRID), TNFRSF10D (TRAIL R4 DcR2, TRUNDD), TNFRSF11A (RANK ODF R, TRANCE R), TNFRSFIlB (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSFIA (TNF RI CD120a, p55-60), TNFRSFIB (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL RI TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSFIA (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-RI, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3), receptors for hormones, and growth factors.

### Developability of Multispecific Antibodies and Antibody Fragments

As described throughout, anti-CD3 antibodies and/or antigen-binding fragments as provided herein possess one or more favorable developability characteristics and are, thus, relatively developable. Therefore, for effectively and efficiently developing a multispecific antibody or antibody fragment comprising an anti-CD3 antibody or antigen-binding fragment according to the present disclosure, once one or more multispecific antibody or antibody fragment candidates are designed (e.g., including the multispecific antibody format and the antibody sequence to provide a second specificity), the developability profile including one or more developability parameters (e.g., polyspecificity, hydrophobicity, self-interaction, viscosity, stability, shelf-life, tendency for heavy-light chain mispairing, propensity to aggregate, and/or tolerance to acidic stress) and/or any other properties (e.g., antigen binding, target cell binding, etc) of the candidates may be tested. Assays which may be used to assess developability and/or other antibody properties may include, but are not limited to, one or more of: PSR assays; CIC; SIC; HIC; SEC; DLS spectroscopy; photon correlation spectroscopy; quasi-elastic light scattering, CD, viscosity measurements; whole cell binding; tissue micro array methodologies; ELISA assays such as BVP ELISA assays; AC-SINS assays; Tm assays; differential scanning calorimetry or DSF; and the like.

### Anti-CD3 Antibody Variable Sequences

In some embodiments, anti-CD3 antibodies and antigen-binding fragments of the present disclosure may comprise one or more CDR sequences contained in a variable domain amino acid sequence of any one of those associated with Antibody numbers A001-A005 and V002-V0019 as shown in Tables 1A and 1B. In some embodiments, anti-CD3 antibodies and antigen-binding fragments of the present disclosure may comprise one or more CDR sequences encoded in a variable domain-encoding nucleic acid sequence of any one of those associated with Antibody numbers A001-A005 and V002-V0019 as shown in Tables 1C and 1D. In some embodiments, anti-CD3 antibodies and antigen-binding fragments of the present disclosure may comprise one or more CDR sequences of those associated with Antibody numbers A001-A005 and V002-V0019 as shown in Tables 2A and 2B.

In certain embodiments, anti-CD3 antibodies and antigen-binding fragments of the present disclosure may comprise CDRH1, CDRH2, and CDRH3 sequences individually selected from the CDRH1, CDRH2, and CDRH3 sequences that are: contained in any VH sequences associated with Antibody numbers A001-A005 and V002-V0019 as shown in Table 1A; encoded in any VH-encoding nucleic acid sequences associated with Antibody numbers A001-A005 and V002-V0019 as shown in Table 1C; or associated with any of Antibody numbers A001-A005 and V002-V0019 as shown in Table 2A.

In certain embodiments, anti-CD3 antibodies and antigen-binding fragments of the present disclosure may comprise a set of CDRH1, CDRH2, and CDRH3 sequences that are: contained in any one of the VH sequences associated with Antibody numbers A001-A005 and V002-V0019 as shown in Table 1A; encoded in any one of the VH-encoding nucleic acid sequences associated with Antibody numbers A001-A005 and V002-V0019 as shown in Table 1C; or associated with Antibody numbers A001-A005 and V002-V0019 as shown in Table 2A.

In certain embodiments, anti-CD3 antibodies and antigen-binding fragments of the present disclosure may comprise CDRL1, CDRL2, and CDRL3 sequences individually selected from the CDRL1, CDRL2, and CDRL3 sequences that are: contained in any VL sequences associated with Antibody numbers A001-A005 and V002-V0019 as shown in Table 1B; encoded in any VL-encoding nucleic acid sequences associated with Antibody numbers A001-A005 and V002-V0019 as shown in Table 1D; or associated with any of Antibody numbers A001-A005 and V002-V0019 as shown in Table 2B.

In certain embodiments, anti-CD3 antibodies and antigen-binding fragments of the present disclosure may comprise a set of CDRL1, CDRL2, and CDRL3 sequences that are: contained in any one of the VL sequences associated with Antibody numbers A001-A005 and V002-V0019 as shown in Table 1B; encoded in any one of the VL-encoding nucleic acid sequences associated with Antibody numbers A001-A005 and V002-V0019 as shown in Table 1D; or associated with Antibody numbers A001-A005 and V002-V0019 as shown in Table 2B.

In certain embodiments, anti-CD3 antibodies and antigen-binding fragments of the present disclosure may comprise a set of CDRH1, CDRH2, CDRH3, CDRL1, CDRL2, and CDRL3 sequences that are: contained in any of the VH and VL sequences associated with Antibody numbers A001-A005 and V002-V0019 as shown in Tables 1A and 1B; encoded in any one of the VH- and VL-encoding nucleic acid sequences associated with Antibody numbers A001-A005 and V002-V0019 as shown in Tables 1C and 1D; or associated with Antibody numbers A001-A005 and V002-V0019 as shown in Tables 2A and 2B.

In some embodiments, anti-CD3 antibodies and antigen-binding fragments of the present disclosure may comprise one or more FR amino sequences contained in any known germline-encoded variable domain sequences (e.g., mammalian germline sequence, e.g., mouse, human, or non-human germline sequence) or variants thereof. Such germline sequences may include human VH1-03 and/or VK4-01 germline sequences.

In some embodiments, anti-CD3 antibodies and antigen-binding fragments of the present disclosure may comprise one or more of the FR sequences contained in a variable domain amino acid sequence of any one of those associated with Antibody numbers A001-A005 and V002-V0019 as shown in Tables 1A and 1B. In some embodiments, anti-CD3 antibodies and antigen-binding fragments of the present disclosure may comprise one or more FR sequences encoded in a variable domain-encoding nucleic acid sequence of any one of those associated with Antibody numbers A001-A005 and V002-V0019 as shown in Tables 1C and 1D. In some embodiments, anti-CD3 antibodies and antigen-binding fragments of the present disclosure may comprise one or more FR sequences of those associated with Antibody numbers A001-A005 and V002-V0019 as shown in Tables 3A and 3B.

In certain embodiments, anti-CD3 antibodies and antigen-binding fragments of the present disclosure may comprise one or more of the FRH1, FRH2, FRH3, and FRH4 sequences individually selected from the FRH1, FRH2, FRH3, and FRH4 sequences that are: contained in any VH sequences associated with Antibody numbers A001-A005 and V002-V0019 as shown in Table 1A; encoded in any VH-encoding nucleic acid sequences associated with Antibody numbers A001-A005 and V002-V0019 as shown in Table 1C; or associated with any of Antibody numbers A001-A005 and V002-V0019 as shown in Table 3A.

In certain embodiments, anti-CD3 antibodies and antigen-binding fragments of the present disclosure may comprise a set of FRH1, FRH2, FRH3, and FRH4 sequences that are: contained in any one of the VH sequences associated with Antibody numbers A001-A005 and V002-V0019 as shown in Table 1A; encoded in any one of the VH-encoding nucleic acid sequences associated with Antibody numbers A001-A005 and V002-V0019 as shown in Table 1C; or associated with Antibody numbers A001-A005 and V002-V0019 as shown in Table 3A.

In certain embodiments, anti-CD3 antibodies and antigen-binding fragments of the present disclosure may comprise one or more of the FRL1, FRL2, FRL3, and FRL4 sequences individually selected from the FRL1, FRL2, FRL3, and FRL4 sequences that are: contained in any VL sequences associated with Antibody numbers A001-A005 and V002-V0019 as shown in Table 1B; encoded in any VL-encoding nucleic acid sequences associated with Antibody numbers A001-A005 and V002-V0019 as shown in Table 1D; or associated with any of Antibody numbers A001-A005 and V002-V0019 as shown in Table 3B.

In certain embodiments, anti-CD3 antibodies and antigen-binding fragments of the present disclosure may comprise a set of FRL1, FRL2, FRL3, and FRL4 sequences that are: contained in any one of the VL sequences associated with Antibody numbers A001-A005 and V002-V0019 as shown in Table 1B; encoded in any one of the VL-encoding nucleic acid sequences associated with Antibody numbers A001-A005 and V002-V0019 as shown in Table 1D; or associated with Antibody numbers A001-A005 and V002-V0019 as shown in Table 3B.

In certain embodiments, anti-CD3 antibodies and antigen-binding fragments of the present disclosure may comprise a set of FRH1, FRH2, FRH3, FRH4, FRL1, FRL2, FRL3, and FRL4 sequences that are: contained in any of the VH and VL sequences associated with Antibody numbers A001-A005 and V002-V0019 as shown in Tables 1A and 1B; encoded in any one of the VH- and VL-encoding nucleic acid sequences associated with Antibody numbers A001-A005 and V002-V0019 as shown in Tables 1C and 1D; or associated with Antibody numbers A001-A005 and V002-V0019 as shown in Tables 2A and 2B.

In certain embodiments, anti-CD3 antibodies and antigen-binding fragments of the present disclosure may comprise a set of FRH1, CDRH1, FR12, CDRH2, FRH3, CDRH3, FRH4, FRL1, CDRL1, FRL2, CDRL2, FRL3, CDRL3, and FRL4 sequences that are: contained in a VH and VL sequence combination associated with any one of Antibody numbers A001-A005 and V002-V0019 as shown in Tables 1A and 1B; encoded in a VH- and VL-encoding nucleic acid sequence combination associated with any one of Antibody numbers A001-A005 and V002-V0019 as shown in Tables 1C and 1D; or associated with any one of Antibody numbers V002-V0019 as shown in Tables 2A, 2B, 3A, and 3B.

In certain embodiments, anti-CD3 antibodies and antigen-binding fragments of the present disclosure may comprise a VH polypeptide sequence associated with any one of Antibody numbers A001-A005 and V002-V0019 as shown in Table 1A, or a sequence at least 80%, at least 85%, at least 90%, at least 92%, at least 94%, at least 95%, at least 96%, at least 97%, at least 98%, at least 99%, or 100% identical thereto. In certain embodiments, anti-CD3 antibodies and antigen-binding fragments of the present disclosure may comprise a VL polypeptide sequence associated with any one of Antibody numbers A001-A005 and V002-V0019 as shown in Table 1B, or a sequence at least 80%, at least 85%, at least 90%, at least 92%, at least 94%, at least 95%, at least 96%, at least 97%, at least 98%, at least 99%, or 100% identical thereto. In certain embodiments, anti-CD3 antibodies and antigen-binding fragments of the present disclosure may comprise a set of VH and VL sequences associated with any one of Antibody numbers V002-V0019 as shown in Tables 1A and 1B.

In certain embodiments, the present disclosure provides nucleic acids encoding anti-CD3 antibodies and antigen-binding fragments of the present disclosure. Nucleic acids may encode VH polypeptide sequences associated with any one of Antibody numbers A001-A005 and V002-V0019 as shown in Table 1A, or a sequence at least 80%, at least 85%, at least 90%, at least 92%, at least 94%, at least 95%, at least 96%, at least 97%, at least 98%, at least 99%, or 100% identical thereto. Such nucleic acids may include VH nucleic acid sequences associated with any one of Antibody numbers A001-A005 and V002-V0019 as shown in Table 1C, or a sequence at least 80%, at least 85%, at least 90%, at least 92%, at least 94%, at least 95%, at least 96%, at least 97%, at least 98%, at least 99%, or 100% identical thereto, or mRNA versions of any of the foregoing. In some embodiments, nucleic acids of the present disclosure include variants of those listed in Table 1C, wherein such variants include alternative codons (e.g., codon-optimized variants). Nucleic acids may encode VL polypeptide sequences associated with any one of Antibody numbers A001-A005 and V002-V0019 as shown in Table 1B, or a sequence at least 80%, at least 85%, at least 90%, at least 92%, at least 94%, at least 95%, at least 96%, at least 97%, at least 98%, at least 99%, or 100% identical thereto. Such nucleic acids may include VL nucleic acid sequences associated with any one of Antibody numbers A001-A005 and V002-V0019 as shown in Table 1D, or a sequence at least 80%, at least 85%, at least 90%, at least 92%, at least 94%, at least 95%, at least 96%, at least 97%, at least 98%, at least 99%, or 100% identical thereto, or mRNA versions of any of the foregoing. In some embodiments, nucleic acids of the present disclosure include variants of those listed in Table 1D, wherein such variants include alternative codons (e.g., codon-optimized variants).

### Modifications to Anti-CD3 Antibody Sequences

The present disclosure also contemplates modification of anti-CD3 antibodies relative to any of the anti-CD3 antibody sequences disclosed herein including but not limited to Antibody numbers V002-V0019, such modifications comprising one or more amino acid substitutions, insertions and/or deletions in the FR and/or CDR regions of the heavy and light chain variable domains. Once obtained, such derivative antibodies and/or antigen-binding fragments can be tested for one or more desired properties such as improved binding specificity, increased binding affinity, improved developability, etc.

In some embodiments, the VH and/or the VL of anti-CD3 antibodies and/or antigen-binding fragments according to the present disclosure may have amino acid sequence identities of at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at about 85%, at least about 84%, at least about 83%, at least about 82%, at least about 80%, at least about 75%, at least about 70%, at least about 65%, at least about 60%, at least about 55%, at least about 50% to the VH and/or the VL, respectively, of any one of Antibody numbers V002-V19 disclosed in Table 1A and/or 1B. In some embodiments, percent identity is measured by any well-known algorithm of sequence identity, such as FASTA, Basic Local Alignment Search Tool (BLAST®) or GAP.

In some embodiments, residue positions that are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. (See, e.g., Pearson (1994) Methods Mol. Biol. 24: 307-331). Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. In some embodiments, conservative amino acids substitution groups are: valine-leucine-isoleucine, phenylalanine-tyrosine, lysine-arginine, alanine-valine, glutamate-aspartate, and asparagine-glutamine. Alternatively, in some embodiments, a conservative replacement comprises any change having a positive value in the PAM250 log-likelihood matrix disclosed in Gonnet et al. (1992) Science 256: 1443 45. In some embodiments, a “moderately conservative” replacement comprises any change having a nonnegative value in a PAM250 log-likelihood matrix.

Substitution of one or more CDR residues or omission of one or more CDRs is also possible. Antibodies have been described in which one or two CDRs can be dispensed to alter binding in the scientific literature. Padlan et al. (1995 FASEB J. 9:133-139) analyzed contact regions between antibodies and their antigens, based on published crystal structures, and concluded that only about one fifth to one third of CDR residues actually contact their associated antigen. Padlan also found many antibodies in which one or two CDRs had zero amino acids in contact with an antigen (see also, Vajdos et al. 2002 J Mol Biol 320:415-428). CDR residues not contacting an antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of an antibody molecule include fusion to the N- or C-terminus of the antibody to an enzyme (e.g. for ADEPT) or a polypeptide which increases serum half-life of the antibody.

As described throughout, anti-CD3 antibodies and/or antigen-binding fragments as provided herein possess favorable developability and are, thus, relatively developable. Therefore, once the sequence of an anti-CD3 antibody (variable and/or constant sequences) is modified, the developability profile including one or more developability parameters (e.g., polyspecificity, hydrophobicity, self-interaction, viscosity, stability, shelf-life, tendency for heavy-light chain mispairing, propensity to aggregate, and/or tolerance to acidic stress) and/or any other properties of the derivative antibody may be tested. Assays which may be used to assess developability and/or other antibody properties may include one or more of but not limited to: PSR assays; CIC; SIC; HIC; SEC; DLS spectroscopy; photon correlation spectroscopy; quasi-elastic light scattering, CD, viscosity measurements; whole cell binding; tissue micro array methodologies; ELISA assays such as BVP ELISA assays; AC-SINS assays; Tm assays; differential scanning calorimetry or DSF; and the like. Further antibody selection may be performed based on antibodies exhibiting a desirable developability parameter or profile.

### Antibody Modification Via Conjugation

In certain embodiments, engineered CD3 binding domains and antibodies comprising them may be further modified to contain additional non-proteinaceous moieties that are known in the art and are readily available. Moieties suitable for derivatization of an antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In some embodiments, anti-CD3 antibodies and/or antigen-binding fragments as described herein are conjugated to a therapeutic moiety thereby forming an immunoconjugate. An “immunoconjugate” is an antibody conjugated to one or more heterologous molecule(s) such as, e.g., an antibiotic, a second anti-CD3 antibody, a vaccine, or a toxoid, or any other therapeutic moiety.

In certain embodiments the other therapeutic agent may be a chemotherapeutic agent, optionally one or more selected from alkylating agents, antimetabolites, plant alkaloids, and anti-cancer antibiotics, further optionally one or more selected from cyclophosphamide, cisplatin, carboplatin, oxaliplatin, etoposide, irinotecan, lurbinectedin, paclitaxel, docetaxel, cabazitaxel, altretamine, capecitabine, gemcitabine, ifosfamide, melphalan, pemetrexed, topotecan, vinorelbine, mitoxantrone, ixabepilone, eribulin, estramustine, vinblastine, vincristine, 5-fluorouracil (5-FU), doxorubicin, epirubicin, dactinomycin, or a derivative thereof.

In certain embodiments the other therapeutic agent may be an immunotherapeutic agent, optionally an immune checkpoint inhibitor or a growth factor or growth factor receptor inhibitor, further optionally an inhibitor of PD-L1, PD-1, CTLA-4, VISTA, EGF, EGFR, VEGF, and/or VEGFR, or an antibody or antigen-binding fragment against PD-L1, PD-1, CTLA-4, VISTA, EGF, EGFR, VEGF, and/or VEGFR, or an antibody or antigen-binding fragment against a cancer antigen.

In certain embodiments the other therapeutic agent may be an anti-emetic agent, optionally one or more selected from a neurokinin-1 receptor antagonist (NK1 RA), serotonin receptor antagonist (5-HT3 RA), dexamnethasone, olanzapine, and palonosetron.

In some embodiments, anti-CD3 antibodies and/or antigen-binding fragments as described herein may comprise a label or moiety that is detected directly (such as fluorescent, chromophoric, electron-dense, chemiluminescent, and radioactive labels or indirectly (such as enzymes or ligands). Non-limiting exemplary labels include, radioisotopes such as 32P, 14C, 1251, 3H, and 1311; fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase; heterocyclic oxidases such as uricase and xanthine oxidase, coupled with an enzyme that employs hydrogen peroxide to oxidize a dye precursor such as HRP, lactoperoxidase, or microperoxidase; biotin/avidin; spin labels; bacteriophage labels; stable free radicals; and the like.

### Sequence Modifications in the Constant Region

In some embodiments, anti-CD3 antibodies and/or antigen-binding fragments as described herein may be further modified to minimize effector function, e.g., a silent Fe or enhance one or more effector functions.

“Effector function” refers to biological activities attributable to the Fe region of an antibody, which varies by antibody isotype. Exemplary effector functions include: complement (e.g., C1q) binding and complement dependent cytotoxicity (CDC); Fe receptor binding; antibody-dependent cell-mediated cytotoxicity (ADCC); phagocytosis; down regulation of cell surface receptors (e.g., B cell receptor); and B cell activation.

In certain embodiments, one or more amino acid modifications may be introduced into the Fe region of an anti-CD3 antibody of the disclosure, thereby generating an Fe region variant (see, e.g., US 2012/0251531). An Fc region variant may comprise a human Fe region sequence (e.g., a human IgG1, IgG2, IgG3 or IgG4 Fe region) comprising an amino acid modification (e.g., a substitution) at one or more amino acid positions.

In certain embodiments, the disclosure contemplates an anti-CD3 antibody variant that possesses some but not all effector functions, which make it a desirable candidate for applications in which the half-life of an antibody in vivo is important yet certain effector functions (such as complement and ADCC) are unnecessary or deleterious. In vitro and/or in vivo cytotoxicity assays can be conducted to confirm reduction/depletion of CDC and/or ADCC activities. For example, Fc receptor (FcR) binding assays can be conducted to ensure that an antibody lacks FcγR binding (hence likely lacking ADCC activity) but retains FcRn binding ability. The primary cells for mediating ADCC (e.g. NK cells), express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see, Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (Cell Technology, Inc. Mountain View, Calif.); and CYTOTOX 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad Sci. USA 95:652-656 (1998). C1q binding assays may also be carried out to confirm that an antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Int'l. Immunol 18(12):1759-1769 (2006)).

In some embodiments, antibodies with reduced effector function include those with substitution of one or more of Fc region residues 238, 265, 269, 270, 297, 327 and 329 (U.S. Pat. Nos. 6,737,056 and 8,219,149). In some embodiments, Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In some embodiments, anti-CD3 antibodies and antigen-binding fragments as described herein may comprise a Fc region derived from a human IgG1 and the Fc region may optionally comprise one or more of the following amino acid modifications: N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V305I, P396L, S239D, I332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y M428L, N434S, L328F, M252Y, S254T, T256E, or any combination thereof, according to EU numbering. In some embodiments, anti-CD3 antibodies and antigen-binding fragments as described herein may comprise a Fc region derived from a human IgG4 and the Fc region may optionally comprise one or more of the following amino acid modifications: E233P, F234V, L235A, G237A, E318A, S228P, L236E, S241P, L248E, T394D, M252Y, S254T, T256E, N297A, N297Q, or any combination thereof, according to EU numbering.

In some embodiments, anti-CD3 antibodies and antigen-binding fragments as described herein may comprise a Fc region derived from a human IgG2 and the Fe region may optionally comprise one or more of the following amino acid modifications: P238S, V234A, G237A, H268A, H268Q, H268E, V309L, N297A, N297Q, A330S, P331S, C232S, C233S, M252Y, S254T, T256E, or any combination thereof, according to EU numbering.

In some embodiments, anti-CD3 antibodies and antigen-binding fragments as described herein may comprise a Fc region derived from a human IgG3 and the Fe region may optionally comprise E235Y, according to EU numbering.

In other embodiments, anti-CD3 antibodies and/or antigen-binding fragments as described herein are further modified to include a masking agent, e.g., a polypeptide mask, attached via a cleavable linker.

In certain embodiments, anti-CD3 antibodies and/or antigen-binding fragments as described herein are altered to increase or decrease the extent to which the antibody is glycosylated. Addition or deletion of glycosylation sites to an anti-CD3 antibody of the disclosure may be conveniently accomplished by altering the amino acid sequence such that one or more glycosylation sites is created or removed. In certain embodiments, addition or deletion of glycosylation sites may not be limited to the constant region of an anti-CD3 antibody or antigen-binding fragment.

### Production of the Anti-CD3 Antibodies and Antigen-Binding Fragments

Anti-CD3 antibodies and/or antigen-binding fragments may be produced using any appropriate methods including but not limited to recombinant methods.

For example, one or more isolated nucleic acids encoding an anti-CD3 antibody or antigen-binding fragment as described herein is provided. Such nucleic acids may encode an amino acid sequence comprising the VL, and/or an amino acid sequence comprising the VH of the antibody (e.g., the light and/or heavy chains of the antibody).

In some embodiments, one nucleic acid molecule may encode both (i) an amino acid sequence comprising the VH and (ii) an amino acid sequence comprising the VL of the antibody. In certain embodiments, (i) and (ii) may be encoded on the same strand of the nucleic acid molecule. In some cases, (i) and (ii) may be encoded under a single promoter. In certain cases, (i) and (ii) may be encoded in the same direction (in some instances, (i) and (ii) may be transcribed into a single transcript, and in some instances (i) and (ii) may be transcribed into two separate transcripts). In certain cases, (i) and (ii) may be encoded in the opposite directions. In some cases, (i) and (ii) may be encoded under separate promoters. In certain embodiments, (i) and (ii) may be encoded on different strands within a nucleic acid molecule.

In some embodiments, the antibody-encoding nucleic acids may comprise: (i) a first nucleic acid encoding an amino acid sequence comprising the VH; and (ii) a second nucleic acid encoding an amino acid sequence comprising the VL.

In the present disclosure, one or more vectors (e.g., expression vectors) comprising such nucleic acids are provided.

In some embodiments, one vector may comprise a nucleic acid which encodes both (i) an amino acid sequence comprising the VH and (ii) an amino acid sequence comprising the VL of the antibody. In certain embodiments, (i) and (ii) may be encoded on the same strand of the nucleic acid molecule. In some cases, (i) and (ii) may be encoded under a single promoter. In certain cases, (i) and (ii) may be encoded in the same direction (in some instances, (i) and (ii) may be transcribed into a single transcript, and in some instances (i) and (ii) may be transcribed into two separate transcripts). In certain cases, (i) and (ii) may be encoded in the opposite directions. In some cases, (i) and (ii) may be encoded under separate promoters. In certain embodiments, (i) and (ii) may be encoded on different strands within a nucleic acid.

In some embodiments, one or more vectors may comprise: (i) a first vector comprising a nucleic acid encoding an amino acid sequence comprising the VH; and (ii) a second vector comprising a nucleic acid encoding an amino acid sequence comprising the VL.

In the present disclosure, an isolated, recombinant, and/or host cell comprising such one or more nucleic acids described above, optionally contained in such one or more vectors described above, is provided.

In one such embodiment, a host cell comprises and/or has been transformed with): (1) a vector comprising a nucleic acid sequence that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody.

In one embodiment, the host cell is eukaryotic, optionally mammalian (e.g. a Chinese Hamster Ovary (CHO) cell, human embryonic kidney (HEK) cell such as HEK293 cell, or lymphoid cell (e.g., Y0, NS0, Sp20 cell)) or yeast.

In the present disclosure, a method of making an anti-CD3 antibody or antigen-binding fragment is provided, wherein the method comprises culturing a cell described herein such as a host cell comprising a nucleic acid encoding the antibody, as provided above, under conditions suitable for expression of the antibody, and optionally recovering the antibody from the host cell (or host cell culture medium).

The term “host cell” refers to cells into which an exogenous nucleic acid sequence has been introduced, including the progeny of such cells. Host cells include transformants and transformed cells, which include the primary transformed cell and progeny derived therefrom without regard to the number of passages.

For recombinant production of an anti-CD3 antibody, nucleic acids encoding an antibody, e.g., as described above, is isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody).

Suitable host cells for cloning and/or expression of antibody-encoding vectors include prokaryotic or eukaryotic cells. For example, antibodies may be produced in bacteria, in particular when glycosylation and Fe effector function are not needed. For expression of antibody fragments and polypeptides in bacteria, see, e.g., U.S. Pat. Nos. 5,648,237, 5,789,199, and 5,840,523. (See also, Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified. In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See, e.g., Gerngross, Nat. Biotech. 22:1409-1414 (2004), and Li et al., Nat. Biotech. 24:210-215 (2006); WO 2009/036379; WO 2010/105256; and WO 2012/009568.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y Acad Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Additional Antibody Identification and/or Characterization

Anti-CD3 antibodies and/or antigen-binding fragments may be identified, screened for, selected for or characterized for their physical/chemical properties and/or biological activities by various assays known in the art, e.g., ELISA, Western blot, etc. or competition assays may be used to identify an antibody that competes with an anti-CD3 antibody of the disclosure for binding to CD3. In an exemplary competition assay, immobilized CD3 is incubated in a solution comprising a first labeled antibody that binds to CD3 and a second unlabeled antibody that is being tested for its ability to compete with the first antibody for binding to CD3. The second antibody may be present in a hybridoma supernatant. As a control, immobilized CD3 is incubated in a solution comprising the first labeled antibody but not the second unlabeled antibody. After incubation under conditions permissive for binding of the first antibody to CD3, excess unbound antibody is removed, and the amount of label associated with immobilized CD3 is measured. If the amount of label associated with immobilized CD3 is substantially reduced in the test sample relative to the control sample, then that indicates that the second antibody is competing with the first antibody for binding to CD3. See, e.g., Harlow and Lane (1988) Antibodies: A Laboratory Manual. Ch. 14 (Cold Spring Harbor Laboratory, Cold Spring Harbor, N.Y.).

Anti-CD3 antibodies and/or antigen-binding fragments possessing biological activity may be identified using standard approaches. Biological activity may include, e.g., binding to CD3 on the surface of a T cell either in vivo, in vitro, or ex vivo. In the case of a multispecific anti-CD3 antibody (such as a bispecific antibody with one arm that binds to CD3 and another arm that binds to a different target, e.g., a cell surface antigen, e.g., a tumor antigen), biological activity may also include effector cell activation (such as CD8+ and/or CD4+ T cell) activation), effector cell population expansion (i.e., an increase in T cell count), target cell population reduction (i.e., a decrease in the population of cells expressing the second biological molecule on their cell surfaces), and/or target cell killing.

### Diagnostic and Therapeutic Uses for the Anti-CD3 Antibodies and Antigen-Binding Fragments

In some embodiments anti-CD3 antibodies and/or antigen-binding fragments described herein may be used for therapy and/or for diagnosis and/or detection.

“Detection” as used herein encompasses quantitative or qualitative detection.

In certain embodiments, anti-CD3 antibodies and antigen-binding fragments comprising a label or detection moiety as described herein may be used.

CD3 antibodies and/or antigen-binding fragments as described herein, as well as pharmaceutical compositions of such antibodies, may be used in therapeutic methods. In one embodiment, anti-CD3 antibodies and/or antigen-binding fragments as described herein or pharmaceutical compositions comprising such antibodies may be used for treating or delaying progression of a cell proliferative disorder or an autoimmune disorder. In some embodiments, the anti-CD3 antibodies and antigen-binding fragments may be used in treating cancers. Tumor cells typically have an extracellular pH of around about 6.3-6.5. In some embodiments, the anti-CD3 antibodies and antigen-binding fragments described herein may promote preferential CD3 binding at low(er) pH values, e.g., around pH 6 or lower, and thereby promote binding and activity in and around the tumor microenvironment. In some embodiments, use of the anti-CD antibodies and antigen-binding fragments may result in selective and sustained cytotoxic activity at or around the tumor site, thereby reducing or eliminating off-target effects.

A “disorder” refers to any condition or disease that would benefit from treatment including, but not limited to, chronic and acute disorders or diseases including those pathological conditions which predispose a mammal to the disorder in question.

The terms “cell proliferative disorder” and “proliferative disorder” refer to disorders that are associated with some degree of abnormal cell proliferation. Cell proliferative disorders include cancer and/or may involve a tumor.

“Tumor” as used herein refers to all neoplastic cell growth and proliferation, whether malignant or benign, and all pre-cancerous and cancerous cells and tissues.

“Cancer” refers to a physiological condition in mammals characterized by unregulated cell growth. Examples of cancer include, but are not limited to, carcinoma, lymphoma, blastoma, sarcoma, and leukemia or lymphoid malignancies; with more particular examples including squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo malignant melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phacomatoses, edema (such as that associated with brain tumors), Meigs' syndrome, brain, as well as head and neck cancer, and associated metastases. In certain embodiments, cancers that are amenable to treatment by antibodies of the disclosure include breast cancer, colorectal cancer, rectal cancer, non-small cell lung cancer, glioblastoma, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, mesothelioma, and multiple myeloma. In some embodiments, the cancer is selected from: small cell lung cancer, glioblastoma, neuroblastomas, melanoma, breast carcinoma, gastric cancer, colorectal cancer (CRC), and hepatocellular carcinoma. Yet, in some embodiments, the cancer is selected from: non-small cell lung cancer, colorectal cancer, glioblastoma and breast carcinoma, including metastatic forms of those cancers. In other embodiments, the cancer is selected from a class of mature B-Cell cancers excluding Hodgkin's Lymphoma but including germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenström macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Heavy chain diseases, a Heavy chain disease, γ Heavy chain disease, p Heavy chain disease, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma.

As used herein, “treatment” or “treat” or “treating” refer to clinical intervention in an attempt to alter the natural course of an individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “prevent,” “preventing,” and “prevention” refer to the prevention or inhibition of the development or onset of a disorder or disease.

As used herein, the terms “ameliorate” and “alleviate” refer to a reduction or diminishment in the severity a condition or any symptoms thereof.

In some embodiments, antibodies of the disclosure are used to delay development of a disorder or disease or to delay the progression of a disorder or disease. As used herein, “delaying progression” of a disorder or disease means to defer, hinder, slow, retard, stabilize, and/or postpone development of the disease or disorder (e.g., a cell proliferative disorder, e.g., cancer). The delay can be of varying lengths of time, depending on the history of the disease and/or individual being treated.

An effective amount of such antibody or composition may be administered to an individual suffering from cancer or arthritis, rheumatoid arthritis, colitis, inflammatory bowel disease, autoimmune type I diabetes, etc. An “effective amount” of an anti-CD3 antibody disclosed herein or a composition (e.g., pharmaceutical composition) comprising such antibody, is at least the minimum amount required to achieve the desired therapeutic or prophylactic result, e.g., a measurable improvement or prevention of a particular disorder, e.g., a cell proliferative disorder, e.g., cancer, preferably with minimal or no toxic or detrimental effects. An effective amount may vary according to inter alia disease state, age, sex, and weight of the patient, and the ability of the antibody (or antigen-binding fragment) to elicit a desired response in the individual and, in some instances, by co-administering one or more additional therapeutic agents.

In some embodiments, anti-CD3 antibodies and/or antigen-binding fragments as described herein may be used to enhance immune function in an individual having a cell proliferative disorder or an autoimmune disorder. Following administration, such antibody or composition may enhance immune function in an individual having a cell proliferative disorder or an autoimmune disorder by activating effector cells (e.g., T cells, e.g., CD8+ and/or CD4+ T cells including Tregs), expanding (increasing) the effector cell population, reducing the population of target cells (e.g., a cell expressing a second biological molecule recognized by an anti-CD3 antibody of the disclosure, such as a bispecific antibody), and/or killing a target cell (e.g., target tumor cell).

Anti-CD3 antibodies and/or antigen-binding fragments as disclosed herein may be used to treat disorders including, but not limited to, a proliferative disorder, an oncological disorder, an immune-oncological disorder, a neurological disorder, a cognitive disorder, a neurodegenerative disorder, an autoimmune disorder. In one embodiment, an effective amount of such anti-CD3 antibody may be administered, alone or in combination with at least one additional agent, to an individual having such disorder. Such “individual” may be a mammal and, in particular, a human.

One or more of the antibodies of the disclosure can be used alone or in combination with other agents in a therapy, e.g., an anti-CD3 antibody and/or antigen-binding fragment may be co-administered with at least one additional therapeutic agent. Non-limiting exemplary additional therapeutic agents include a chemotherapy agent, an antibody-drug conjugate (ADC), and/or a biological modifier.

Chemotherapy agents may be selected from cyclophosphamide, doxorubicin, vincristine, and prednisolone (CHOP).

ADC may be selected from an anti-CD79b antibody drug conjugate (such as anti-CD79b-MC-ve-PAB-MMAE or the anti-CD79b antibody drug conjugate described in any one of U.S. Pat. No. 8,088,378 and/or US 2014/0030280, or polatuzumab vedotin), an anti-CD19 antibody drug conjugate, an anti-CD22 antibody drug conjugate, an anti-CD45 antibody drug conjugate, and an anti-CD32 drug conjugate.

A biological modifier may be selected from a BCL-2 inhibitor (such as GDC-0199/ABT-199), lenalidomide (Revlimid®), a PI3K-delta inhibitor (such as idelalisib (ZYDELIG®)), a PD-1 axis binding antagonist, an agonist, e.g., agonist antibody, directed against an activating co-stimulatory molecule, e.g., CD40, CD226, CD28, OX40 (e.g., AgonOX), GITR, CD137 (also known as TNFRSF9, 4-1 BB, or ILA), CD27 (e.g., CDX-1127), HVEM, or CD127, an antagonist, e.g., antagonist antibody, directed against an inhibitory co-stimulatory molecule, e.g., CTLA-4 (also known as CD152), PD-1, TIM-3, BTLA, VISTA, LAG-3, B7-H3, B7-H4, IDO (e.g., 1-methyl-D-tryptophan (also known as 1-D-MT)), TIGIT, MICA/B, GITR (e.g., TRX518) or arginase, ipilimumab (also known as MDX-010, MDX-101, or YERVOY®), tremelimumab (also known as ticilimumab or CP-675,206, urelumab (also known as BMS-663513), MGA271, an antagonist directed against a TGF beta, e.g., metelimumab (also known as CAT-192), fresolimumab (also known as GC1008), LY2157299k, and an adoptive transfer of a T cell (e.g., a cytotoxic T cell or CTL) expressing a chimeric antigen receptor (CAR), e.g., adoptive transfer of a T cell comprising a dominant-negative TGF beta receptor, e.g, a dominant-negative TGF beta type II receptor.

Some more non-limiting exemplary additional therapeutic agents include growth inhibitory agent, cytotoxic agent, agent used in radiation therapy, anti-angiogenesis agent, apoptotic agent, anti-tubulin agent, or other agent, such as a epidermal growth factor receptor (EGFR) antagonist (e.g., a tyrosine kinase inhibitor), HER1/EGFR inhibitor (e.g., erlotinib (TARCEVA™)), platelet derived growth factor inhibitor (e.g., GLEEVAC™ (Imatinib Mesylate)), a COX-2 inhibitor (e.g., celecoxib), interferon, cytokine, antibody other than the anti-CD3 antibody of the disclosure, such as an antibody that bind to one or more of the following targets ErbB2, ErbB3, ErbB4, PDGFR-beta, BIyS, APRIL, BCMA VEGF, or VEGF receptor(s), TRAIL/Apo2, PD-1, PD-L1, or PD-L2, or another bioactive or organic chemical agent.

In some embodiments, the disclosure provides a method wherein the additional therapeutic agent is a glucocorticoid. In one embodiment, the glucocorticoid is dexamethasone.

Anti-CD3 antibodies and/or antigen-binding fragments as disclosed herein may be used to enhancing immune function in an individual, e.g., a human, having a disorder in an individual having such disorder. In one embodiment, a method of enhancing immune function comprises administering to an individual an effective amount of an anti-CD3 antibody to activate effector cells (e.g., T cells, e.g., CD8+ and/or CD4+ T cells), expand (increase) an effector cell population, reduce a target cell population, and/or kill a target cell (e.g., target tumor cell).

In a further aspect, pharmaceutical formulations comprising anti-CD3 antibodies and/or antigen-binding fragments as described herein are also provided, e.g., for use in any of the above therapeutic and/or diagnostic methods. A “pharmaceutical formulation” refers to a preparation in such form as to permit the biological activity of an active ingredient contained therein, such as the anti-CD3 antibodies described herein, to be effective and which preferably contains no additional components which are unacceptably toxic to a subject to which the formulation would be administered.

In one embodiment, a pharmaceutical formulation comprises any of the anti-CD3 antibodies disclosed herein and a pharmaceutically acceptable carrier.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some embodiments, the at least one additional therapeutic agent administered in a therapeutic method may be comprised in a pharmaceutical composition or formulation together with an anti-CD3 antibody. Therefore, in certain embodiments, a pharmaceutical formulation comprises any of the anti-CD3 antibodies provided herein and at least one additional therapeutic agent. In certain embodiments, the at least one additional therapeutic agent may be one or more of the additional therapeutic agents described above.

Such combination therapies noted above encompass combined administration (where two or more therapeutic agents are included in the same or separate formulations), and separate administration, in which case, administration of the antibody of the disclosure can occur prior to, simultaneously, and/or following, administration of additional therapeutic agent or agents. In one embodiment, administration of the anti-CD3 antibody and administration of an additional therapeutic agent occur within about one month, or within about one, two or three weeks, or within about one, two, three, four, five, or six days, of each other. Anti-CD3 antibodies of the disclosure (e.g., bispecific anti-CD3 antibodies of the disclosure that bind to CD3 and a second biological molecule, e.g., a cell surface antigen, e.g., a tumor antigen, such as a TDB antibody of the disclosure or variant thereof) can also be used in combination with radiation therapy.

An antibody of the disclosure (and/or any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. In some embodiments, the antibody is administered by subcutaneous administration. In some embodiments, an anti-CD3 antibody administered by subcutaneous injection exhibits a less toxic response in a patient than the same anti-CD3 antibody administered by intravenous injection. Dosing can be by any suitable route, for example, by injections, such as intravenous or subcutaneous injections, depending in part on whether the administration is brief or chronic. Various dosing schedules including but not limited to single or multiple administrations over various time-points, bolus administration, and pulse infusion are contemplated herein. In some embodiments, antibodies of the present disclosure may be administered via delivery of mRNA encoding such antibodies. Such administration may include formulation of mRNAs into lipid nanoparticles to facilitate administration and delivery to cells of a subject receiving treatment.

Antibodies of the disclosure would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not, but may optionally be, formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the disclosure (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

As a general proposition, a therapeutically effective amount of the anti-CD3 antibody administered to human will be in the range of about 0.01 to about 100 mg/kg of patient body weight whether by one or more administrations. In some embodiments, an antibody used is administered in about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg daily, for example. In one embodiment, an anti-CD3 antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg, or 10 mg/kg (or any combination thereof) may be administered to the patient. Such doses may be administered intermittently, for example, every week or every three weeks (e.g., such that the patient receives from about two to about twenty, or, for example, about six doses of the anti-CD3 antibody). An initial higher loading dose, followed by one or more lower doses, may be administered. The progress of this therapy is easily monitored by conventional techniques and assays.

In some embodiments, methods of the disclosure may further comprise an additional therapy. The additional therapy may be radiation therapy, surgery, chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy may be a separate administration of one or more of the therapeutic agents described above.

In another aspect of the disclosure, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the disclosure. The label or package insert indicates that the composition is used for treating the condition of choice. Moreover, the article of manufacture may comprise (a) a first container with a composition contained therein, wherein the composition comprises an antibody of the disclosure; and (b) a second container with a composition contained therein, wherein the composition comprises a further cytotoxic or otherwise therapeutic agent. The article of manufacture in this embodiment of the disclosure may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Accordingly, manufacture and/or preparation of a pharmaceutical composition comprising anti-CD3 antibodies and/or antigen-binding fragments as disclosed herein is also contemplated. The composition may be used alone or in combination with other active agents to treat a cell proliferative disorder (e.g., cancer) or an autoimmune disorder (e.g., arthritis, rheumatoid arthritis, colitis, inflammatory bowel disease, autoimmune type I diabetes, etc.).

In some embodiments, pharmaceutical compositions comprising anti-CD3 antibodies and/or antigen-binding fragments as described herein are prepared, e.g., by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions, optionally prepared for modified (e.g., sustained) release. Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968.

Such formulations may contain more than one active ingredient as necessary for the particular indication being treated, preferably those with complementary activities that do not adversely affect each other and present in amounts that are effective for the purpose intended. For example, it may be desirable to further provide an additional therapeutic agent (e.g., a chemotherapeutic agent, a cytotoxic agent, a growth inhibitory agent, and/or an anti-hormonal agent).

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Unless defined otherwise, all technical and scientific terms used herein have the same meaning as commonly understood by one of ordinary skill in the art to which this disclosure belongs.

It must also be noted that, unless the context clearly dictates otherwise, the singular forms “a,” “an,” and “the” as used herein and in the appended claims include plural refence. Thus, the reference to “a cell” refers to one or more cells and equivalents thereof known to those skilled in the art, and so forth. Unless defined otherwise, all technical and scientific terms used herein have the same meanings as commonly understood by a person of skilled in the art.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

It is understood that aspects and embodiments of the disclosure described herein include “comprising,” “consisting,” and “consisting essentially of” aspects and embodiments.

Although various embodiments and examples of the present invention have been described referring to certain molecules, compositions, methods, or protocols, it is to be understood that the present invention is not limited to the particular molecules, compositions, methods, or protocols described herein, as these may vary. It should be understood that, unless clearly indicated otherwise, in any methods disclosed or claimed herein that comprise more than one step, the order of the steps to be performed is not restricted by the order of the steps cited.

It is also to be understood that the terminology used in the description is for the purpose of describing the particular versions or embodiments only and is not intended to limit the scope of the present invention which will be limited only by the appended claims.

All references cited herein, including patent documents and non-patent documents, are hereby incorporated by reference in their entirety. Nothing herein is to be construed as an admission that the present invention is not entitled to antedate such disclosure by virtue of prior invention.

Examples are provided below to illustrate embodiments of the present disclosure. These examples are not meant to constrain the present invention to any particular application or theory of operation.

### EXAMPLES

### Example 1: Anti-CD3 Antibodies

Anti-CD3 antibodies A001 and A002 were isolated based on affinity for CD3. A001 is described in United States Publication Number US2020/0190189 (referred to therein by clone number ADI-26906), which is hereby incorporated by reference herein in its entirety. Antibodies V002-V007 include various amino acid substitutions relative to A001. These substitutions were also used to prepare variants of parental antibody A002 to yield antibodies V008-V013. Amino acid and nucleic acid sequences associated with antibodies A001, A002, and V002-V013 are provided in Tables 1-4.

### Affinity

Affinity of the anti-CD3 antibodies for CD3 was determined by measuring their kinetic constants (kₒₙ, k_{off}, K_{D}) on ForteBio Octet®. ForteBio affinity measurements were performed generally as previously described (Estep et al., MAbs. 2013 5(2):270-8). Briefly, ForteBio affinity measurements were performed by loading human or cynomolgus (cyno) CD3εδ Fe (300 nM) on-line onto AHC sensors. Sensors were equilibrated off-line in assay buffer for 30 minutes and then monitored on-line for 60 seconds for baseline establishment. For monovalent binding measurement, sensors with loaded CD3εδ Fc were exposed to 100 nM anti-CD3 Fab, as further described in Example 2 below] for 3 minutes, afterwards they were transferred to assay buffer for 3 minutes for off-rate measurement. Kinetics data were fit using a 1:1 binding model in the data analysis software provided by ForteBio. Resulting kinetic values are provided in the Table 5.

All antibodies tested demonstrated affinity for both human and Cyno CD3.

### Cell Binding

Specificity of the anti-CD3 antibodies for human CD3+ Jurkat cells and Cynomolgus monkey HSC-F cells was determined using a FACS cell binding assay. Briefly, cells were thawed and washed with cold PBSF buffer (PBS+0.1% BSA, pH 7.4). About 200,000 cells were aliquoted per well of a 96-well plate and pelleted by centrifugation (5 minutes at 500×g). The cells were washed and resuspended in 100 μl PBSF with CHO-produced anti-CD3 IgG antibody (100 nM). The mixture (cells+antibody) was incubated for 20 minutes on ice, then washed twice with PBSF. Cells were resuspended in 50 μl of propidium iodide (1:500 dilution) and anti-human IgG-RPE (1:100 dilution) prepared in PBSF, then incubated for 20 minutes on ice in the dark before cells were washed twice with PBSF. Binding was analyzed on FACS Canto II. Mean fluorescence intensities (MFI) are shown in Table 6. Also shown are results using cells without CD3 [CHO-S cells, Jurkat CD3 knockout (CD3−) cells, and HEK cells] for comparison, including results with Jurkat CD3-cells combined with anti-CD3 antibody Fab fragments (100 nM).

The results demonstrate preferred binding by antibodies tested to cells expressing CD3 as indicated by fluorescence intensity values that were multifold higher than those associated with non-CD3 expressing cells.

Further FACS analysis was conducted using 3-fold serial dilutions of anti-CD3 antibody Fab fragments. Fab fragments were generated from whole IgG antibodies by papain digestion and purified over KappaSelect or CaptureSelect IgG-CH1 (GE Healthcare LifeSciences) before preparation of serial dilutions. Binding to human CD3+ Jurkat cells and Cynomolgus monkey CD3 HSC-F cells was assessed with each diluted sample and results were used to build a titration curve and calculate half maximal effective concentration (EC50) values for Fab binding. Results are presented in Table 7.

Fab fragments from all antibodies tested demonstrated binding to both human and Cyno CD3 as indicated by low EC50 values.

### Developability—PSR

Anti-CD3 antibody developability was determined using multiple assessments, including polyspecificity analysis. Antibodies with high affinity for a target may otherwise fail in clinical settings where they also exhibit binding to multiple non-target entities. Antibody polyspecificity was assessed by measuring interaction with polyspecificity reagent (PSR). PSR was prepared as described in, e.g., WO 2014/179363 and Xu et. al., Protein Eng Des Sel, 26(10):663-670 (2013). In brief, 2.5 liters CHO-S cells were used as starting material. The cells were pelleted at 2,400×g for 5 min in 500 mL centrifuge bottles filled to 400 mL. Cell pellets were combined and then resuspended in 25 ml Buffer B and pelleted at 2,400×g for 3 min. The buffer was decanted and the wash repeated one time. Cell pellets were resuspended in 3× the pellet volume of Buffer B containing 1× protease inhibitors (Roche, Complete, EDTA-free) using a polytron homogenizer with the cells maintained on ice. The homogenate was then centrifuged at 2,400×g for 5 min and the supernatant retained and pelleted one additional time (2,400×g/5 min) to ensure the removal of unbroken cells, cell debris and nuclei; the resultant supernatant is the total protein preparation. The supernatant was then transferred into two Nalgene Oak Ridge 45 mL centrifuge tubes and pelleted at 40,000×g for 40 min at 4° C. The supernatants containing the Separated Cytosolic Proteins (SCPs) were then transferred into clean Oak Ridge tubes, and centrifuged at 40,000×g one more time. In parallel, the pellets containing the membrane fraction (EMF) were retained and centrifuged at 40,000 for 20 min to remove residual supernatant. The EMF pellets were then rinsed with Buffer B. 8 mL Buffer B was then added to the membrane pellets to dislodge the pellets and transfer into a Dounce Homogenizer. After the pellets were homogenized, they were transferred to a 50 mL conical tube and represented the final EMF preparation.

One billion mammalian cells (e.g. CHO, HEK293, Sf9) at ˜10⁶-10⁷ cells/mL were transferred from tissue culture environment into 4×250 mL conical tubes and pelleted at 550×g for 3 min. All subsequent steps were performed at 4° C. or on ice with ice-cold buffers. Cells were washed with 100 mL of PBSF (lx PBS+1 mg/mL BSA) and combined into one conical tube. After removing the supernatant, the cell pellet was then re-suspended in 30 mL Buffer B (50 mM HEPES, 0.15 M NaCl, 2 mM CaCl2), 5 mM KCl, 5 mM MgCl2, 10% Glycerol, pH 7.2) and pelleted at 550×g for 3 min. Buffer B supernatant was decanted and cells re-suspended in 3× pellet volume of Buffer B plus 2.5× protease inhibitor (Roche, cOmplete, EDTA-free). Protease inhibitors in Buffer B were included from here on forward. Cells were homogenized four times for 30 see pulses (Polyton homogenizer, PT1200E) and the membrane fraction was pelleted at 40,000×g for 1 hour at 4° C. The pellet is rinsed with 1 mL Buffer B; the supernatant is retained and represents the s. The pellet is transferred into a Dounce homogenizer with 3 mL of Buffer B and re-suspended by moving the pestle slowly up and down for 30-35 strokes. The enriched membrane fraction (EMF) is moved into a new collection tube, rinsing the pestle to collect all potential protein. Determine the protein concentration of the purified EMF using the Dc-protein assay kit (BioRad). To solubilize the EMF, transfer into Solubilization Buffer (50 mM HEPES, 0.15 M NaCl, 2 mM CaCl2), 5 mM KCl, 5 mM MgCl2, 1% n-Dodecyl-b-D-Maltopyranoside (DDM), 1× protease inhibitor, pH 7.2) to a final concentration of 1 mg/mL. Rotate the mixture overnight at 4° C. rotating followed by centrifugation in a 50 mL Oak Ridge tube (Fisher Scientific, 050529-ID) at 40,000×g for 1 hour. Collect the supernatant which represents the soluble membrane proteins (SMPs) and quantify the protein yield as described above.

For biotinylation, prepare the NHS-LC-Biotin stock solution according to manufacturer's protocol (Pierce, ThermoFisher). In brief, 20 μl of biotin reagent is added for every 1 mg of EMF sample and incubated at 4° C. for 3 hours with gentle agitation. Adjust the volume to 25 mL with Buffer B and transfer to an Oak Ridge centrifuge tube. Pellet the biotinylated EMF (b-EMF) at 40,000×g for 1 hour, and rinse two times with 3 mL of Buffer C (Buffer B minus the glycerol) without disturbing the pellet. Remove the residual solution. Re-suspended the pellet with a Dounce homogenizer in 3 mL of Buffer C as described previously. The re-suspended pellet now represents biotinylated EMF (b-EMF). Solubilized as described above to prepare b-SMPs.

PSR Binding Analyses. Assays were performed generally as described in, e.g., Xu et al. Protein Eng Des Sel, 26(10):663-670 (2013). To characterize the PSR profile of monoclonal antibodies presented on yeast, two million IgG-presenting yeast were transferred into a 96-well assay plate and pellet at 3000×g for 3 min to remove supernatant. Re-suspend the pellet in 50 μl of freshly prepared 1:10 dilution of stock biotinylated PSRs (b-PSRs) and incubate on ice for 20 minutes. Wash the cells twice with 200 μl of cold PBSF and pellet re-suspended in 50 μl of secondary labeling mix (Extravidin-R-PE, anti-human LC-FITC, and propidium iodide). Incubate the mix on ice for 20 minutes followed by two washes with 200 μl ice-cold PBSF. Re-suspend the cells in 100 μl of ice-cold PBSF and run the plate on a FACSCanto (BD Biosciences) using HTS sample injector. Flow cytometry data was analyzed for mean fluorescence intensity in the R-PE channel and normalized to proper controls (antibodies with established PSR scores) in order to assess non-specific binding. Numerous methods for presentation or display of antibodies or antibody fragments on the surface of yeast have been described previously, all of which are consistent with this protocol (Blaise et al., Gene, 342(2):211-8 (2004), Boder and Wittrup, Nat Biotechnol., 15(6):553-7 (1997), Kuroda and Ueda, Biotechnol Lett., 33(1):1-9 (2011), Orcutt and Wittrup, Springer Protocols: Antibody Engineering, 1:207-233 (2010), Rakestraw et al., Protein Eng Des Sel., 24(6):525-30 (2011), Sazinsky et al., Proc Natl Acad Sci USA., 105(51):20167-72 (2008), Tasumi et al., Proc Natl Acad Sci USA., 106(31):12891-6 (2009). Final PSR scores for antibodies tested are listed in Table 8.

Parental antibodies A001 and A002 had the highest PSR scores, indicating high levels of non-target-specific binding. A001 variants V002-V007 and A002 variants V008-V013 all demonstrated reduced PSR score relative to A001 and A002, respectively, with many demonstrating PSR scores ≤0.1, a “clean” PSR score, indicating very low levels of non-target-specific binding. Among the new variant antibodies, V002-V004, V006, and V007 exhibited a “clear” PSR score, and V005, V008, V010, V012, and V013 exhibited a “low” PSR score.

### Example 2. Methods

The following paragraphs include methods used in anti-CD3 antibody assessments, including many of those described above.

Hu and Cy CD3εδ Fc heterodimer antigen production. Recombinant heterodimeric CD3 Fe fusion antigens were produced in HEK 293 cells by co-transfection of plasmids encoding Hu CD3ε Fc (ectodomain, ECD, residues 22-126) and CD3δ Fe-HIS (ECD residues 22-100) or Cy CD3ε Fe (ECD residues 22-117) and CD3δ Fc-HIS (ECD residues 22-100) utilizing a heterologous signal peptide sequence. Chromatographic separations were performed on a computer controlled ÅKTA Avant 150 preparative chromatography system (GE Healthcare Life Sciences) equipped with an integrated conductivity sensor, enabling in-line salt concentration monitoring during the run. Clarified culture supernatants were purified by Ni Sepharose 6 Fast Flow (GE Healthcare Life Sciences), which removes the CD3δδ Fc homodimer. CD3εδ Fc-HIS heterodimer was resolved from CD3εε Fe-HIS homodimer by Mono Q 10/100 GL by a linear Tris-buffered KCl gradient at pH 8.5. Rhesus and mouse CD3εδ Fe heterodimers were produced in a similar manner.

Peptides. C-terminally biotinylated CD3ε N-terminal peptides were obtained from New England Peptide. All peptides were delivered with a purity of ≥95%. Peptides were designed based on the primary sequence of Hu CD3ε and the crystal structure of Hu CD3εδ bound to OKT3 (Kjer-Nielsen L. et al. PNAS 2004). The CD3εN27 peptide has the sequence H2N-QDGNEEMGSITQTPYQVSISGTTVILT[K/SCBiot(dPEG4)]-amide (SEQ ID NO: 40) and the CD3εN13 peptide has the sequence H2N-QDGNEEMGGITQT[K/SCBiot(dPEG4)]-amide (SEQ ID NO: 41).

Antigen biotinylation. CD3 antigens were biotinylated using the EZ-Link Sulfo-NHS-Biotinylation Kit from Pierce. Goat anti-human F(ab′)2 kappa-FITC (LC-FITC), Extravidin-PE (EA-PE) and streptavidin-633 (SA-633) were obtained from Southern Biotech, Sigma and Molecular Probes, respectively. Streptavidin MicroBeads and MACS LC separation columns were purchased from Miltenyi Biotec.

Cell line propagation and cell labeling assays. Human Jurkat CD3+ cells (ATCC TIB-152) and Jurkat CD3− cells (ATCC TIB-153) were obtained from ATCC. Cyno HSC-F cells were obtained from the NIH Non-human Primate Reagent Resource. All cell lines were cultured in RPMI 1640 GlutaMax media supplemented with 10% fetal bovine serum (FBS).

Cell labeling was conducted by aliquoting 100,000-200,000 cells per well in a 96-well assay plate. Cells were centrifugated at 500×g for 5 min at 4° C., then resuspended in 100 μl of 100 nM IgG and incubated at room temperature for 20 min. Cells were then washed in buffer (phosphate-buffered saline (PBS)/0.1% bovine serum albumin (BSA) three times and resuspended in secondary reagent, typically goat anti-human R-PE (Southern Biotech). The plate was assayed on a FACSCanto (BD Biosciences) using an HTS sample injector. Flow cytometry data was analyzed for median fluorescence intensity in the R-PE channel.

FACS affinity pressured selection methods. Briefly, yeast cells (at least ˜2×10⁷ cells/labeling condition) were incubated with a volume of biotinylated antigen sufficient to represent a stoichiometric excess with respect to the average IgG presentation number. Antigen labeling conditions are 100 to 1 nM under equilibrium conditions, typically carried out for 20 min to several hours at room temperature in FACS wash buffer (phosphate-buffered saline (PBS)/0.1% bovine serum albumin (BSA)). After washing three times with wash buffer, yeast are then stained with secondary reagents anti-human light chain FITC conjugate (LC-FITC) diluted 1:100 and either streptavidin-633 (SA-633) diluted 1:500 or extravidin-phycoerythrin (EA-PE) diluted 1:50 for 15 min at 4° C. After washing twice with ice-cold wash buffer, the cell pellets are resuspended in wash buffer in a typical volume of at least 1 mL per 1×10⁷ yeast and transferred to strainer-capped sort tubes. Sorting is performed using a FACS ARIA sorter (BD Biosciences) and sort gates are determined to select for binders. After the final round of sorting, yeast cells were plated and individual colonies picked for characterization.

Antibody yeast production and purification. Yeast clones were grown to saturation and then induced for 48 h at 30° C. with shaking. After induction, yeast cells were pelleted and the supernatants were harvested for purification. IgGs were purified using a Protein A column and eluted with acetic acid, pH 2.0. Fab fragments were generated by papain digestion and purified over KappaSelect or CaptureSelect IgG-CH1 (GE Healthcare LifeSciences).

Antibody HEK production and purification. Mammalian expression of IgG was done by sub-cloning antibodies into a new expression vector followed by transient transfection and expression in HEK293ADI1, a monoclonal cell line derived from HEK293 (DSMZ) selected for clump-free growth, growth rate, and transfectability. Briefly, expression vectors containing the antibody of interest were transfected by complexing with a transfection reagent followed by exposure to HEK cells for one hour followed by dilution of culture media to a final density of 4 million cells per mL. The cells were then cultured for 7 days with fresh feed media every 48 hours. After 7 days, the supernatant was collected following centrifugation and purification was performed using protein A. If necessary, a CHT column purification was added to reach >95% monomer.

Antibody production in CHO cells and purification. Antibodies were produced as IgG1 by sub-cloning antibodies into a new expression vector followed by transfection and expression in CHO cells. Fab fragments were generated by papain digestion and purified over KappaSelect or CaptureSelect IgG-CH1 (GE Healthcare LifeSciences). The VH- and VL-encoding gene fragments (Integrated DNA Technologies) were subcloned into heavy- and light-chain pcDNA 3.4+ vectors (ThermoFisher). The corresponding vectors were transiently co-transfected into CHO-K1 suspension cells using standard methods well known in the art. In general, CHO-K1 cells grown to about 4×10^{*6} cells/mL were pelleted and resuspended in transfection medium. DNA plasmids (1.5 ug total DNA/mL) were incubated with PEIpro (1:2 final, PolyPlus, Cat #115-100) in transfection medium at room temperature before addition to the CHO-K1 cell suspension. Transfected cultures were fed and maintained at 32° C., shaking, until supernatant was harvested (at day 9) for purification. The cell culture supernatant was harvested by centrifugation and passed over Protein A agarose (MabSelect SuRe; GE Healthcare Life Sciences). The bound antibodies were then washed with PBS and eluted with buffer (200 mM acetic acid/50 mM NaCl, pH 3.5) into 1/8 volume 2 M Hepes, pH 8.0. The final products were buffer-exchanged into 25 mM Hepes and 150 mM sodium chloride, pH 7.3. Fab was generated using an overnight papain digest, followed by a CH1-resin purification step.

Cell binding assays. CD3+ human Jurkat cells (ATCC) and CHO-S cells (Invitrogen/ThermoFisher) were thawed and washed with cold PBSF buffer, pH 7.4 (PBS+0.1% BSA, pH 7.4). About 200,000 cells were aliquoted per well of a 96-well plate (FACS Assay Plate VWR BD 353263) and pelleted by centrifugation (5 minutes at 500×g). The cells were washed with either PBSF pH 7.4 or PBSF pH 6.0 (PBS+0.1% BSA, pH 6.0), and then resuspended in 100 μl in either PBSF pH 7.4 or PBSF pH 6.0 with IgG1 antibody (100 nM) produced in yeast as described above or in CHO cells. The mixture (cells+antibody) was incubated for 20 minutes on ice, then washed twice with either PBSF pH 7.4 or PBSF pH 6.0. Cells were resuspended in 50 μl of propidium iodide (Roche; 1:500 dilution) and anti-human IgG-RPE (Southern Biotech; 1:100 dilution) prepared in either PBSF pH 7.4 or PBSF pH 6.0, then incubated for 20 minutes on ice in the dark before cells were washed twice with either PBSF pH 7.4 or PBSF pH 6.0. Binding was analyzed on FACS Canto II.

ForteBio K_{D} measurements (Biolayer interferometry; BLI). ForteBio affinity measurements were performed generally as previously described (Estep, P., et al., High throughput solution-based measurement of antibody-antigen affinity and epitope binning. MAbs, 2013. 5(2): p. 270-8.). Briefly, ForteBio affinity measurements were performed by loading IgGs or CD3εδ Fe-HIS heterodimer onto AHC sensors. Sensors were equilibrated off-line in assay buffer for 30 min and then monitored on-line for 60 seconds for baseline establishment. Sensors with loaded IgGs were exposed to 100 nM antigen (e.g., CD3) for 3 min, afterwards they were transferred to assay buffer for 10 min for off-rate measurement. Sensors loaded with CD3εδ Fe-HIS heterodimer were exposed to 100 nM anti-CD3 Fab for 3 min, afterwards they were transferred to assay buffer for 3 min or 10 min for off-rate measurement. Kinetics were analyzed using the 1:1 binding model. For measuring binding at different pHs, measurements were performed at pre-determined pHs (e.g., pH 7.4 and pH 6.0).

ForteBio Kinetics. FortBio Octet® HTX instruments were used in 12 channel mode (8 sensors per channel, 96 sensors per experiment) with either AHC, SA, or AHQ sensors. Instrumentation was driven by manufacturer supplied software (versions 8.2 and 9.0). Sample names and concentrations were input into the plate data page, and sensor associated proteins were identified in the “information” column on the sensor data page. Kinetic experiments were collected with either a 90 or 180 s baseline, 180 s association phase, and 180 s or 600 s dissociation phase. All files were saved into a shared network drive with a naming convention that identifies the format of the experiment.

HIC. IgG1 samples were buffer exchanged into 1 M ammonium sulfate and 0.1 M sodium phosphate at pH 6.5 using a Zeba 40 kDa 0.5 mL spin column (Thermo Pierce, cat #87766). A salt gradient was established on a Dionex ProPac HIC-10 column from 1.8 M ammonium sulfate, 0.1 M sodium phosphate at pH 6.5 to the same condition without ammonium sulfate. The gradient ran for 17 min at a flow rate of 0.75 ml/min. An acetonitrile wash step was added at the end of the run to remove any remaining protein and the column was re-equilibrated over 7 column volumes before the next injection cycle. Peak retention times were monitored at A280 absorbance and concentrations of ammonium sulfate at elution were calculated based on gradient and flow rate.

LC-MS. IgG1 samples were reduced by DTT, followed by middle down liquid chromatography-mass spectrometry (LC-MS) analysis on a Bruker maXis4G mass spectrometer coupled with an Agilent 1100 HPLC (Agilent). A POROS R2 10 μm (2.1×30 mm) reversed phase column was used to remove salt in the samples. A fast LC flow at 2 mL/min allows the separation between sample and salt and elution of samples and regeneration of column to finish within a 2.1 min cycle. A T-junction is used to deliver only 0.15 mL/min sample flow into the mass spectrometer for sample analysis. The Bruker maXis 4G mass spectrometer was run in positive ion mode with detection in the range of 750 to 2500 m/z. The remaining source parameters were set as follows; the capillary was set at 5500V, the Nebulizer at 4.0 Bar, dry gas at 4.0 l/min, and dry temp set at 200° C.

The MS spectra were analyzed using Bruker Data Analysis version 4.1 and the deconvolution was accomplished using maximum entropy deconvolution with a mass range of 20 to 30 kDa.

### Example 3: Additional Anti-CD3 Antibodies

Anti-CD3 antibodies A003, A004, and A005 were previously isolated as described in United States Publication Number US2020/0190189 (referred to therein as ADI-26913, ADI-26920, and ADI-26921), which is hereby incorporated by reference herein in its entirety.

Antibodies V014 and V015 were newly obtained by incorporating various amino acid substitutions to one or more of the CDRs of A003. Antibodies V016 and V017 were newly obtained by incorporating various amino acid substitutions to one or more of the CDRs of A004. Antibodies V018 and V019 were newly obtained by incorporating various amino acid substitutions to one or more of the CDRs of A005. Amino acid and nucleic acid sequences associated with antibodies A003-A005 and V014-V019 are provided in Tables 1-4.

### Example 4: Monovalent Binding Affinity at Different pHs—Fab, SPR

Anti-CD3 antibodies A001-A005, V002, V004, V007, V009, V010, V013, and V014-V019 were produced in CHO cells, and Fab fragments were generated from IgG1 antibodies by papain digestion and purification as described in Example 2. Monovalent binding affinity at pH 7.4 and pH 6.0 was measured via surface plasmon resonance (SPR). Briefly, human or cynomolgus CD3εδ Fe heterodimer produced as described in Example 2 was immobilized to a CM5 sensor chip in a Biacore® X100 (Cytiva, previously GE Healthcare Life Sciences) to a response level of ˜500 RUs. Fabs were then injected at 3-fold increasing concentrations, ranging from 0.74-180 nM. The sensor chip was doubly regenerated between cycles using 0.35 M EDTA and 0.1 M NaOH. The resulting data were double reference subtracted and fit to a 1:1 binding model using Biacore® Evaluation Software.

Results are shown in Table 9. As shown in the table, all tested Fabs bound to both human and cynomolgus CD3εδ at both pH 7.4 and 6.0.

### Example 5: Affinity and Avidity at Different pHs—Fab or IgG1, BLI

Anti-CD3 antibodies A001-A005, V002, V004, V007, V009, V010, V013, and V014-V019 were produced as IgG1s in CHO cells, and Fab fragments were generated from IgG1 antibodies by papain digestion and purification as described in Example 2. Human, cynomolgus, rhesus, and mouse CD3εδ Fc heterodimers were produced, and monovalent binding affinity and avidity at pH 7.4 and pH 6.0 were measured using the Octet® HTX instrument (ForteBio) as described in Example 2.

Results are shown in Table 10. As shown in the table, all tested Fabs bound to both human and cynomolgus CD3εδ at both pH 7.4 and 6.0, and all tested Fabs (except that K_{D} was undeterminable for V017) bound to rhesus CD3εδ at pH 7.4. While some of the tested IgGs (A001-A003, V002, V004, V009, V010, and V013-V015) bound to mouse CD3εδ to some extent at pH 7.4, some of the tested IgGs (A004, A005, V007, and V016-V019) did not bind to mouse CD3εδ at pH 7.4.

### Example 6: Cell Binding—IgG1, Flowcytometry

Anti-CD3 antibodies A001-A005, V002, V004, V007, V009, V010, V013, and V014-V019 were produced as IgG1s in CHO cells, and binding to human CD3+ Jurkat cells and cynomolgus CD3+ HSC-F cells at pH 7.4 was analyzed by flow cytometry as described in Example 2. NCB (Normalized Cell Binding) values were calculated based on control and sample MFI values as NCB={(Sample MFI)−(Secondary only MFI)}/(Secondary only MFI).

NCB values for the tested IgG1s are shown in Table 11. As shown in the table, all tested antibodies bound to both human and cyno CD3-expressing cells. Increased binding to human CD3+ Jurkat cells was observed with V014-V019 compared to their parent antibodies. For example, V014 and V015 showed higher binding than their parent antibody A003, V016 and V017 showed higher binding than their parent antibody A004, and V018 and V019 showed higher binding than their parent antibody A005.

### Example 7: Developability—Fab or IgG1, Chromatography

Anti-CD3 antibodies A001-A005, V002, V004, V007, V009, V010, V013, and V014-V019 were produced as IgG1s in CHO cells and Fab fragments were generated from IgG1 antibodies by papain digestion and purification as described in Example 2.

### Sequence Confirmation

To test whether heavy and light chain mass matched expected mass based on amino acid sequences, IgG1 samples were subjected to LC-MS analyses as described in Example 2. Samples that matched expected mass are marked, as shown in Table 12, as “PASS”.

### Purity

To test the purity of the produced IgG1s and Fabs (as determined by the percentage of whole IgG monomer or Fab monomer, respectively, (i.e., existing without aggregation or multimerization) among all antibody products), IgG1 and Fab production samples were subjected to size exclusion chromatography (SEC)-high-performance liquid chromatography (HPLC) analyses. Briefly, an Agilent 1260 HPLC was employed to monitor the column chromatography (TSKgel Super SW mAb HTP column). The column was equilibrated with wash buffer (200 mM Sodium Phosphate, 250 mM Sodium Chloride pH 6.8) at a flow rate adjusted to 0.400 mL/min prior to use. Approximately 2-5 μg of an IgG1 or Fab protein sample was injected onto column. Protein migration was monitored at wavelength 280 nm. Total assay time was approximately 6 minutes. Data was analyzed using ChemStation software.

% Monomer values obtained by SEC-HPLC are shown in Table 12. As shown in the table, over 95% of % monomer was observed with all Fabs tested and almost all of the tested IgG1s.

### pH Stress Tolerance

To test tolerability to low pH stress, IgG1 samples were incubated at an acidic pH or a physiological pH and subjected to SEC-HPLC analyses. Briefly, IgG1 samples at 15 mg/mL were buffer exchanged into PBS (200 mM phosphate buffered with 250 mM sodium chloride, pH 7.0) or pH 3.5 buffer (50 mM sodium chloride, 200 mM acetic acid, pH 3.5). After 1 hour at room temperature (25° C.), buffer exchanged samples were diluted to 1 mg/mL in PBS (200 mM phosphate buffered with 250 mM sodium chloride, pH 7.0), and 2 μg of sample was injected into an Agilent 1260 Infinity analytical HPLC (Agilent, Santa Clara, CA) fitted with a TSKgel SuperSW mAb HTP column (TOSOH Bioscience, King of Prussia, PA, Product Code 22855). SEC data was collected and subjected to analysis using Agilent ChemStation software (Agilent, Santa Clara, CA).

% Monomer values obtained by SEC-HPLC are shown in Table 12. As shown in the table, % monomer values were not significantly affected by low pH and remained >95% in all IgG1s tested, indicating great tolerability to low pH stress.

### Example 8: Developability—PSR

### Polyspecificity by PSR-Binding Assay

Anti-CD3 antibodies A001-A005, V002, V004, V007, V009, V010, V013, and V014-V019 were subjected to PSR-binding analyses as described in Example 1.

PSR scores are shown in Table 13. As shown in the table, many of the tested IgG1s (A003-A005, V002, V004, V007, and V014-V019) showed clean PSR scores, indicating no to negligible polyspecificity, and some of the tested IgGs (A001, V009, V010, and V013) showed low PSR scores, indicating low polyspecificity.

### Example 9: Developability—HIC, AC-SINS, and DLS

Anti-CD3 antibodies A001-A005 and V002, V004, V007, V009, V010, V013, and V014-V019 were produced as IgG1s in CHO cells as described in Example 2. Hydrophobicity by HIC

Hydrophobicity of an antibody is one cause of antibody aggregation. IgG1s production samples were subjected to hydrophobic interaction chromatography (HIC) analyses as described in Example 2.

HIC retention times observed are shown in Table 13. As shown in the table, all of the tested IgG1s had a retention time of <10.5 min, indicating clean to low hydrophobicity, i.e., a highly desirable developability profile.

### Self-Interaction by AC-SINS

Self-interaction was be measured in vitro by affinity-capture self-interaction nanoparticle spectroscopy (AC-SINS) using a previously described protocol (Liu y et al., MAbs. March-April 2014; 6(2):483-92). Briefly, polyclonal goat anti-human IgG Fe antibodies (capture; Jackson ImmunoResearch Laboratories) and polyclonal goat non-specific antibodies (non-capture; Jackson ImmunoResearch Laboratories) were buffer exchanged into 20 mM sodium acetate (pH 4.3) and concentrated to 0.4 mg/ml. A 4:1 volume ratio of capture:non-capture may be prepared and further incubated at a 1:9 volume ratio with 20 nm gold nanoparticles (AuNP; Ted Pella Inc.) for 1 hour at room temperature. Thiolated PEG (Sigma-Aldrich) was then be used to block empty sites on the AuNP and filtered via a 0.22 m PVDF membrane (Millipore). Coated particles were subsequently added to the test IgG1 antibody solution and incubated for 2 hours at room temperature before measuring absorbance from 510 to 570 nm on a plate reader. Data points were fit with a second-order polynomial in Excel to obtain wavelengths at maximum absorbance. Values were reported as the difference between plasmon wavelengths of the sample and background (Δλmax). Self-interaction levels were determined based on Δλmax. Self-interaction may be considered: low when Δλmax<5.0 nm; medium when Δλmax≥5.0 nm and <20.0 nm; and high when Δλmax≥20.0 nm.

Δλmax obtained are shown in Table 13. As shown in the table, many of the tested IgG1s (A003, V004, V007, V010, and V013-V015) had Δλmax of <5.0, and many of the tested IgG1s (A001, A002, A004, A005, V002, V007, V009, and V016-V019) had Δλmax≥5.0 nm and <20.0 nM.

### Self-Interaction by DLS

Self-interaction was measured by dynamic light scattering (DLS). Diffusion Interaction Parameter (kD) of monoclonal antibodies, measured at concentrations lower than 12 mg/mL, has strong correlation with their solution behavior in very high concentrations (>100 mg/mL). Positive kD values indicate repulsive interaction among the molecules and has positive correlation with low viscosity at high concentration, in the same formulation buffer. kD values were obtained by measuring mutual diffusion coefficient for a series of different concentrations, by DLS. Specifically, DLS kD measurements at multiple concentrations between 0.5-12 mg/mL, in 10 mM Histidine buffer, pH 6.0 were taken. kD values <20 mL/g were considered as being associated with high viscosity or high opalescence.

kD values obtained are shown in Table 13. As shown in the table, many of the tested IgG1s (A001-A005 and V014-V016) had kD values ≥20 mL/g.

### Example 10: Developability—Fab Tm by DSF

Anti-CD3 antibodies A001-A005 and V002, V004, V007, V009, V010, V013, and V014-V019 were produced as IgG1s in CHO cells, and Fab fragments were generated from IgG1 antibodies by papain digestion and purification as described in Example 2. Melting temperature (Tm) was measured by differential scanning fluorometry (DSF) using a CFX96 Real-Time System from Bio-Rad. Briefly, 20 μL of 1 mg/mL sample was mixed with L of 20×SYPRO orange. The plate was scanned from 40° C. to 95° C. at a rate of 0.5° C./2 min in a C1000 thermocycler (BioRad) to collect Fret signal. The Fab Tm was assigned using the first derivative of the raw data from the Bio-Rad analysis software.

Tm values obtained are shown in Table 14. As shown in the table, all Fabs had a Tm value much higher than 65° C., indicating high stability and thus desirable developability.

### Exemplary Embodiments

Described herein below are some exemplary embodiments according to the present disclosure.

Embodiment 1. An antibody or antigen-binding fragment comprising: a complementarity determining region (CDR) comprising an amino acid sequence selected from any of those listed in Table 2 (Tables 2A and 2B).

Embodiment 2. An antibody or antigen-binding fragment comprising: a heavy chain variable domain (VH) comprising an amino acid sequence selected from any of those listed in Table 1 (Table 1A); and/or a light chain variable domain (VL) comprising an amino acid sequence selected from any of those listed in Table 1 (Table 1B).

Embodiment 3. The antibody or antigen-binding fragment of Embodiment 2, wherein: the VH comprises the amino acid sequence of SEQ ID NO: 1 and the VL comprises the amino acid sequence of SEQ ID NO: 15; the VH comprises the amino acid sequence of SEQ ID NO: 2 and the VL comprises the amino acid sequence of SEQ ID NO: 16; the VH comprises the amino acid sequence of SEQ ID NO: 3 and the VL comprises the amino acid sequence of SEQ ID NO: 15; the VH comprises the amino acid sequence of SEQ ID NO: 4 and the VL comprises the amino acid sequence of SEQ ID NO: 15; the VH comprises the amino acid sequence of SEQ ID NO: 5 and the VL comprises the amino acid sequence of SEQ ID NO: 15; the VH comprises the amino acid sequence of SEQ ID NO: 6 and the VL comprises the amino acid sequence of SEQ ID NO: 15; the VH comprises the amino acid sequence of SEQ ID NO: 7 and the VL comprises the amino acid sequence of SEQ ID NO: 15; the VH comprises the amino acid sequence of SEQ ID NO: 8 and the VL comprises the amino acid sequence of SEQ ID NO: 15; the VH comprises the amino acid sequence of SEQ ID NO: 9 and the VL comprises the amino acid sequence of SEQ ID NO: 16; the VH comprises the amino acid sequence of SEQ ID NO: 10 and the VL comprises the amino acid sequence of SEQ ID NO: 16; the VH comprises the amino acid sequence of SEQ ID NO: 11 and the VL comprises the amino acid sequence of SEQ ID NO: 16; the VH comprises the amino acid sequence of SEQ ID NO: 12 and the VL comprises the amino acid sequence of SEQ ID NO: 16; the VH comprises the amino acid sequence of SEQ ID NO: 13 and the VL comprises the amino acid sequence of SEQ ID NO: 16; or the VH comprises the amino acid sequence of SEQ ID NO: 14 and the VL comprises the amino acid sequence of SEQ ID NO: 16.

Embodiment 4. The antibody or antigen-binding fragment of Embodiment 1 comprising: a VH CDR3 (CDRH3) comprising an amino acid sequence selected from the group consisting of SEQ ID NO: 33 and 34; a VH CDR2 (CDRH2) comprising an amino acid sequence selected from the group consisting of SEQ ID NO: 21-32; a VH CDR1 (CDRH1) comprising an amino acid sequence selected from the group consisting of SEQ ID NO: 17-20; a VH comprising a CDR set according to any those listed in Table 2A; a VL CDR3 (CDRL3) comprising an amino acid sequence selected from the group consisting of SEQ ID NO: 38 or 39; a VL CDR2 (CDRL2) comprising an amino acid sequence selected from the group consisting of SEQ ID NO: 36 or 37; a VL CDR1 (CDRL1) comprising an amino acid sequence selected from the group consisting of SEQ ID NO: 33 or 34; and/or a VL comprising a CDR set according to any of those listed in Table 2B.

Embodiment 5. The antibody or antigen-binding fragment of any one of Embodiments 1-4, wherein the antibody or antigen-binding fragment binds to Cluster of Differentiation 3 (CD3).

Embodiment 6. The antibody or antigen-binding fragment of Embodiment 5, wherein the antibody or antigen-binding fragment binds to human and/or non-human primate CD3.

Embodiment 7. The antibody or antigen-binding fragment of any one of Embodiments 1-6, wherein: the antibody or antigen-binding fragment elicits T cell activation or T cell killing while displaying a decreased propensity to elicit cytokine production to levels capable of inducing cytokine release syndrome; the antibody or antigen-binding fragment comprises a multispecific antibody; the antibody or antigen-binding fragment comprises a bispecific antibody; the antibody or antigen-binding fragment comprises an scFv; the antibody or antigen-binding fragment comprises at least a second antigen-binding domain that specifically binds to an oncology target; an immune-oncology target; a neurodegenerative disease targets; an autoimmune disorder target; an infectious disease target; a metabolic disease target; a cognitive disorder target; a blood-brain barrier target; or a blood disease target; the antibody or antigen-binding fragment comprises at least a second antigen-binding domain having any one or more of the second binding specificities described herein; the antibody or antigen-binding fragment comprises at least a second antigen-binding domain that specifically binds to one or more of: BCMA, CTLA4 (cytotoxic T lymphocyte antigen-4), PD1 (programmed cell death protein 1), PD-L1 (programmed cell death ligand 1), LAG-3 (lymphocyte activation gene-3), TIM-3, CD20, CD2, CD19, Her2, EGFR, EpCAM, FcyRIIIa (CD16), FcyRIIa (CD32a), FcyRIIb (CD32b), FcγRI (CD64), Toll-like receptors (TLRs), TLR4, TLR9, cytokines, IL-2, IL-5, IL-13, IL-6, IL-17, IL-12, IL-23, TNFα, TGFβ, cytokine receptors, IL-2R, chemokines, chemokine receptors, growth factors, VEGF, and HGF; the antibody or antigen-binding fragment is comprised in a chimeric antigen receptor (CAR), which optionally comprises at least one transmembrane domain, and at least one intracellular domain from a T-cell receptor, optionally a CD3ζ subunit, and at least one co-stimulatory domain; the antibody or antigen-binding fragment comprises an scFv2-Fc2 and/or scFv-IgG; the antibody or antigen-binding fragment comprises an IgG constant domain; and/or the antibody or antigen-binding fragment comprises at least a second antigen-binding domain that specifically binds to an antigen, wherein the antibody or antigen-binding fragment comprises a multispecific format selected from the group consisting of: Fab-Fc-scFv, “bottle-opener”, Mab-scFv, Mab-Fv, Dual scFv, central Fv, central scFv, one-arm central scFv, Fab-Fab, Fab-Fv, mAb-Fv, mAb-Fab, DART, BiTE, common light chain-IgG, TandAb, Cross-Mab, SEED, BEAT, TrioMab, and DuetMab.

Embodiment 8. The antibody or antigen-binding fragment of any one of Embodiments 1-7, wherein the antibody or antigen-binding fragment exhibits a reduced PSR score relative to antibody A001 and/or A002.

Embodiment 9. A nucleic acid encoding an antibody or antigen-binding fragment according to any one of Embodiments 1-8.

Embodiment 10. A construct comprising a nucleic acid sequence according to Embodiment 9.

Embodiment 11. A cell comprising a nucleic acid according to Embodiment 9 and/or a construct according to Embodiment 10, wherein the cell is optionally a mammalian cell or a yeast cell.

Embodiment 12. A pharmaceutical composition comprising: an antibody or antigen-binding fragment according to any one of Embodiments 1-7 or a cell according to Embodiment 11; and a pharmaceutically acceptable carrier and/or excipient.

Embodiment 13. A method of treating a disorder in a mammal in need of such treatment, the method comprising administering an effective amount of: an antibody or antigen-binding fragment according to any one of Embodiments 1-7; a cell according to Embodiment 11; and optionally, an immune cell, T cell, and/or a natural killer (NK) cell.

Embodiment 14. The method of Embodiment 13, wherein the disorder comprises one or more of a proliferative disorder, an oncological disorder, an immuno-oncological disorder, a neurological disorder, a neurodegenerative disorder, and an autoimmune disorder.

Embodiment 15. The method of Embodiment 13 or 14, wherein the method further comprises administering an additional therapeutic agent.

Embodiment 16. The method of any one of Embodiments 13-15, wherein the mammal is a human.

### APPENDIX

TABLE 1A
VH amino acid sequence

| | | VH | |
|---|---|---|---|
| | | | SEQ |
| Antibody | ADI | | ID |
| No. | Name | Sequence | NO: |
| A001 | ADI- | QVQLVQSGAEVKKPGASVKVSCKASGFNIKDYYMHWVRQAPGQRLEWMGWIDLENANTIYDAKFQGRVTITRDTS | 2110 |
| | 26906 | ASTAYMELSSLRSEDTAVYYCARDAYGRYFYDVWGQGTLVTVSS | |
| V002 | ADI- | QVQLVQSGAEVKKPGASVKVSCKASGFNIKDYYMHWVRQAPGQRLEWMGWIDLENAETIYDAKFQDRVTITRDTS | 210 |
| | 67445 | ASTAYMELSSLRSEDTAVYYCARDAYGRYFYDEWGQGTLVTVSS | |
| V003 | ADI- | QVQLVQSGAEVKKPGASVKVSCKASGFDIKDYYMHWVRQAPGQRLEWMGWIELENADTIYDAKFQGRVTITRDTS | 310 |
| | 67446 | ASTAYMELSSLRSEDTAVYYCARDAYGRYFYDVWGQGTLVTVSS | |
| V004 | ADI- | QVQLVQSGAEVKKPGASVKVSCKASGFNIDDYYMHWVRQAPGQRLEWMGWIDLENDNTIYDAKFQGRVTITRDTS | 410 |
| | 67447 | ASTAYMELSSLRSEDTAVYYCARDAYGRYFYDVWGQGTLVTVSS | |
| V005 | ADI- | QVQLVQSGAEVKKPGASVKVSCKASGFNIDDYYMHWVRQAPGQRLEWMGWIDLENDNTIYDAKFQGRVTITRDTS | 510 |
| | 67448 | ASTAYMELSSLRSEDTAVYYCARDAYGRYFYDVWGQGTLVTVSS | |
| V006 | ADI- | QVQLVQSGAEVKKPGASVKVSCKASGFNDKDYYMHWVRQAPGQRLEWMGWIDLENAETIYDAKFQDRVTITRDT | 610 |
| | 67449 | SASTAYMELSSLRSEDTAVYYCARDAYGRYFYDVWGQGTLVTVSS | |
| V007 | ADI- | QVQLVQSGAEVKKPGASVKVSCKASGFNIDDYYMHWVRQAPGQRLEWMGWIDLENANTIDDAKFQGRVTITRDT | 710 |
| | 67450 | SASTAYMELSSLRSEDTAVYYCARDAYGRYFYDVWGQGTLVTVSS | |
| A002 | ADI- | QVQLVQSGAEVKKPGASVKVSCKASGFNIKDYYMHWVRQAPGQRLEWIGWIDLENANTVYDAKFQGRVTITRDTS | 2210 |
| | 50024 | ASTAYMELSSLRSEDTAVYYCARDAYGRYFYDVWGQGTLVTVSS | |
| V008 | ADI- | QVQLVQSGAEVKKPGASVKVSCKASGFNIKDYYMHWVRQAPGQRLEWIGWIDLENAETVYDAKFQDRVTITRDTSA | 810 |
| | 70325 | STAYMELSSLRSEDTAVYYCARDAYGRYFYDVWGQGTLVTVSS | |
| V009 | ADI- | QVQLVQSGAEVKKPGASVKVSCKASGFDIKDYYMHWVRQAPGQRLEWIGWIELENADTVYDAKFQGRVTITRDTSA | 910 |
| | 70326 | STAYMELSSLRSEDTAVYYCARDAYGRYFYDVWGQGTLVTVSS | |
| V010 | ADI- | QVQLVQSGAEVKKPGASVKVSCKASGFDIKDYYMHWVRQAPGQRLEWIGWIELENDDTVYDAKFQGRVTITRDTSA | 1010 |
| | 70327 | STAYMELSSLRSEDTAVYYCARDAYGRYFYDVWGQGTLVTVSS | |
| V011 | ADI- | QVQLVQSGAEVKKPGASVKVSCKASGFNIDDYYMHWVRQAPGQRLEWIGWIDLENDNTVYDAKFQGRVTITRDTS | 1110 |
| | 70328 | ASTAYMELSSLRSEDTAVYYCARDAYGRYFYDVWGQGTLVTVSS | |
| V012 | ADI- | QVQLVQSGAEVKKPGASVKVSCKASGFNDKDYYMHWVRQAPGQRLEWIGWIDLENAETVYDAKFQDRVTITRDTS | 1210 |
| | 70329 | ASTAYMELSSLRSEDTAVYYCARDAYGRYFYDVWGQGTLVTVSS | |
| V013 | ADI- | QVQLVQSGAEVKKPGASVKVSCKASGFNIDDYYMHWVRQAPGQRLEWIGWIDLENANTVDDAKFQGRVTITRDTS | 1310 |
| | 70330 | ASTAYMELSSLRSEDTAVYYCARDAYGRYFYDVWGQGTLVTVSS | |
| A003 | ADI- | QVQLVQSGAEVKKPGASVKVSCKASGFNIKDYYMHWVRQAPGQRLEWMGWIDLENANTIYDAKFQGRVTITRDTS | 2310 |
| | 26913 | ASTAYMELSSLRSEDTAVYYCARDQYGRYFYDVWGQGTLVTVSS | |
| A004 | ADI- | QVQLVQSGAEVKKPGASVKVSCKASGFNIKDYYMHWVRQAPGQRLEWMGWIDLENANTIYDAKFQGRVTITRDTS | 2410 |
| | 26920 | ASTAYMELSSLRSEDTAVYYCGRDAYGRYFYDVWGQGTLVTVSS | |
| A005 | ADI- | QVQLVQSGAEVKKPGASVKVSCKASGFNIKDYYMHWVRQAPGQRLEWMGWIDLEEGNTIYDAKFQGRVTITRDTS | 2510 |
| | 26921 | ASTAYMELSSLRSEDTAVYYCARDAYGRYFYDVWGQGTLVTVSS | |
| V014 | ADI- | QVQLVQSGAEVKKPGASVKVSCKASGFDIKDYYMHWVRQAPGQRLEWMGWIDLENANTIYDAKFQGRVTITRDTS | 1410 |
| | 76357 | ASTAYMELSSLRSEDTAVYYCARDQYGRYFYDVWGQGTLVTVSS | |
| V015 | ADI- | QVQLVQSGAEVKKPGASVKVSCKASGFNIKDYYMHWVRQAPGQRLEWMGWIDLENADTIYDAKFQGRVTITRDTS | 1510 |
| | 76358 | ASTAYMELSSLRSEDTAVYYCARDQYGRYFYDVWGQGTLVTVSS | |
| V016 | ADI- | QVQLVQSGAEVKKPGASVKVSCKASGFDIKDYYMHWVRQAPGQRLEWMGWIDLENANTIYDAKFQGRVTITRDTS | 1610 |
| | 76359 | ASTAYMELSSLRSEDTAVYYCGRDAYGRYFYDVWGQGTLVTVSS | |
| V017 | ADI- | QVQLVQSGAEVKKPGASVKVSCKASGFNIKDYYMHWVRQAPGQRLEWMGWIDLENADTIYDAKFQGRVTITRDTS | 1710 |
| | 76360 | ASTAYMELSSLRSEDTAVYYCGRDAYGRYFYDVWGQGTLVTVSS | |
| V018 | ADI- | QVQLVQSGAEVKKPGASVKVSCKASGFDIKDYYMHWVRQAPGQRLEWMGWIDLEEGNTIYDAKFQGRVTITRDTS | 1810 |
| | 76361 | ASTAYMELSSLRSEDTAVYYCARDAYGRYFYDVWGQGTLVTVSS | |
| V019 | ADI- | QVQLVQSGAEVKKPGASVKVSCKASGFNIKDYYMHWVRQAPGQRLEWMGWIDLEEGDTIYDAKFQGRVTITRDTS | 1910 |
| | 76362 | ASTAYMELSSLRSEDTAVYYCARDAYGRYFYDVWGQGTLVTVSS | |

Underlined residues correspond to CDRH1, CDRH2, and CDRH3 in the order of appearance.

TABLE 1B
VL amino acid sequence

| | | VL | |
|---|---|---|---|
| | | | SEQ |
| Antibody | ADI | | ID |
| No. | Name | Sequence | NO: |
| A001 | ADI- | DIVMTQSPDSLAVSLGERATINCKSSQSLLNARTGKNYLAWYQQKPGQPPKLLIYWASTRESGVPDRFSGSGSGTDF | 2120 |
| | 26906 | TLTISSLQAEDVAVYYCKQSYSRRTFGGGTKVEIK | |
| V002 | ADI- | DIVMTQSPDSLAVSLGERATINCKSSQSLLNARTGKNYLAWYQQKPGQPPKLLIYWASTRESGVPDRFSGSGSGTDF | 220 |
| | 67445 | TLTISSLQAEDVAVYYCKQSYSRRTFGGGTKVEIK | |
| V003 | ADI- | DIVMTQSPDSLAVSLGERATINCKSSQSLLNARTGKNYLAWYQQKPGQPPKLLIYWASTRESGVPDRFSGSGSGTDF | 320 |
| | 67446 | TLTISSLQAEDVAVYYCKQSYSRRTFGGGTKVEIK | |
| V004 | ADI- | DIVMTQSPDSLAVSLGERATINCKSSQSLLNARTGKNYLAWYQQKPGQPPKLLIYWASTRESGVPDRFSGSGSGTDF | 420 |
| | 67447 | TLTISSLQAEDVAVYYCKQSYSRRTFGGGTKVEIK | |
| V005 | ADI- | DIVMTQSPDSLAVSLGERATINCKSSQSLLNARTGKNYLAWYQQKPGQPPKLLIYWASTRESGVPDRFSGSGSGTDF | 520 |
| | 67448 | TLTISSLQAEDVAVYYCKQSYSRRTFGGGTKVEIK | |
| V006 | ADI- | DIVMTQSPDSLAVSLGERATINCKSSQSLLNARTGKNYLAWYQQKPGQPPKLLIYWASTRESGVPDRFSGSGSGTDF | 620 |
| | 67449 | TLTISSLQAEDVAVYYCKQSYSRRTFGGGTKVEIK | |
| V007 | ADI- | DIVMTQSPDSLAVSLGERATINCKSSQSLLNARTGKNYLAWYQQKPGQPPKLLIYWASTRESGVPDRFSGSGSGTDF | 720 |
| | 67450 | TLTISSLQAEDVAVYYCKQSYSRRTFGGGTKVEIK | |
| A002 | ADI- | DIVMSQSPDSLAVSLGERATINCKSSQSLLNARTGKNYLAWYQQKPGQPPKLLIYWASTRSSGVPDRFSGSGSGTDF | 2220 |
| | 50024 | TLTISSLQAEDVAVYYCVQSYFRRTFGGGTKVEIK | |
| V008 | ADI- | DIVMSQSPDSLAVSLGERATINCKSSQSLLNARTGKNYLAWYQQKPGQPPKLLIYWASTRSSGVPDRFSGSGSGTDF | 820 |
| | 70325 | TLTISSLQAEDVAVYYCVQSYFRRTFGGGTKVEIK | |
| V009 | ADI- | DIVMSQSPDSLAVSLGERATINCKSSQSLLNARTGKNYLAWYQQKPGQPPKLLIYWASTRSSGVPDRFSGSGSGTDF | 920 |
| | 70326 | TLTISSLQAEDVAVYYCVQSYFRRTFGGGTKVEIK | |
| V010 | ADI- | DIVMSQSPDSLAVSLGERATINCKSSQSLLNARTGKNYLAWYQQKPGQPPKLLIYWASTRSSGVPDRFSGSGSGTDF | 1020 |
| | 70327 | TLTISSLQAEDVAVYYCVQSYFRRTFGGGTKVEIK | |
| V011 | ADI- | DIVMSQSPDSLAVSLGERATINCKSSQSLLNARTGKNYLAWYQQKPGQPPKLLIYWASTRSSGVPDRFSGSGSGTDF | 1120 |
| | 70328 | TLTISSLQAEDVAVYYCVQSYFRRTFGGGTKVEIK | |
| V012 | ADI- | DIVMSQSPDSLAVSLGERATINCKSSQSLLNARTGKNYLAWYQQKPGQPPKLLIYWASTRSSGVPDRFSGSGSGTDF | 1220 |
| | 70329 | TLTISSLQAEDVAVYYCVQSYFRRTFGGGTKVEIK | |
| V013 | ADI- | DIVMSQSPDSLAVSLGERATINCKSSQSLLNARTGKNYLAWYQQKPGQPPKLLIYWASTRSSGVPDRFSGSGSGTDF | 1320 |
| | 70330 | TLTISSLQAEDVAVYYCVQSYFRRTFGGGTKVEIK | |
| A003 | ADI- | DIVMTQSPDSLAVSLGERATINCKSSQSLLNARTGKNYLAWYQQKPGQPPKLLIYWASTRESGVPDRFSGSGSGTDF | 2320 |
| | 26913 | TLTISSLQAEDVAVYYCKQSYSRRTFGGGTKVEIK | |
| A004 | ADI- | DIVMTQSPDSLAVSLGERATINCKSSQSLLNARTGKNYLAWYQQKPGQPPKLLIYWASTRESGVPDRFSGSGSGTDF | 2420 |
| | 26920 | TLTISSLQAEDVAVYYCKQSYSLRTFGGGTKVEIK | |
| A005 | ADI- | DIVMTQSPDSLAVSLGERATINCKSSQSLLNARTGKNYLAWYQQKPGQPPKLLIYWASTRESGVPDRFSGSGSGTDF | 2520 |
| | 26921 | TLTISSLQAEDVAVYYCKQSYSLRTFGGGTKVEIK | |
| V014 | ADI- | DIVMTQSPDSLAVSLGERATINCKSSQSLLNARTGKNYLAWYQQKPGQPPKLLIYWASTRESGVPDRFSGSGSGTDF | 1420 |
| | 76357 | TLTISSLQAEDVAVYYCKQSYSRRTFGGGTKVEIK | |
| V015 | ADI- | DIVMTQSPDSLAVSLGERATINCKSSQSLLNARTGKNYLAWYQQKPGQPPKLLIYWASTRESGVPDRFSGSGSGTDF | 1520 |
| | 76358 | TLTISSLQAEDVAVYYCKQSYSRRTFGGGTKVEIK | |
| V016 | ADI- | DIVMTQSPDSLAVSLGERATINCKSSQSLLNARTGKNYLAWYQQKPGQPPKLLIYWASTRESGVPDRFSGSGSGTDF | 1620 |
| | 76359 | TLTISSLQAEDVAVYYCKQSYSLRTFGGGTKVEIK | |
| V017 | ADI- | DIVMTQSPDSLAVSLGERATINCKSSQSLLNARTGKNYLAWYQQKPGQPPKLLIYWASTRESGVPDRFSGSGSGTDF | 1720 |
| | 76360 | TLTISSLQAEDVAVYYCKQSYSLRTFGGGTKVEIK | |
| V018 | ADI- | DIVMTQSPDSLAVSLGERATINCKSSQSLLNARTGKNYLAWYQQKPGQPPKLLIYWASTRESGVPDRFSGSGSGTDF | 1820 |
| | 76361 | TLTISSLQAEDVAVYYCKQSYSLRTFGGGTKVEIK | |
| V019 | ADI- | DIVMTQSPDSLAVSLGERATINCKSSQSLLNARTGKNYLAWYQQKPGQPPKLLIYWASTRESGVPDRFSGSGSGTDF | 1920 |
| | 76362 | TLTISSLQAEDVAVYYCKQSYSLRTFGGGTKVEIK | |

Underlined residues correspond to CDRL1, CDRL2, and CDRL3 in the order of appearance.

TABLE 1C
VH nucleic acid sequence

| | | VH | |
|---|---|---|---|
| Antibody | ADI | | SEQ ID |
| No. | Name | Sequence | NO: |
| A001 | ADI- | CAGGTGCAGCTGGTGCAGTCTGGGGCTGAGGTGAAGAAGCCTGGGGCCTCAGTGAAGGTCTCCTGCAA | 2150 |
| | 26906 | GGCTTCTGGATTCAACATCAAGGACTACTATATGCACTGGGTGCGACAGGCTCCTGGACAAAGGCTTGA | |
| | | GTGGATGGGATGGATCGACCTTGAAAATGCTAACACAATCTATGACGCTAAGTTTCAGGGCAGGGTCAC | |
| | | CATAACCAGGGACACGTCCGCCAGCACAGCCTACATGGAGCTGAGCAGCCTGAGATCTGAAGACACGGC | |
| | | GGTGTACTACTGCGCGAGAGATGCTTACGGGAGATATTTTTACGACGTGTGGGGCCAAGGAACCCTGGT | |
| | | CACCGTCTCCTCA | |
| V002 | ADI- | CAGGTGCAGCTGGTGCAGTCTGGGGCTGAGGTGAAGAAGCCTGGGGCCTCAGTGAAGGTCTCCTGCAA | 250 |
| | 67445 | GGCTTCTGGATTCAACATCAAGGACTACTATATGCACTGGGTGCGACAGGCTCCTGGACAAAGGCTTGA | |
| | | GTGGATGGGATGGATCGACCTTGAAAATGCTGAAACAATCTATGACGCTAAGTTTCAGGACAGGGTCAC | |
| | | CATAACCAGGGACACGTCCGCCAGCACAGCCTACATGGAGCTGAGCAGCCTGAGATCTGAAGACACGGC | |
| | | GGTGTACTACTGCGCGAGAGATGCTTACGGGAGATATTTTTACGACGAGTGGGGCCAAGGAACCCTGGT | |
| | | CACCGTCTCCTCA | |
| V003 | ADI- | CAGGTGCAGCTGGTGCAGTCTGGGGCTGAGGTGAAGAAGCCTGGGGCCTCAGTGAAGGTCTCCTGCAAGGCTTCTGG | 350 |
| | 67446 | ATTCGACATCAAGGACTACTATATGCACTGGGTGCGACAGGCTCCTGGACAAAGGCTTGAGTGGATGGGATGGATCG | |
| | | AGCTTGAAAATGCTGATACAATCTATGACGCTAAGTTTCAGGGCAGGGTCACCATAACCAGGGACACGTCCGCCAGC | |
| | | ACAGCCTACATGGAGCTGAGCAGCCTGAGATCTGAAGACACGGCGGTGTACTACTGCGCGAGAGATGCTTACGGGAG | |
| | | ATATTTTTACGACGTGTGGGGCCAAGGAACCCTGGTCACCGTCTCCTCA | |
| V004 | ADI- | CAGGTGCAGCTGGTGCAGTCTGGGGCTGAGGTGAAGAAGCCTGGGGCCTCAGTGAAGGTCTCCTGCAA | 450 |
| | 67447 | GGCTTCTGGATTCAACATCGATGACTACTATATGCACTGGGTGCGACAGGCTCCTGGACAAAGGCTTGA | |
| | | GTGGATGGGATGGATCGACCTTGAAAATGACAACACAATCTATGACGCTAAGTTTCAGGGCAGGGTCAC | |
| | | CATAACCAGGGACACGTCCGCCAGCACAGCCTACATGGAGCTGAGCAGCCTGAGATCTGAAGACACGGC | |
| | | GGTGTACTACTGCGCGAGAGATGCTTACGGGAGATATTTTTACGACGTGTGGGGCCAAGGAACCCTGGT | |
| | | CACCGTCTCCTCA | |
| V005 | ADI- | CAGGTGCAGCTGGTGCAGTCTGGGGCTGAGGTGAAGAAGCCTGGGGCCTCAGTGAAGGTCTCCTGCAA | 550 |
| | 67448 | GGCTTCTGGATTCAACATCGATGACTACTATATGCACTGGGTGCGACAGGCTCCTGGACAAAGGCTTGA | |
| | | GTGGATGGGATGGATCGACCTTGAAAATGACAACACAATCTATGACGCTAAGTTTCAGGGCAGGGTCAC | |
| | | CATAACCAGGGACACGTCCGCCAGCACAGCCTACATGGAGCTGAGCAGCCTGAGATCTGAAGACACGGC | |
| | | GGTGTACTACTGCGCGAGAGATGCTTACGGGAGATATTTTTACGACGTGTGGGGCCAAGGAACCCTGGT | |
| | | CACCGTCTCCTCA | |
| V006 | ADI- | CAGGTGCAGCTGGTGCAGTCTGGGGCTGAGGTGAAGAAGCCTGGGGCCTCAGTGAAGGTCTCCTGCAA | 650 |
| | 67449 | GGCTTCTGGATTCAACGATAAGGACTACTATATGCACTGGGTGCGACAGGCTCCTGGACAAAGGCTTGA | |
| | | GTGGATGGGATGGATCGACCTTGAAAATGCTGAGACAATCTATGACGCTAAGTTTCAGGATAGGGTCAC | |
| | | CATAACCAGGGACACGTCCGCCAGCACAGCCTACATGGAGCTGAGCAGCCTGAGATCTGAAGACACGGC | |
| | | GGTGTACTACTGCGCGAGAGATGCTTACGGGAGATATTTTTACGACGTGTGGGGCCAAGGAACCCTGGT | |
| | | CACCGTCTCCTCA | |
| V007 | ADI- | CAGGTGCAGCTGGTGCAGTCTGGGGCTGAGGTGAAGAAGCCTGGGGCCTCAGTGAAGGTCTCCTGCAA | 750 |
| | 67450 | GGCTTCTGGATTCAACATCGATGACTACTATATGCACTGGGTGCGACAGGCTCCTGGACAAAGGCTTGA | |
| | | GTGGATGGGATGGATCGACCTTGAAAATGCTAACACAATCGATGACGCTAAGTTTCAGGGCAGGGTCAC | |
| | | CATAACCAGGGACACGTCCGCCAGCACAGCCTACATGGAGCTGAGCAGCCTGAGATCTGAAGACACGGC | |
| | | GGTGTACTACTGCGCGAGAGATGCTTACGGGAGATATTTTTACGACGTGTGGGGCCAAGGAACCCTGGT | |
| | | CACCGTCTCCTCA | |
| A002 | ADI- | CAGGTGCAGCTGGTGCAGTCTGGGGCTGAGGTGAAGAAGCCTGGGGCCTCAGTGAAGGTCTCCTGCAA | 2250 |
| | 50024 | GGCTTCTGGATTCAACATCAAGGACTACTATATGCACTGGGTGCGACAGGCTCCTGGACAAAGGCTTGA | |
| | | GTGGATAGGATGGATCGACCTTGAAAATGCTAACACAGTCTATGACGCTAAGTTTCAGGGCAGGGTCAC | |
| | | CATAACCAGGGACACGTCCGCCAGCACAGCCTACATGGAGCTGAGCAGCCTGAGATCTGAAGACACGGC | |
| | | GGTGTACTACTGCGCGAGAGATGCTTACGGGAGATATTTTTACGACGTGTGGGGCCAAGGAACCCTGGT | |
| | | CACCGTCTCCTCA | |
| V008 | ADI- | CAGGTGCAGCTGGTGCAGTCTGGGGCTGAGGTGAAGAAGCCTGGGGCCTCAGTGAAGGTCTCCTGCAAGGCTTCT | 850 |
| | 70325 | GGATTCAACATCAAGGACTACTATATGCACTGGGTGAGGCAGGCTCCTGGACAAAGGCTTGAGTGGATAGGATGG | |
| | | ATCGACCTTGAAAATGCTGAGACAGTCTATGACGCTAAGTTTCAGGATAGGGTCACCATAACCAGGGACACGTCCG | |
| | | CCAGCACAGCCTACATGGAGCTGAGCAGCCTGAGATCTGAAGACACGGCGGTGTACTACTGCGCGAGAGATGCTT | |
| | | ACGGGAGATATTTTTACGACGTGTGGGGCCAAGGAACCCTGGTCACCGTCTCCTCA | |
| V009 | ADI- | CAGGTGCAGCTGGTGCAGTCTGGGGCTGAGGTGAAGAAGCCTGGGGCCTCAGTGAAGGTCTCCTGCAA | 950 |
| | 70326 | GGCTTCTGGATTCgacATCAAGGACTACTATATGCACTGGGTGaggCAGGCTCCTGGACAAAGGCTTGAGT | |
| | | GGATAGGATGGATCgagCTTGAAAATGCTgacACAGTCTATGACGCTAAGTTTCAGGGCAGGGTCACCAT | |
| | | AACCAGGGACACGTCCGCCAGCACAGCCTACATGGAGCTGAGCAGCCTGAGATCTGAAGACACGGCGG | |
| | | TGTACTACTGCGCGAGAGATGCTTACGGGAGATATTTTTACGACGTGTGGGGCCAAGGAACCCTGGTCA | |
| | | CCGTCTCCTCA | |
| V010 | ADI- | CAGGTGCAGCTGGTGCAGTCTGGGGCTGAGGTGAAGAAGCCTGGGGCCTCAGTGAAGGTCTCCTGCAA | 1050 |
| | 70327 | GGCTTCTGGATTCgacATCAAGGACTACTATATGCACTGGGTGagaCAGGCTCCTGGACAAAGGCTTGAGT | |
| | | GGATAGGATGGATCgagCTTGAAAATgacgacACAGTCTATGACGCTAAGTTTCAGGGCAGGGTCACCATA | |
| | | ACCAGGGACACGTCCGCCAGCACAGCCTACATGGAGCTGAGCAGCCTGAGATCTGAAGACACGGCGGT | |
| | | GTACTACTGCGCGAGAGATGCTTACGGGAGATATTTTTACGACGTGTGGGGCCAAGGAACCCTGGTCAC | |
| | | CGTCTCCTCA | |
| V011 | ADI- | CAGGTGCAGCTGGTGCAGTCTGGGGCTGAGGTGAAGAAGCCTGGGGCCTCAGTGAAGGTCTCCTGCAAGGCTTCT | 1150 |
| | 70328 | GGATTCAACATCGATGACTACTATATGCACTGGGTGAGACAGGCTCCTGGACAAAGGCTTGAGTGGATAGGATGG | |
| | | ATCGACCTTGAAAATGATAACACAGTCTATGACGCTAAGTTTCAGGGCAGGGTCACCATAACCAGGGACACGTCCG | |
| | | CCAGCACAGCCTACATGGAGCTGAGCAGCCTGAGATCTGAAGACACGGCGGTGTACTACTGCGCGAGAGATGCTT | |
| | | ACGGGAGATATTTTTACGACGTGTGGGGCCAAGGAACCCTGGTCACCGTCTCCTCA | |
| V012 | ADI- | CAGGTGCAGCTGGTGCAGTCTGGGGCTGAGGTGAAGAAGCCTGGGGCCTCAGTGAAGGTCTCCTGCAAGGCTTCT | 1250 |
| | 70329 | GGATTCAACGACAAGGACTACTATATGCACTGGGTGAGGCAGGCTCCTGGACAAAGGCTTGAGTGGATAGGATG | |
| | | GATCGACCTTGAAAATGCTGAGACAGTCTATGACGCTAAGTTTCAGGACAGGGTCACCATAACCAGGGACACGTCC | |
| | | GCCAGCACAGCCTACATGGAGCTGAGCAGCCTGAGATCTGAAGACACGGCGGTGTACTACTGCGCGAGAGATGCT | |
| | | TACGGGAGATATTTTTACGACGTGTGGGGCCAAGGAACCCTGGTCACCGTCTCCTCA | |
| V013 | ADI- | CAGGTGCAGCTGGTGCAGTCTGGGGCTGAGGTGAAGAAGCCTGGGGCCTCAGTGAAGGTCTCCTGCAA | 1350 |
| | 70330 | GGCTTCTGGATTCAACATCgatGACTACTATATGCACTGGGTGaggCAGGCTCCTGGACAAAGGCTTGAGT | |
| | | GGATAGGATGGATCGACCTTGAAAATGCTAACACAGTCgatGACGCTAAGTTTCAGGGCAGGGTCACCAT | |
| | | AACCAGGGACACGTCCGCCAGCACAGCCTACATGGAGCTGAGCAGCCTGAGATCTGAAGACACGGCGG | |
| | | TGTACTACTGCGCGAGAGATGCTTACGGGAGATATTTTTACGACGTGTGGGGCCAAGGAACCCTGGTCA | |
| | | CCGTCTCCTCA | |
| A003 | ADI- | CAGGTGCAGCTGGTGCAGTCTGGGGCTGAGGTGAAGAAGCCTGGGGCCTCAGTGAAGGTCTCCTGCAAGGCTTCTGG | 2350 |
| | 26913 | ATTCAACATCAAGGACTACTATATGCACTGGGTGCGACAGGCTCCTGGACAAAGGCTTGAGTGGATGGGATGGATCG | |
| | | ACCTTGAAAATGCTAACACAATCTATGACGCTAAGTTTCAGGGCAGGGTCACCATAACCAGGGACACGTCCGCCAGC | |
| | | ACAGCCTACATGGAGCTGAGCAGCCTGAGATCTGAAGACACGGCGGTGTACTACTGCGCGAGAGATCAGTACGGGAG | |
| | | ATATTTTTACGACGTGTGGGGCCAAGGAACCCTGGTCACCGTCTCCTCA | |
| A004 | ADI- | CAGGTGCAGCTGGTGCAGTCTGGGGCTGAGGTGAAGAAGCCTGGGGCCTCAGTGAAGGTCTCCTGCAAGGCTTCTGG | 2450 |
| | 26920 | ATTCAACATCAAGGACTACTATATGCACTGGGTGCGACAGGCTCCTGGACAAAGGCTTGAGTGGATGGGATGGATCG | |
| | | ACCTTGAAAATGCTAACACAATCTATGACGCTAAGTTTCAGGGCAGGGTCACCATAACCAGGGACACGTCCGCCAGC | |
| | | ACAGCCTACATGGAGCTGAGCAGCCTGAGATCTGAAGACACGGCGGTGTACTACTGCGGTAGAGATGCGTACGGGAG | |
| | | ATATTTTTACGACGTGTGGGGCCAAGGAACCCTGGTCACCGTCTCCTCA | |
| A005 | ADI- | CAGGTGCAGCTGGTGCAGTCTGGGGCTGAGGTGAAGAAGCCTGGGGCCTCAGTGAAGGTCTCCTGCAAGGCTTCTGG | 2550 |
| | 26921 | ATTCAACATCAAGGACTACTATATGCACTGGGTGCGACAGGCTCCTGGACAAAGGCTTGAGTGGATGGGATGGATCG | |
| | | ACCTTGAAGAGGGCAACACAATCTATGACGCTAAGTTTCAGGGCAGGGTCACCATAACCAGGGACACGTCCGCCAGC | |
| | | ACAGCCTACATGGAGCTGAGCAGCCTGAGATCTGAAGACACGGCGGTGTACTACTGCGCGAGAGATGCTTACGGGAG | |
| | | ATATTTTTACGACGTGTGGGGCCAAGGAACCCTGGTCACCGTCTCCTCA | |
| V014 | ADI- | CAGGTGCAGCTGGTGCAGTCTGGGGCTGAGGTGAAGAAGCCTGGGGCCTCAGTGAAGGTTTCCTGCAA | 1450 |
| | 76357 | GGCTTCTGGATTTGACATTAAAGATTATTACATGCATTGGGTGCGCCAGGCCCCCGGACAAAGGCTTGAG | |
| | | TGGATGGGATGGATCGACTTGGAGAACGCGAACACAATCTATGATGCTAAGTTCCAGGGCAGAGTCACC | |
| | | ATTACCAGGGACACATCCGCGAGCACAGCCTACATGGAGCTGAGCAGCCTGAGATCTGAAGACACGGC | |
| | | GGTGTACTACTGCGCACGTGACCAGTATGGCCGGTACTTCTATGACGTTTGGGGCCAGGGCACCCTGGT | |
| | | CACCGTCTCCTCA | |
| V015 | ADI- | CAGGTGCAGCTGGTGCAGTCTGGGGCTGAGGTGAAGAAGCCTGGGGCCTCAGTGAAGGTTTCCTGCAA | 1550 |
| | 76358 | GGCTTCTGGATTTAATATCAAAGATTATTATATGCATTGGGTGCGCCAGGCCCCCGGACAAAGGCTTGAG | |
| | | TGGATGGGATGGATCGATCTCGAAAATGCGGATACAATCTATGATGCAAAGTTCCAGGGCAGAGTCACC | |
| | | ATTACCAGGGACACATCCGCGAGCACAGCCTACATGGAGCTGAGCAGCCTGAGATCTGAAGACACGGC | |
| | | GGTGTACTACTGCGCCCGCGATCAATACGGAAGGTATTTCTATGATGTCTGGGGCCAGGGCACCCTGGT | |
| | | CACCGTCTCCTCA | |
| V016 | ADI- | CAGGTGCAGCTGGTGCAGTCTGGGGCTGAGGTGAAGAAGCCTGGGGCCTCAGTGAAGGTTTCCTGCAA | 1650 |
| | 76359 | GGCTTCTGGATTCGACATCAAAGATTATTACATGCATTGGGTGCGCCAGGCCCCCGGACAAAGGCTTGA | |
| | | GTGGATGGGATGGATCGATCTAGAAAATGCTAACACAATATATGATGCGAAGTTCCAGGGCAGAGTCAC | |
| | | CATTACCAGGGACACATCCGCGAGCACAGCCTACATGGAGCTGAGCAGCCTGAGATCTGAAGACACGGC | |
| | | GGTGTACTACTGCGGCCGTGACGCCTATGGCCGATATTTCTATGACGTCTGGGGCCAGGGCACCCTGGT | |
| | | CACCGTCTCCTCA | |
| V017 | ADI- | CAGGTGCAGCTGGTGCAGTCTGGGGCTGAGGTGAAGAAGCCTGGGGCCTCAGTGAAGGTTTCCTGCAA | 1750 |
| | 76360 | GGCTTCTGGATTTAACATAAAGGACTATTATATGCATTGGGTGCGCCAGGCCCCCGGACAAAGGCTTGA | |
| | | GTGGATGGGATGGATCGACCTGGAAAACGCAGACACAATATATGACGCTAAGTTCCAGGGCAGAGTCA | |
| | | CCATTACCAGGGACACATCCGCGAGCACAGCCTACATGGAGCTGAGCAGCCTGAGATCTGAAGACACGG | |
| | | CGGTGTACTACTGCGGCCGGGACGCCTATGGTAGATATTTTTATGATGTCTGGGGCCAGGGCACCCTGG | |
| | | TCACCGTCTCCTCA | |
| V018 | ADI- | CAGGTGCAGCTGGTGCAGTCTGGGGCTGAGGTGAAGAAGCCTGGGGCCTCAGTGAAGGTTTCCTGCAA | 1850 |
| | 76361 | GGCTTCTGGATTCGATATCAAGGATTATTATATGCATTGGGTGCGCCAGGCCCCCGGACAAAGGCTTGA | |
| | | GTGGATGGGATGGATCGATCTAGAAGAAGGTAACACAATCTATGACGCGAAGTTCCAGGGCAGAGTCA | |
| | | CCATTACCAGGGACACATCCGCGAGCACAGCCTACATGGAGCTGAGCAGCCTGAGATCTGAAGACACGG | |
| | | CGGTGTACTACTGCGCTCGAGATGCTTACGGTCGGTACTTTTATGACGTTTGGGGCCAGGGCACCCTGGT | |
| | | CACCGTCTCCTCA | |
| V019 | ADI- | CAGGTGCAGCTGGTGCAGTCTGGGGCTGAGGTGAAGAAGCCTGGGGCCTCAGTGAAGGTTTCCTGCAA | 1950 |
| | 76362 | GGCTTCTGGATTTAACATCAAGGATTATTATATGCATTGGGTGCGCCAGGCCCCCGGACAAAGGCTTGAG | |
| | | TGGATGGGATGGATCGATCTTGAGGAAGGTGATACAATTTATGATGCCAAGTTCCAGGGCAGAGTCACC | |
| | | ATTACCAGGGACACATCCGCGAGCACAGCCTACATGGAGCTGAGCAGCCTGAGATCTGAAGACACGGC | |
| | | GGTGTACTACTGCGCACGTGATGCATATGGACGTTATTTTTATGACGTTTGGGGCCAGGGCACCCTGGTC | |
| | | ACCGTCTCCTCA | |

The closest germline is VH1-03.

TABLE 1D
VL nucleic acid sequence

| | | VL | |
|---|---|---|---|
| Anti- | | | SEQ |
| body | | | ID |
| No. | ADI Name | Sequence | NO: |
| A001 | ADI-26906 | GACATCGTGATGACCCAGTCTCCAGACTCCCTGGCTGTGTCTCTGGGCGAGAGGGCCACCATCAACTGCA | 2160 |
| | | AGTCCAGCCAGAGTCTTTTAAACGCTAGAACCGGAAAGAACTACTTAGCTTGGTACCAGCAGAAACCAG | |
| | | GACAGCCTCCTAAGCTGCTCATTTACTGGGCATCTACCCGGGAATCCGGGGTCCCTGACCGATTCAGTGG | |
| | | CAGCGGGTCTGGGACAGACTTCACTCTCACTATCAGTAGCCTGCAGGCTGAAGATGTGGCAGTTTATTAC | |
| | | TGTAAGCAATCTTATTCTCGGAGAACTTTCGGCGGAGGGACCAAGGTCGAGATCAAA | |
| V002 | ADI-67445 | GACATCGTGATGACCCAGTCTCCAGACTCCCTGGCTGTGTCTCTGGGCGAGAGGGCCACCATCAACTGCA | 260 |
| | | AGTCCAGCCAGAGTCTTTTAAACGCTAGAACCGGAAAGAACTACTTAGCTTGGTACCAGCAGAAACCAG | |
| | | GACAGCCTCCTAAGCTGCTCATTTACTGGGCATCTACCCGGGAATCCGGGGTCCCTGACCGATTCAGTGG | |
| | | CAGCGGGTCTGGGACAGACTTCACTCTCACTATCAGTAGCCTGCAGGCTGAAGATGTGGCAGTTTATTAC | |
| | | TGTAAGCAATCTTATTCTCGGAGAACTTTCGGCGGAGGGACCAAGGTCGAGATCAAA | |
| V003 | ADI-67446 | GACATCGTGATGACCCAGTCTCCAGACTCCCTGGCTGTGTCTCTGGGCGAGAGGGCCACCATCAACTGCAAGTCCAG | 360 |
| | | CCAGAGTCTTTTAAACGCTAGAACCGGAAAGAACTACTTAGCTTGGTACCAGCAGAAACCAGGACAGCCTCCTAAGC | |
| | | TGCTCATTTACTGGGCATCTACCCGGGAATCCGGGGTCCCTGACCGATTCAGTGGCAGCGGGTCTGGGACAGACTTC | |
| | | ACTCTCACTATCAGTAGCCTGCAGGCTGAAGATGTGGCAGTTTATTACTGTAAGCAATCTTATTCTCGGAGAACTTT | |
| | | CGGCGGAGGGACCAAGGTCGAGATCAAA | |
| V004 | ADI-67447 | GACATCGTGATGACCCAGTCTCCAGACTCCCTGGCTGTGTCTCTGGGCGAGAGGGCCACCATCAACTGCA | 460 |
| | | AGTCCAGCCAGAGTCTTTTAAACGCTAGAACCGGAAAGAACTACTTAGCTTGGTACCAGCAGAAACCAG | |
| | | GACAGCCTCCTAAGCTGCTCATTTACTGGGCATCTACCCGGGAATCCGGGGTCCCTGACCGATTCAGTGG | |
| | | CAGCGGGTCTGGGACAGACTTCACTCTCACTATCAGTAGCCTGCAGGCTGAAGATGTGGCAGTTTATTAC | |
| | | TGTAAGCAATCTTATTCTCGGAGAACTTTCGGCGGAGGGACCAAGGTCGAGATCAAA | |
| V005 | ADI-67448 | GACATCGTGATGACCCAGTCTCCAGACTCCCTGGCTGTGTCTCTGGGCGAGAGGGCCACCATCAACTGCAAGTCCAG | 560 |
| | | CCAGAGTCTTTTAAACGCTAGAACCGGAAAGAACTACTTAGCTTGGTACCAGCAGAAACCAGGACAGCCTCCTAAGC | |
| | | TGCTCATTTACTGGGCATCTACCCGGGAATCCGGGGTCCCTGACCGATTCAGTGGCAGCGGGTCTGGGACAGACTTC | |
| | | ACTCTCACTATCAGTAGCCTGCAGGCTGAAGATGTGGCAGTTTATTACTGTAAGCAATCTTATTCTCGGAGAACTTT | |
| | | CGGCGGAGGGACCAAGGTCGAGATCAAA | |
| V006 | ADI-67449 | GACATCGTGATGACCCAGTCTCCAGACTCCCTGGCTGTGTCTCTGGGCGAGAGGGCCACCATCAACTGCAAGTCCAG | 660 |
| | | CCAGAGTCTTTTAAACGCTAGAACCGGAAAGAACTACTTAGCTTGGTACCAGCAGAAACCAGGACAGCCTCCTAAGC | |
| | | TGCTCATTTACTGGGCATCTACCCGGGAATCCGGGGTCCCTGACCGATTCAGTGGCAGCGGGTCTGGGACAGACTTC | |
| | | ACTCTCACTATCAGTAGCCTGCAGGCTGAAGATGTGGCAGTTTATTACTGTAAGCAATCTTATTCTCGGAGAACTTT | |
| | | CGGCGGAGGGACCAAGGTCGAGATCAAA | |
| V007 | ADI-67450 | GACATCGTGATGACCCAGTCTCCAGACTCCCTGGCTGTGTCTCTGGGCGAGAGGGCCACCATCAACTGCA | 760 |
| | | AGTCCAGCCAGAGTCTTTTAAACGCTAGAACCGGAAAGAACTACTTAGCTTGGTACCAGCAGAAACCAG | |
| | | GACAGCCTCCTAAGCTGCTCATTTACTGGGCATCTACCCGGGAATCCGGGGTCCCTGACCGATTCAGTGG | |
| | | CAGCGGGTCTGGGACAGACTTCACTCTCACTATCAGTAGCCTGCAGGCTGAAGATGTGGCAGTTTATTAC | |
| | | TGTAAGCAATCTTATTCTCGGAGAACTTTCGGCGGAGGGACCAAGGTCGAGATCAAA | |
| A002 | ADI-50024 | GACATCGTGATGTCCCAGTCTCCAGACTCCCTGGCTGTGTCTCTGGGCGAGAGGGCCACCATCAACTGCA | 2260 |
| | | AGTCCAGCCAGAGTCTTTTAAACGCTAGAACCGGAAAGAACTACTTAGCTTGGTACCAGCAGAAACCAG | |
| | | GACAGCCTCCTAAGCTGCTCATTTACTGGGCATCTACCCGGTCGTCCGGGGTCCCTGACCGATTCAGCGG | |
| | | CAGCGGGTCTGGGACAGACTTCACTCTCACTATCAGTAGCCTGCAGGCTGAAGATGTGGCAGTTTATTAC | |
| | | TGTGTGCAATCTTATTTTCGGAGAACTTTCGGCGGAGGGACCAAGGTCGAGATCAAA | |
| V008 | ADI-70325 | GACATCGTGATGTCCCAGTCTCCAGACTCCCTGGCTGTGTCTCTGGGCGAGAGGGCCACCATCAACTGCAAGTCCA | 860 |
| | | GCCAGAGTCTTTTAAACGCTAGAACCGGAAAGAACTACTTAGCTTGGTACCAGCAGAAACCAGGACAGCCTCCTAA | |
| | | GCTGCTCATTTACTGGGCATCTACCCGGTCGTCCGGGGTCCCTGACCGATTCAGCGGCAGCGGGTCTGGGACAGAC | |
| | | TTCACTCTCACTATCAGTAGCCTGCAGGCTGAAGATGTGGCAGTTTATTACTGTGTGCAATCTTATTTTCGGAGAAC | |
| | | TTTCGGCGGAGGGACCAAGGTCGAGATCAAA | |
| V009 | ADI-70326 | GACATCGTGATGTCCCAGTCTCCAGACTCCCTGGCTGTGTCTCTGGGCGAGAGGGCCACCATCAACTGCA | 960 |
| | | AGTCCAGCCAGAGTCTTTTAAACGCTAGAACCGGAAAGAACTACTTAGCTTGGTACCAGCAGAAACCAG | |
| | | GACAGCCTCCTAAGCTGCTCATTTACTGGGCATCTACCCGGTCGTCCGGGGTCCCTGACCGATTCAGCGG | |
| | | CAGCGGGTCTGGGACAGACTTCACTCTCACTATCAGTAGCCTGCAGGCTGAAGATGTGGCAGTTTATTAC | |
| | | TGTGTGCAATCTTATTTTCGGAGAACTTTCGGCGGAGGGACCAAGGTCGAGATCAAA | |
| V010 | ADI-70327 | GACATCGTGATGTCCCAGTCTCCAGACTCCCTGGCTGTGTCTCTGGGCGAGAGGGCCACCATCAACTGCA | 1060 |
| | | AGTCCAGCCAGAGTCTTTTAAACGCTAGAACCGGAAAGAACTACTTAGCTTGGTACCAGCAGAAACCAG | |
| | | GACAGCCTCCTAAGCTGCTCATTTACTGGGCATCTACCCGGTCGTCCGGGGTCCCTGACCGATTCAGCGG | |
| | | CAGCGGGTCTGGGACAGACTTCACTCTCACTATCAGTAGCCTGCAGGCTGAAGATGTGGCAGTTTATTAC | |
| | | TGTGTGCAATCTTATTTTCGGAGAACTTTCGGCGGAGGGACCAAGGTCGAGATCAAA | |
| V011 | ADI-70328 | GACATCGTGATGTCCCAGTCTCCAGACTCCCTGGCTGTGTCTCTGGGCGAGAGGGCCACCATCAACTGCAAGTCCA | 1160 |
| | | GCCAGAGTCTTTTAAACGCTAGAACCGGAAAGAACTACTTAGCTTGGTACCAGCAGAAACCAGGACAGCCTCCTAA | |
| | | GCTGCTCATTTACTGGGCATCTACCCGGTCGTCCGGGGTCCCTGACCGATTCAGCGGCAGCGGGTCTGGGACAGAC | |
| | | TTCACTCTCACTATCAGTAGCCTGCAGGCTGAAGATGTGGCAGTTTATTACTGTGTGCAATCTTATTTTCGGAGAAC | |
| | | TTTCGGCGGAGGGACCAAGGTCGAGATCAAA | |
| V012 | ADI-70329 | GACATCGTGATGTCCCAGTCTCCAGACTCCCTGGCTGTGTCTCTGGGCGAGAGGGCCACCATCAACTGCAAGTCCA | 1260 |
| | | GCCAGAGTCTTTTAAACGCTAGAACCGGAAAGAACTACTTAGCTTGGTACCAGCAGAAACCAGGACAGCCTCCTAA | |
| | | GCTGCTCATTTACTGGGCATCTACCCGGTCGTCCGGGGTCCCTGACCGATTCAGCGGCAGCGGGTCTGGGACAGAC | |
| | | TTCACTCTCACTATCAGTAGCCTGCAGGCTGAAGATGTGGCAGTTTATTACTGTGTGCAATCTTATTTTCGGAGAAC | |
| | | TTTCGGCGGAGGGACCAAGGTCGAGATCAAA | |
| V013 | ADI-70330 | GACATCGTGATGTCCCAGTCTCCAGACTCCCTGGCTGTGTCTCTGGGCGAGAGGGCCACCATCAACTGCA | 1360 |
| | | AGTCCAGCCAGAGTCTTTTAAACGCTAGAACCGGAAAGAACTACTTAGCTTGGTACCAGCAGAAACCAG | |
| | | GACAGCCTCCTAAGCTGCTCATTTACTGGGCATCTACCCGGTCGTCCGGGGTCCCTGACCGATTCAGCGG | |
| | | CAGCGGGTCTGGGACAGACTTCACTCTCACTATCAGTAGCCTGCAGGCTGAAGATGTGGCAGTTTATTAC | |
| | | TGTGTGCAATCTTATTTTCGGAGAACTTTCGGCGGAGGGACCAAGGTCGAGATCAAA | |
| A003 | ADI-26913 | GACATCGTGATGACCCAGTCTCCAGACTCCCTGGCTGTGTCTCTGGGCGAGAGGGCCACCATCAACTGCAAGTCCAG | 2360 |
| | | CCAGAGTCTTTTAAACGCTAGAACCGGAAAGAACTACTTAGCTTGGTACCAGCAGAAACCAGGACAGCCTCCTAAGC | |
| | | TGCTCATTTACTGGGCATCTACCCGGGAATCCGGGGTCCCTGACCGATTCAGTGGCAGCGGGTCTGGGACAGACTTC | |
| | | ACTCTCACTATCAGTAGCCTGCAGGCTGAAGATGTGGCAGTTTATTACTGTAAGCAATCTTATTCTCGGAGAACTTT | |
| | | CGGCGGAGGGACCAAGGTCGAGATCAAA | |
| A004 | ADI-26920 | GACATCGTGATGACCCAGTCTCCAGACTCCCTGGCTGTGTCTCTGGGCGAGAGGGCCACCATCAACTGCAAGTCCAG | 2460 |
| | | CCAGAGTCTTTTAAACGCTAGAACCGGAAAGAACTACTTAGCTTGGTACCAGCAGAAACCAGGACAGCCTCCTAAGC | |
| | | TGCTCATTTACTGGGCATCTACCCGGGAATCCGGGGTCCCTGACCGATTCAGTGGCAGCGGGTCTGGGACAGACTTC | |
| | | ACTCTCACTATCAGTAGCCTGCAGGCTGAAGATGTGGCAGTTTATTACTGTAAGCAATCTTATTCTCTGAGAACTTT | |
| | | CGGCGGAGGGACCAAGGTCGAGATCAAA | |
| A005 | ADI-26921 | GACATCGTGATGACCCAGTCTCCAGACTCCCTGGCTGTGTCTCTGGGCGAGAGGGCCACCATCAACTGCAAGTCCAG | 2560 |
| | | CCAGAGTCTTTTAAACGCTAGAACCGGAAAGAACTACTTAGCTTGGTACCAGCAGAAACCAGGACAGCCTCCTAAGC | |
| | | TGCTCATTTACTGGGCATCTACCCGGGAATCCGGGGTCCCTGACCGATTCAGTGGCAGCGGGTCTGGGACAGACTTC | |
| | | ACTCTCACTATCAGTAGCCTGCAGGCTGAAGATGTGGCAGTTTATTACTGTAAGCAATCTTATTCTCTGAGAACTTT | |
| | | CGGCGGAGGGACCAAGGTCGAGATCAAA | |
| V014 | ADI-76357 | GACATCGTGATGACCCAGTCTCCAGACTCCCTGGCTGTGTCTCTGGGCGAGAGGGCCACCATCAACTGCAAGTCCA | 1460 |
| | | GCCAGAGTCTTTTAAACGCTAGAACCGGAAAGAACTACTTAGCTTGGTACCAGCAGAAACCAGGACAGCCTCCTAA | |
| | | GCTGCTCATTTACTGGGCATCTACCCGGGAATCCGGGGTCCCTGACCGATTCAGTGGCAGCGGGTCTGGGACAGA | |
| | | CTTCACTCTCACTATCAGTAGCCTGCAGGCTGAAGATGTGGCAGTTTATTACTGTAAGCAATCTTATTCTCGGAGAA | |
| | | CTTTCGGCGGAGGGACCAAGGTCGAGATCAAA | |
| V015 | ADI-76358 | GACATCGTGATGACCCAGTCTCCAGACTCCCTGGCTGTGTCTCTGGGCGAGAGGGCCACCATCAACTGCAAGTCCA | 1560 |
| | | GCCAGAGTCTITTAAACGCTAGAACCGGAAAGAACTACTTAGCTTGGTACCAGCAGAAACCAGGACAGCCTCCTAA | |
| | | GCTGCTCATTTACTGGGCATCTACCCGGGAATCCGGGGTCCCTGACCGATTCAGTGGCAGCGGGTCTGGGACAGA | |
| | | CTTCACTCTCACTATCAGTAGCCTGCAGGCTGAAGATGTGGCAGTTTATTACTGTAAGCAATCTTATTCTCGGAGAA | |
| | | CTTTCGGCGGAGGGACCAAGGTCGAGATCAAA | |
| V016 | ADI-76359 | GACATCGTGATGACCCAGTCTCCAGACTCCCTGGCTGTGTCTCTGGGCGAGAGGGCCACCATCAACTGCAAGTCCA | 1660 |
| | | GCCAGAGTCTTTTAAACGCTAGAACCGGAAAGAACTACTTAGCTTGGTACCAGCAGAAACCAGGACAGCCTCCTAA | |
| | | GCTGCTCATTTACTGGGCATCTACCCGGGAATCCGGGGTCCCTGACCGATTCAGTGGCAGCGGGTCTGGGACAGA | |
| | | CTTCACTCTCACTATCAGTAGCCTGCAGGCTGAAGATGTGGCAGTTTATTACTGTAAGCAATCTTATTCTCTGAGAA | |
| | | CTTTCGGCGGAGGGACCAAGGTCGAGATCAAA | |
| V017 | ADI-76360 | GACATCGTGATGACCCAGTCTCCAGACTCCCTGGCTGTGTCTCTGGGCGAGAGGGCCACCATCAACTGCAAGTCCA | 1760 |
| | | GCCAGAGTCTTTTAAACGCTAGAACCGGAAAGAACTACTTAGCTTGGTACCAGCAGAAACCAGGACAGCCTCCTAA | |
| | | GCTGCTCATTTACTGGGCATCTACCCGGGAATCCGGGGTCCCTGACCGATTCAGTGGCAGCGGGTCTGGGACAGA | |
| | | CTTCACTCTCACTATCAGTAGCCTGCAGGCTGAAGATGTGGCAGTTTATTACTGTAAGCAATCTTATTCTCTGAGAA | |
| | | CTTTCGGCGGAGGGACCAAGGTCGAGATCAAA | |
| V018 | ADI-76361 | GACATCGTGATGACCCAGTCTCCAGACTCCCTGGCTGTGTCTCTGGGCGAGAGGGCCACCATCAACTGCAAGTCCA | 1860 |
| | | GCCAGAGTCTTTTAAACGCTAGAACCGGAAAGAACTACTTAGCTTGGTACCAGCAGAAACCAGGACAGCCTCCTAA | |
| | | GCTGCTCATTTACTGGGCATCTACCCGGGAATCCGGGGTCCCTGACCGATTCAGTGGCAGCGGGTCTGGGACAGA | |
| | | CTTCACTCTCACTATCAGTAGCCTGCAGGCTGAAGATGTGGCAGTTTATTACTGTAAGCAATCTTATTCTCTGAGAA | |
| | | CTTTCGGCGGAGGGACCAAGGTCGAGATCAAA | |
| V019 | ADI-76362 | GACATCGTGATGACCCAGTCTCCAGACTCCCTGGCTGTGTCTCTGGGCGAGAGGGCCACCATCAACTGCAAGTCCA | 1960 |
| | | GCCAGAGTCTTTTAAACGCTAGAACCGGAAAGAACTACTTAGCTTGGTACCAGCAGAAACCAGGACAGCCTCCTAA | |
| | | GCTGCTCATTTACTGGGCATCTACCCGGGAATCCGGGGTCCCTGACCGATTCAGTGGCAGCGGGTCTGGGACAGA | |
| | | CTTCACTCTCACTATCAGTAGCCTGCAGGCTGAAGATGTGGCAGTTTATTACTGTAAGCAATCTTATTCTCTGAGAA | |
| | | CTTTCGGCGGAGGGACCAAGGTCGAGATCAAA | |

The closest germline is VK4-01.

TABLE 2A
VH CDR amino acid sequence

| | | | | | | | SEQ |
|---|---|---|---|---|---|---|---|
| Antibody | | CDRH1 | SEQ ID | CDRH2 | SEQ ID | CDRH3 | ID |
| No. | ADI Name | Sequence | NO: | Sequence | NO: | Sequence | NO: |
| A001 | ADI-26906 | FNIKDYYMH | 2112 | WIDLENANTIYDAKFQG | 2114 | ARDAYGRYFYDV | 2116 |
| V002 | ADI-67445 | FNIKDYYMH | 212 | WIDLENAETIYDAKFQD | 214 | ARDAYGRYFYDE | 216 |
| V003 | ADI-67446 | FDIKDYYMH | 312 | WIELENADTIYDAKFQG | 314 | ARDAYGRYFYDV | 316 |
| V004 | ADI-67447 | FNIDDYYMH | 412 | WIDLENDNTIYDAKFQG | 414 | ARDAYGRYFYDV | 416 |
| V005 | ADI-67448 | FNIDDYYMH | 512 | WIDLENDNTIYDAKFQG | 514 | ARDAYGRYFYDV | 516 |
| V006 | ADI-67449 | FNDKDYYMH | 612 | WIDLENAETIYDAKFQD | 614 | ARDAYGRYFYDV | 616 |
| V007 | ADI-67450 | FNIDDYYMH | 712 | WIDLENANTIDDAKFQG | 714 | ARDAYGRYFYDV | 716 |
| A002 | ADI-50024 | FNIKDYYMH | 2212 | WIDLENANTVYDAKFQG | 2214 | ARDAYGRYFYDV | 2216 |
| V008 | ADI-70325 | FNIKDYYMH | 812 | WIDLENAETVYDAKFQD | 814 | ARDAYGRYFYDV | 816 |
| V009 | ADI-70326 | FDIKDYYMH | 912 | WIELENADTVYDAKFQG | 914 | ARDAYGRYFYDV | 916 |
| V010 | ADI-70327 | FDIKDYYMH | 1012 | WIELENDDTVYDAKFQG | 1014 | ARDAYGRYFYDV | 1016 |
| V011 | ADI-70328 | FNIDDYYMH | 1112 | WIDLENDNTVYDAKFQG | 1114 | ARDAYGRYFYDV | 1116 |
| V012 | ADI-70329 | FNDKDYYMH | 1212 | WIDLENAETVYDAKFQD | 1214 | ARDAYGRYFYDV | 1216 |
| V013 | ADI-70330 | FNIDDYYMH | 1312 | WIDLENANTVDDAKFQG | 1314 | ARDAYGRYFYDV | 1316 |
| A003 | ADI-26913 | FNIKDYYMH | 2312 | WIDLENANTIYDAKFQG | 2314 | ARDQYGRYFYDV | 2316 |
| A004 | ADI-26920 | FNIKDYYMH | 2412 | WIDLENANTIYDAKFQG | 2414 | GRDAYGRYFYDV | 2416 |
| A005 | ADI-26921 | FNIKDYYMH | 2512 | WIDLEEGNTIYDAKFQG | 2514 | ARDAYGRYFYDV | 2516 |
| V014 | ADI-76357 | FDIKDYYMH | 1412 | WIDLENANTIYDAKFQG | 1414 | ARDQYGRYFYDV | 1416 |
| V015 | ADI-76358 | FNIKDYYMH | 1512 | WIDLENADTIYDAKFQG | 1514 | ARDQYGRYFYDV | 1516 |
| V016 | ADI-76359 | FDIKDYYMH | 1612 | WIDLENANTIYDAKFQG | 1614 | GRDAYGRYFYDV | 1616 |
| V017 | ADI-76360 | FNIKDYYMH | 1712 | WIDLENADTIYDAKFQG | 1714 | GRDAYGRYFYDV | 1716 |
| V018 | ADI-76361 | FDIKDYYMH | 1812 | WIDLEEGNTIYDAKFQG | 1814 | ARDAYGRYFYDV | 1816 |
| V019 | ADI-76362 | FNIKDYYMH | 1912 | WIDLEEGDTIYDAKFQG | 1914 | ARDAYGRYFYDV | 1916 |

| | | | | | | |
|---|---|---|---|---|---|---|
| Consensus sequence | FX₁X₂X₃DYYMH | 112 | WIX₄LEX₅X₆X₇TX₈X₉DAKFQX₁₀ | 114 | X₁₁RDX₁₂YGRYFYDX₁₃ | 116 |
| | X₁ = N or D; | | X₄ = D or E; | | X₁₁ = A or G; | |
| | X₂ = I or D; | | X₅ = N or E; | | X₁₂ = A or Q; | |
| | and | | X₆ = A, D, or G; | | and | |
| | X₃ = K or D. | | X₇ = N, E, or D; | | X₁₃ = V or E. | |
| | | | X₈ = I or V; | | | |
| | | | X₉ = Y or D; | | | |
| | | | and | | | |
| | | | X₁₀ = G or D. | | | |

Residues in bold indicate differences from the sequence of ADI-26906.
CDRH1 and CDRH2 residues in italics indicate differences from the closest germline (VH1-03)-encoded sequence. Note that each sequence could also be associated with another germline-encoded sequence.

TABLE 2B
VL CDR amino acid sequence

| | | CDRL1 | SEQ ID | CDRL2 | SEQ ID | CDRL3 | SEQ ID |
|---|---|---|---|---|---|---|---|
| Antibody No. | ADI Name | Sequence | NO: | Sequence | NO: | Sequence | NO: |
| A001 | ADI-26906 | KSSQSLLNARTGKNYLA | 2122 | WASTRES | 2124 | KQSYSRRT | 2126 |
| V002 | ADI-67445 | KSSQSLLNARTGKNYLA | 222 | WASTRES | 224 | KQSYSRRT | 226 |
| V003 | ADI-67446 | KSSQSLLNARTGKNYLA | 322 | WASTRES | 324 | KQSYSRRT | 326 |
| V004 | ADI-67447 | KSSQSLLNARTGKNYLA | 422 | WASTRES | 424 | KQSYSRRT | 426 |
| V005 | ADI-67448 | KSSQSLLNARTGKNYLA | 522 | WASTRES | 524 | KQSYSRRT | 526 |
| V006 | ADI-67449 | KSSQSLLNARTGKNYLA | 622 | WASTRES | 624 | KQSYSRRT | 626 |
| V007 | ADI-67450 | KSSQSLLNARTGKNYLA | 722 | WASTRES | 724 | KQSYSRRT | 726 |
| A002 | ADI-50024 | KSSQSLLNARTGKNYLA | 2222 | WASTRSS | 2224 | VQSYFRRT | 2226 |
| V008 | ADI-70325 | KSSQSLLNARTGKNYLA | 822 | WASTRSS | 824 | VQSYFRRT | 826 |
| V009 | ADI-70326 | KSSQSLLNARTGKNYLA | 922 | WASTRSS | 924 | VQSYFRRT | 926 |
| V010 | ADI-70327 | KSSQSLLNARTGKNYLA | 1022 | WASTRSS | 1024 | VQSYFRRT | 1026 |
| V011 | ADI-70328 | KSSQSLLNARTGKNYLA | 1122 | WASTRSS | 1124 | VQSYFRRT | 1126 |
| V012 | ADI-70329 | KSSQSLLNARTGKNYLA | 1222 | WASTRSS | 1224 | VQSYFRRT | 1226 |
| V013 | ADI-70330 | KSSQSLLNARTGKNYLA | 1322 | WASTRSS | 1324 | VQSYFRRT | 1326 |
| A003 | ADI-26913 | KSSQSLLNARTGKNYLA | 2322 | WASTRES | 2324 | KQSYSRRT | 2326 |
| A004 | ADI-26920 | KSSQSLLNARTGKNYLA | 2422 | WASTRES | 2424 | KQSYSLRT | 2426 |
| A005 | ADI-26921 | KSSQSLLNARTGKNYLA | 2522 | WASTRES | 2524 | KQSYSLRT | 2526 |
| V014 | ADI-76357 | KSSQSLLNARTGKNYLA | 1422 | WASTRES | 1424 | KQSYSRRT | 1426 |
| V015 | ADI-76358 | KSSQSLLNARTGKNYLA | 1522 | WASTRES | 1524 | KQSYSRRT | 1526 |
| V016 | ADI-76359 | KSSQSLLNARTGKNYLA | 1622 | WASTRES | 1624 | KQSYSLRT | 1626 |
| V017 | ADI-76360 | KSSQSLLNARTGKNYLA | 1722 | WASTRES | 1724 | KQSYSLRT | 1726 |
| V018 | ADI-76361 | KSSQSLLNARTGKNYLA | 1822 | WASTRES | 1824 | KQSYSLRT | 1826 |
| V019 | ADI-76362 | KSSQSLLNARTGKNYLA | 1922 | WASTRES | 1924 | KQSYSLRT | 1926 |

| | | | | | | |
|---|---|---|---|---|---|---|
| Consensus sequence | KSSQSLLNARTGKNYLA | 122 | WASTRX₁₄S | 124 | X₁₅QSYX₁₆X₁₇RT | 126 |
| | | | X₁₄ = E or S. | | X₁₅ = K or V; | |
| | | | | | X₁₆ = S or F; and | |
| | | | | | X₁₇ = R or L. | |

Residues in bold indicate differences from the sequence of ADI-26906.
CDRL1 and CDRL2 residues in italics indicate differences from the closest germline (VK4-01)-encoded sequence. Note that each sequence could also be associated with another germline-encoded sequence.

TABLE 3A
VH FR amino acid sequence

| | | | SEQ | | SEQ | | SEQ | | |
|---|---|---|---|---|---|---|---|---|---|
| Antibody | ADI | FRH1 | ID | FRH2 | ID | FRH3 | ID | FRH4 | ID |
| No. | Name | Sequence | NO: | Sequence | NO: | Sequence | NO: | Sequence | NO: |
| A001 | ADI- | QVQLVQSGAEVKKPGA | 2111 | WVRQAPGQR | 2113 | RVTITRDTSASTAYMELSS | 2115 | WGQGTL | 2117 |
| | 26906 | SVKVSCKASG | | LEWMG | | LRSEDTAVYYC | | VTVSS | |
| V002 | ADI- | QVQLVQSGAEVKKPGA | 211 | WVRQAPGQR | 213 | RVTITRDTSASTAYMELSS | 215 | WGQGTL | 217 |
| | 67445 | SVKVSCKASG | | LEWMG | | LRSEDTAVYYC | | VTVSS | |
| V003 | ADI- | QVQLVQSGAEVKKPGA | 311 | WVRQAPGQR | 313 | RVTITRDTSASTAYMELSS | 315 | WGQGTL | 317 |
| | 67446 | SVKVSCKASG | | LEWMG | | LRSEDTAVYYC | | VTVSS | |
| V004 | ADI- | QVQLVQSGAEVKKPGA | 411 | WVRQAPGQR | 413 | RVTITRDTSASTAYMELSS | 415 | WGQGTL | 417 |
| | 67447 | SVKVSCKASG | | LEWMG | | LRSEDTAVYYC | | VTVSS | |
| V005 | ADI- | QVQLVQSGAEVKKPGA | 511 | WVRQAPGQR | 513 | RVTITRDTSASTAYMELSS | 515 | WGQGTL | 517 |
| | 67448 | SVKVSCKASG | | LEWMG | | LRSEDTAVYYC | | VTVSS | |
| V006 | ADI- | QVQLVQSGAEVKKPGA | 611 | WVRQAPGQR | 613 | RVTITRDTSASTAYMELSS | 615 | WGQGTL | 617 |
| | 67449 | SVKVSCKASG | | LEWMG | | LRSEDTAVYYC | | VTVSS | |
| V007 | ADI- | QVQLVQSGAEVKKPGA | 711 | WVRQAPGQR | 713 | RVTITRDTSASTAYMELSS | 715 | WGQGTL | 717 |
| | 67450 | SVKVSCKASG | | LEWMG | | LRSEDTAVYYC | | VTVSS | |
| A002 | ADI- | QVQLVQSGAEVKKPGA | 2211 | WVRQAPGQR | 2213 | RVTITRDTSASTAYMELSS | 2215 | WGQGTL | 2217 |
| | 50024 | SVKVSCKASG | | LEWIG | | LRSEDTAVYYC | | VTVSS | |
| V008 | ADI- | QVQLVQSGAEVKKPGA | 811 | WVRQAPGQR | 813 | RVTITRDTSASTAYMELSS | 815 | WGQGTL | 817 |
| | 70325 | SVKVSCKASG | | LEWIG | | LRSEDTAVYYC | | VTVSS | |
| V009 | ADI- | QVQLVQSGAEVKKPGA | 911 | WVRQAPGQR | 913 | RVTITRDTSASTAYMELSS | 915 | WGQGTL | 917 |
| | 70326 | SVKVSCKASG | | LEWIG | | LRSEDTAVYYC | | VTVSS | |
| V010 | ADI- | QVQLVQSGAEVKKPGA | 1011 | WVRQAPGQR | 1013 | RVTITRDTSASTAYMELSS | 1015 | WGQGTL | 1017 |
| | 70327 | SVKVSCKASG | | LEWIG | | LRSEDTAVYYC | | VTVSS | |
| V011 | ADI- | QVQLVQSGAEVKKPGA | 1111 | WVRQAPGQR | 1113 | RVTITRDTSASTAYMELSS | 1115 | WGQGTL | 1117 |
| | 70328 | SVKVSCKASG | | LEWIG | | LRSEDTAVYYC | | VTVSS | |
| V012 | ADI- | QVQLVQSGAEVKKPGA | 1211 | WVRQAPGQR | 1213 | RVTITRDTSASTAYMELSS | 1215 | WGQGTL | 1217 |
| | 70329 | SVKVSCKASG | | LEWIG | | LRSEDTAVYYC | | VTVSS | |
| V013 | ADI- | QVQLVQSGAEVKKPGA | 1311 | WVRQAPGQR | 1313 | RVTITRDTSASTAYMELSS | 1315 | WGQGTL | 1317 |
| | 70330 | SVKVSCKASG | | LEWIG | | LRSEDTAVYYC | | VTVSS | |
| A003 | ADI- | QVQLVQSGAEVKKPGA | 2311 | WVRQAPGQR | 2313 | RVTITRDTSASTAYMELSS | 2315 | WGQGTL | 2317 |
| | 26913 | SVKVSCKASG | | LEWMG | | LRSEDTAVYYC | | VTVSS | |
| A004 | ADI- | QVQLVQSGAEVKKPGA | 2411 | WVRQAPGQR | 2413 | RVTITRDTSASTAYMELSS | 2415 | WGQGTL | 2417 |
| | 26920 | SVKVSCKASG | | LEWMG | | LRSEDTAVYYC | | VTVSS | |
| A005 | ADI- | QVQLVQSGAEVKKPGA | 2511 | WVRQAPGQR | 2513 | RVTITRDTSASTAYMELSS | 2515 | WGQGTL | 2517 |
| | 26921 | SVKVSCKASG | | LEWMG | | LRSEDTAVYYC | | VTVSS | |
| V014 | ADI- | QVQLVQSGAEVKKPGA | 1411 | WVRQAPGQR | 1413 | RVTITRDTSASTAYMELSS | 1415 | WGQGTL | 1417 |
| | 76357 | SVKVSCKASG | | LEWMG | | LRSEDTAVYYC | | VTVSS | |
| V015 | ADI- | QVQLVQSGAEVKKPGA | 1511 | WVRQAPGQR | 1513 | RVTITRDTSASTAYMELSS | 1515 | WGQGTL | 1517 |
| | 76358 | SVKVSCKASG | | LEWMG | | LRSEDTAVYYC | | VTVSS | |
| V016 | ADI- | QVQLVQSGAEVKKPGA | 1611 | WVRQAPGQR | 1613 | RVTITRDTSASTAYMELSS | 1615 | WGQGTL | 1617 |
| | 76359 | SVKVSCKASG | | LEWMG | | LRSEDTAVYYC | | VTVSS | |
| V017 | ADI- | QVQLVQSGAEVKKPGA | 1711 | WVRQAPGQR | 1713 | RVTITRDTSASTAYMELSS | 1715 | WGQGTL | 1717 |
| | 76360 | SVKVSCKASG | | LEWMG | | LRSEDTAVYYC | | VTVSS | |
| V018 | ADI- | QVQLVQSGAEVKKPGA | 1811 | WVRQAPGQR | 1813 | RVTITRDTSASTAYMELSS | 1815 | WGQGTL | 1817 |
| | 76361 | SVKVSCKASG | | LEWMG | | LRSEDTAVYYC | | VTVSS | |
| V019 | ADI- | QVQLVQSGAEVKKPGA | 1911 | WVRQAPGQR | 1913 | RVTITRDTSASTAYMELSS | 1915 | WGQGTL | 1917 |
| | 76362 | SVKVSCKASG | | LEWMG | | LRSEDTAVYYC | | VTVSS | |

| | | | | | | | | |
|---|---|---|---|---|---|---|---|---|
| Consensus | QVQLVQSGAEVKKPGA | 111 | WVRQAPGQR | 113 | RVTITRDTSASTAYMELSS | 115 | WGQGTL | 117 |
| sequence | SVKVSCKASG | | LEWX₁₈G | | LRSEDTAVYYC | | VTVSS | |
| | | | X₁₈ = M or I. | | | | | |

Residues in bold indicate differences from the sequence of ADI-26906.
FRH2 residues in italics indicate differences from the closest germline (VH1-03)-encoded sequence. Note that each sequence could also be associated with another germline-encoded sequence.

TABLE 3B
VL FR amino acid sequence

| | | | SEQ | | SEQ | | SEQ | | |
|---|---|---|---|---|---|---|---|---|---|
| Antibody | ADI | FRL1 | ID | FRL2 | ID | FRL3 | ID | FRL4 | SEQ ID |
| No. | Name | Sequence | NO: | Sequence | NO: | Sequence | NO: | Sequence | NO: |
| A001 | ADI- | DIVMTQSPDSLAVSL | 2121 | WYQQKPGQ | 2123 | GVPDRFSGSGSGTDFTLTISS | 2125 | FGGGTK | 2127 |
| | 26906 | GERATINC | | PPKLLIY | | LQAEDVAVYYC | | VEIK | |
| V002 | ADI- | DIVMTQSPDSLAVSL | 221 | WYQQKPGQ | 223 | GVPDRFSGSGSGTDFTLTISS | 225 | FGGGTK | 227 |
| | 67445 | GERATINC | | PPKLLIY | | LQAEDVAVYYC | | VEIK | |
| V003 | ADI- | DIVMTQSPDSLAVSL | 321 | WYQQKPGQ | 323 | GVPDRFSGSGSGTDFTLTISS | 325 | FGGGTK | 327 |
| | 67446 | GERATINC | | PPKLLIY | | LQAEDVAVYYC | | VEIK | |
| V004 | ADI- | DIVMTQSPDSLAVSL | 421 | WYQQKPGQ | 423 | GVPDRFSGSGSGTDFTLTISS | 425 | FGGGTK | 427 |
| | 67447 | GERATINC | | PPKLLIY | | LQAEDVAVYYC | | VEIK | |
| V005 | ADI- | DIVMTQSPDSLAVSL | 521 | WYQQKPGQ | 523 | GVPDRFSGSGSGTDFTLTISS | 525 | FGGGTK | 527 |
| | 67448 | GERATINC | | PPKLLIY | | LQAEDVAVYYC | | VEIK | |
| V006 | ADI- | DIVMTQSPDSLAVSL | 621 | WYQQKPGQ | 623 | GVPDRFSGSGSGTDFTLTISS | 625 | FGGGTK | 627 |
| | 67449 | GERATINC | | PPKLLIY | | LQAEDVAVYYC | | VEIK | |
| V007 | ADI- | DIVMTQSPDSLAVSL | 721 | WYQQKPGQ | 723 | GVPDRFSGSGSGTDFTLTISS | 725 | FGGGTK | 727 |
| | 67450 | GERATINC | | PPKLLIY | | LQAEDVAVYYC | | VEIK | |
| A002 | ADI- | DIVMSQSPDSLAVSL | 2221 | WYQQKPGQ | 2223 | GVPDRFSGSGSGTDFTLTISS | 2225 | FGGGTK | 2227 |
| | 50024 | GERATINC | | PPKLLIY | | LQAEDVAVYYC | | VEIK | |
| V008 | ADI- | DIVMSQSPDSLAVSL | 821 | WYQQKPGQ | 823 | GVPDRFSGSGSGTDFTLTISS | 825 | FGGGTK | 827 |
| | 70325 | GERATINC | | PPKLLIY | | LQAEDVAVYYC | | VEIK | |
| V009 | ADI- | DIVMSQSPDSLAVSL | 921 | WYQQKPGQ | 923 | GVPDRFSGSGSGTDFTLTISS | 925 | FGGGTK | 927 |
| | 70326 | GERATINC | | PPKLLIY | | LQAEDVAVYYC | | VEIK | |
| V010 | ADI- | DIVMSQSPDSLAVSL | 1021 | WYQQKPGQ | 1023 | GVPDRFSGSGSGTDFTLTISS | 1025 | FGGGTK | 1027 |
| | 70327 | GERATINC | | PPKLLIY | | LQAEDVAVYYC | | VEIK | |
| V011 | ADI- | DIVMSQSPDSLAVSL | 1121 | WYQQKPGQ | 1123 | GVPDRFSGSGSGTDFTLTISS | 1125 | FGGGTK | 1127 |
| | 70328 | GERATINC | | PPKLLIY | | LQAEDVAVYYC | | VEIK | |
| V012 | ADI- | DIVMSQSPDSLAVSL | 1221 | WYQQKPGQ | 1223 | GVPDRFSGSGSGTDFTLTISS | 1225 | FGGGTK | 1227 |
| | 70329 | GERATINC | | PPKLLIY | | LQAEDVAVYYC | | VEIK | |
| V013 | ADI- | DIVMSQSPDSLAVSL | 1321 | WYQQKPGQ | 1323 | GVPDRFSGSGSGTDFTLTISS | 1325 | FGGGTK | 1327 |
| | 70330 | GERATINC | | PPKLLIY | | LQAEDVAVYYC | | VEIK | |
| A003 | ADI- | DIVMTQSPDSLAVSL | 2321 | WYQQKPGQ | 2323 | GVPDRFSGSGSGTDFTLTISS | 2325 | FGGGTK | 2327 |
| | 26913 | GERATINC | | PPKLLIY | | LQAEDVAVYYC | | VEIK | |
| A004 | ADI- | DIVMTQSPDSLAVSL | 2421 | WYQQKPGQ | 2423 | GVPDRFSGSGSGTDFTLTISS | 2425 | FGGGTK | 2427 |
| | 26920 | GERATINC | | PPKLLIY | | LQAEDVAVYYC | | VEIK | |
| A005 | ADI- | DIVMTQSPDSLAVSL | 2521 | WYQQKPGQ | 2523 | GVPDRFSGSGSGTDFTLTISS | 2525 | FGGGTK | 2527 |
| | 26921 | GERATINC | | PPKLLIY | | LQAEDVAVYYC | | VEIK | |
| V014 | ADI- | DIVMTQSPDSLAVSL | 1421 | WYQQKPGQ | 1423 | GVPDRFSGSGSGTDFTLTISS | 1425 | FGGGTK | 1427 |
| | 76357 | GERATINC | | PPKLLIY | | LQAEDVAVYYC | | VEIK | |
| V015 | ADI- | DIVMTQSPDSLAVSL | 1521 | WYQQKPGQ | 1523 | GVPDRFSGSGSGTDFTLTISS | 1525 | FGGGTK | 1527 |
| | 76358 | GERATINC | | PPKLLIY | | LQAEDVAVYYC | | VEIK | |
| V016 | ADI- | DIVMTQSPDSLAVSL | 1621 | WYQQKPGQ | 1623 | GVPDRFSGSGSGTDFTLTISS | 1625 | FGGGTK | 1627 |
| | 76359 | GERATINC | | PPKLLIY | | LQAEDVAVYYC | | VEIK | |
| V017 | ADI- | DIVMTQSPDSLAVSL | 1721 | WYQQKPGQ | 1723 | GVPDRFSGSGSGTDFTLTISS | 1725 | FGGGTK | 1727 |
| | 76360 | GERATINC | | PPKLLIY | | LQAEDVAVYYC | | VEIK | |
| V018 | ADI- | DIVMTQSPDSLAVSL | 1821 | WYQQKPGQ | 1823 | GVPDRFSGSGSGTDFTLTISS | 1825 | FGGGTK | 1827 |
| | 76361 | GERATINC | | PPKLLIY | | LQAEDVAVYYC | | VEIK | |
| V019 | ADI- | DIVMTQSPDSLAVSL | 1921 | WYQQKPGQ | 1923 | GVPDRFSGSGSGTDFTLTISS | 1925 | FGGGTK | 1927 |
| | 76362 | GERATINC | | PPKLLIY | | LQAEDVAVYYC | | VEIK | |

| | | | | | | | | |
|---|---|---|---|---|---|---|---|---|
| Consensus | DIVMX₁₉QSPDSLAVS | 121 | WYQQKPGQ | 123 | GVPDRFSGSGSGTDFTLTISS | 125 | FGGGTK | 127 |
| sequence | LGERATINC | | PPKLLIY | | LQAEDVAVYYC | | VEIK | |
| | X₁₉ = T or S. | | | | | | | |

Residues in bold indicate differences from the sequence of ADI-26906.
FRL1 residues in italics indicate differences from the closest germline (VK4-01)-encoded sequence. Note that each sequence could also be associated with another germline-encoded sequence.

TABLE 4A
Heavy Chain Variable Region SEQ ID NO summary

| | | Amino Acid | | | | | | | | Nucleic Acid |
|---|---|---|---|---|---|---|---|---|---|---|
| Antibody No. | ADI Name | VH | FRH1 | CDRH1 | FRH2 | CDRH2 | FRH3 | CDRH3 | FRH4 | VH |
| A001 | ADI-26906 | 2110 | 2111 | 2112 | 2113 | 2114 | 2115 | 2116 | 2117 | 2150 |
| V002 | ADI-67445 | 210 | 211 | 212 | 213 | 214 | 215 | 216 | 217 | 250 |
| V003 | ADI-67446 | 310 | 311 | 312 | 313 | 314 | 315 | 316 | 317 | 350 |
| V004 | ADI-67447 | 410 | 411 | 412 | 413 | 414 | 415 | 416 | 417 | 450 |
| V005 | ADI-67448 | 510 | 511 | 512 | 513 | 514 | 515 | 516 | 517 | 550 |
| V006 | ADI-67449 | 610 | 611 | 612 | 613 | 614 | 615 | 616 | 617 | 650 |
| V007 | ADI-67450 | 710 | 711 | 712 | 713 | 714 | 715 | 716 | 717 | 750 |
| A002 | ADI-50024 | 2210 | 2211 | 2212 | 2213 | 2214 | 2215 | 2216 | 2217 | 2250 |
| V008 | ADI-70325 | 810 | 811 | 812 | 813 | 814 | 815 | 816 | 817 | 850 |
| V009 | ADI-70326 | 910 | 911 | 912 | 913 | 914 | 915 | 916 | 917 | 950 |
| V010 | ADI-70327 | 1010 | 1011 | 1012 | 1013 | 1014 | 1015 | 1016 | 1017 | 1050 |
| V011 | ADI-70328 | 1110 | 1111 | 1112 | 1113 | 1114 | 1115 | 1116 | 1117 | 1150 |
| V012 | ADI-70329 | 1210 | 1211 | 1212 | 1213 | 1214 | 1215 | 1216 | 1217 | 1250 |
| V013 | ADI-70330 | 1310 | 1311 | 1312 | 1313 | 1314 | 1315 | 1316 | 1317 | 1350 |
| A003 | ADI-26913 | 2310 | 2311 | 2312 | 2313 | 2314 | 2315 | 2316 | 2317 | 2350 |
| A004 | ADI-26920 | 2410 | 2411 | 2412 | 2413 | 2414 | 2415 | 2416 | 2417 | 2450 |
| A005 | ADI-26921 | 2510 | 2511 | 2512 | 2513 | 2514 | 2515 | 2516 | 2517 | 2550 |
| V014 | ADI-76357 | 1410 | 1411 | 1412 | 1413 | 1414 | 1415 | 1416 | 1417 | 1450 |
| V015 | ADI-76358 | 1510 | 1511 | 1512 | 1513 | 1514 | 1515 | 1516 | 1517 | 1550 |
| V016 | ADI-76359 | 1610 | 1611 | 1612 | 1613 | 1614 | 1615 | 1616 | 1617 | 1650 |
| V017 | ADI-76360 | 1710 | 1711 | 1712 | 1713 | 1714 | 1715 | 1716 | 1717 | 1750 |
| V018 | ADI-76361 | 1810 | 1811 | 1812 | 1813 | 1814 | 1815 | 1816 | 1817 | 1850 |
| V019 | ADI-76362 | 1910 | 1911 | 1912 | 1913 | 1914 | 1915 | 1916 | 1917 | 1950 |

| | | | | | | | |
|---|---|---|---|---|---|---|---|
| Consensus sequence | 111 | 112 | 113 | 114 | 115 | 116 | 117 |

TABLE 4B
Light Chain Variable Region SEQ ID NO summary

| | | Amino Acid | | | | | | | | Nucleic Acid |
|---|---|---|---|---|---|---|---|---|---|---|
| Antibody No. | ADI Name | VL | FRL1 | CDRL1 | FRL2 | CDRL2 | FRL3 | CDRL3 | FRL4 | VL |
| A001 | ADI-26906 | 2120 | 2121 | 2122 | 2123 | 2124 | 2125 | 2126 | 2127 | 2160 |
| V002 | ADI-67445 | 220 | 221 | 222 | 223 | 224 | 225 | 226 | 227 | 260 |
| V003 | ADI-67446 | 320 | 321 | 322 | 323 | 324 | 325 | 326 | 327 | 360 |
| V004 | ADI-67447 | 420 | 421 | 422 | 423 | 424 | 425 | 426 | 427 | 460 |
| V005 | ADI-67448 | 520 | 521 | 522 | 523 | 524 | 525 | 526 | 527 | 560 |
| V006 | ADI-67449 | 620 | 621 | 622 | 623 | 624 | 625 | 626 | 627 | 660 |
| V007 | ADI-67450 | 720 | 721 | 722 | 723 | 724 | 725 | 726 | 727 | 760 |
| A002 | ADI-50024 | 2220 | 2221 | 2222 | 2223 | 2224 | 2225 | 2226 | 2227 | 2260 |
| V008 | ADI-70325 | 820 | 821 | 822 | 823 | 824 | 825 | 826 | 827 | 860 |
| V009 | ADI-70326 | 920 | 921 | 922 | 923 | 924 | 925 | 926 | 927 | 960 |
| V010 | ADI-70327 | 1020 | 1021 | 1022 | 1023 | 1024 | 1025 | 1026 | 1027 | 1060 |
| V011 | ADI-70328 | 1120 | 1121 | 1122 | 1123 | 1124 | 1125 | 1126 | 1127 | 1160 |
| V012 | ADI-70329 | 1220 | 1221 | 1222 | 1223 | 1224 | 1225 | 1226 | 1227 | 1260 |
| V013 | ADI-70330 | 1320 | 1321 | 1322 | 1323 | 1324 | 1325 | 1326 | 1327 | 1360 |
| A003 | ADI-26913 | 2320 | 2321 | 2322 | 2323 | 2324 | 2325 | 2326 | 2327 | 2360 |
| A004 | ADI-26920 | 2420 | 2421 | 2422 | 2423 | 2424 | 2425 | 2426 | 2427 | 2460 |
| A005 | ADI-26921 | 2520 | 2521 | 2522 | 2523 | 2524 | 2525 | 2526 | 2527 | 2560 |
| V014 | ADI-76357 | 1420 | 1421 | 1422 | 1423 | 1424 | 1425 | 1426 | 1427 | 1460 |
| V015 | ADI-76358 | 1520 | 1521 | 1522 | 1523 | 1524 | 1525 | 1526 | 1527 | 1560 |
| V016 | ADI-76359 | 1620 | 1621 | 1622 | 1623 | 1624 | 1625 | 1626 | 1627 | 1660 |
| V017 | ADI-76360 | 1720 | 1721 | 1722 | 1723 | 1724 | 1725 | 1726 | 1727 | 1760 |
| V018 | ADI-76361 | 1820 | 1821 | 1822 | 1823 | 1824 | 1825 | 1826 | 1827 | 1860 |
| V019 | ADI-76362 | 1920 | 1921 | 1922 | 1923 | 1924 | 1925 | 1926 | 1927 | 1960 |

| | | | | | | | |
|---|---|---|---|---|---|---|---|
| Consensus sequence | 121 | 122 | 123 | 124 | 125 | 126 | 127 |

TABLE 4C
Exemplary Constant Region amino acid sequences

| | | SEQ |
|---|---|---|
| | | ID |
| | Sequences | NO: |
| Human IgG1 CH1, reference 1 | ASTKGPSVFPLAPSSKSTSGGTAALGCLVKDYFPEPVTVSWNSGALTSGVHTFPAVLQSSGLYSL | 91 |
| | SSVVTVPSSSLGTQTYICNVNHKPSNTKVDKKV | |
| Human IgG1 CH1, reference 2 | ASTKGPSVFPLAPSSKSTSGGTAALGCLVKDYFPEPVTVSWNSGALTSGVHTFPAVLQSSGLYSL | 92 |
| | SSVVTVPSSSLGTQTYICNVNHKPSNTKVDKRV | |
| Human IgG1 hinge, reference | EPKSCDKTHTCPPCP | 51 |
| Human IgG1 CH2, reference | APELLGGPSVFLFPPKPKDTLMISRTPEVTCVVVDVSHEDPEVKFNWYVDGVEVHNAKTKPREE | 61 |
| | QYNSTYRVVSVLTVLHQDWLNGKEYKCKVSNKALPAPIEKTISKAK | |
| Human IgG1 CH3 (356D and 358L) | GQPREPQVYTLPPSRDELTKNQVSLTCLVKGFYPSDIAVEWESNGQPENNYKTTPPVLDSDGSF | 71 |
| | FLYSKLTVDKSRWQQGNVFSCSVMHEALHNHYTQKSLSLSPG | |
| Human IgG1 CH3 (356E and 358M) | GQPREPQVYTLPPSREEMTKNQVSLTCLVKGFYPSDIAVEWESNGQPENNYKTTPPVLDSDGSF | 72 |
| | FLYSKLTVDKSRWQQGNVFSCSVMHEALHNHYTQKSLSLSPG | |
| Human IgG1 CH3 (356D, 358L, | GQPREPQVYTLPPSRDELTKNQVSLTCLVKGFYPSDIAVEWESNGQPENNYKTTPPVLDSDGSF | 73 |
| and V422I) | FLYSKLTVDKSRWQQGNIFSCSVMHEALHNHYTQKSLSLSPG | |
| Human IgG1 CH3 (356D, 358L, | GQPREPQVYTLPPSRDELTKNQVSLTCLVKGFYPSDIAVEWESNGQPENNYKTTPPVLDSDGSF | 74 |
| and A431G) | FLYSKLTVDKSRWQQGNVFSCSVMHEGLHNHYTQKSLSLSPG | |
| Human IgG CK | RTVAAPSVFIFPPSDEQLKSGTASVVCLLNNFYPREAKVQWKVDNALQSGNSQESVTEQDSKDS | 81 |
| | TYSLSSTLTLSKADYEKHKVYACEVTHQGLSSPVTKSFNRGEC | |
| Human IgG CL | GQPKAAPSVTLFPPSSEELQANKATLVCLISDFYPGAVTVAWKADSSPVKAGVETTTPSKQSNN | 82 |
| | KYAASSYLSLTPEQWKSHRSYSCQVTHEGSTVEKTVAPTECS | |

TABLE 5
Anti-CD3 antibody kinetic values

| Antibody No. | Human K_{D} (nM) | Cyno K_{D} (nM) |
|---|---|---|
| A001 | 2.81 | 2.90 |
| V002 | 7.84 | 7.33 |
| V003 | 4.57 | 4.18 |
| V004 | 19.19 | 16.26 |
| V005 | 6.98 | 6.06 |
| V006 | 7.97 | 8.15 |
| V007 | 5.39 | 5.03 |
| A002 | 0.50 | 0.57 |
| V008 | 0.95 | 0.91 |
| V009 | 0.81 | 0.81 |
| V010 | 1.92 | 1.91 |
| V011 | 0.82 | 0.86 |
| V012 | 1.65 | 1.64 |
| V013 | 1.16 | 1.23 |

TABLE 6
Cell binding analysis

| | | | | Jurkat CD3− | | Jurkat CD3− |
|---|---|---|---|---|---|---|
| | Human CD3+ | Cyno CD3 HSC-F | CHO-S cell | cell | HEK background | cell binding - |
| Antibody | Jurkat cell | cell binding | binding | binding | binding | Fab fragment |
| No. | binding (MFI) | (MFI) | (MFI) | (MFI) | (MFI) | (MFI) |
| A001 | 3153.45 | 6045.53 | 72.92 | 134.48 | 177.3 | 4.89 |
| V002 | 3043.5 | 5956.56 | 64.32 | 105.9 | 137.11 | 4.47 |
| V003 | 3022.71 | 6010.01 | 62.99 | 113.24 | 143.9 | 4.73 |
| V004 | 2806.73 | 5825.33 | 51.42 | 102.95 | 101.07 | 3.11 |
| V005 | 3149.29 | 5943.09 | 85.37 | 314.14 | 170.73 | 4.47 |
| V006 | 3044.92 | 5748.37 | 55.95 | 112.64 | 130.62 | 4.52 |
| V007 | 2930.01 | 5492.55 | 55.31 | 112.25 | 115.54 | 4.21 |
| A002 | 3407.12 | 6307.62 | 95.62 | 214.42 | 391.75 | 7.61 |
| V008 | 3200.41 | 6399.01 | 186.99 | 327.03 | 787.37 | 6.87 |
| V009 | 3067.01 | 6230.39 | 93.09 | 207.23 | 337.87 | 6.92 |
| V010 | 2907.52 | 5864.57 | 64.97 | 144.41 | 155.47 | 4.83 |
| V011 | 3000.74 | 5909.57 | 76.67 | 169.37 | 229.83 | 6.51 |
| V012 | 3020.45 | 5804.75 | 63.17 | 163.95 | 230.33 | 6.56 |
| V013 | 2953.06 | 5743.32 | 48.71 | 138.25 | 177.47 | 5.98 |

TABLE 7
Titration curve analysis

| | Human CD3+ Jurkat | Cyno CD3 HSC-F |
|---|---|---|
| Antibody No. | cell binding (EC50) | cell binding (EC50) |
| A001 | 1.7*10⁻⁹ | 1.64*10⁻⁹ |
| V002 | 4.49*10⁻⁹ | 4.34*10⁻⁹ |
| V003 | 4.71*10⁻⁹ | 3.29*10⁻⁹ |
| V004 | 1.52*10⁻⁸ | 1.43*10⁻⁸ |
| V005 | 5.85*10⁻⁹ | 4.46*10⁻⁹ |
| V006 | 4.56*10⁻⁹ | 4.2*10⁻⁹ |
| V007 | 5.97*10⁻⁹ | 7.22*10⁻⁹ |
| A002 | 8.15*10⁻¹⁰ | 6.96*10⁻¹⁰ |
| V008 | 1.64*10⁻⁹ | 1.05*10⁻⁹ |
| V009 | 2.18*10⁻⁹ | 1.39*10⁻⁹ |
| V010 | 6.99*10⁻⁹ | 5.05*10⁻⁹ |
| V011 | 2.7.7*10⁻⁹ | 1.39*10⁻⁹ |
| V012 | 3.8*10⁻⁹ | 2.5*10⁻⁹ |
| V013 | 4.78*10⁻⁹ | 3.91*10⁻⁹ |

TABLE 8
PSR score

| Antibody No. | PSR Score |
|---|---|
| A001 | 0.34 |
| V002 | 0.03 |
| V003 | 0.04 |
| V004 | 0.00 |
| V005 | 0.12 |
| V006 | 0.02 |
| V007 | 0.01 |
| A002 | 0.48 |
| V008 | 0.31 |
| V009 | 0.24 |
| V010 | 0.10 |
| V011 | 0.34 |
| V012 | 0.22 |
| V013 | 0.15 |

TABLE 9
Monovalent binding affinity to human or cynomolgus CD3εδ -
SPR (Biacore ®) with Fab (CHO produced)

| | | Monovalent Fab K_{D} (M) | | | |
|---|---|---|---|---|---|
| | | pH 7.4 | | pH 6.0 | |
| Antibody No. | ADI Name | Hu CD3εδ-Fc | Cy CD3εδ-Fc | Hu CD3εδ-Fc | Cy CD3εδ-Fc |
| A002 | ADI-50024 | 5.9E−11 | 7.6E−11 | 3.1E−10 | 2.0E−10 |
| V009 | ADI-70326 | 1.4E−10 | 1.5E−10 | 1.1E−09 | 2.4E−10 |
| V010 | ADI-70327 | 3.9E−10 | 4.5E−10 | 3.1E−09 | 8.2E−10 |
| V013 | ADI-70330 | 1.8E−10 | 2.2E−10 | 1.6E−09 | 2.7E−10 |
| A001 | ADI-26906 | 1.1E−09 | 1.1E−09 | 1.3E−09 | 1.9E−09 |
| V007 | ADI-67450 | 4.0E−09 | 2.7E−09 | 2.8E−09 | 2.1E−09 |
| V002 | ADI-67445 | P.F. | P.F. | 4.2E−09 | 3.3E−09 |
| V004 | ADI-67447 | 1.9E−09 | 2.5E−09 | 5.4E−09 | 2.2E−09 |
| A003 | ADI-26913 | 9.9E−09 | 9.3E−09 | 1.1E−08 | 1.1E−08 |
| A004 | ADI-26920 | 1.0E−08 | 9.0E−09 | 1.6E−08 | 1.5E−08 |
| A005 | ADI-26921 | 2.4E−08 | 3.3E−08 | 2.0E−08 | 2.4E−08 |
| V014 | ADI-76357 | 1.1E−08 | 1.1E−08 | 1.2E−08 | 1.2E−08 |
| V015 | ADI-76358 | 1.8E−08 | 1.9E−08 | 1.4E−08 | 1.1E−08 |
| V016 | ADI-76359 | 1.4E−08 | 1.6E−08 | 2.2E−08 | 2.3E−08 |
| V017 | ADI-76360 | 1.9E−08 | 1.6E−08 | 2.5E−08 | 2.6E−08 |
| V018 | ADI-76361 | 4.2E−08 | 3.6E−08 | 3.1E−08 | 2.9E−08 |
| V019 | ADI-76362 | P.F | P.F. | 3.3E−08 | 2.9E−08 |

K_{D} (equilibrium dissociation constant) = k_{off}/kₒₙ; data fit to a 1:1 binding model.
P.F. = Poor Fit; binding observed under the conditions of this assay but unable to be adequately fit to a 1:1 binding model.

TABLE 10
Binding affinity and avidity -BLI (Octet ®) with Fab and IgG1 (CHO produced)

| | | | | | | | IgG Avidity | IgG Monovalent |
|---|---|---|---|---|---|---|---|---|
| | | Fab Monovalent K_{D} (M) | | | | | K_{D} (M) | K_{D} (M) |
| Antibody | ADI | pH 7.4 | | pH 6.0 | | pH 7.4 | pH 7.4 | |
| No. | Name | Hu CD3εδ-Fc | Cy CD3εδ-Fc | Hu CD3εδ-Fc | Cy CD3εδ-Fc | Rh CD3εδ-Fc | Mo CD3εδ-Fc | Mo CD3εδ-Fc |
| A002 | ADI- | 1.0E−09 | 1.3E−09 | 1.3E−09 | 1.7E−09 | 1.2E−09 | 9.1E−08 | N.B. |
| | 50024 | | | | | | | |
| V009 | ADI- | 1.6E−09 | 1.9E−09 | 1.2E−09 | 1.6E−09 | 1.8E−09 | 3.6E−07 | N.B. |
| | 70326 | | | | | | | |
| V010 | ADI- | 4.1E−09 | 3.4E−09 | 2.0E−09 | 1.9E−09 | 4.4E−09 | 5.1E−07 | N.B. |
| | 70327 | | | | | | | |
| V013 | ADI- | 2.4E−09 | 2.4E−09 | 1.6E−09 | 1.9E−09 | 2.5E−09 | 5.3E−07 | N.B. |
| | 70330 | | | | | | | |
| A001 | ADI- | 6.0E−09 | 5.9E−09 | 5.8E−09 | 5.9E−09 | 6.1E−09 | 2.9E−07 | N.B. |
| | 26906 | | | | | | | |
| V007 | ADI- | 7.4E−09 | 8.7E−09 | 6.9E−09 | 6.9E−09 | 9.2E−09 | N.B. | N.B. |
| | 67450 | | | | | | | |
| V002 | ADI- | 1.3E−08 | 1.2E−08 | 8.8E−09 | 8.0E−09 | 1.5E−08 | 1.1E−06 | N.B. |
| | 67445 | | | | | | | |
| V004 | ADI- | 1.3E−08 | 1.1E−08 | 7.7E−09 | 7.9E−09 | 1.3E−08 | 8.6E−07 | N.B. |
| | 67447 | | | | | | | |
| A003 | ADI- | 3.5E−08 | 2.8E−08 | 2.9E−08 | 3.2E−08 | 3.3E−08 | 6.4E−07 | N.B. |
| | 26913 | | | | | | | |
| A004 | ADI- | 4.2E−08 | 5.0E−08 | 2.9E−08 | 2.3E−08 | 5.1E−08 | N.B. | N.B. |
| | 26920 | | | | | | | |
| A005 | ADI- | 7.8E−08 | 3.9E−08 | 3.5E−08 | 4.5E−08 | 3.6E−08 | N.B. | N.B. |
| | 26921 | | | | | | | |
| V014 | ADI- | 4.4E−08 | 3.9E−08 | 2.4E−08 | 2.1E−08 | 3.9E−08 | 5.7E−07 | N.B. |
| | 76357 | | | | | | | |
| V015 | ADI- | 5.6E−08 | 5.8E−08 | 3.4E−08 | 2.9E−08 | 5.3E−08 | 5.1E−07 | N.B. |
| | 76358 | | | | | | | |
| V016 | ADI- | 7.7E−08 | 7.5E−08 | 3.7E−08 | 4.9E−08 | 8.7E−08 | N.B. | N.B. |
| | 76359 | | | | | | | |
| V017 | ADI- | 4.2E−07 | 9.6E−08 | 5.8E−08 | 3.4E−08 | P.F | N.B. | N.B. |
| | 76360 | | | | | | | |
| V018 | ADI- | 3.7E−08 | 2.8E−08 | 3.3E−08 | 1.7E−08 | 2.9E−08 | N.B. | N.B. |
| | 76361 | | | | | | | |
| V019 | ADI- | 6.4E−08 | 3.4E−08 | 3.6E−08 | 2.1E−08 | 7.9E−08 | N.B. | N.B. |
| | 76362 | | | | | | | |

K_{D} (equilibrium dissociation constant) = k_{off}/kₒₙ; data fit to a 1:1 binding model.
P.F. = Poor Fit; binding observed under the conditions of this assay but unable to be adequately fit to a 1:1 binding model.
N.B. = No Binding; no observable binding under the conditions of this assay; unable to assign a K_{D}.

TABLE 11
Cell binding - flowcytometry with IgG1 (CHO produced)

NCB with 100 nM IgG

| | | Human CD3+ | Cyno CD3+ |
|---|---|---|---|
| Antibody No. | ADI Name | Jurkat cells | HSC-F cells |
| A002 | ADI-50024 | 366.2 | 417.3 |
| V009 | ADI-70326 | 309.4 | 396.0 |
| V010 | ADI-70327 | 298.0 | 378.2 |
| V013 | ADI-70330 | 295.3 | 373.8 |
| A001 | ADI-26906 | 288.7 | 399.5 |
| V007 | ADI-67450 | 286.7 | 367.5 |
| V002 | ADI-67445 | 319.5 | 387.7 |
| V004 | ADI-67447 | 267.7 | 391.3 |
| A003 | ADI-26913 | 209.3 | 366.7 |
| A004 | ADI-26920 | 240.3 | 347.9 |
| A005 | ADI-26921 | 206.2 | 364.2 |
| V014 | ADI-76357 | 348.9 | 376.9 |
| V015 | ADI-76358 | 334.4 | 369.0 |
| V016 | ADI-76359 | 313.5 | 374.2 |
| V017 | ADI-76360 | 325.0 | 375.5 |
| V018 | ADI-76361 | 223.0 | 378.4 |
| V019 | ADI-76362 | 327.9 | 373.0 |

NCB (Normalized Cell Binding) value = {(Sample MFI) − (Secondary only MFI)}/(Secondary only MFI).
MFI = Median Fluorescence Intensity.

TABLE 12
Chromatographic analyses - LC-MS and SEC-HPLC with IgG1 (CHO produced)

| | | | SEC-HPLC % Monomer | | | | |
|---|---|---|---|---|---|---|---|
| | | | IgG | | | IgG | |
| | | LC-MS QC | after | | | Pre pH 3.5 | Post pH 3.5 |
| Antibody No. | ADI Name | IgG | ProA | IgG | Fab | Stress | Stress |
| A002 | ADI-50024 | PASS | 97.6 | 97.4 | 97.2 | 97.0 | 97.3 |
| V009 | ADI-70326 | PASS | 97.8 | 92.9 | 95.6 | 91.4 | 93.0 |
| V010 | ADI-70327 | PASS | 98.9 | 98.8 | 97.4 | 97.9 | 98.3 |
| V013 | ADI-70330 | PASS | 99.3 | 99.2 | 97.5 | 98.7 | 99.0 |
| A001 | ADI-26906 | PASS | 98.7 | 98.6 | 97.7 | 98.1 | 98.1 |
| V007 | ADI-67450 | PASS | 99.3 | 99.2 | 97.6 | 98.9 | 98.7 |
| V002 | ADI-67445 | PASS | 99.4 | 99.3 | 98.0 | 98.9 | 98.7 |
| V004 | ADI-67447 | PASS* | N.D. | 99.3 | 98.0 | 98.9 | 98.3 |
| A003 | ADI-26913 | PASS | 99.7 | 99.6 | 98.5 | 99.3 | 98.9 |
| A004 | ADI-26920 | PASS | 99.4 | 99.3 | 97.9 | 98.9 | 98.8 |
| A005 | ADI-26921 | PASS | 93.2 | 99.8 | 94.7 | 99.5 | 98.3 |
| V014 | ADI-76357 | PASS | 98.2 | 97.7 | 97.2 | 97.0 | 96.7 |
| V015 | ADI-76358 | PASS | 99.5 | 99.5 | 97.9 | 99.1 | 98.8 |
| V016 | ADI-76359 | PASS | 99.4 | 99.3 | 98.0 | 98.9 | 98.7 |
| V017 | ADI-76360 | PASS | 99.4 | 99.2 | 97.7 | 98.8 | 98.5 |
| V018 | ADI-76361 | PASS | 99.5 | 99.5 | 98.5 | 99.3 | 98.8 |
| V019 | ADI-76362 | PASS | 99.3 | 99.3 | 98.0 | 98.5 | 98.3 |

QC = Quality Control; PASS = Antibodies were produced cleanly with no additional peaks. ADI-67447 had 28% ADI-26913 heavy chain
SEC = Size exclusion chromatography.
Pre pH 3.5 Stress = incubated at 15 mg/ml at room temperature for 1 hour in PBS.
Post pH 3.5 Stress = incubated at 15 mg/ml at room temperature for 1 hour at pH 3.5.
ADI-26921 was ceramic hydroxyapatite (CHT) polished

TABLE 13
Developability - PSR, HIC, AC-SINS and DLS with IgG1 (CHO produced)

| Antibody No. | ADI Name | PSR Score (0-1) | HIC Retention Time (min) | AC-SINS Δλmax (nm) | DLS kD (mL/g) |
|---|---|---|---|---|---|
| A002 | ADI-50024 | 0.42 | 8.8 | 14.4 | 38.7 |
| V009 | ADI-70326 | 0.17 | 8.7 | 7.0 | −0.3 |
| V010 | ADI-70327 | 0.11 | 8.6 | 2.8 | 11.3 |
| V013 | ADI-70330 | 0.15 | 8.9 | 2.5 | 9.6 |
| A001 | ADI-26906 | 0.30 | 8.5 | 10.0 | 36.6 |
| V007 | ADI-67450 | 0.01 | 8.7 | 0.6 | 6.4 |
| V002 | ADI-67445 | 0.07 | 8.3 | 5.6 | 3.6 |
| V004 | ADI-67447 | 0.04 | 8.5 | 4.2 | −28.9 |
| A003 | ADI-26913 | 0.07 | 8.3 | 2.1 | 36.6 |
| A004 | ADI-26920 | 0.06 | 8.7 | 11.2 | 24.1 |
| A005 | ADI-26921 | 0.08 | 8.7 | 7.0 | 21.4 |
| V014 | ADI-76357 | 0.01 | 8.3 | 0.9 | 29.7 |
| V015 | ADI-76358 | 0.00 | 8.3 | 3.9 | 25.3 |
| V016 | ADI-76359 | 0.03 | 8.7 | 8.5 | 26.8 |
| V017 | ADI-76360 | 0.03 | 8.6 | 9.8 | 19.3 |
| V018 | ADI-76361 | 0.06 | 8.7 | 6.5 | 13.9 |
| V019 | ADI-76362 | 0.06 | 8.6 | 7.4 | 9.7 |

PSR = Poly-specificity reagent, score determined by normalizing values against a set of control IgGs (Clean: <0.10, Low: ≥0.10 and <0.33, Medium: >0.33 and <0.66, and High: ≥0.66 and ≤1.00).
HIC = Hydrophobic interaction chromatography, retention time may be categorized (Clean to Low: <10.5 min, Medium: ≥10.5 and <11.5 min, and High: ≥11.5 min).
AC-SINS = Affinity-capture self-interaction nanoparticle spectroscopy (Δλmax <5.0 nm implies low self-interaction, Δλmax ≥5.0 nm and <20.0 nm implies medium self-interaction, and Δλmax ≥20.0 nm implies high self-interaction).
DLS = dynamic light scattering (kD values <20 mL/g are associated with high viscosity or high opalescence).

TABLE 14
Developability - Tₘ by DSF with Fab (CHO produced)

| Antibody No. | ADI Name | Fab Tₘ by DSF (° C.) |
|---|---|---|
| A002 | ADI-50024 | 81.5 |
| V009 | ADI-70326 | 81.0 |
| V010 | ADI-70327 | 79.5 |
| V013 | ADI-70330 | 76.5 |
| A001 | ADI-26906 | 80.0 |
| V007 | ADI-67450 | 77.0 |
| V002 | ADI-67445 | 77.0 |
| V004 | ADI-67447 | 78.0 |
| A003 | ADI-26913 | 80.5 |
| A004 | ADI-26920 | 75.5 |
| A005 | ADI-26921 | 82.0 |
| V014 | ADI-76357 | 79.5 |
| V015 | ADI-76358 | 80.5 |
| V016 | ADI-76359 | 75.5 |
| V017 | ADI-76360 | 75.5 |
| V018 | ADI-76361 | 81.5 |
| V019 | ADI-76362 | 81.5 |

DSF = differential scanning fluorimetry.

What is claimed is:

1. An anti-cluster of Differentiation 3 (CD3) antibody or antigen-binding fragment, which comprises a complementarity determining region (CDR) comprising:
(i) the amino acid sequence of a CDR contained in any of the variable domain sequences listed in Table 1A or 1B; and/or
(ii) an amino acid sequence selected from any of those listed in Table 2A or 2B.

2. An anti-CD3 antibody or antigen-binding fragment, comprising:
(A) a heavy chain variable domain (VH) polypeptide comprising:
(a) a VH CDR1 (CDRH1) comprising the amino acid sequence of:
(i) the CDRH1 contained in any one of Antibody Nos. V002-V019 and A001-A005;
(ii) the CDRH1 contained in any one of SEQ ID NOS: 210, 310, 410, 510, 610, 710, 810, 910, 1010, 1110, 1210, 1310, 1410, 1510, 1610, 1710, 1810, 1910, 2110, 2210, 2310, 2410, and 2510;
(iii) FX₁X₂X₃DYYMH (SEQ ID NO: 112), wherein X₁ is N or D, X₂ is I or D, and X₃ is K or D; and/or
(iv) any one of SEQ ID NOS: 212, 312, 412, 512, 612, 712, 812, 912, 1012, 1112, 1212, 1312, 1412, 1512, 1612, 1712, 1812, 1912, 2112, 2212, 2312, 2412, and 2512;
(b) a VH CDR2 (CDRH2) comprising the amino acid sequence of:
(i) the CDRH2 contained in any one of Antibody Nos. V002-V019 and A001-A005;
(ii) the CDRH2 contained in any one of SEQ ID NOS: 210, 310, 410, 510, 610, 710, 810, 910, 1010, 1110, 1210, 1310, 1410, 1510, 1610, 1710, 1810, 1910, 2110, 2210, 2310, 2410, and 2510;
(iii) WIX₄LEX₅X₆X₇TX₈X₉DAKFQX₁₀ (SEQ ID NO: 114), wherein X₄ is D or E, X₅ is N or E, X₆ is A, D, or G, X₇ is N, E, or D, X₈ is I or V, X₉ is Y or D, and X₁₀ is G or D; and/or
(iv) any one of SEQ ID NOS: 214, 314, 414, 514, 614, 714, 814, 914, 1014, 1114, 1214, 1314, 1414, 1514, 1614, 1714, 1814, 1914, 2114, 2214, 2314, 2414, and 2514; and/or
(c) a VH CDR3 (CDRH3) comprising the amino acid sequence of:
(i) the CDRH3 contained in any one of Antibody Nos. V002-V019 and A001-A005;
(ii) the CDRH3 contained in any one of SEQ ID NOS: 210, 310, 410, 510, 610, 710, 810, 910, 1010, 1110, 1210, 1310, 1410, 1510, 1610, 1710, 1810, 1910, 2110, 2210, 2310, 2410, and 2510;
(iii) X₁₁RDX₁₂YGRYFYDX₁₃ (SEQ ID NO: 116), wherein X₁₁ is A or G, X₁₂ is A or Q, and X₁₃ is V or E; and/or
(iv) any one of SEQ ID NOS: 216, 316, 416, 516, 616, 716, 816, 916, 1016, 1116, 1216, 1316, 1416, 1516, 1616, 1716, 1816, 1916, 2116, 2216, 2316, 2416, and 2516; and/or
(B) a light chain variable domain (VL) polypeptide comprising:
(a) a VL CDR1 (CDRL1) comprising the amino acid sequence of:
(i) the CDRL1 contained in any one of Antibody Nos. V002-V019 and A001-A005;
(ii) the CDRL1 contained in any one of SEQ ID NOS: 220, 320, 420, 520, 620, 720, 820, 920, 1020, 1120, 1220, 1320, 1420, 1520, 1620, 1720, 1820, 1920, 2120, 2220, 2320, 2420, and 2520; and/or
(iii) any one of SEQ ID NOS: 122, 222, 322, 422, 522, 622, 722, 822, 922, 1022, 1122, 1222, 1322, 1422, 1522, 1622, 1722, 1822, 1922, 2122, 2222, 2322, 2422, and 2522;
(b) a VL CDR2 (CDRL2) comprising the amino acid sequence of:
(i) the CDRL2 contained in any one of Antibody Nos. V002-V019 and A001-A005;
(ii) the CDRL2 contained in any one of SEQ ID NOS: 220, 320, 420, 520, 620, 720, 820, 920, 1020, 1120, 1220, 1320, 1420, 1520, 1620, 1720, 1820, 1920, 2120, 2220, 2320, 2420, and 2520;
(iii) WASTRX₁₄S (SEQ ID NO: 124), wherein X₁₄ is E or S; and/or
(iv) any one of SEQ ID NOS: 224, 324, 424, 524, 624, 724, 824, 924, 1024, 1124, 1224, 1324, 1424, 1524, 1624, 1724, 1824, 1924, 2124, 2224, 2324, 2424, and 2524; and/or
(c) a VL CDR3 (CDRL3) comprising the amino acid sequence of:
(i) the CDRL3 contained in any one of Antibody Nos. V002-V019 and A001-A005;
(ii) the CDRL3 contained in any one of SEQ ID NOS: 220, 320, 420, 520, 620, 720, 820, 920, 1020, 1120, 1220, 1320, 1420, 1520, 1620, 1720, 1820, 1920, 2120, 2220, 2320, 2420, and 2520;
(iii) X₁₅QSYX₁₆X₁₇RT (SEQ ID NO: 126), wherein X₁₅ is K or V, X₁₆ is S or F, and X₁₇ is R or L; and/or
(iv) any one of SEQ ID NOS: 226, 326, 426, 526, 626, 726, 826, 926, 1026, 1126, 1226, 1326, 1426, 1526, 1626, 1726, 1826, 1926, 2126, 2226, 2326, 2426, and 2526,
or an anti-CD3 antibody or antigen-binding fragment which comprises a combination of one or more of the foregoing CDRs,
optionally wherein the anti-CD3 antibody or antigen-binding fragment:
(I) does not comprise:
(i) at least one of the CDR-H1, CDR-H2, CDR-H3, CDR-L1, CDR-L2, and CDR-L3 contained in A001; or
(ii) at least one of SEQ ID NOS: 2112, 2114, 2116, 2122, 2124, and 2126,
further optionally does not comprise:
(i) at least one of the CDRH1 and CDRH2 contained in A001; or
(ii) at least one of SEQ ID NOS: 2112 and 2114;
(II) does not comprise:
(i) at least one of the CDR-H1, CDR-H2, CDR-H3, CDR-L1, CDR-L2, and CDR-L3 contained in A002; or
(ii) at least one of SEQ ID NOS: 2212, 2214, 2216, 2222, 2224, and 2226,
further optionally does not comprise:
(i) at least one of the CDRH1 and CDRH2 contained in A002; or
(ii) at least one of SEQ ID NOS: 2212 and 2214;
(III) does not comprise:
(i) at least one of the CDR-H1, CDR-H2, CDR-H3, CDR-L1, CDR-L2, and CDR-L3 contained in A003; or
(ii) at least one of SEQ ID NOS: 2312, 2314, 2316, 2322, 2324, and 2326,
further optionally does not comprise:
(i) at least one of the CDRH1 and CDRH2 contained in A003; or
(ii) at least one of SEQ ID NOS: 2312 and 2314;
(IV) does not comprise:
(i) at least one of the CDR-H1, CDR-H2, CDR-H3, CDR-L1, CDR-L2, and CDR-L3 contained in A004; or
(ii) at least one of SEQ ID NOS: 2412, 2414, 2416, 2422, 2424, and 2426,
further optionally does not comprise:
(i) at least one of the CDRH1 and CDRH2 contained in A004; or
(ii) at least one of SEQ ID NOS: 2412 and 2414; and
(V) does not comprise:
(i) at least one of the CDR-H1, CDR-H2, CDR-H3, CDR-L1, CDR-L2, and CDR-L3 contained in A005; or
(ii) at least one of SEQ ID NOS: 2512, 2514, 2516, 2522, 2524, and 2526,
further optionally does not comprise:
(i) at least one of the CDRH1 and CDRH2 contained in A005; or
(ii) at least one of SEQ ID NOS: 2512 and 2514.

3. The anti-CD3 antibody or antigen-binding fragment of claim 2, comprising:
(A) a VH polypeptide comprising said CDRH1, said CDRH2, and said CDRH3; and/or
(B) a VL polypeptide comprising said CDRL1, said CDRL2, and said CDRL3.

4. The anti-CD3 antibody or antigen-binding fragment of claim 2, comprising:
(A) a VH polypeptide comprising said CDRH1, said CDRH2, and said CDRH3; and
(B) a VL polypeptide comprising said CDRL1, said CDRL2, and said CDRL3.

5. The anti-CD3 antibody or antigen-binding fragment of any one of claims 2-4, comprising:
(I) (i) the CDRH1, the CDRH2, the CDRH3, the CDRL1, the CDRL2, and the CDR-L3 contained in Antibody No. V002; or
(ii) a CDRH1 comprising SEQ ID NO: 212, a CDRH2 comprising SEQ ID NO: 214, a CDRH3 comprising SEQ ID NO: 216, a CDRL1 comprising SEQ ID NO: 222, a CDRL2 comprising SEQ ID NO: 224, and a CDRL3 comprising SEQ ID NO: 226;
(II) (i) the CDRH1, the CDRH2, the CDRH3, the CDRL1, the CDRL2, and the CDR-L3 contained in Antibody No. V003; or
(ii) a CDRH1 comprising SEQ ID NO: 312, a CDRH2 comprising SEQ ID NO: 314, a CDRH3 comprising SEQ ID NO: 316, a CDRL1 comprising SEQ ID NO: 322, a CDRL2 comprising SEQ ID NO: 324, and a CDRL3 comprising SEQ ID NO: 326;
(III) (i) the CDRH1, the CDRH2, the CDRH3, the CDRL1, the CDRL2, and the CDR-L3 contained in Antibody No. V004; or
(ii) a CDRH1 comprising SEQ ID NO: 412, a CDRH2 comprising SEQ ID NO: 414, a CDRH3 comprising SEQ ID NO: 416, a CDRL1 comprising SEQ ID NO: 422, a CDRL2 comprising SEQ ID NO: 424, and a CDRL3 comprising SEQ ID NO: 426;
(IV) (i) the CDRH1, the CDRH2, the CDRH3, the CDRL1, the CDRL2, and the CDR-L3 contained in Antibody No. V005; or
(ii) a CDRH1 comprising SEQ ID NO: 512, a CDRH2 comprising SEQ ID NO: 514, a CDRH3 comprising SEQ ID NO: 516, a CDRL1 comprising SEQ ID NO: 522, a CDRL2 comprising SEQ ID NO: 524, and a CDRL3 comprising SEQ ID NO: 526;
(V) (i) the CDRH1, the CDRH2, the CDRH3, the CDRL1, the CDRL2, and the CDR-L3 contained in Antibody No. V006; or
(ii) a CDRH1 comprising SEQ ID NO: 612, a CDRH2 comprising SEQ ID NO: 614, a CDRH3 comprising SEQ ID NO: 616, a CDRL1 comprising SEQ ID NO: 622, a CDRL2 comprising SEQ ID NO: 624, and a CDRL3 comprising SEQ ID NO: 626;
(VI) (i) the CDRH1, the CDRH2, the CDRH3, the CDRL1, the CDRL2, and the CDR-L3 contained in Antibody No. V007; or
(ii) a CDRH1 comprising SEQ ID NO: 712, a CDR-H2 comprising SEQ ID NO: 714, a CDRH3 comprising SEQ ID NO: 716, a CDRL1 comprising SEQ ID NO: 722, a CDRL2 comprising SEQ ID NO: 724, and a CDRL3 comprising SEQ ID NO: 726;
(VII) (i) the CDRH1, the CDRH2, the CDRH3, the CDRL1, the CDRL2, and the CDR-L3 contained in Antibody No. V008; or
(ii) a CDRH1 comprising SEQ ID NO: 812, a CDRH2 comprising SEQ ID NO: 814, a CDRH3 comprising SEQ ID NO: 816, a CDRL1 comprising SEQ ID NO: 822, a CDRL2 comprising SEQ ID NO: 824, and a CDRL3 comprising SEQ ID NO: 826;
(VIII) (i) the CDRH1, the CDRH2, the CDRH3, the CDRL1, the CDRL2, and the CDR-L3 contained in Antibody No. V009; or
(ii) a CDRH1 comprising SEQ ID NO: 912, a CDRH2 comprising SEQ ID NO: 914, a CDRH3 comprising SEQ ID NO: 916, a CDRL1 comprising SEQ ID NO: 922, a CDRL2 comprising SEQ ID NO: 924, and a CDRL3 comprising SEQ ID NO: 926;
(IX) (i) the CDRH1, the CDRH2, the CDRH3, the CDRL1, the CDRL2, and the CDR-L3 contained in Antibody No. V010; or
(ii) a CDRH1 comprising SEQ ID NO: 1012, a CDRH2 comprising SEQ ID NO: 1014, a CDRH3 comprising SEQ ID NO: 1016, a CDRL1 comprising SEQ ID NO: 1022, a CDRL2 comprising SEQ ID NO: 1024, and a CDRL3 comprising SEQ ID NO: 1026;
(X) (i) the CDRH1, the CDRH2, the CDRH3, the CDRL1, the CDRL2, and the CDR-L3 contained in Antibody No. V011; or
(ii) a CDRH1 comprising SEQ ID NO: 1112, a CDRH2 comprising SEQ ID NO: 1114, a CDRH3 comprising SEQ ID NO: 1116, a CDRL1 comprising SEQ ID NO: 1122, a CDRL2 comprising SEQ ID NO: 1124, and a CDRL3 comprising SEQ ID NO: 1126;
(XI) (i) the CDRH1, the CDRH2, the CDRH3, the CDRL1, the CDRL2, and the CDR-L3 contained in Antibody No. V012; or
(ii) a CDRH1 comprising SEQ ID NO: 1212, a CDRH2 comprising SEQ ID NO: 1214, a CDRH3 comprising SEQ ID NO: 1216, a CDRL1 comprising SEQ ID NO: 1222, a CDRL2 comprising SEQ ID NO: 1224, and a CDRL3 comprising SEQ ID NO: 1226;
(XII) (i) the CDRH1, the CDRH2, the CDRH3, the CDRL1, the CDRL2, and the CDR-L3 contained in Antibody No. V013; or
(ii) a CDRH1 comprising SEQ ID NO: 1312, a CDRH2 comprising SEQ ID NO: 1314, a CDRH3 comprising SEQ ID NO: 1316, a CDRL1 comprising SEQ ID NO: 1322, a CDRL2 comprising SEQ ID NO: 1324, and a CDRL3 comprising SEQ ID NO: 1326;
(XIII) (i) the CDRH1, the CDRH2, the CDRH3, the CDRL1, the CDRL2, and the CDR-L3 contained in Antibody No. V014; or
(ii) a CDRH1 comprising SEQ ID NO: 1412, a CDRH2 comprising SEQ ID NO: 1414, a CDRH3 comprising SEQ ID NO: 1416, a CDRL1 comprising SEQ ID NO: 1422, a CDRL2 comprising SEQ ID NO: 1424, and a CDRL3 comprising SEQ ID NO: 1426;
(XIV) (i) the CDRH1, the CDRH2, the CDRH3, the CDRL1, the CDRL2, and the CDR-L3 contained in Antibody No. V015; or
(ii) a CDRH1 comprising SEQ ID NO: 1512, a CDRH2 comprising SEQ ID NO: 1514, a CDRH3 comprising SEQ ID NO: 1516, a CDRL1 comprising SEQ ID NO: 1522, a CDRL2 comprising SEQ ID NO: 1524, and a CDRL3 comprising SEQ ID NO: 1526;
(XV) (i) the CDRH1, the CDRH2, the CDRH3, the CDRL1, the CDRL2, and the CDR-L3 contained in Antibody No. V016; or
(ii) a CDRH1 comprising SEQ ID NO: 1612, a CDRH2 comprising SEQ ID NO: 1614, a CDRH3 comprising SEQ ID NO: 1616, a CDRL1 comprising SEQ ID NO: 1622, a CDRL2 comprising SEQ ID NO: 1624, and a CDRL3 comprising SEQ ID NO: 1626;
(XVI) (i) the CDRH1, the CDRH2, the CDRH3, the CDRL1, the CDRL2, and the CDR-L3 contained in Antibody No. V017; or
(ii) a CDR-H1 comprising SEQ ID NO: 1712, a CDRH2 comprising SEQ ID NO: 1714, a CDRH3 comprising SEQ ID NO: 1716, a CDRL1 comprising SEQ ID NO: 1722, a CDRL2 comprising SEQ ID NO: 1724, and a CDRL3 comprising SEQ ID NO: 1726;
(XVII) (i) the CDRH1, the CDRH2, the CDRH3, the CDRL1, the CDRL2, and the CDR-L3 contained in Antibody No. V018; or
(ii) a CDRH1 comprising SEQ ID NO: 1812, a CDRH2 comprising SEQ ID NO: 1814, a CDRH3 comprising SEQ ID NO: 1816, a CDRL1 comprising SEQ ID NO: 1822, a CDRL2 comprising SEQ ID NO: 1824, and a CDRL3 comprising SEQ ID NO: 1826; or
(XVIII) (i) the CDRH1, the CDRH2, the CDRH3, the CDRL1, the CDRL2, and the CDR-L3 contained in Antibody No. V019; or
(ii) a CDRH1 comprising SEQ ID NO: 1912, a CDRH2 comprising SEQ ID NO: 1914, a CDRH3 comprising SEQ ID NO: 1916, a CDRL1 comprising SEQ ID NO: 1922, a CDRL2 comprising SEQ ID NO: 1924, and a CDRL3 comprising SEQ ID NO: 1926.

6. The anti-CD3 antibody or antigen-binding fragment of any one of claims 2-5, wherein:
(A) the VH polypeptide comprises:
(a) a VH framework region 1 (FRH1) comprising the amino acid sequence of:
(i) the FRH1 contained in any one of Antibody Nos. V002-V019 and A001-A005;
(ii) the FRH1 contained in any one of SEQ ID NOS: 210, 310, 410, 510, 610, 710, 810, 910, 1010, 1110, 1210, 1310, 1410, 1510, 1610, 1710, 1810, 1910, 2110, 2210, 2310, 2410, and 2510; and/or
(iii) any one of SEQ ID NOS: 111, 211, 311, 411, 511, 611, 711, 811, 911, 1011, 1111, 1211, 1311, 1411, 1511, 1611, 1711, 1811, 1911, 2111, 2211, 2311, 2411, and 2511;
(b) a VH framework region 2 (FRH2) comprising the amino acid sequence of:
(i) the FRH2 contained in any one of Antibody Nos. V002-V019 and A001-A005;
(ii) the FRH2 contained in any one of SEQ ID NOS: 210, 310, 410, 510, 610, 710, 810, 910, 1010, 1110, 1210, 1310, 1410, 1510, 1610, 1710, 1810, 1910, 2110, 2210, 2310, 2410, and 2510;
(iii) WVRQAPGQRLEWX₁₈G (SEQ ID NO: 113), wherein X₁₈ is M or I; and/or
(iv) any one of SEQ ID NOS: 213, 313, 413, 513, 613, 713, 813, 913, 1013, 1113, 1213, 1313, 1413, 1513, 1613, 1713, 1813, 1913, 2113, 2213, 2313, 2413, and 2513;
(c) a VH framework region 3 (FRH3) comprising the amino acid sequence of:
(i) the FRH3 contained in any one of Antibody Nos. V002-V019 and A001-A005;
(ii) the FRH3 contained in any one of SEQ ID NOS: 210, 310, 410, 510, 610, 710, 810, 910, 1010, 1110, 1210, 1310, 1410, 1510, 1610, 1710, 1810, 1910, 2110, 2210, 2310, 2410, and 2510; and/or
(iii) any one of SEQ ID NOS: 115, 215, 315, 415, 515, 615, 715, 815, 915, 1015, 1115, 1215, 1315, 1415, 1515, 1615, 1715, 1815, 1915, 2115, 2215, 2315, 2415, and 2515; and/or
(d) a VH framework region 4 (FRH4) comprising the amino acid sequence of:
(i) the FRH4 contained in any one of Antibody Nos. V002-V019 and A001-A005;
(ii) the FRH4 contained in any one of SEQ ID NOS: 210, 310, 410, 510, 610, 710, 810, 910, 1010, 1110, 1210, 1310, 1410, 1510, 1610, 1710, 1810, 1910, 2110, 2210, 2310, 2410, and 2510; and/or
(iii) any one of SEQ ID NOS: 117, 217, 317, 417, 517, 617, 717, 817, 917, 1017, 1117, 1217, 1317, 1417, 1517, 1617, 1717, 1817, 1917, 2117, 2217, 2317, 2417, and 2517; and/or
(B) the VL polypeptide comprises:
(a) a VL framework region 1 (FRL1) comprising the amino acid sequence of:
(i) the FRL1 contained in any one of Antibody Nos. V002-V019 and A001-A005;
(ii) the FRL1 contained in any one of SEQ ID NOS: 220, 320, 420, 520, 620, 720, 820, 920, 1020, 1120, 1220, 1320, 1420, 1520, 1620, 1720, 1820, 1920, 2120, 2220, 2320, 2420, and 2520;
(iii) DIVMX₁₉QSPDSLAVSLGERATINC (SEQ ID NO: 121), wherein X₁₉ is T or S; and/or
(iv) any one of SEQ ID NOS: 221, 321, 421, 521, 621, 721, 821, 921, 1021, 1121, 1221, 1321, 1421, 1521, 1621, 1721, 1821, 1921, 2121, 2221, 2321, 2421, and 2521;
(b) a VL framework region 2 (FRL2) comprising the amino acid sequence of:
(i) the FRL2 contained in any one of Antibody Nos. V002-V019 and A001-A005;
(ii) the FRL2 contained in any one of SEQ ID NOS: 220, 320, 420, 520, 620, 720, 820, 920, 1020, 1120, 1220, 1320, 1420, 1520, 1620, 1720, 1820, 1920, 2120, 2220, 2320, 2420, and 2520; and/or
(iii) any one of SEQ ID NOS: 123, 223, 323, 423, 523, 623, 723, 823, 923, 1023, 1123, 1223, 1323, 1423, 1523, 1623, 1723, 1823, 1923, 2123, 2223, 2323, 2423, and 2523;
(c) a VL framework region 3 (FRL3) comprising the amino acid sequence of:
(i) the FRL3 contained in any one of Antibody Nos. V002-V019 and A001-A005;
(ii) the FRL3 contained in any one of SEQ ID NOS: 220, 320, 420, 520, 620, 720, 820, 920, 1020, 1120, 1220, 1320, 1420, 1520, 1620, 1720, 1820, 1920, 2120, 2220, 2320, 2420, and 2520; and/or
(iii) any one of SEQ ID NOS: 125, 225, 325, 425, 525, 625, 725, 825, 925, 1025, 1125, 1225, 1325, 1425, 1525, 1625, 1725, 1825, 1925, 2125, 2225, 2325, 2425, and 2525; and/or
(d) a VL framework region 4 (FRL4) comprising the amino acid sequence of:
(i) the FRL4 contained in any one of Antibody Nos. V002-V019 and A001-A005;
(ii) the FRL4 contained in any one of SEQ ID NOS: 220, 320, 420, 520, 620, 720, 820,920, 1020, 1120, 1220, 1320, 1420, 1520, 1620, 1720, 1820, 1920, 2120, 2220, 2320, 2420, and 2520; and/or
(iii) any one of SEQ ID NOS: 127, 227, 327, 427, 527, 627, 727, 827, 927, 1027, 1127, 1227, 1327, 1427, 1527, 1627, 1727, 1827, 1927, 2127, 2227, 2327, 2427, and 2527,
or the anti-CD3 antibody or antigen-binding fragment comprising a VH and/or a VL that comprises any combination of the foregoing VH and VL framework regions.

7. The anti-CD3 antibody or antigen-binding fragment of any one of claims 2-6, comprising:
(I) (i) the FRH1, the FRH2, the FRH3, the FLRH4, the FRL1, the FRL2, the FRL3, and the FRL4 contained in any one of Antibody Nos. A001, V002-V007, A003-A005, and V014-V019; and/or
(ii) a FR-H1 comprising any one of SEQ ID NOS: 111, 211, 311, 411, 511, 611, 711, 1411, 1511, 1611, 1711, 1811, 1911, 2111, 2311, 2411, and 2511, a FR-H2 comprising any one of SEQ ID NOS: 213, 313, 413, 513, 613, 713, 1413, 1513, 1613, 1713, 1813, 1913, 2113, 2313, 2413, and 2513, a FR-H3 comprising any one of SEQ ID NOS: 115, 215, 315, 415, 515, 615, 715, 1415, 1515, 1615, 1715, 1815, 1915, 2115, 2315, 2415, and 2515, a FR-H4 comprising any one of SEQ ID NOS: 117, 217, 317, 417, 517, 617, 717, 1417, 1517, 1617, 1717, 1817, 1917, 2117, 2317, 2417, and 2517, a FR-L1 comprising any one of SEQ ID NOS: 221, 321, 421, 521, 621, 721, 1421, 1521, 1621, 1721, 1821, 1921, 2121, 2321, 2421, and 2521, a FR-L2 comprising any one of SEQ ID NOS: 123, 223, 323, 423, 523, 623, 723, 1423, 1523, 1623, 1723, 1823, 1923, 2123, 2323, 2423, and 2523, a FR-L3 comprising any one of SEQ ID NOS: 125, 225, 325, 425, 525, 625, 725, 1425, 1525, 1625, 1725, 1825, 1925, 2125, 2325, 2425, and 2525, and a FR-L4 comprising any one of SEQ ID NOS: 127, 227, 327, 427, 527, 627, 727, 1427, 1527, 1627, 1727, 1827, 1927, 2127, 2327, 2427, and 2527; or
(II) (i) the FRH1, the FRH2, the FRH3, the FLRH4, the FRL1, the FRL2, the FRL3, and the FRL4 contained in any one of Antibody Nos. A002 and V008-V013; and/or
(ii) a FR-H1 comprising any one of SEQ ID NOS: 111, 811, 911, 1011, 1111, 1211, 1311, and 2211, a FR-H2 comprising any one of SEQ ID NOS: 813, 913, 1013, 1113, 1213, 1313, and 2213, a FR-H3 comprising any one of SEQ ID NOS: 115, 815, 915, 1015, 1115, 1215, 1315, and 2215, a FR-H4 comprising anyone of SEQ ID NOS: 117, 817, 917, 1017, 1117, 1217, 1317, and 2217, a FR-L1 comprising any one of SEQ ID NOS: 821, 921, 1021, 1121, 1221, 1321, and 2221, a FR-L2 comprising any one of SEQ ID NOS: 123, 823, 923, 1023, 1123, 1223, 1323, and 2223, a FR-L3 comprising any one of SEQ ID NOS: 125, 825, 925, 1025, 1125, 1225, 1325, and 2225, and a FR-L4 comprising any one of SEQ ID NOS: 127, 827, 927, 1027, 1127, 1227, 1327, and 2227.

8. The anti-CD3 antibody or antigen-binding fragment of any one of claims 2-7, comprising:
(I) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V002; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 211, 212, 213, 214, 215, 216, 217, 221, 222, 223, 224, 225, 226, and 227, respectively;
(II) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V003; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 311, 312, 313, 314, 315, 316, 317, 321, 322, 323, 324, 325, 326, and 327, respectively;
(III) (i) the FRH1, the CDRH1, the FRH2, the CDRH2, the FR13, the CDRH3, the FRH4, the FRL1, the CDRL1, the FRL2, the CDRL2, the FRL3, the CDRL3, and the FRL4 contained in Antibody No. V004; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 411, 412, 413, 414, 415, 416, 417, 421, 422, 423, 424, 425, 426, and 427, respectively;
(IV) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V005; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 511, 512, 513, 514, 515, 516, 517, 521, 522, 523, 524, 525, 526, and 527, respectively;
(V) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V006; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 611, 612, 613, 614, 615, 616, 617, 621, 622, 623, 624, 625, 626, and 627, respectively;
(VI) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V007; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 711, 712, 713, 714, 715, 716, 717, 721, 722, 723, 724, 725, 726, and 727, respectively;
(VII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V008; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 811, 812, 813, 814, 815, 816, 817, 821, 822, 823, 824, 825, 826, and 827, respectively;
(VIII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V009; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 911, 912, 913, 914, 915, 916, 917, 921, 922, 923, 924, 925, 926, and 927, respectively;
(IX) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V010; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1011, 1012, 1013, 1014, 1015, 1016, 1017, 1021, 1022, 1023, 1024, 1025, 1026, and 1027, respectively;
(X) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V011; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1111, 1112, 1113, 1114, 1115, 1116, 1117, 1121, 1122, 1123, 1124, 1125, 1126, and 1127, respectively;
(XI) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V012; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1211, 1212, 1213, 1214, 1215, 1216, 1217, 1221, 1222, 1223, 1224, 1225, 1226, and 1127, respectively;
(XII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V013; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1311, 1312, 1313, 1314, 1315, 1316, 1317, 1321, 1322, 1323, 1324, 1325, 1326, and 1327, respectively;
(XIII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V014; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1411, 1412, 1413, 1414, 1415, 1416, 1417, 1421, 1422, 1423, 1424, 1425, 1426, and 1427, respectively;
(XIV) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V015; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1511, 1512, 1513, 1514, 1515, 1516, 1517, 1521, 1522, 1523, 1524, 1525, 1526, and 1527, respectively;
(XV) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V016; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1611, 1612, 1613, 1614, 1615, 1616, 1617, 1621, 1622, 1623, 1624, 1625, 1626, and 1627, respectively;
(XVI) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V017; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1711, 1712, 1713, 1714, 1715, 1716, 1717, 1721, 1722, 1723, 1724, 1725, 1726, and 1727, respectively;
(XVII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V018; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1811, 1812, 1813, 1814, 1815, 1816, 1817, 1821, 1822, 1823, 1824, 1825, 1826, and 1827, respectively; or
(XVIII) (i) the FRH1, the CDRH1, the FRH2, the CDRH2, the FRH3, the CDRH3, the FRH4, the FRL1, the CDRL1, the FRL2, the CDRL2, the FRL3, the CDRL3, and the FRL4 contained in Antibody No. V019; or
(ii) a FRH1, a CDRH1, a FRH2, a CDRH2, a FRH3, a CDRH3, a FRH4, a FRL1, a CDRL1, a FRL2, a CDRL2, a FRL3, a CDRL3, and a FRL4 comprising SEQ ID NOS: 1911, 1912, 1913, 1914, 1915, 1916, 1917, 1921, 1922, 1923, 1924, 1925, 1926, and 1927, respectively.

9. The anti-CD3 antibody or antigen-binding fragment of claim 5, wherein:
in (I), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 210; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 220;
in (II), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 310; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 320;
in (III), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 410; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 420;
in (IV), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 510; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 520;
in (V), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 610; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 620;
in (VI), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 710; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 720;
in (VII), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 810; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 820;
in (VIII), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 910; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 920;
in (IX), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1010; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1020;
in (X), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1110; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1120;
in (XI), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1210; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1220;
in (XII), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1310; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1320;
in (XIII), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1410; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1420;
in (XIV), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1510; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1520;
in (XV), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1610; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1620;
in (XVI), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1710; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1720;
in (XVII), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1810; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1820; or
in (XVIII), (A) the VH polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1910; and/or
(B) the VL polypeptide comprises an amino acid sequence at least 80%, at least 85%, at least 90%, at least 92%, at least 94%, at least 95%, at least 96%, at least 97%, at least 98%, at least 99%, or 100% identical to SEQ ID NO: 1920.

10. The anti-CD3 antibody or antigen-binding fragment of any one of claims 2-9, wherein the VH polypeptide and the VL polypeptide comprise the amino acid sequences of:
(I) SEQ ID NOS: 210 and 220, respectively;
(II) SEQ ID NOS: 310 and 320, respectively;
(III) SEQ ID NOS: 410 and 420, respectively;
(IV) SEQ ID NOS: 510 and 520, respectively;
(V) SEQ ID NOS: 610 and 620, respectively;
(VI) SEQ ID NOS: 710 and 720, respectively;
(VII) SEQ ID NOS: 810 and 820, respectively;
(VIII) SEQ ID NOS: 910 and 920, respectively;
(IX) SEQ ID NOS: 1010 and 1020, respectively;
(X) SEQ ID NOS: 1110 and 1120, respectively;
(XI) SEQ ID NOS: 1210 and 1220, respectively;
(XII) SEQ ID NOS: 1310 and 1320, respectively;
(XIII) SEQ ID NOS: 1410 and 1420, respectively;
(XIV) SEQ ID NOS: 1510 and 1520, respectively;
(XV) SEQ ID NOS: 1610 and 1620, respectively;
(XVI) SEQ ID NOS: 1710 and 1720, respectively;
(XVII) SEQ ID NOS: 1810 and 1820, respectively; or
(XVIII) SEQ ID NOS: 1910 and 1920, respectively.

11. The anti-CD3 antibody or antigen-binding fragment of any one of claims 2-10, which comprises any one or more of the following:
(i) one or more antibody constant regions, a CH1 domain, a hinge, a CH2 domain, and/or a CH3 domain, which optionally is/are individually of, or derived from an IgG or human IgG, further optionally of or derived from a human IgG1, IgG4, IgG2, or IgG3;
(ii) a fragment crystallizable (Fc) region, optionally of, or derived from:
(1) a human IgG1, further optionally comprising one or more of the following amino acid modifications: N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V305I, P396L, S239D, 1332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y M428L, N434S, L328F, M252Y, S254T, T256E, or any combination thereof, according to EU numbering;
(2) a human IgG4, further optionally comprising one or more of the following amino acid modifications: E233P, F234V, L235A, G237A, E318A, S228P, L236E, S241P, L248E, T394D, M252Y, S254T, T256E, N297A, N297Q, or any combination thereof, according to EU numbering;
(3) a human IgG2, further optionally comprising one or more of the following amino acid modifications: P238S, V234A, G237A, H268A, H268Q, H268E, V309L, N297A, N297Q, A330S, P331S, C232S, C233S, M252Y, S254T, T256E, or any combination thereof, according to EU numbering; and/or
(4) a human IgG3, further optionally comprising E235Y, according to EU numbering;
(iii) an IgG, IgA, IgE, IgD, or IgM, optionally IgG1, IgG4, IgG2, or IgG3; and/or
(iv) an antibody fragment selected from the group consisting of: a fragment, antigen-binding (Fab) region; an Fab₂; an Fab₃; an Fab′ fragment; an F(ab′)₂; a variable fragment (Fv); a single-chain Fv (scFv) fragment; a diabody; a triabody; a minibody; a scFv-Fe; a scFv2-Fc2; scFv-IgG; a monovalent IgG (or a half IgG); and/or a chimeric antigen receptor (CAR) comprising an antigen-binding region comprising said VH polypeptide and/or said VL polypeptide, a transmembrane domain, and at least one intracellular signaling domain (optionally derived from a T-cell receptor, further optionally CD3ζ).

12. The anti-CD3 antibody or antigen-binding fragment of any one of claims 2-11, which binds to:
(i) human CD3:
(ii) non-human primate CD3, optionally monkey, further optionally cynomolgus and/or rhesus CD3; and/or
(iii) rodent CD3, optionally mouse CD3,
optionally wherein the CD3 is CD3εδ.

13. The anti-CD3 antibody or antigen-binding fragment of any one of claims 2-12, which comprises or is comprised in a multispecific antibody or antibody fragment having at least (a) a first antigen-binding region specific to CD3, comprising said VH polypeptide and/or said VL polypeptide, and (b) a second antigen-binding region, optionally comprising one or more of the following features:
(i) the second antigen-binding region is specific to: an oncology target; a target molecule expressed on cancer cells; an immune-oncology target; a target molecule expressed on immune cells; a neurodegenerative disease targets; an autoimmune disorder target (optionally a self-reactive immune molecule or a target molecule expressed on an immune cell expressing a self-reactive immune molecule); an inflammatory disease target (optionally an inflammatory cytokine or chemokine or a receptor thereof); an infectious disease target (optionally a target molecule of a virus, bacterium, or a fungus); a target molecule expressed on infected cells (optionally infected with a virus, a bacterium, or fungus); a metabolic disease target; a cognitive disorder target; a blood-brain barrier target; or a blood disease target;
(ii) the second antigen-binding region is specific to one or more of: 17-IA, 4-1BB, 4Dc, 6-keto-PGF1a, 8-iso-PGF2a, 8-oxo-dG, Al Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM 15, ADAM 17/T ACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BFM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL 14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-1 (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxin1, EpCAM, Ephrin B2/EphB4, EPO, ERCC, E-selectin, ET-1, Factor II a, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-IR, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin, alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein LI, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, a metalloprotease, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibiting substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL RI Apo-2, DR4), TNFRSFIOB (TRAIL R2 DR5, KILLER, TRICK-2A, TRICK-B), TNFRSF10C (TRAIL R3 DcRI, LIT, TRID), TNFRSF10D (TRAIL R4 DcR2, TRUNDD), TNFRSF11A (RANK ODF R, TRANCE R), TNFRSFIlB (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF 18 (GITR AITR), TNFRSF19 (TROY TAJ, TRADE), TNFRSF19L (RELT), TNFRSFIA (TNF RI CD120a, p55-60), TNFRSFIB (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF 5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DCTRAIL R2 TNFRH2), TNFRST23 (DcTRAIL RI TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TLIA/VEGI), TNFSF18 (GITR Ligand AITR Ligand, TL6), TNFSFIA (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VFM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3), a hormone receptor, and a growth factor;
(iii) the second antigen-binding region is specific to one or more of: BCMA, CTLA4 (cytotoxic T lymphocyte antigen-4), PD1 (programmed cell death protein 1), PD-L1 (programmed cell death ligand 1), LAG-3 (lymphocyte activation gene-3), TIM-3, CD20, CD2, CD19, Her2, EGFR, EpCAM, FcyRIIIa (CD16), FcyRIIa (CD32a), FcyRIIb (CD32b), FcyRI (CD64), Toll-like receptors (TLRs), TLR4, TLR9, cytokines, IL-2, IL-5, IL-13, IL-6, IL-17, IL-12, IL-23, TNFα, TGFβ, cytokine receptors, IL-2R, chemokines, chemokine receptors, growth factors, VEGF, and HGF;
(iv) the multispecific antibody or antibody fragment is bispecific;
(v) the multispecific antibody or antibody fragment further comprises a third antigen-binding region;
(vi) the multispecific antibody or antibody fragment is trispecific;
(vii) the multispecific antibody or antibody fragment comprises a multispecific format selected from the group consisting of: Fab-Fc-scFv, scFv2-Fc2, scFv-IgG, “bottle-opener”, Mab-scFv, Mab-Fv, Dual scFv, central Fv, central scFv, one-arm central scFv, Fab-Fab, Fab-Fv, mAb-Fv, mAb-Fab, DART, BiTE, common light chain-IgG, TandAb, Cross-Mab, SEED, BEAT, TrioMab, and DuetMab; and/or
(viii) the multispecific antibody or antibody fragment comprises one or more of the following: at least one CLκ-preferring variant CH1 domain, optionally a CLκ-preferring variant CH1 domain described in WO2021067404; at least one CLλ-preferring variant CH1 domain, optionally a CLλ-preferring variant CH1 domain described in WO2021067404; at least one pair of a variant CH1 domain and a variant CL domain which preferentially pair with each other, optionally a pair described in WO2022150787; and/or at least one pair of a variant CH3 domain and another variant CH3 domain which preferentially pair with each other, optionally a pair described in WO2022150785.

14. The anti-CD3 antibody or antigen-binding fragment of any one of claims 1-13, which comprises one or more of the following features:
(I) exhibits:
(i) a reduced PSR score relative to any one or more of or all of Antibody Nos. A001-A005; and/or
(ii) a PSR score lower than 0.33, lower than about 0.30, lower than about 0.20, lower than about 0.15, lower than about 0.12, lower than about 0.10, lower than about 0.08, lower than about 0.06, lower than about 0.05, lower than about 0.04, lower than about 0.03, lower than about 0.02, or lower than about 0.01; and/or
(iii) a PSR score of about ≥0.10 and <0.33 or a PSR score of about <0.10;
(II) exhibits a hydrophobic interaction chromatography (HIC) retention time of < about 10.5 minutes, < about 10.0 minutes, < about 9.5 minutes, < about 9.0 minutes, or < about 8.5 minutes;
(III) exhibits:
(i) a reduced affinity-capture self-interaction nanoparticle spectroscopy (AC-SINS) Δλmax relative to any one or more of or all of Antibody Nos. A001-A005;
(ii) an AC-SINS Δλmax lower than about 20.0 nm, lower than about 15.0 nm, lower than about 12.0 nm, lower than about 10.0 nm, lower than about 8.0 nm, lower than about 6.0 nm, lower than about 5.0 nm, lower than about 4.0 un, lower than about 3.0 nm, lower than about 2.0 nm, or lower than about 1.0 nm; and/or
(iii) an AC-SINS Δλmax of about ≥5.0 nm and ≤20.0 or an AC-SINS Δλmax of about <5.0 nm;
(IV) exhibits:
(i) a higher dynamic light scattering (DLS) diffusion interaction parameter (kD) relative to any one or more of or all of Antibody Nos. A001-A005;
(ii) a DLS kD of about 5 mL/g or higher, about 10 mL/g or higher, about 15 mL/g or higher, about 20 mL/g or higher, about 25 mL/g or higher, about 30 mL/g or higher, or about 35 mL/g or higher; and/or
(iii) a DLS kD of between about 10 mL/g and about 40 mL/g, between about 15 mL/g and about 40 mL/g, or between about 20 mL/g and about 40 mL/g,
optionally wherein the DLS kD is measured using a 10 mM histidine buffer, optionally of pH 6.0;
(V) comprises:
(i) a melting temperature (Tm) of about 65° C. or higher; or
(ii) a Tm of about 70° C. or higher, about 75° C. or higher, about 80° C. or higher; and/or
(iii) a Tm of between about 70° C. and about 90° C., between about 75° C. and about 85° C.,
optionally wherein the anti-CD3 antibody or antigen-binding fragment is a Fab;
(VI) does not significantly aggregate or multimerize, optionally as determined by:
(i) % monomer of about 90% or higher, about 95% or higher, about 97% or higher, about 98% or higher, about 99% or higher, by size exclusion chromatography (SEC); and/or
(ii) % monomer between about 90% and about 100%, between about 95% and about 100%, between about 97% and about 100%, between about 98% and about 100%, between about 99% and about 100%, by SEC,
optionally wherein the anti-CD3 antibody or antigen-binding fragment is a Fab or an IgG, optionally IgG1,
and optionally wherein the anti-CD3 antibody or antigen-binding fragment does not significantly aggregate or multimerize in an acidic condition, optionally in a pH of about 6 or lower, about 5 or lower, about 4 or lower, or about 3.5 or lower;
(VII) does not exhibit significant amount of heavy-light chain mispairing, optionally as determined by absence of a peak of unpaired heavy chain and/or a peak of unpaired light chain measured by liquid chromatography-mass spectrometry (LC-MS), optionally when the anti-CD3 antibody or antigen-binding fragment is produced recombinantly, further optionally in mammalian cells, yet further optionally Chinese hamster ovary (CHO) cells;
(VIII) binds to CD3-expressing cells, optionally wherein the CD3-expressing cells are:
(i) T cells;
(ii) human cells, non-human primate (optionally monkey, further optionally cynomolgus and/or rhesus) cells, and/or rodent (optionally mouse) cells; and/or
(iii) primary cells or cell line cells:
(IX) binds to CD3 with an equilibrium dissociation constant (Kd) of:
(i) about 1.0×10⁶ M or lower, about 5.0×10⁷ M or lower, about 1.0×10⁷ M or lower, about 5.0×10⁸ M or lower, about 1.0×10⁸ M or lower, about 5.0×10⁹ M or lower, about 1.0×10⁹ M or lower, about 5.0×10¹⁰ M or lower, or about 1.0×10¹⁰ M or lower;
(ii) between about 1.0×10⁶ M and about 1.0×10¹¹ M, between about 1.0×10⁷ M and about 1.0×10¹¹ M, or between about 1.0×10⁸ M and about 1.0×10¹⁰ M.
optionally as measured by surface plasmon resonance (SPR), further optionally using a BIACORE® system, or optionally as measured by bio-layer interferometry (BLI), further optionally using an Octet® system,
and optionally wherein the CD3 is human, non-human primate (optionally monkey, further optionally cynomolgus and/or rhesus), and/or rodent (optionally mouse) CD3;
(X) upon binding to CD3 on a cell, elicits activation of and/or enhances cytotoxic function(s) of the cell, optionally a T cell;
(X) upon binding to CD3 on a cell, does not elicit cytokine production by the cell to levels capable of inducing cytokine release syndrome (CRS); and/or
(XI) comprises or is comprised in a multispecific antibody or antibody fragment having at least (a) a first antigen-binding region specific to CD3, comprising said VH polypeptide and/or said VL polypeptide, and (b) a second antigen-binding region specific to a second antigen; and upon binding to (i) CD3 on a first cell, optionally a T cell, and (ii) the second antigen expressed on a second cell, the first cell exhibits cytotoxicity to the second cell.

15. A nucleic acid encoding the anti-CD3 antibody or antigen-binding fragment according to any one of claims 1-14, optionally comprising:
(A) a VH polypeptide-encoding nucleic acid sequence at least 80%, at least 85%, at least 90%, at least 92%, at least 94%, at least 95%, at least 96%, at least 97%, at least 98%, at least 99%, or 100% identical to SEQ ID NO: 250, 350, 450, 550, 650, 750, 850, 950, 1050, 1150, 1250, 1350, 1450, 1550, 1650, 1750, 1850, or 1950; and/or
(B) a VL polypeptide-encoding nucleic acid sequence at least 80%, at least 85%, at least 90%, at least 92%, at least 94%, at least 95%, at least 96%, at least 97%, at least 98%, at least 99%, or 100% identical to SEQ ID NO: 260, 360, 460, 560, 660, 760, 860, 960, 1060, 1160, 12560, 1360, 1460, 1560, 1660, 1760, 1860, or 1960,
further optionally comprising VH polypeptide-encoding and VL polypeptide-encoding nucleic acid sequences of:
(I) SEQ ID NOS: 250 and 260, respectively;
(II) SEQ ID NOS: 350 and 360, respectively;
(III) SEQ ID NOS: 450 and 460, respectively;
(IV) SEQ ID NOS: 550 and 560, respectively;
(V) SEQ ID NOS: 650 and 660, respectively;
(VI) SEQ ID NOS: 750 and 760, respectively;
(VII) SEQ ID NOS: 850 and 860, respectively;
(VIII) SEQ ID NOS: 950 and 960, respectively;
(IX) SEQ ID NOS: 1050 and 1060, respectively;
(X) SEQ ID NOS: 1150 and 1160, respectively;
(XI) SEQ ID NOS: 1250 and 1260, respectively;
(XII) SEQ ID NOS: 1350 and 1360, respectively;
(XIII) SEQ ID NOS: 1450 and 1460, respectively;
(XIV) SEQ ID NOS: 1550 and 1560, respectively;
(XV) SEQ ID NOS: 1650 and 1660, respectively;
(XVI) SEQ ID NOS: 1750 and 1760, respectively;
(XVII) SEQ ID NOS: 1850 and 1860, respectively; or
(XVIII) SEQ ID NOS: 1950 and 1960, respectively.

16. A vector comprising the nucleic acid of claim 15, optionally wherein:
(i) the vector is an expression vector; and/or
(ii) the vector comprises a plasmid, a viral vector (optionally adenoviral, lentiviral, or retroviral), a lipid-based vector, a self-replicating RNA vector, a virus-like particle, a polymer-based vector, and/or a nanoparticle, optionally a lipid-based nanoparticle.

17. An isolated or recombinant cell comprising, transfected with, transformed with, or transduced with the nucleic acid of claim 15 and/or the vector of claim 16, optionally wherein the isolated or recombinant cell is:
(i) mammalian, optionally human, non-human primate, monkey, rabbit, rodent, hamster, rat, or mouse; or
(ii) non-mammalian, optionally plant, bacterial, fungal, yeast, protozoa, or insect, and optionally wherein the isolated or recombinant cell is an immune cell or a hybridoma.

18. A pharmaceutical composition comprising:
(A) the anti-CD3 antibody or antigen-binding fragment according to any one of claims 1-14, the nucleic acid of claim 15, the vector of claim 16, and/or the isolated or recombinant cell of claim 17; and
(B) a pharmaceutically acceptable carrier and/or excipient.

19. A method of treating a subject in need of such treatment, comprising administering to the subject an effective amount of:
(i) the anti-CD3 antibody or antigen-binding fragment of any one of claims 1-14;
(ii) the nucleic acid of claim 15;
(iii) the vector of claim 16;
(iv) the isolated or recombinant cell of claim 17; and/or
(v) the pharmaceutical composition of claim 18,
optionally wherein:
(a) the subject is
(i) a mammal, optionally a human, a non-human primate, a monkey, a horse, a cow, sheep, a goat, a pig, a dog, a cat, a rabbit, a rodent, a hamster, a rat, or a mouse; or
(ii) a non-mammalian vertebrate, optionally a bird, fish, an amphibian, or a reptile;
(b) the subject comprises or has a risk of developing a disease, disorder, or a condition; and/or
(c) the method further comprises administering to the subject an additional agent, optionally an adjuvant or a therapeutic agent.

20. A method of treating or preventing a disease, disorder, or a condition in a subject in need of such treatment, the method comprising administering an effective amount of:
(i) the anti-CD3 antibody or antigen-binding fragment of any one of claims 1-14;
(ii) the nucleic acid of claim 15;
(iii) the vector of claim 16;
(iv) the isolated or recombinant cell of claim 17; optionally, an immune cell, T cell, and/or a natural killer (NK) cell; and/or
(v) the pharmaceutical composition of claim 18,
optionally wherein:
(a) the subject is:
(i) a mammal, optionally a human, a non-human primate, a monkey, a horse, a cow, sheep, a goat, a pig, a dog, a cat, a rabbit, a rodent, a hamster, a rat, or a mouse; or
(ii) a non-mammalian vertebrate, optionally a bird, fish, an amphibian, or a reptile; and/or
(b) the method further comprises administering to the subject an additional agent, optionally an adjuvant or a therapeutic agent.

21. A method of eliciting cytotoxicity to a cell expressing a target molecule of interest, comprising administering to the subject an effective amount of:
(i) the anti-CD3 antibody or antigen-binding fragment of claim 13;
(ii) a nucleic acid encoding the anti-CD3 antibody or antigen-binding fragment of claim 13;
(iii) a vector comprising the nucleic acid;
(iv) an isolated or recombinant cell comprising, transfected with, transformed with, or transduced with said nucleic acid or vector; and/or
(v) a pharmaceutical composition comprising (A) the anti-CD3 antibody or antigen-binding fragment of claim 13, the nucleic acid encoding the anti-CD3 antibody or antigen-binding fragment of claim 13, a vector comprising said nucleic acid, and/or an isolated or recombinant cell comprising, transfected with, transformed with, or transduced with said nucleic acid or vector; and/or (B) a pharmaceutically acceptable carrier and/or excipient,
optionally wherein:
(a) the subject is
(i) a mammal, optionally a human, a non-human primate, a monkey, a horse, a cow, sheep, a goat, a pig, a dog, a cat, a rabbit, a rodent, a hamster, a rat, or a mouse; or
(ii) a non-mammalian vertebrate, optionally a bird, fish, an amphibian, or a reptile;
(b) the subject comprises or has a risk of developing a disease, disorder, or a condition; and/or
(c) the method further comprises administering to the subject an additional agent, optionally an adjuvant or a therapeutic agent.

22. The method of any one of claims 19-21, wherein the disease, disorder, or condition comprises one or more of a proliferative disorder, an oncological disorder, cancer or a neoplastic condition, an immuno-oncological disorder, a neurological disorder, a neurodegenerative disorder, an infectious disease, an inflammatory disease, an autoimmune disorder, or another disease, optionally wherein:
(i) the cancer is:
(i-1) a solid cancer, optionally chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof; and/or
(i-2) a liquid cancer, optionally chosen from: chronic lymphocytic leukemia (CLL), mantle cell lymphoma (MCL), multiple myeloma, acute lymphoid leukemia (ALL), Hodgkin's lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin's lymphoma, plasmablastic lymphoma, plasmacytoid dendritic cell neoplasm, Waldenström's macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma;
(ii) the autoimmune or inflammatory disease is psoriasis, rheumatoid arthritis, autoimmune arthritis, type I diabetes, systemic lupus erythematosus, myasthenia gravis, multiple sclerosis, scleroderma, inflammatory bowel disease, Crohn's disease, ulcerative colitis, Guillain-Barre syndrome, chronic inflammatory demyelinating polyneuropathy, pemphigus vulgaris, Sjogren syndrome, Addison disease, Behçet's disease, Schmidt syndrome, celiac disease, dermatomyositis, autoimmune vitiligo, Graves' disease, Hashimoto thyroiditis, Kawasaki disease, pernicious anemia, autoimmune vasculitis, or fibrosis;
(iii) the neurodegenerative disease is Alzheimer's disease, Huntington's disease, Parkinson's disease, amyotrophic lateral sclerosis, Friedreich ataxia, Lewy body disease, spinal muscular atrophy, motor neuron disease, multiple sclerosis, Batten disease, Creutzfeldt-Jakob disease;
(iv) the infectious disease is a viral, bacterial, fungal, yeast, protozoan, prion or parasitic disease, optionally wherein (1) the viral disease is human immunodeficiency virus (HIV), hepatitis virus (optionally hepatitis A, B, or C virus), human papillomavirus (HPV), herpes simplex virus (HSV) (optionally HSV-1 or HSV-2), enterovirus, human cytomegalovirus, adenovirus, rhinovirus, Pox virus, Influenza virus, coronavirus (optionally MERS-CoV, SARS-CoV, or SARS-CoV-2, or common human coronavirus), norovirus, West Nile Virus, Zika virus, poliovirus, Ebola virus, or dengue virus (DENV) infection, (2) the bacterial disease is Salmonella, Escherichia coli, Mycobacterium tuberculosis, methicillin-resistant Staphylococcus aureus (MRSA), Clostridium difficile, Streptococcus pneumoniae, Klebsiella pneumoniae, Pseudomonas aeruginosa, Helicobacter pylori, Neisseria gonorrhoeae, Vibrio vulnificus, and/or (3) the fungal disease is Aspergillosis, Candida, Candida auris, Cryptococcus neoformans, Pneumocystis jiroveccii, Mucoromycetes, Taloromyces, ringworm, Blastomyces, Coccidioides, Cryptococcus gattii, Histoplasma, Paracoccidioides, or Sporothrix infection.

23. A method of manufacturing the anti-CD3 antibody or antigen-binding fragment of any one of claims 1-14, comprising:
(a) culturing cells comprising the nucleic acid of claim 16 in a condition that allows for expression of said antibody or antigen-binding fragment, and
(b) harvesting and purifying the antibody or antigen-binding fragment from the cell culture from (a).

24. A method of manufacturing the isolated or recombinant cell of claim 17 or a population of such cells, comprising introducing the nucleic acid of claim 15 and/or the vector of claim 16 into one or more cells, optionally wherein the introducing occurs in vitro, ex vivo, or in vivo.

25. The anti-CD3 antibody or antigen-binding fragment of any one of claims 1-14, the nucleic acid of claim 15, the vector of claim 16, the isolated or recombinant cell of claim 17 or a population of such cells, and/or the pharmaceutical composition of claim 18, for use in medicine.

26. The anti-CD3 antibody or antigen-binding fragment of any one of claims 1-14, the nucleic acid of claim 16, the vector of claim 16, the isolated or recombinant cell of claim 17 or a population of such cells, and/or the pharmaceutical composition of claim 18, for use in treating a disease, disorder, or condition, optionally wherein the disease, disorder, or condition comprises cancer or a neoplastic condition, an autoimmune disease, a neurodegenerative disease, an infectious disease, an inflammatory disease, or another disease, further optionally wherein:
(i) the cancer is:
(i-1) a solid cancer, optionally chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof; and/or
(i-2) a liquid cancer, optionally chosen from: chronic lymphocytic leukemia (CLL), mantle cell lymphoma (MCL), multiple myeloma, acute lymphoid leukemia (ALL), Hodgkin's lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, mycloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin's lymphoma, plasmablastic lymphoma, plasmacytoid dendritic cell neoplasm, Waldenström's macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma;
(ii) the autoimmune or inflammatory disease is psoriasis, rheumatoid arthritis, autoimmune arthritis, type I diabetes, systemic lupus erythematosus, myasthenia gravis, multiple sclerosis, scleroderma, inflammatory bowel disease, Crohn's disease, ulcerative colitis, Guillain-Barre syndrome, chronic inflammatory demyelinating polyneuropathy, pemphigus vulgaris, Sjogren syndrome, Addison disease, Behçet's disease, Schmidt syndrome, celiac disease, dermatomyositis, autoimmune vitiligo, Graves' disease, Hashimoto thyroiditis, Kawasaki disease, pernicious anemia, autoimmune vasculitis, or fibrosis;
(iii) the neurodegenerative disease is Alzheimer's disease, Huntington's disease, Parkinson's disease, amyotrophic lateral sclerosis, Friedreich ataxia, Lewy body disease, spinal muscular atrophy, motor neuron disease, multiple sclerosis, Batten disease, Creutzfeldt-Jakob disease;
(iv) the infectious disease is a viral, bacterial, fungal, yeast, protozoan, prion or parasitic disease, optionally wherein (1) the viral disease is human immunodeficiency virus (HIV), hepatitis virus (optionally hepatitis A, B, or C virus), human papillomavirus (HPV), herpes simplex virus (HSV) (optionally HSV-1 or HSV-2), enterovirus, human cytomegalovirus, adenovirus, rhinovirus, Pox virus, Influenza virus, coronavirus (optionally MERS-CoV, SARS-CoV, or SARS-CoV-2, or common human coronavirus), norovirus, West Nile Virus, Zika virus, poliovirus, Ebola virus, or dengue virus (DENV) infection, (2) the bacterial disease is Salmonella, Escherichia coli, Mycobacterium tuberculosis, methicillin-resistant Staphylococcus aureus (MRSA), Clostridium difficile, Streptococcus pneumoniae, Klebsiella pneumoniae, Pseudomonas aeruginosa, Helicobacter® pylori, Neisseria gonorrhoeae, Vibrio vulnificus, and/or (3) the fungal disease is Aspergillosis, Candida, Candida auris, Cryptococcus neoformans, Pneumocystis jiroveccii, Mucoromycetes, Taloromyces, ringworm, Blastomyces, Coccidioides, Cryptococcus gattii, Histoplasma, Paracoccidioides, or Sporothrix infection.

27. Use of the anti-CD3 antibody or antigen-binding fragment of any one of claims 1-14, the nucleic acid of claim 16, the vector of claim 16, the isolated or recombinant cell of claim 17 or a population of such cells, and/or the pharmaceutical composition of claim 18 for the manufacture of a medicament for treatment of a disease, disorder, or condition, optionally wherein the disease, disorder, or condition comprises cancer or a neoplastic condition, an autoimmune disease, a neurodegenerative disease, an infectious disease, an inflammatory disease, or another disease, further optionally wherein:
(i) the cancer is:
(i-1) a solid cancer, optionally chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof; and/or
(i-2) a liquid cancer, optionally chosen from: chronic lymphocytic leukemia (CLL), mantle cell lymphoma (MCL), multiple myeloma, acute lymphoid leukemia (ALL), Hodgkin's lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin's lymphoma, plasmablastic lymphoma, plasmacytoid dendritic cell neoplasm, Waldenström's macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma;
(ii) the autoimmune or inflammatory disease is psoriasis, rheumatoid arthritis, autoimmune arthritis, type I diabetes, systemic lupus erythematosus, myasthenia gravis, multiple sclerosis, scleroderma, inflammatory bowel disease, Crohn's disease, ulcerative colitis, Guillain-Barre syndrome, chronic inflammatory demyelinating polyneuropathy, pemphigus vulgaris, Sjogren syndrome, Addison disease, Behçet's disease, Schmidt syndrome, celiac disease, dermatomyositis, autoimmune vitiligo, Graves' disease, Hashimoto thyroiditis, Kawasaki disease, pernicious anemia, autoimmune vasculitis, or fibrosis;
(iii) the neurodegenerative disease is Alzheimer's disease, Huntington's disease, Parkinson's disease, amyotrophic lateral sclerosis, Friedreich ataxia, Lewy body disease, spinal muscular atrophy, motor neuron disease, multiple sclerosis, Batten disease, Creutzfeldt-Jakob disease;
(iv) the infectious disease is a viral, bacterial, fungal, yeast, protozoan, prion or parasitic disease, optionally wherein (1) the viral disease is human immunodeficiency virus (HIV), hepatitis virus (optionally hepatitis A, B, or C virus), human papillomavirus (HPV), herpes simplex virus (HSV) (optionally HSV-1 or HSV-2), enterovirus, human cytomegalovirus, adenovirus, rhinovirus, Pox virus, Influenza virus, coronavirus (optionally MERS-CoV, SARS-CoV, or SARS-CoV-2, or common human coronavirus), norovirus, West Nile Virus, Zika virus, poliovirus, Ebola virus, or dengue virus (DENV) infection, (2) the bacterial disease is Salmonella, Escherichia coli, Mycobacterium tuberculosis, methicillin-resistant Staphylococcus aureus (MRSA), Clostridium difficile, Streptococcus pneumoniae, Klebsiella pneumoniae, Pseudomonas aeruginosa, Helicobacter pylori, Neisseria gonorrhoeae, Vibrio vulnificus, and/or (3) the fungal disease is Aspergillosis, Candida, Candida auris, Cryptococcus neoformans, Pneumocystis jiroveccii, Mucoromycetes, Taloromyces, ringworm, Blastomyces, Coccidioides, Cryptococcus gattii, Histoplasma, Paracoccidioides, or Sporothrix infection.
